(19) United States
(12) Patent Application Publication
(10) Pub. No.: US 2021/0363189 A1
(43) Pub. Date: Nov. 25, 2021

(54) COMBINATION THERAPY OF PEPTIDOMIMETIC MACROCYCLES

(71) Applicant: Aileron Therapeutics, Inc., Watertown, MA (US)

(72) Inventors: Vincent GUERLAVAIS, Arlington, MA (US); David Allen ANNIS, Cambridge, MA (US)

(21) Appl. No.: 17/333,609

(22) Filed: May 28, 2021

Related U.S. Application Data
(63) Continuation of application No. PCT/US2019/063397, filed on Nov. 26, 2019.

(51) Int. Cl. C07K 7/64; A61K 31/337; A61K 9/00; A61P 35/00

(52) U.S. Cl. CPC C07K 7/64; A61K 31/337; A61K 38/00; A61P 35/00; A61K 9/0019

(57) ABSTRACT

The present disclosure describes the synthesis of peptidomimetic macrocycles and methods of using peptidomimetic macrocycles to treat a condition. The present disclosure also describes methods of using peptidomimetic macrocycles in combination with at least one additional pharmaceutically-active agent for the treatment of a condition, for example, cancer.

### CROSS REFERENCE

This application is a continuation of PCT/US19/63397 filed Nov. 26, 2019, which application claims the benefit of U.S. Provisional Application No. 62/773,540, filed on Nov. 30, 2018, the content of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on May 21, 2021, is named 352248333011 SL.TXT and is 1,195,187 bytes in size

### BACKGROUND

The human transcription factor protein p53 induces cell cycle arrest and apoptosis in response to DNA damage and cellular stress, and thereby plays a critical role in protecting cells from malignant transformation. The E3 ubiquitin ligase MDM2, also known as HDM2, negatively regulates p53 function through a direct binding interaction, which neutralizes the p53 transactivation activity. Loss of p53 activity, either by deletion, mutation, or MDM2 overexpression, is the most common defect in human cancers.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the invention provides a method of treating a condition in a subject in need thereof, comprising administering to the subject a combination therapy comprising a therapeutically-effective amount of a peptidomimetic macrocycle and a therapeutically-effective amount of paclitaxel, wherein the combination therapy has a combination index of less than 1.

### BRIEF DESCRIPTION OF THE FIGURES

FIG. 1 PANEL A shows cell viability data in response to varying concentrations of paclitaxel (arrows denote concentrations chosen for combination studies). PANEL B shows cell proliferation data of MCF-7 cells treated with the indicated dose of paclitaxel and varying concentrations of AP1. PANEL C shows combination indices for the drug combination of AP1 and paclitaxel.

FIG. 2 PANEL A shows data collected from athymic nude mice with established tumors (n=10 per group) that were treated for 4 weeks with AP1 twice-weekly alone or in combination with weekly doses of nab-paclitaxel. Compounds were co-administered intravenously at the indicated doses. PANEL B shows objective tumor responses on d32 (partial regression=3 consecutive measurements<50% of starting volume).

FIG. 3 shows a scatter plot of time to endpoint values for individual athymic nude mice by treatment group.

FIG. 4 PANEL A shows median tumor growth versus time by treatment group. FIG. 4 PANEL B shows mean tumor growth versus time by treatment group.

FIG. 5 shows a Kaplan-Meier plot of the percentage of animals in each group remaining in the study versus time.

FIG. 6 shows percent group mean body weight changes from Day 1 by treatment group.

### DETAILED DESCRIPTION

The human transcription factor protein p53 induces cell cycle arrest and apoptosis in response to DNA damage and cellular stress, and thereby plays a critical role in protecting cells from malignant transformation. The E3 ubiquitin ligase MDM2, also known as HDM2, negatively regulates p53 function through a direct binding interaction that neutralizes the p53 transactivation activity. Neutralization of p53 transactivation activity leads to export from the nucleus of p53 protein, which targets p53 for degradation via the ubiquitylation-proteasomal pathway. Loss of p53 activity, either by deletion, mutation, or MDM2 overexpression, is the most common defect in human cancers. Tumors that express wild type p53 are vulnerable to pharmacologic agents that stabilize or increase the concentration of active p53.

MDMX (MDM4) is a negative regulator of p53, and there is significant structural homology between the p53 binding interfaces of MDM2 and MDMX. The p53-MDM2 and p53-MDMX protein-protein interactions are mediated by the same 15-residue alpha-helical transactivation domain of p53, which inserts into hydrophobic clefts on the surface of MDM2 and MDMX. Three residues within this domain of p53 (F19, W23, and L26) are essential for binding to MDM2 and MDMX.

Paclitaxel is one of the most widely used chemotherapeutic agents that promotes the assembly of microtubules from tubulin dimers. Paclitaxel stabilizes microtubules by preventing depolymerization, which results in the inhibition of the normal dynamic reorganization of the microtubule network that is essential for vital interphase and mitotic cellular functions. Protein-bound paclitaxel or nanoparticle albumin-bound paclitaxel (Abraxane) is an injectable formulation of paclitaxel used to treat breast cancer, lung cancer, and pancreatic cancer.

Provided herein are p53-based peptidomimetic macrocycles that modulate an activity of p53 and p53-based peptidomimetic macrocycles that inhibit the interactions between p53 and MDM2 and/or p53 and MDMX proteins. Also provided herein are the use of p53-based peptidomimetic macrocycles and paclitaxel for the treatment of a condition. Further, provided herein are p53-based peptidomimetic macrocycles and paclitaxel that can be used for treating diseases, for example, cancer.

### Definitions

As used herein, the term “macrocycle” refers to a molecule having a chemical structure including a ring or cycle formed by at least 9 covalently bonded atoms.

As used herein, the term “peptidomimetic macrocycle” or “crosslinked polypeptide” refers to a compound comprising a plurality of amino acid residues joined by a plurality of peptide bonds and at least one macrocycle-forming linker which forms a macrocycle between a first naturally-occurring or non-naturally-occurring amino acid residue (or analogue) and a second naturally-occurring or non-naturally-occurring amino acid residue (or analogue) within the same molecule. Peptidomimetic macrocycle include embodiments where the macrocycle-forming linker connects the α-carbon of the first amino acid residue (or analogue) to the α-carbon of the second amino acid residue (or analogue). The peptidomimetic macrocycles optionally include one or more non-peptide bonds between one or more amino acid residues and/or amino acid analogue residues, and optionally include one or more non-naturally-occurring amino acid residues or amino acid analogue residues in addition to any which form the macrocycle. A “corresponding uncrosslinked polypeptide” when referred to in the context of a peptidomimetic macrocycle is understood to relate to a polypeptide of the same length as the macrocycle and comprising the equivalent natural amino acids of the wild-type sequence corresponding to the macrocycle.

AP1 is an alpha helical hydrocarbon crosslinked polypeptide macrocycle with an amino acid sequence less than 20 amino acids long that is derived from the transactivation domain of wild type human p53 protein. The N-terminus is acetylated, and the C-terminus is capped as a primary amide. AP1 contains a phenylalanine, a tryptophan and a leucine amino acid in the same positions relative to each other as in the transactivation domain of wild type human p53 protein. AP1 has a single cross link spanning amino acids in the i to the i+7 position of the amino acid sequence and has more than three amino acids between the i+7 position and the carboxyl terminus. AP1 binds to human MDM2 and MDM4 and has an observed mass of 950-975 m/e as measured by electrospray ionization-mass spectrometry.

As used herein, the term “stability” refers to the maintenance of a defined secondary structure in solution by a peptidomimetic macrocycle as measured by circular dichroism, NMR or another biophysical measure, or resistance to proteolytic degradation in vitro or in vivo. Non-limiting examples of secondary structures contemplated herein are α-helices, 3₁₀ helices, β-turns, and β-pleated sheets.

As used herein, the term “helical stability” refers to the maintenance of an α-helical structure by a peptidomimetic macrocycle as measured by circular dichroism or NMR. In some embodiments, a peptidomimetic macrocycle can exhibit at least a 1.25, 1.5, 1.75, or 2-fold increase in α-helicity as determined by circular dichroism compared to a corresponding uncrosslinked macrocycle.

The term “amino acid” refers to a molecule containing both an amino group and a carboxyl group. Suitable amino acids include, without limitation, both the D-and L-isomers of the naturally-occurring amino acids, as well as non-naturally-occurring amino acids prepared by organic synthesis or other metabolic routes. The term amino acid, as used herein, includes, without limitation, α-amino acids, natural amino acids, non-natural amino acids, and amino acid analogues.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

The term “naturally-occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | | | | Side-chain | Hydro- |
|---|---|---|---|---|---|
| | 3-Letter | 1-Letter | Side-chain | charge | pathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | Positive (10%) | −3.2 |
| | | | | Neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acids” are glycine, alanine, proline, and analogues thereof “Large hydrophobic amino acids” are valine, leucine, isoleucine, phenylalanine, methionine, tryptophan, and analogues thereof. “Polar amino acids” are serine, threonine, asparagine, glutamine, cysteine, tyrosine, and analogues thereof. “Charged amino acids” are lysine, arginine, histidine, aspartate, glutamate, and analogues thereof.

The term “amino acid analogue” refers to a molecule which is structurally similar to an amino acid and which can be substituted for an amino acid in the formation of a peptidomimetic macrocycle. Amino acid analogues include, without limitation, β-amino acids and amino acids wherein the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y, and V. Non-natural amino acids or amino acid analogues include, without limitation, structures according to the following:

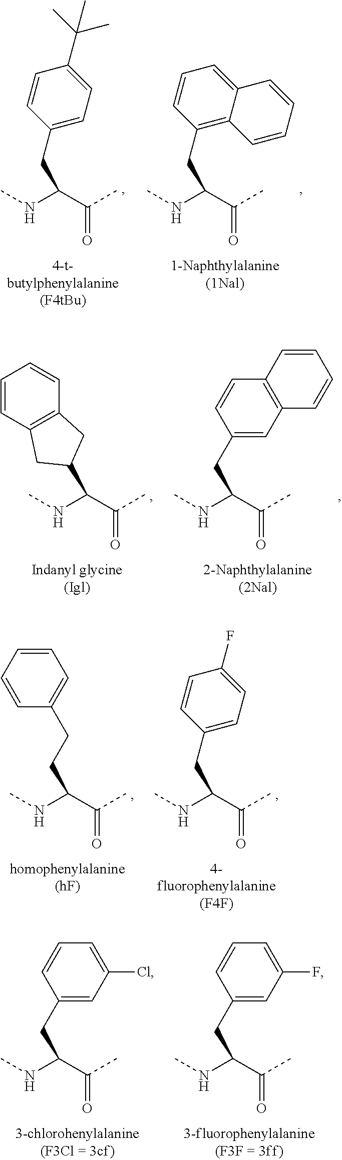

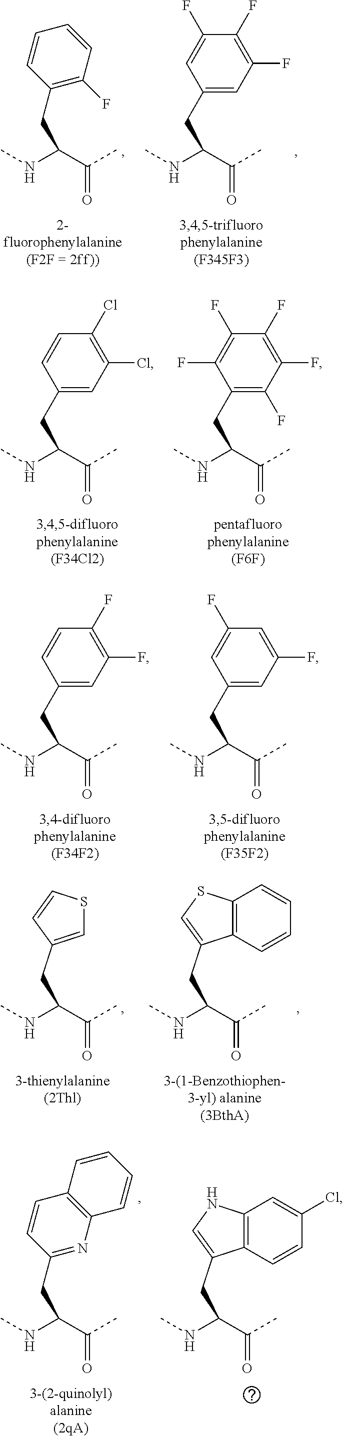

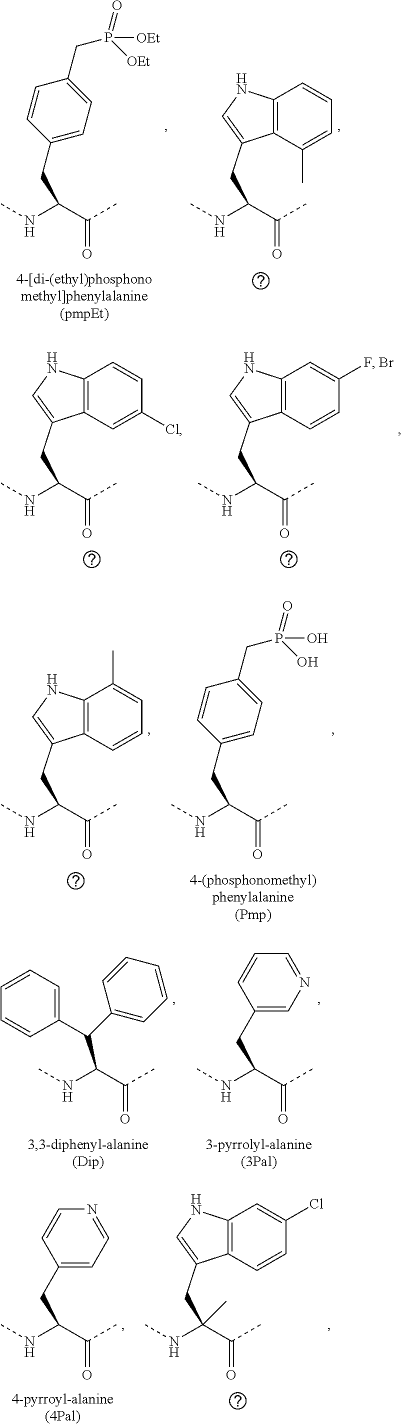

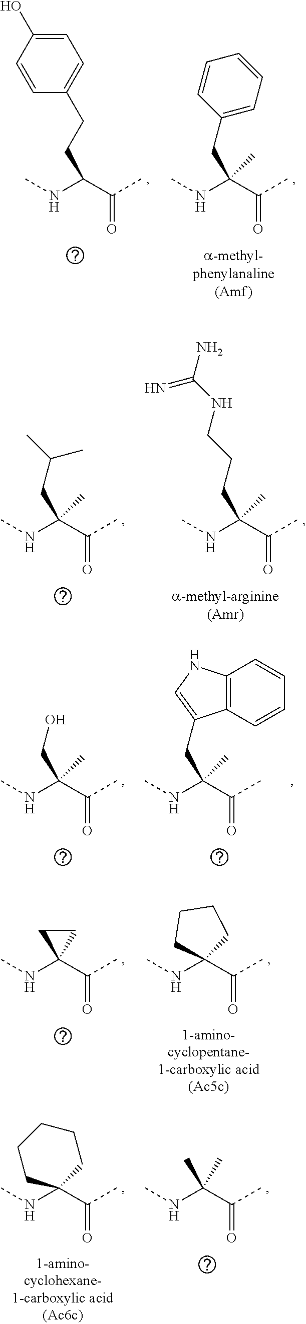

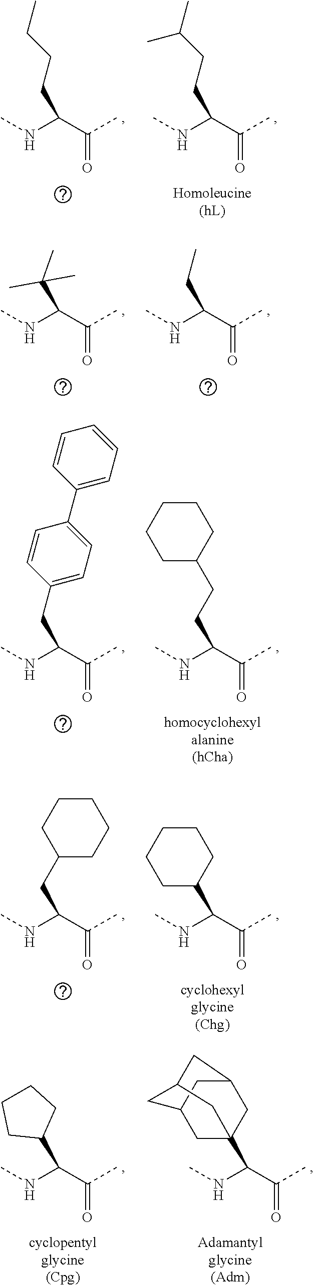

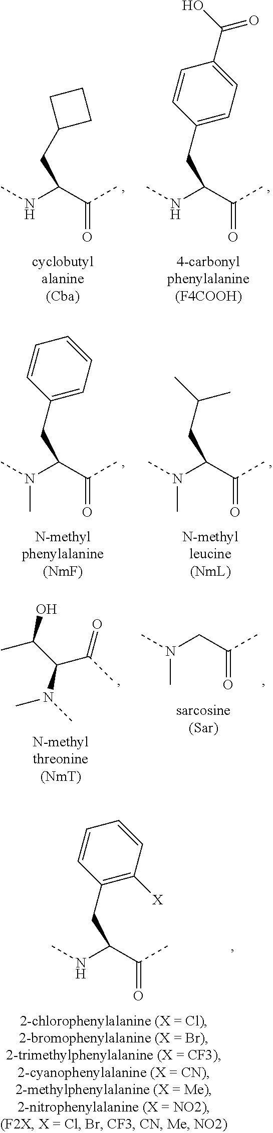

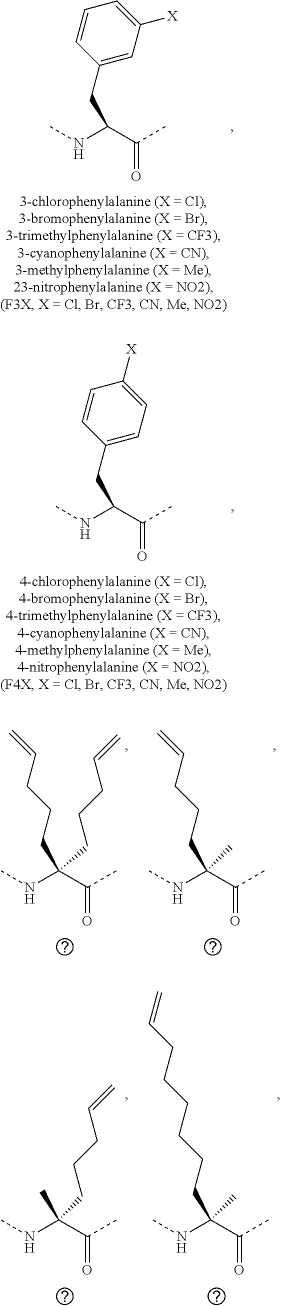

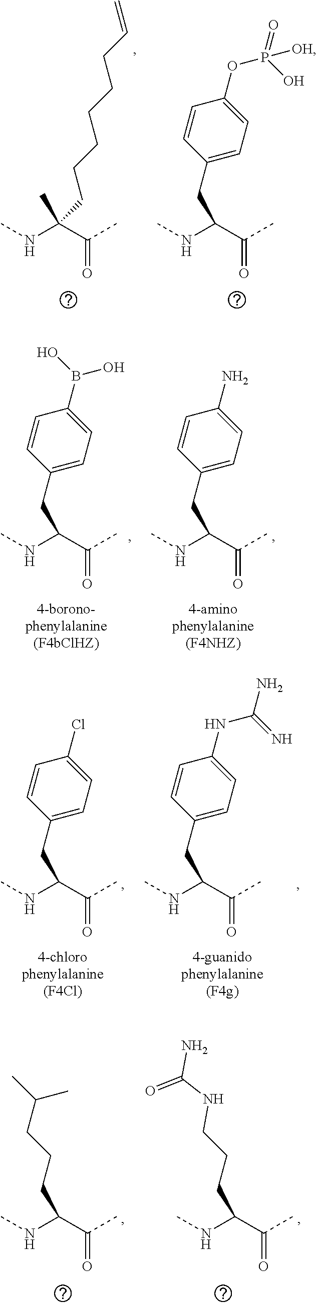

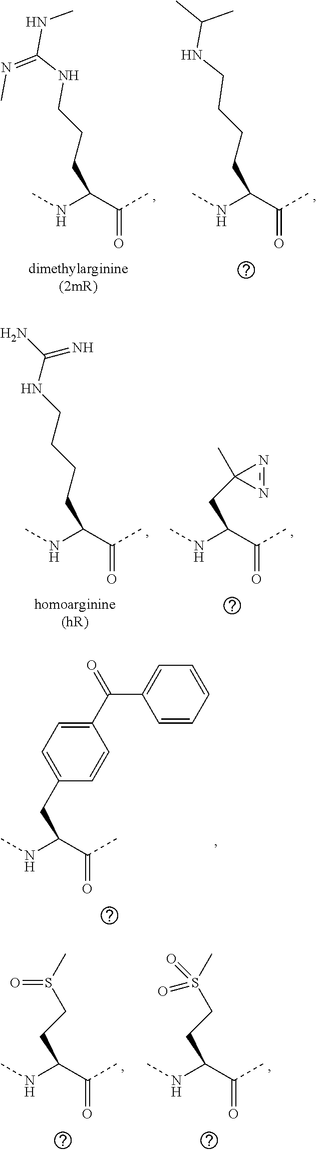

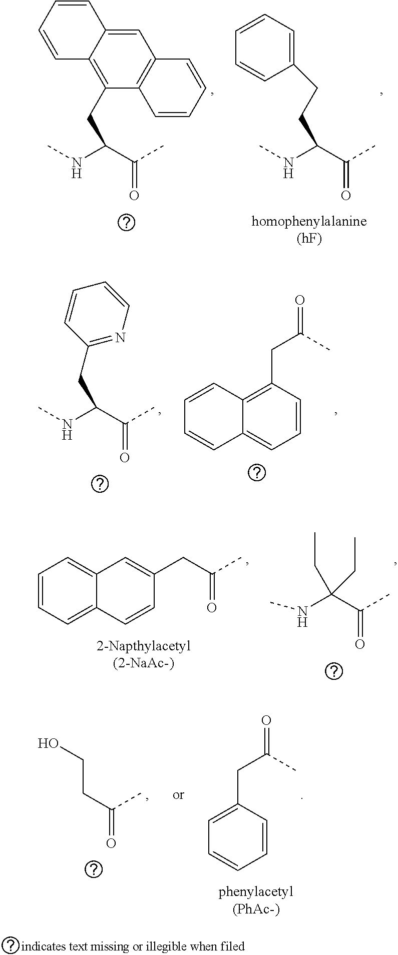

Amino acid analogues include β-amino acid analogues. Examples of β-amino acid analogues include, but are not limited to, the following: cyclic β-amino acid analogues; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogues include analogues of alanine, valine, glycine or leucine. Examples of amino acid analogues of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L-Ala-OH⋅dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH⋅dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH⋅dicyclohexylammonium salt; cyclopentyl-Gly-OH⋅dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine⋅dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine⋅dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogues include analogues of arginine or lysine. Examples of amino acid analogues of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)₂-OH (symmetrical); Lys(ivDde)-OH; Lys(Me)₂-OH.HCl; Lys(Me₃)-OH chloride; Nω-nitro-D-arginine; and Nω-nitro-L-arginine.

Amino acid analogues include analogues of aspartic or glutamic acids. Examples of amino acid analogues of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)—OH; and pyroglutamic acid.

Amino acid analogues include analogues of cysteine and methionine. Examples of amino acid analogues of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogues include analogues of phenylalanine and tyrosine. Examples of amino acid analogues of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D -phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogues include analogues of proline. Examples of amino acid analogues of proline include, but are not limited to, 3,4-dehydro-proline, 4-fluoro-proline, cis-4-hydroxy-proline, thiazolidine-2-carboxylic acid, and trans-4-fluoro-proline.

Amino acid analogues include analogues of serine and threonine. Examples of amino acid analogues of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogues include analogues of tryptophan. Examples of amino acid analogues of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogues are racemic. In some embodiments, the D isomer of the amino acid analogue is used. In some embodiments, the L isomer of the amino acid analogue is used. In other embodiments, the amino acid analogue comprises chiral centers that are in the R or S configuration. In still other embodiments, the amino group(s) of a β-amino acid analogue is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analogue is protected, e.g., as its ester derivative. In some embodiments the salt of the amino acid analogue is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, e.g., is replaced with another amino acid residue from the same side chain family. Other examples of acceptable substitutions are substitutions based on isosteric considerations (e.g., norleucine for methionine) or other properties (e.g., 2-thienylalanine for phenylalanine, or 6-Cl-tryptophan for tryptophan).

The term “capping group” refers to the chemical moiety occurring at either the carboxy or amino terminus of the polypeptide chain of the subject peptidomimetic macrocycle. The capping group of a carboxy terminus includes an unmodified carboxylic acid (i.e. —COOH) or a carboxylic acid with a substituent. For example, the carboxy terminus can be substituted with an amino group to yield a carboxamide at the C-terminus. Various substituents include but are not limited to primary, secondary, and tertiary amines, including pegylated secondary amines. Representative secondary amine capping groups for the C-terminus include:

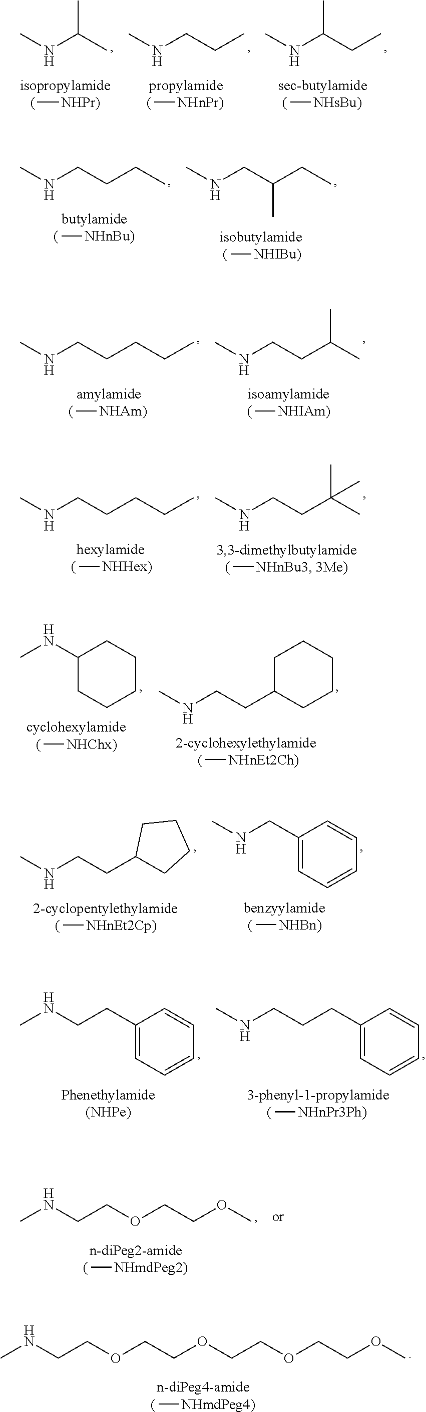

The capping group of an amino terminus includes an unmodified amine (i.e. —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates. Representative capping groups for the N-terminus include, but are not limited to, 4-FBzl (4-fluoro-benzyl) and the following:

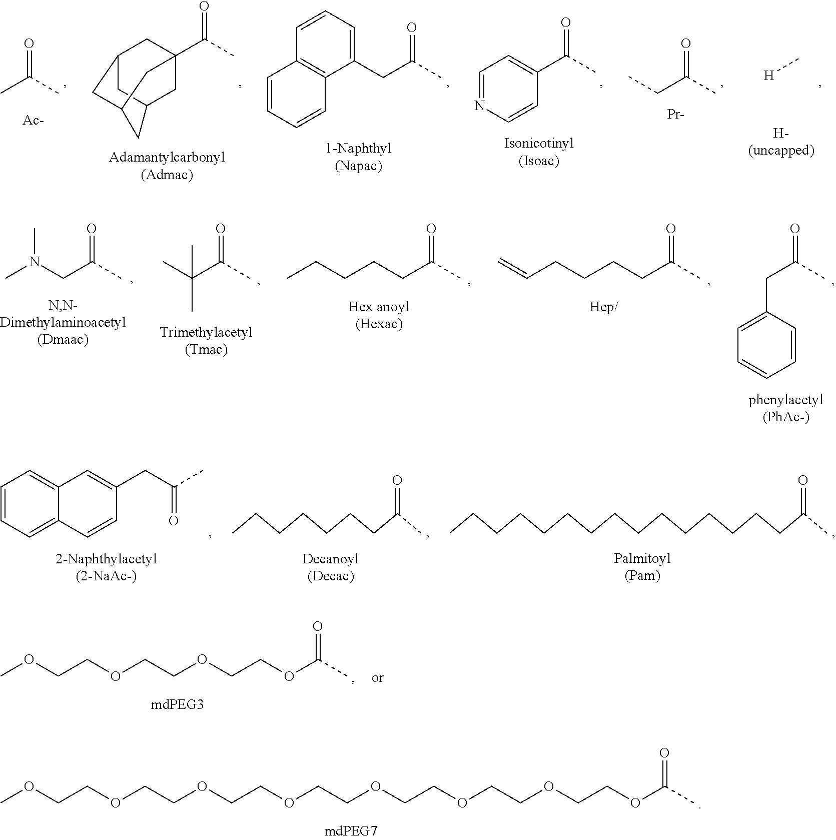

The term “member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

The symbol “ [symbol] ” when used as part of a molecular structure refers to a single bond or a trans or cis double bond.

The term “amino acid side chain” refers to a moiety attached to the α-carbon (or another backbone atom) in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally-occurring amino acid side chains are also included, for example, those that occur in nature (e.g., an amino acid metabolite) or those that are made synthetically (e.g., an a,a di-substituted amino acid).

The term “α,α di-substituted amino” acid refers to a molecule or moiety containing both an amino group and a carboxyl group bound to a carbon (the α-carbon) that is attached to two natural or non-natural amino acid side chains.

The term “polypeptide” encompasses two or more naturally-or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “first C-terminal amino acid” refers to the amino acid which is closest to the C-terminus. The term “second C-terminal amino acid” refers to the amino acid attached at the N-terminus of the first C-terminal amino acid.

The term “macrocyclization reagent” or “macrocycle-forming reagent” as used herein refers to any reagent which can be used to prepare a peptidomimetic macrocycle by mediating the reaction between two reactive groups. Reactive groups can be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents can additionally include, for example, Ru reagents known in the art such as Cp*RuCl(PPh₃)₂, [Cp*RuCl]₄ or other Ru reagents which can provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. In other examples, catalysts have W or Mo centers. In some embodiments, the reactive groups are thiol groups. In some embodiments, the macrocyclization reagent is, for example, a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1 to 10 (inclusive) carbon atoms in it. In the absence of any numerical designation, “alkyl” is a chain (straight or branched) having 1 to 20 (inclusive) carbon atoms.

The term “alkylene” refers to a divalent alkyl (i.e., —R—).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2 to 10 (inclusive) carbon atoms. The term “lower alkenyl” refers to a C₂-C₆ alkenyl chain. In the absence of any numerical designation, “alkenyl” is a chain (straight or branched) having 2 to 20 (inclusive) carbon atoms.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2 to 10 (inclusive) carbon atoms. The term “lower alkynyl” refers to a C₂-C₆ alkynyl chain. In the absence of any numerical designation, “alkynyl” is a chain (straight or branched) having 2 to 20 (inclusive) carbon atoms.

The term “aryl” refers to a 6-carbon monocyclic or 10-carbon bicyclic aromatic ring system wherein 0, 1, 2, 3, or 4 atoms of each ring are substituted by a substituent. Examples of aryl groups include phenyl, naphthyl and the like. The term “arylalkoxy” refers to an alkoxy substituted with aryl.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH₂-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl.

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)₂CH₂C(O)NH₂, —CH₂—CH₂—NH—C(O)—CH₃, —CH₂—CH₂—NH—C(O)—CH₃—CH₃, and —CH₂—CH₂—NH—C(O)—CH═CH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂ CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, the compounds disclosed herein contain one or more asymmetric centers and thus occur as racemates and racemic mixtures, single enantiomers, individual diastereomers and diastereomeric mixtures. All such isomeric forms of these compounds are included unless expressly provided otherwise. In some embodiments, the compounds disclosed herein are also represented in multiple tautomeric forms, in such instances, the compounds include all tautomeric forms of the compounds described herein (e.g., if alkylation of a ring system results in alkylation at multiple sites, the invention includes all such reaction products). All such isomeric forms of such compounds are included unless expressly provided otherwise. All crystal forms of the compounds described herein are included unless expressly provided otherwise.

As used herein, the terms “increase” and “decrease” mean, respectively, to cause a statistically significantly (i.e., p<0.1) increase or decrease of at least 5%.

As used herein, the recitation of a numerical range for a variable is intended to convey that the variable is equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values≥0 and ≤2 if the variable is inherently continuous.

As used herein, unless specifically indicated otherwise, the word “or” is used in the inclusive sense of “and/or” and not the exclusive sense of “either/or”.

The term “on average” represents the mean value derived from performing at least three independent replicates for each data point.

The term “biological activity” encompasses structural and functional properties of a macrocycle. Biological activity is, for example, structural stability, alpha-helicity, affinity for a target, resistance to proteolytic degradation, cell penetrability, intracellular stability, in vivo stability, or any combination thereof.

The term “binding affinity” refers to the strength of a binding interaction, for example between a peptidomimetic macrocycle and a target. Binding affinity can be expressed, for example, as equilibrium dissociation constant (“K_{D}”), which is expressed in units which are a measure of concentration (e.g. M, mM, μM, nM etc). Numerically, binding affinity and K_{D} values vary inversely, such that a lower binding affinity corresponds to a higher K_{D} value, while a higher binding affinity corresponds to a lower K_{D} value. Where high binding affinity is desirable, “improved” binding affinity refers to higher binding affinity and therefore lower K_{D} values.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The terms “combination therapy” or “combined treatment” or in “combination” as used herein denotes any form of concurrent or parallel treatment with at least two distinct therapeutic agents.

The term “in vitro efficacy” refers to the extent to which a test compound, such as a peptidomimetic macrocycle, produces a beneficial result in an in vitro test system or assay. In vitro efficacy can be measured, for example, as an “IC₅₀” or “EC₅₀” value, which represents the concentration of the test compound which produces 50% of the maximal effect in the test system.

The term “ratio of in vitro efficacies” or “in vitro efficacy ratio” refers to the ratio of IC₅₀ or EC₅₀ values from a first assay (the numerator) versus a second assay (the denominator). Consequently, an improved in vitro efficacy ratio for Assay 1 versus Assay 2 refers to a lower value for the ratio expressed as IC₅₀(Assay 1)/IC₅₀(Assay 2) or alternatively as EC₅₀(Assay 1)/EC₅₀(Assay 2). This concept can also be characterized as “improved selectivity” in Assay 1 versus Assay 2, which can be due either to a decrease in the IC₅₀ or EC₅₀ value for Target 1 or an increase in the value for the IC₅₀ or EC₅₀ value for Target 2.

As used in the present application, “biological sample” means any fluid or other material derived from the body of a normal or diseased subject, such as blood, serum, plasma, lymph, urine, saliva, tears, cerebrospinal fluid, milk, amniotic fluid, bile, ascites fluid, pus, and the like. Also included within the meaning of the term “biological sample” is an organ or tissue extract and culture fluid in which any cells or tissue preparation from a subject has been incubated. The biological samples can be any samples from which genetic material can be obtained. Biological samples can also include solid or liquid cancer cell samples or specimens. The cancer cell sample can be a cancer cell tissue sample. In some embodiments, the cancer cell tissue sample can obtained from surgically excised tissue. Exemplary sources of biological samples include fine needle aspiration, core needle biopsy, vacuum assisted biopsy, incisional biopsy, excisional biopsy, punch biopsy, shave biopsy or skin biopsy. In some cases, the biological samples comprise fine needle aspiration samples. In some embodiments, the biological samples comprise tissue samples, including, for example, excisional biopsy, incisional biopsy, or other biopsy. The biological samples can comprise a mixture of two or more sources; for example, fine needle aspirates and tissue samples. Tissue samples and cellular samples can also be obtained without invasive surgery, for example by punctuating the chest wall or the abdominal wall or from masses of breast, thyroid or other sites with a fine needle and withdrawing cellular material (fine needle aspiration biopsy). In some embodiments, a biological sample is a bone marrow aspirate sample. A biological sample can be obtained by methods known in the art such as the biopsy methods provided herein, swabbing, scraping, phlebotomy, or any other suitable method.

The term “solid tumor” or “solid cancer” as used herein refers to tumors that usually do not contain cysts or liquid areas. Solid tumors as used herein include sarcomas, carcinomas and lymphomas. In various embodiments, leukemia (cancer of blood) is not solid tumor.

Solid tumor cancers that can be treated by the methods provided herein include, but are not limited to, sarcomas, carcinomas, and lymphomas. In specific embodiments, solid tumors that can be treated in accordance with the methods described include, but are not limited to, cancer of the breast, liver, neuroblastoma, head, neck, eye, mouth, throat, esophagus, esophagus, chest, bone, lung, kidney, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. Solid tumors that can be treated by the instant methods include tumors and/or metastasis (wherever located) other than lymphatic cancer, for example brain and other central nervous system tumors (including but not limited to tumors of the meninges, brain, spinal cord, cranial nerves and other parts of central nervous system, e.g. glioblastomas or medulla blastemas); head and/or neck cancer; breast tumors; circulatory system tumors (including but not limited to heart, mediastinum and pleura, and other intrathoracic organs, vascular tumors and tumor-associated vascular tissue); excretory system tumors (including but not limited to tumors of kidney, renal pelvis, ureter, bladder, other and unspecified urinary organs); gastrointestinal tract tumors (including but not limited to tumors of the esophagus, stomach, small intestine, colon, colorectal, rectosigmoid junction, rectum, anus and anal canal, tumors involving the liver and intrahepatic bile ducts, gall bladder, other and unspecified parts of biliary tract, pancreas, other and digestive organs); oral cavity tumors (including but not limited to tumors of lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (including but not limited to tumors of vulva, vagina, Cervix uteri, Corpus uteri, uterus, ovary, and other sites associated with female genital organs, placenta, penis, prostate, testis, and other sites associated with male genital organs); respiratory tract tumors (including but not limited to tumors of nasal cavity and middle ear, accessory sinuses, larynx, trachea, bronchus and lung, e.g. small cell lung cancer or non-small cell lung cancer); skeletal system tumors (including but not limited to tumors of bone and articular cartilage of limbs, bone articular cartilage and other sites); skin tumors (including but not limited to malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

In some examples, the solid tumor treated by the methods of the instant disclosure is pancreatic cancer, bladder cancer, colon cancer, liver cancer, colorectal cancer (colon cancer or rectal cancer), breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, skin cancer, ocular tumor, choriocarcinoma (tumor of the placenta), sarcoma or soft tissue cancer.

In some examples, the solid tumor to be treated by the methods of the instant disclosure is selected bladder cancer, bone cancer, breast cancer, cervical cancer, CNS cancer, colon cancer, ocular tumor, renal cancer, liver cancer, lung cancer, pancreatic cancer, choriocarcinoma (tumor of the placenta), prostate cancer, sarcoma, skin cancer, soft tissue cancer or gastric cancer.

In some examples, the solid tumor treated by the methods of the instant disclosure is breast cancer. Non limiting examples of breast cancer that can be treated by the instant methods include ductal carcinoma in situ (DCIS or intraductal carcinoma), lobular carcinoma in situ (LCIS), invasive (or infiltrating) ductal carcinoma, invasive (or infiltrating) lobular carcinoma, inflammatory breast cancer, triple-negative breast cancer, paget disease of the nipple, phyllodes tumor (phylloides tumor or cystosarcoma phyllodes), angiosarcoma, adenoid cystic (or adenocystic) carcinoma, low-grade adenosquamous carcinoma, medullary carcinoma, papillary carcinoma, tubular carcinoma, metaplastic carcinoma, micropapillary carcinoma, and mixed carcinoma.

In some examples, the solid tumor treated by the methods of the instant disclosure is bone cancer. Non limiting examples of bone cancer that can be treated by the instant methods include osteosarcoma, chondrosarcoma, the Ewing Sarcoma Family of Tumors (ESFTs).

In some examples, the solid tumor treated by the methods of the instant disclosure is skin cancer. Non limiting examples of skin cancer that can be treated by the instant methods include melanoma, basal cell skin cancer, and squamous cell skin cancer.

In some examples, the solid tumor treated by the methods of the instant disclosure is ocular tumor. Non limiting examples of ocular tumor that can be treated by the methods of the instant disclosure include ocular tumor is choroidal nevus, choroidal melanoma, choroidal metastasis, choroidal hemangioma, choroidal osteoma, iris melanoma, uveal melanoma, intraocular lymphoma, melanocytoma, metastasis retinal capillary hemangiomas, congenital hypertrophy of the RPE, RPE adenoma or retinoblastoma.

In some embodiments solid tumors treated by the methods disclosed herein exclude cancers that are known to be associated with HPV (Human papillomavirus). The excluded group includes HPV positive cervical cancer, HPV positive anal cancer, and HPV head and neck cancers, such as oropharyngeal cancers.

The term “liquid cancer” as used herein refers to cancer cells that are present in body fluids, such as blood, lymph and bone marrow. Liquid cancers include leukemia, myeloma and liquid lymphomas. Liquid lymphomas include lymphomas that contain cysts or liquid areas. Liquid cancers as used herein do not include solid tumors, such as sarcomas and carcinomas or solid lymphomas that do not contain cysts or liquid areas.

Liquid cancer cancers that can be treated by the methods provided herein include, but are not limited to, leukemias, myelomas, and liquid lymphomas. In specific embodiments, liquid cancers that can be treated in accordance with the methods described include, but are not limited to, liquid lymphomas, leukemias, and myelomas. Exemplary liquid lymphomas and leukemias that can be treated in accordance with the methods described include, but are not limited to, chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma.

Examples of liquid cancers include cancers involving hyperplastic/neoplastic cells of hematopoietic origin, e.g., arising from myeloid, lymphoid or erythroid lineages, or precursor cells thereof. Exemplary disorders include: acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), multiple mylenoma, hairy cell leukemia (HLL) and Waldenstrom's macroglobulinemia (WM). Additional forms of malignant liquid lymphomas include, but are not limited to non-Hodgkin lymphoma and variants thereof, adult T cell leukemia/lymphoma (ATL), cutaneous T-cell lymphoma (CTCL), periphieral T-cell lymphoma (PTCL), large granular lymphocytic leukemia (LGF), Hodgkin's disease and Reed-Sternberg disease. For example, liquid cancers include, but are not limited to, acute lymphocytic leukemia (ALL); T-cell acute lymphocytic leukemia (T-ALL); anaplastic large cell lymphoma (ALCL); chronic myelogenous leukemia (CML); acute myeloid leukemia (AML); chronic lymphocytic leukemia (CLL); B-cell chronic lymphocytic leukemia (B-CLL); diffuse large B-cell lymphomas (DLBCL); hyper eosinophilia/chronic eosinophilia; and Burkitt's lymphoma.

In some embodiments, the cancer comprises an acute lymphoblastic leukemia; acute myeloid leukemia; AIDS-related cancers; AIDS-related lymphoma; chronic lymphocytic leukemia; chronic myelogenous leukemia; chronic myeloproliferative disorders; adult T cell leukemia/lymphoma (ATL), cutaneous T-cell lymphoma (CTCL), peripheral T-cell lymphoma (PTCL); Hodgkin lymphoma; multiple myeloma; multiple myeloma/plasma cell neoplasm; Non-Hodgkin lymphoma; or primary central nervous system (CNS) lymphoma. In various embodiments, the liquid cancer can be B-cell chronic lymphocytic leukemia, B-cell lymphoma-DLBCL, B-cell lymphoma-DLBCL-germinal center-like, B-cell lymphoma-DLBCL-activated B-cell-like, or Burkitt's lymphoma.

In some embodiments, a subject treated in accordance with the methods provided herein is a human who has or is diagnosed with cancer lacking p53 deactivating mutation and/or expressing wild type p53. In some embodiments, a subject treated for cancer in accordance with the methods provided herein is a human predisposed or susceptible to cancer lacking p53 deactivating mutation and/or expressing wild type p53. In some embodiments, a subject treated for cancer in accordance with the methods provided herein is a human at risk of developing cancer lacking p53 deactivating mutation and/or expressing wild type p53. A p53 deactivating mutation in some example can be a mutation in DNA-binding domain of the p53 protein. In some examples the p53 deactivating mutation can be a missense mutation. In various examples, the cancer can be determined to lack one or more p53 deactivating mutations selected from mutations at one or more of residues R175, G245, R248, R249, R273, and R282. The lack of p53 deactivating mutation and/or the presence of wild type p53 in the cancer can be determined by any suitable method known in art, for example by sequencing, array-based testing, RNA analysis and amplifications methods like PCR.

In certain embodiments, the human subject is refractory and/or intolerant to one or more other standard treatment of the cancer known in art. In some embodiments, the human subject has had at least one unsuccessful prior treatment and/or therapy of the cancer.

In some embodiments, a subject treated for tumor in accordance with the methods provided herein is a human, who has or is diagnosed with a tumor. In other embodiments, a subject treated for tumor in accordance with the methods provided herein is a human, predisposed or susceptible to a tumor. In some embodiments, a subject treated for tumor in accordance with the methods provided herein is a human, at risk of developing a tumor.

In some embodiments, a subject treated for tumor in accordance with the methods provided herein is a human, who has or is diagnosed with a tumor, determined to lack a p53 deactivating mutation and/or expressing wild type p53. In other embodiments, a subject treated for tumor in accordance with the methods provided herein is a human, predisposed or susceptible to a tumor, determined to lack a p53 deactivating mutation and/or expressing wild type p53. In some embodiments, a subject treated for tumor in accordance with the methods provided herein is a human, at risk of developing a tumor, determined to lack a p53 deactivating mutation and/or expressing wild type p53. A p53 deactivating mutation, as used herein is any mutation that leads to loss of (or a decrease in) the in vitro apoptotic activity of p53.

In some embodiments, the subject treated for tumor in accordance with the methods provided herein is a human, who has or is diagnosed with a tumor, determined to have a p53 gain of function mutation. In other embodiments, a subject treated for tumor in accordance with the methods provided herein is a human, predisposed or susceptible to a tumor, determined to have a p53 gain of function mutation. In some embodiments, a subject treated for tumor in accordance with the methods provided herein is a human, at risk of developing a tumor, determined to have a p53 gain of function mutation. A p53 gain of function mutation, as used herein is any mutation such that the mutant p53 exerts oncogenic functions beyond their negative domination over the wild-type p53 tumor suppressor functions. The p53 gain of function mutant protein mat exhibit new activities that can contribute actively to various stages of tumor progression and to increased resistance to anticancer treatments. Accordingly, in some embodiments, a subject with a tumor in accordance with the composition as provided herein is a human who has or is diagnosed with a tumor that is determined to have a p53 gain of function mutation.

In some embodiments, the subject treated for tumor in accordance with the methods provided herein is a human, who has or is diagnosed with a tumor that is not p53 negative. In other embodiments, a subject treated for tumor in accordance with the methods provided herein is a human, predisposed or susceptible to a tumor that is not p53 negative. In some embodiments, a subject treated for tumor in accordance with the methods provided herein is a human, at risk of developing a tumor that is not p53 negative.

In some embodiments, the subject treated for tumor in accordance with the methods provided herein is a human, who has or is diagnosed with a tumor that expresses p53 with partial loss of function mutation. In other embodiments, a subject treated for tumor in accordance with the methods provided herein is a human, predisposed or susceptible to a tumor that expresses p53 with partial loss of function mutation. In some embodiments, a subject treated for tumor in accordance with the methods provided herein is a human, at risk of developing a tumor that expresses p53 with partial loss of function mutation. As used herein “a partial loss of p53 function” mutation means that the mutant p53 exhibits some level of function of normal p53, but to a lesser or slower extent. For example, a partial loss of p53 function can mean that the cells become arrested in cell division to a lesser or slower extent.

In some embodiments, the subject treated for tumor in accordance with the methods provided herein is a human, who has or is diagnosed with a tumor that expresses p53 with a copy loss mutation and a deactivating mutation. In other embodiments, a subject treated for tumor in accordance with the methods provided herein is a human, predisposed or susceptible to a tumor that expresses p53 with a copy loss mutation and a deactivating mutation. In some embodiments, a subject treated for tumor in accordance with the methods provided herein is a human, at risk of developing a tumor that expresses p53 with a copy loss mutation and a deactivating mutation.

In some embodiments, the subject treated for tumor in accordance with the methods provided herein is a human, who has or is diagnosed with a tumor that expresses p53 with a copy loss mutation. In other embodiments, a subject treated for tumor in accordance with the methods provided herein is a human, predisposed or susceptible to a tumor that expresses p53 with a copy loss mutation. In some embodiments, a subject treated for tumor in accordance with the methods provided herein is a human, at risk of developing a tumor that expresses p53 with a copy loss mutation.

In some embodiments, the subject treated for tumor in accordance with the methods provided herein is a human, who has or is diagnosed with a tumor that expresses p53 with one or more silent mutations. In other embodiments, a subject treated for tumor in accordance with the methods provided herein is a human, predisposed or susceptible to a tumor that expresses p53 with one or more silent mutations. In some embodiments, a subject treated for tumor in accordance with the methods provided herein is a human, at risk of developing a tumor that expresses p53 with one or more silent mutations. Silent mutations as used herein are mutations which cause no change in the encoded p53 amino acid sequence.

In some embodiments, a subject treated for tumor in accordance with the methods provided herein is a human, who has or is diagnosed with a tumor, determined to lack a dominant p53 deactivating mutation. Dominant p53 deactivating mutation or dominant negative mutation, as used herein, is a mutation wherein the mutated p53 inhibits or disrupt the activity of the wild-type p53 gene.

### Peptidomimetic Macrocycles

In some embodiments, a peptidomimetic macrocycle has the Formula (I):

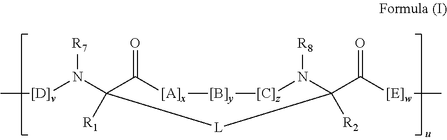

wherein:
each A, C, D, and E is independently a natural or non-natural amino acid or an amino acid analog, and each terminal D and E independently optionally includes a capping group;
each B is independently a natural or non-natural amino acid, an amino acid analog,

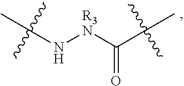

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R4 is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently —H, alkyl, alkenyl, alkynyl, arylalkyl, cycloalkylalkyl, heterocycloalkyl, a fluorescent moiety, a radioisotope, or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000, for example 1-500, 1-200, 1-100, 1-50, 1-30, 1-20, or 1-10;
u is an integer from 1-10, for example 1-5, 1-3, or 1-2;
each x, y, and z is independently an integer from 0-10, for example the sum of x+y+z is 2, 3, or 6; and
n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In an embodiment of any of the Formulas described herein, L₁ and L₂, either alone or in combination, do not form a triazole or a thioether.

In one example, at least one of R₁ and R₂ is alkyl that is unsubstituted or substituted with halo-. In another example, both R₁ and R₂ are independently alkyl that is unsubstituted or substituted with halo-. In some embodiments, at least one of R₁ and R₂ is methyl. In other embodiments, R₁ and R₂ are methyl.

In some embodiments, x+y+z is at least 3. In other embodiments, x+y+z is 1, 2, 3, 4, 5, 6, 7, 8, 9, or 10. In some embodiments, the sum of x+y+z is 3 or 6. In some embodiments, the sum of x+y+z is 3. In other embodiments, the sum of x+y+z is 6. Each occurrence of A, B, C, D, or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is an α-helix and R₈ is —H, allowing for intra-helical hydrogen bonding. In some embodiments, at least one of A, B, C, D, or E is an α,α-disubstituted amino acid. In one example, B is an α,α-disubstituted amino acid. For instance, at least one of A, B, C, D, or E is 2-aminoisobutyric acid. In other embodiments, at least one of A, B, C, D, or E is

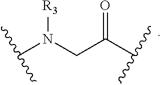

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, peptidomimetic macrocycles are also provided of the formula:

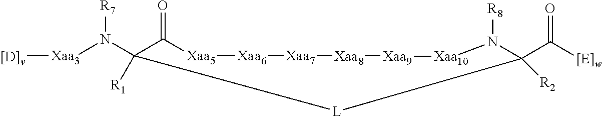

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂, wherein each X is an amino acid;
each D and E is independently a natural or non-natural amino acid or an amino acid analog;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent; —each R₆ is independently —H, alkyl, alkenyl, alkynyl, arylalkyl, cycloalkylalkyl, heterocycloalkyl, a fluorescent moiety, a radioisotope, or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000, for example 1-500, 1-200, 1-100, 1-50, 1-30, 1-20, or 1-10;
w is an integer from 3-1000, for example 3-500, 3-200, 3-100, 3-50, 3-30, 3-20, or 3-10; and
n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments of any of the Formulas described herein, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂. In other embodiments, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂. In other embodiments, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂. In other embodiments, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂. In other embodiments, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂.

In some embodiments, a peptidomimetic macrocycle has the Formula:

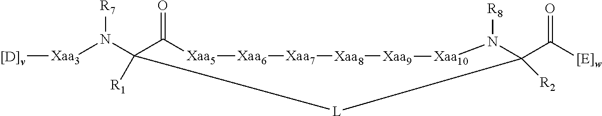

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂, wherein each X is an amino acid;
each D is independently a natural or non-natural amino acid or an amino acid analog;
each E is independently a natural or non-natural amino acid or an amino acid analog, for example an amino acid selected from Ala (alanine), D-Ala (D-alanine), Aib (α-aminoisobutyric acid), Sar (N-methyl glycine), and Ser (serine);
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula —L₁-L₂-;
each L₁ and L₂ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently —H, alkyl, alkenyl, alkynyl, arylalkyl, cycloalkylalkyl, heterocycloalkyl, a fluorescent moiety, a radioisotope, or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000, for example 1-500, 1-200, 1-100, 1-50, 1-30, 1-20, or 1-10;
w is an integer from 3-1000, for example 3-500, 3-200, 3-100, 3-50, 3-30, 3-20, or 3-10; and
n is an integer from 1-5.

In some embodiments of the above Formula, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂. In other embodiments of the above Formula, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂. In other embodiments of the above Formula, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂. In other embodiments of the above Formula, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln9-Leu₁₀/Cba₁₀-X₁₁-Ala₁₂. In other embodiments of the above Formula, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂.

In some embodiments, w is an integer from 3-10, for example 3-6, 3-8, 6-8, or 6-10. In some embodiments, w is 3. In other embodiments, w is 6. In some embodiments, v is an integer from 1-10. In some embodiments, v is 2.

In an embodiment of any of the Formulas described herein, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 3. In other embodiments, x+y+z is 1, 2, 3, 4, 5, 6, 7, 8, 9, or 10. In some embodiments, the sum of x+y+z is 3 or 6. In some embodiments, the sum of x+y+z is 3. In other embodiments, the sum of x+y+z is 6. Each occurrence of A, B, C, D, or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is an α-helix and R₈ is —H, allowing intra-helical hydrogen bonding. In some embodiments, at least one of A, B, C, D, or E is an α,α-disubstituted amino acid. In one example, B is an α,α-disubstituted amino acid. For instance, at least one of A, B, C, D, or E is 2-aminoisobutyric acid. In other embodiments, at least one of A, B, C, D, or E is

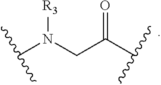

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, a peptidomimetic macrocycle of Formula (I) has Formula (Ia):

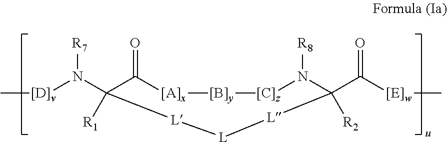

wherein:
each of A, C, D, and E is independently a natural or non-natural amino acid or an amino acid analog;
each B is independently a natural or non-natural amino acid, amino acid analog,

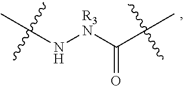

[—NH-L₃-CO—], [—NH-L₃-SO₂—], or [—NH-L₃-];
each L is independently a macrocycle-forming linker;
each L′ is independently alkylene, alkenylene, alkynylene, heteroalkylene, cycloalkylene, heterocycloalkylene, arylene, or heteroarylene, each being optionally substituted with R₅, or a bond, or together with R₁ and the atom to which both R₁ and L′ are bound forms a ring;
each L″ is independently alkylene, alkenylene, alkynylene, heteroalkylene, cycloalkylene, heterocycloalkylene, arylene, or heteroarylene, each being optionally substituted with R₅, or a bond, or together with R₂ and the atom to which both R₂ and L″ are bound forms a ring;
each R₁ is independently —H, alkyl, alkenyl, alkynyl, arylalkyl, cycloalkyl, cycloalkylalkyl, heteroalkyl, or heterocycloalkyl, unsubstituted or substituted with halo-, or together with L′ and the atom to which both R₁ and L′ are bound forms a ring;
each R₂ is independently —H, alkyl, alkenyl, alkynyl, arylalkyl, cycloalkyl, cycloalkylalkyl, heteroalkyl, or heterocycloalkyl, unsubstituted or substituted with halo-, or together with L″ and the atom to which both R₂ and L″ are bound forms a ring;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
n is an integer from 1-5;
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently —H, alkyl, alkenyl, alkynyl, arylalkyl, cycloalkylalkyl, heterocycloalkyl, a fluorescent moiety, a radioisotope, or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000, for example 1-500, 1-200, 1-100, 1-50, 1-40, 1-25, 1-20, 1-15, or 1-10;
each x, y and z is independently an integer from 0-10, for example x+y+z is 2, 3, or 6; and
u is an integer from 1-10, for example 1-5, 1-3, or 1-2.

In some embodiments, L is a macrocycle-forming linker of the formula -L₁-L₂-. In some embodiments, each L₁ and L₂ is independently alkylene, alkenylene, alkynylene, heteroalkylene, cycloalkylene, heterocycloalkylene, arylene, heteroarylene, or [—R₄—K—R₄—]ₙ, each being optionally substituted with R₅; each R₄ is independently alkylene, alkenylene, alkynylene, heteroalkylene, cycloalkylene, heterocycloalkylene, arylene, or heteroarylene; each K is independently O, S, SO, SO₂, CO, CO₂, or CONR₃; and n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 2. In other embodiments, x+y+z is 1, 2, 3, 4, 5, 6, 7, 8, 9 or 10. Each occurrence of A, B, C, D, or E in a macrocycle or macrocycle precursor is independently selected. For example, a sequence represented by the formula [A]ₓ, when x is 3, encompasses embodiments where the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is a helix and R₈ is —H, allowing intra-helical hydrogen bonding. In some embodiments, at least one of A, B, C, D, or E is an α,α-disubstituted amino acid. In one example, B is an α,α-disubstituted amino acid. For instance, at least one of A, B, C, D, or E is 2-aminoisobutyric acid. In other embodiments, at least one of A, B, C, D, or E is

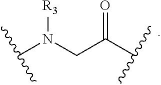

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Cα.

In one embodiment, the peptidomimetic macrocycle of Formula (I) is:

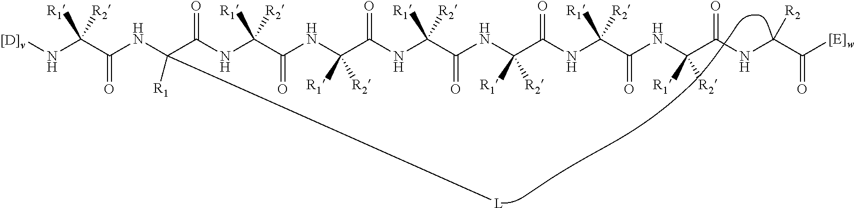

wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle of Formula (I) is:

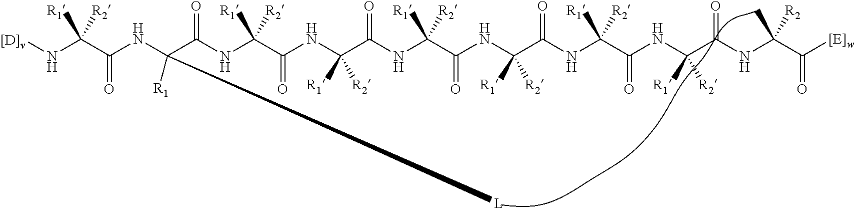

wherein each R₁′ and R₂′ is independently an amino acid.

In other embodiments, the peptidomimetic macrocycle of Formula (I) is a compound of any of the formulas shown below:

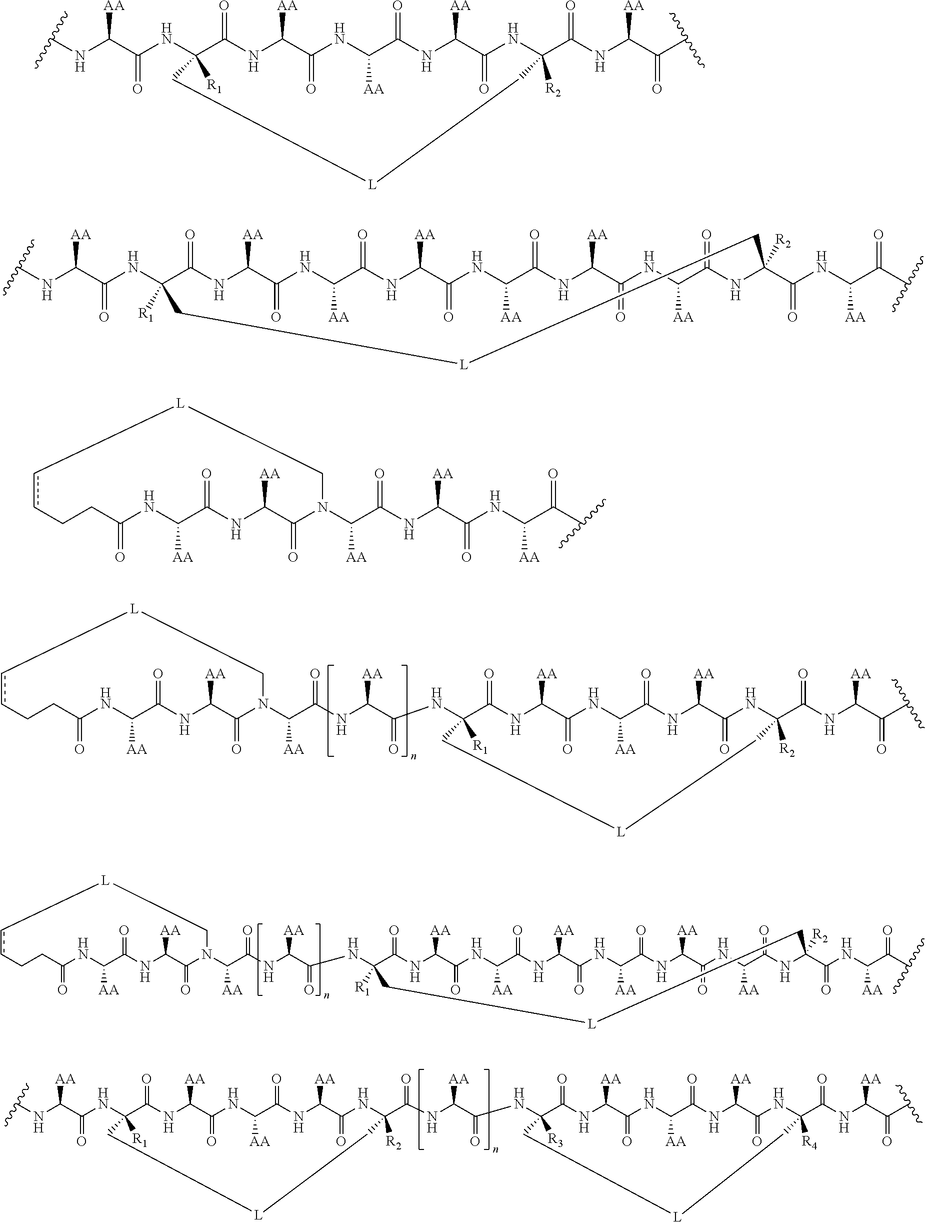

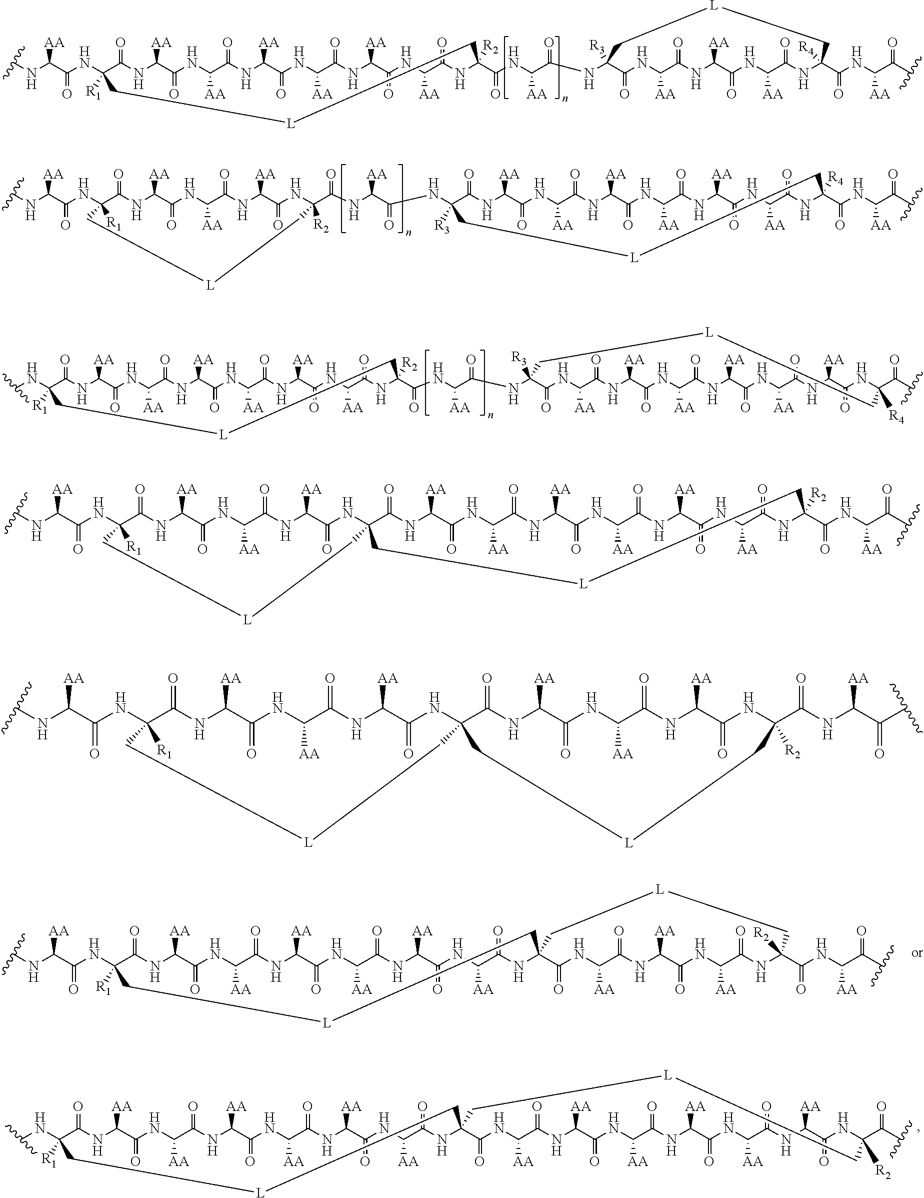

wherein “AA” represents any natural or non-natural amino acid side chain and “ [symbol] ” is [D]ᵥ, [E]_{w} as defined above, and n is an integer between 0 and 20, 50, 100, 200, 300, 400 or 500. In some embodiments, n is 0. In other embodiments, n is less than 50.

Exemplary embodiments of the macrocycle-forming linker L are shown below.

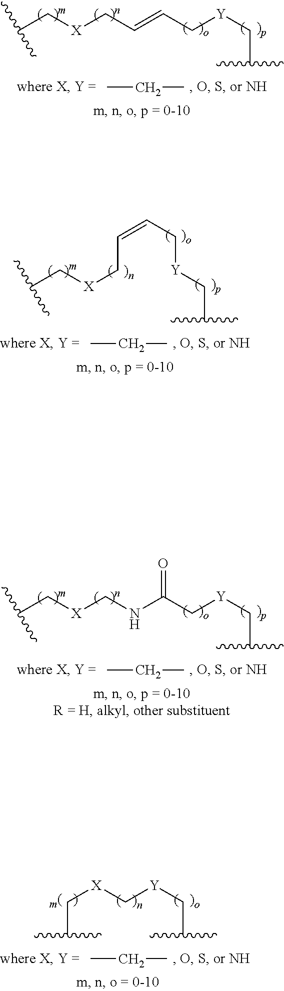

In other embodiments, D and/or E in the compound of Formula I are further modified to facilitate cellular uptake. In some embodiments, lipidating or PEGylating a peptidomimetic macrocycle facilitates cellular uptake, increases bioavailability, increases blood circulation, alters pharmacokinetics, decreases immunogenicity and/or decreases the needed frequency of administration.

In other embodiments, at least one of [D] and [E] in the compound of Formula I represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers. In an embodiment, u is 2.

In some embodiments, the peptidomimetic macrocycles have the Formula (I):

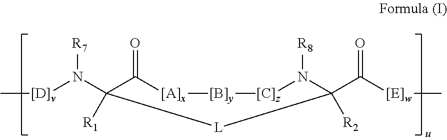

wherein:
each A, C, D, and E is independently a natural or non-natural amino acid or an amino acid analog;
each B is independently a natural or non-natural amino acid, amino acid analog,

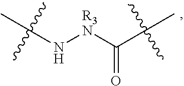

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently macrocycle-forming linker of the formula

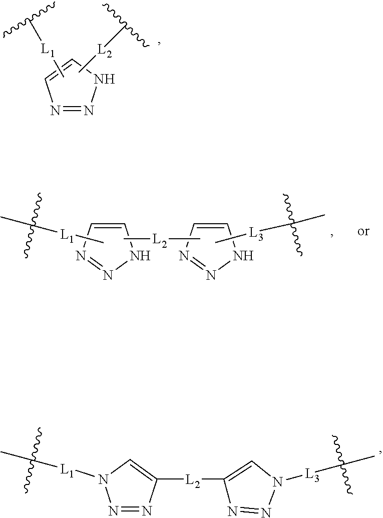

wherein each L₁, L₂ and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently —H, alkyl, alkenyl, alkynyl, arylalkyl, cycloalkylalkyl, heterocycloalkyl, a fluorescent moiety, a radioisotope, or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000;
each x, y and z is independently an integer from 0-10;
us is an integer from 1-10; and
n is an integer from 1-5.

In one example, at least one of R₁ and R₂ is alkyl that is unsubstituted or substituted with halo-. In another example, both R₁ and R₂ are independently alkyl that are unsubstituted or substituted with halo-. In some embodiments, at least one of R₁ and R₂ is methyl. In other embodiments, R₁ and R₂ are methyl.

In some embodiments, x+y+z is at least 2. In other embodiments, x+y+z is 1, 2, 3, 4, 5, 6, 7, 8, 9 or 10. Each occurrence of A, B, C, D, or E in a macrocycle or macrocycle precursor is independently selected. For example, a sequence represented by the formula [A]ₓ, when x is 3, encompasses embodiments where the amino acids are not identical, e.g. Gln-Asp-Ala as well as embodiments wherein the amino acids are identical, e.g. Gln-Gln-Gln. This applies for any value of x, y, or z in the indicated ranges.

In some embodiments, each of the first two amino acid represented by E comprises an uncharged side chain or a negatively charged side chain. In some embodiments, each of the first three amino acid represented by E comprises an uncharged side chain or a negatively charged side chain. In some embodiments, each of the first four amino acid represented by E comprises an uncharged side chain or a negatively charged side chain. In some embodiments, one or more or each of the amino acid that is i+1, i+2, i+3, i+4, i+5, and/or i+6 with respect to Xaa₁₃ represented by E comprises an uncharged side chain or a negatively charged side chain.

In some embodiments, the first C-terminal amino acid and/or the second C-terminal amino acid represented by E comprise a hydrophobic side chain. For example, the first C-terminal amino acid and/or the second C-terminal amino acid represented by E comprises a hydrophobic side chain, for example a small hydrophobic side chain. In some embodiments, the first C-terminal amino acid, the second C-terminal amino acid, and/or the third C-terminal amino acid represented by E comprise a hydrophobic side chain. For example, the first C-terminal amino acid, the second C-terminal amino acid, and/or the third C-terminal amino acid represented by E comprises a hydrophobic side chain, for example a small hydrophobic side chain. In some embodiments, one or more or each of the amino acid that is i+1, i+2, i+3, i+4, i+5, and/or i+6 with respect to Xaan represented by E comprises an uncharged side chain or a negatively charged side chain.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, the peptidomimetic macrocycle comprises a secondary structure which is a helix and R₈ is —H, allowing intra-helical hydrogen bonding. In some embodiments, at least one of A, B, C, D, or E is an α,α-disubstituted amino acid. In one example, B is an α,α-disubstituted amino acid. For instance, at least one of A, B, C, D, or E is 2-aminoisobutyric acid. In other embodiments, at least one of A, B, C, D, or E is

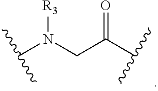

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Cα.

In some embodiments, L is a macrocycle-forming linker of the formula

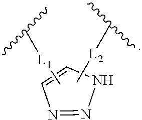

In some embodiments, L is a macrocycle-forming linker of the formula

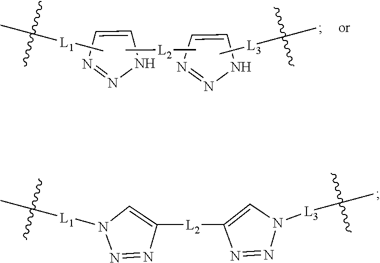

or a tautomer thereof.

Exemplary embodiments of the macrocycle-forming linker L are shown below:

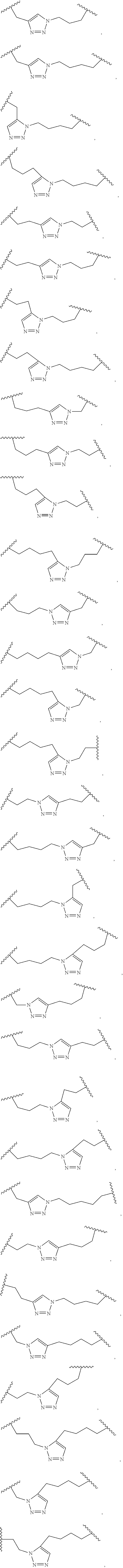

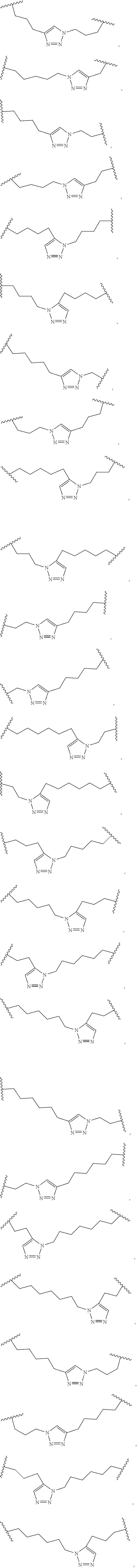

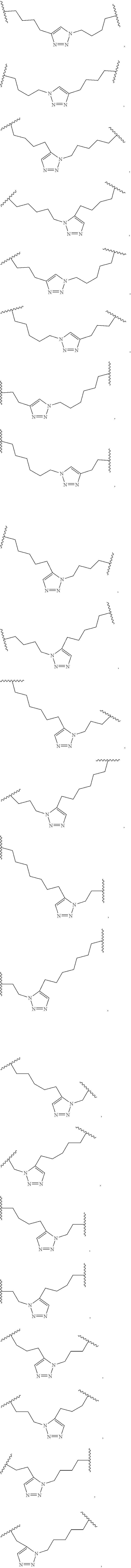

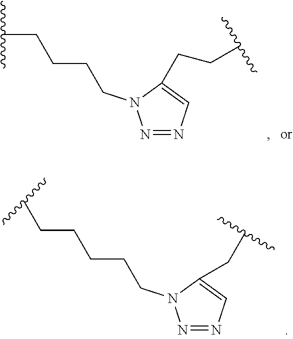 [SMILES: *CCCCn1nncc1CC* ; *CCCCCn1nncc1C*]

Amino acids which are used in the formation of triazole crosslinkers are represented according to the legend indicated below. Stereochemistry at the alpha position of each amino acid is S unless otherwise indicated. For azide amino acids, the number of carbon atoms indicated refers to the number of methylene units between the alpha carbon and the terminal azide. For alkyne amino acids, the number of carbon atoms indicated is the number of methylene units between the alpha position and the triazole moiety plus the two carbon atoms within the triazole group derived from the alkyne.
$5a5 Alpha-Me alkyne 1,5 triazole (5 carbon)
$5n3 Alpha-Me azide 1,5 triazole (3 carbon)
$4rn6 Alpha-Me R-azide 1,4 triazole (6 carbon)
$4a5 Alpha-Me alkyne 1,4 triazole (5 carbon)

In some embodiments, any of the macrocycle-forming linkers described herein can be used in any combination with any of the sequences shown in TABLE 1, TABLE 1a, TABLE 1b, or TABLE 1c and also with any of the R— substituents indicated herein.

In some embodiments, the peptidomimetic macrocycle comprises at least one α-helix motif. For example, A, B and/or C in the compound of Formula I include one or more α-helices. As a general matter, α-helices include between 3 and 4 amino acid residues per turn. In some embodiments, the α-helix of the peptidomimetic macrocycle includes 1 to 5 turns and, therefore, 3 to 20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix.

In some embodiments, the macrocycle-forming linker spans from 1 turn to 5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5 Å to 9 Å per turn of the α-helix, or approximately 6 Å to 8 Å per turn of the α-helix.

Where the macrocycle-forming linker spans approximately 1 turn of an α-helix, the length is equal to approximately 5 carbon-carbon bonds to 13 carbon-carbon bonds, approximately 7 carbon-carbon bonds to 11 carbon-carbon bonds, or approximately 9 carbon-carbon bonds. Where the macrocycle-forming linker spans approximately 2 turns of an α-helix, the length is equal to approximately 8 carbon-carbon bonds to 16 carbon-carbon bonds, approximately 10 carbon-carbon bonds to 14 carbon-carbon bonds, or approximately 12 carbon-carbon bonds. Where the macrocycle-forming linker spans approximately 3 turns of an α-helix, the length is equal to approximately 14 carbon-carbon bonds to 22 carbon-carbon bonds, approximately 16 carbon-carbon bonds to 20 carbon-carbon bonds, or approximately 18 carbon-carbon bonds. Where the macrocycle-forming linker spans approximately 4 turns of an α-helix, the length is equal to approximately 20 carbon-carbon bonds to 28 carbon-carbon bonds, approximately 22 carbon-carbon bonds to 26 carbon-carbon bonds, or approximately 24 carbon-carbon bonds. Where the macrocycle-forming linker spans approximately 5 turns of an α-helix, the length is equal to approximately 26 carbon-carbon bonds to 34 carbon-carbon bonds, approximately 28 carbon-carbon bonds to 32 carbon-carbon bonds, or approximately 30 carbon-carbon bonds. Where the macrocycle-forming linker spans approximately 1 turn of an α-helix, the linkage contains approximately 4 atoms to 12 atoms, approximately 6 atoms to 10 atoms, or approximately 8 atoms. Where the macrocycle-forming linker spans approximately 2 turns of the α-helix, the linkage contains approximately 7 atoms to 15 atoms, approximately 9 atoms to 13 atoms, or approximately 11 atoms. Where the macrocycle-forming linker spans approximately 3 turns of the α-helix, the linkage contains approximately 13 atoms to 21 atoms, approximately 15 atoms to 19 atoms, or approximately 17 atoms. Where the macrocycle-forming linker spans approximately 4 turns of the α-helix, the linkage contains approximately 19 atoms to 27 atoms, approximately 21 atoms to 25 atoms, or approximately 23 atoms. Where the macrocycle-forming linker spans approximately 5 turns of the α-helix, the linkage contains approximately 25 atoms to 33 atoms, approximately 27 atoms to 31 atoms, or approximately 29 atoms.

Where the macrocycle-forming linker spans approximately 1 turn of the α-helix, the resulting macrocycle forms a ring containing approximately 17 members to 25 members, approximately 19 members to 23 members, or approximately 21 members. Where the macrocycle-forming linker spans approximately 2 turns of the α-helix, the resulting macrocycle forms a ring containing approximately 29 members to 37 members, approximately 31 members to 35 members, or approximately 33 members. Where the macrocycle-forming linker spans approximately 3 turns of the α-helix, the resulting macrocycle forms a ring containing approximately 44 members to 52 members, approximately 46 members to 50 members, or approximately 48 members. Where the macrocycle-forming linker spans approximately 4 turns of the α-helix, the resulting macrocycle forms a ring containing approximately 59 members to 67 members, approximately 61 members to 65 members, or approximately 63 members. Where the macrocycle-forming linker spans approximately 5 turns of the α-helix, the resulting macrocycle forms a ring containing approximately 74 members to 82 members, approximately 76 members to 80 members, or approximately 78 members.

In other embodiments, provided are peptidomimetic macrocycles of Formula (II) or (IIa):

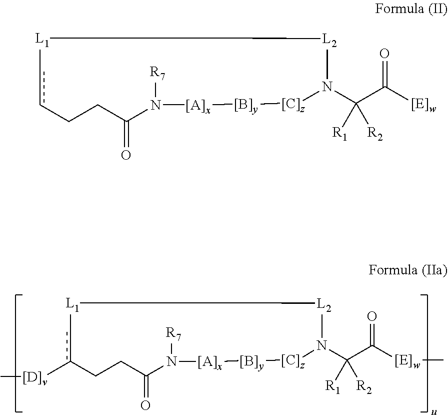

wherein:
each A, C, D, and E is independently a natural or non-natural amino acid or an amino acid analog, and the terminal D and E independently optionally include a capping group;
each B is independently a natural or non-natural amino acid, amino acid analog,

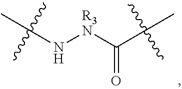

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently —H, alkyl, alkenyl, alkynyl, arylalkyl, cycloalkylalkyl, heterocycloalkyl, a fluorescent moiety, a radioisotope, or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅;
each v and w is independently an integer from 1-1000;
u is an integer from 1-10;
each x, y, and z is independently integers from 0-10; and
n is an integer from 1-5.

In one example, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 1. In other embodiments, x+y+z is at least 2. In other embodiments, x+y+z is 1, 2, 3, 4, 5, 6, 7, 8, 9, or 10. Each occurrence of A, B, C, D, or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges.

In some embodiments, the peptidomimetic macrocycle comprises a secondary structure which is an α-helix and R₈ is —H, allowing intra-helical hydrogen bonding. In some embodiments, at least one of A, B, C, D, or E is an α,α-disubstituted amino acid. In one example, B is an α,α-disubstituted amino acid. For example, at least one of A, B, C, D, or E is 2-aminoisobutyric acid. In other embodiments, at least one of A, B, C, D, or E is

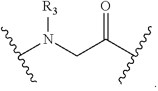

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

Exemplary embodiments of the macrocycle-forming linker —L₁-L₂- are shown below.

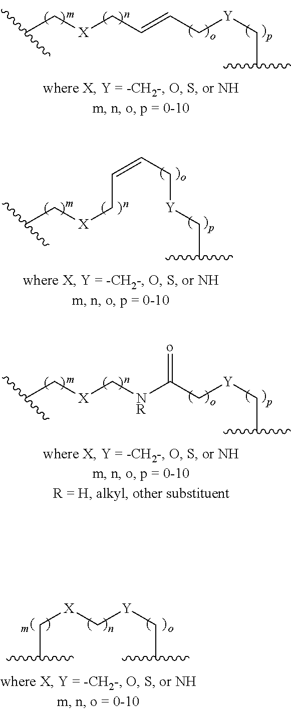

In some embodiments, the peptidomimetic macrocycle has the Formula (III) or Formula (IIIa):

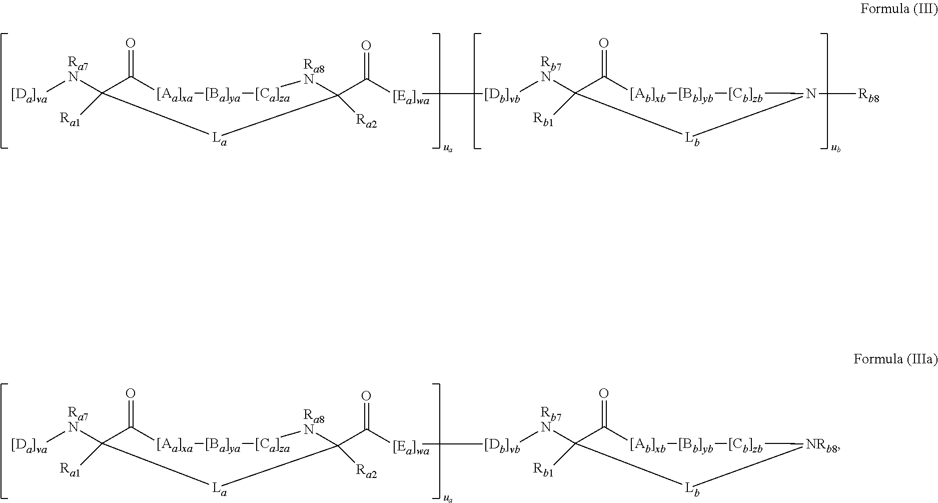

wherein:
each Aₐ, Cₐ, Dₐ, Eₐ, A_{b}, C_{b}, and D_{b} is independently a natural or non-natural amino acid or an amino acid analog;
each Bₐ and B_{b} is independently a natural or non-natural amino acid, amino acid analog,

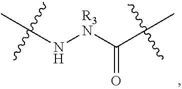

[—NH-L₄-CO—], [—NH-L₄-SO₂—], or [—NH-L₄-];
each Rₐ₁ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₁ forms a macrocycle-forming linker L′ connected to the alpha position of one of the Dₐ or Eₐ amino acids; or together with Lₐ forms a ring that is unsubstituted or substituted;
each Rₐ₂ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₂ forms a macrocycle-forming linker L′ connected to the alpha position of one of the Dₐ or Eₐ amino acids; or together with Lₐ forms a ring that is unsubstituted or substituted;
each R_{b1} is independently alkyl, alkenyl, alkynyl, arylalkyl, cycloalkyl, cycloalkylalkyl, heteroalkyl, or heterocycloalkyl, any of which is unsubstituted or substituted; or H; or R_{b1} forms a macrocycle-forming linker L′ connected to the alpha position of one of the D_{b} amino acids; or together with L_{b} forms a ring that is unsubstituted or substituted;
each R₃ is independently alkyl, alkenyl, alkynyl, arylalkyl, heteroalkyl, cycloalkyl, heterocycloalkyl, cycloalkylalkyl, cycloaryl, or heterocycloaryl, any of which is unsubstituted or substituted, or H;
each Lₐ is independently a macrocycle-forming linker, and optionally forms a ring with Rₐ₁ or Rₐ₂ that is unsubstituted or substituted;
each L_{b} is independently a macrocycle-forming linker, and optionally forms a ring with R_{b1} that is unsubstituted or substituted;
each L′ is independently a macrocycle-forming linker;
each L₄ is independently alkylene, alkenylene, alkynylene, heteroalkylene, cycloalkylene, heterocycloalkylene, cycloarylene, heterocycloarylene, or [—R₄—K—R₄—]ₙ, any of which is unsubstituted or substituted;
each R₄ is independently alkylene, alkenylene, alkynylene, heteroalkylene, cycloalkylene, heterocycloalkylene, arylene, or heteroarylene, any of which is unsubstituted or substituted;
each K is independently O, S, SO, SO₂, CO, CO₂, CO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
Rₐ₇ is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with a Dₐ amino acid;
R_{b7} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with a D_{b} amino acid;
Rₐ₈ is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with an Eₐ amino acid;
R_{b8} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or an amino acid sequence of 1-1000 amino acid residues;
each va and vb is independently an integer from 0-1000;
each wa and wb is independently an integer from 0-1000;
each ua and ub is independently 0, 1, 2, 3, 4, 5, 6, 7, 8, 9, or 10, wherein ua+ub is at least 1;
each xa and xb is independently 0, 1, 2, 3, 4, 5, 6, 7, 8, 9, or 10;
each ya and yb is independently 0, 1, 2, 3, 4, 5, 6, 7, 8, 9, or 10;
each za and zb is independently 0, 1, 2, 3, 4, 5, 6, 7, 8, 9, or 10; and
each n is independently 1, 2, 3, 4, or 5,
   or a pharmaceutically-acceptable salt thereof.

In some embodiments, the peptidomimetic macrocycle has the Formula (III) or Formula (IIIa):

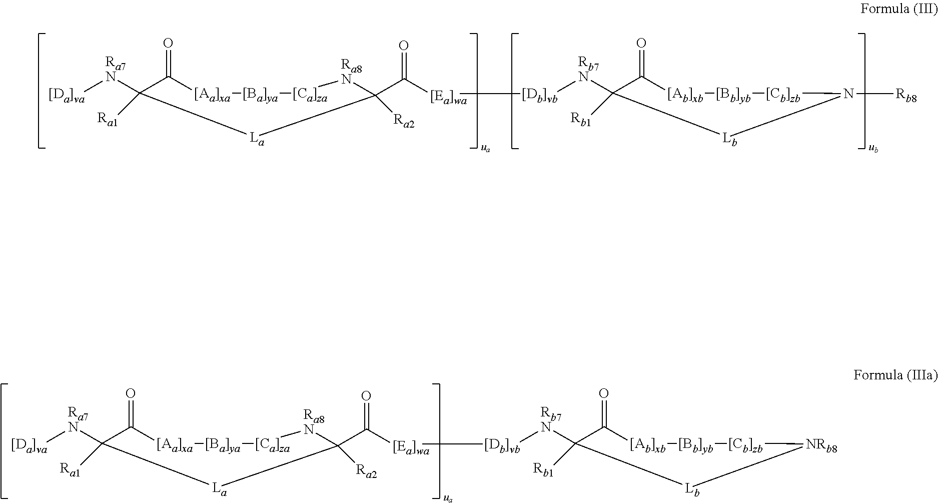

wherein:
each Aₐ, Cₐ, Dₐ, Eₐ, A_{b}, C_{b}, and D_{b} is independently a natural or non-natural amino acid or an amino acid analogue;
each Bₐ -and B_{b} is independently a natural or non-natural amino acid, amino acid analog,

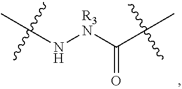

[—NH-L₄-CO—], [—NH-L₄-SO₂—], or [—NH-L₄-];
each Rₐ₁ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₁ forms a macrocycle-forming linker L′ connected to the alpha position of one of the Dₐ or Eₐ amino acids; or together with Lₐ forms a ring that is unsubstituted or substituted;
each Rₐ₂ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₂ forms a macrocycle-forming linker L′ connected to the alpha position of one of the Dₐ or Eₐ amino acids; or together with Lₐ forms a ring that is unsubstituted or substituted;
each R_{b1} is independently alkyl, alkenyl, alkynyl, arylalkyl, cycloalkyl, cycloalkylalkyl, heteroalkyl, or heterocycloalkyl, any of which is unsubstituted or substituted; or H; or R_{b1} forms a macrocycle-forming linker L′ connected to the alpha position of one of the D_{b} amino acids; or together with L_{b} forms a ring that is unsubstituted or substituted;
each R3 is independently alkyl, alkenyl, alkynyl, arylalkyl, heteroalkyl, cycloalkyl, heterocycloalkyl, cycloalkylalkyl, cycloaryl, or heterocycloaryl, any of which is unsubstituted or substituted with R₅, or H;
each Lₐ is independently a macrocycle-forming linker, and optionally forms a ring with Rₐ₁ or Rₐ₂ that is unsubstituted or substituted;
each L_{b} is independently a macrocycle-forming linker, and optionally forms a ring with R_{b1} that is unsubstituted or substituted;
each L′ is independently a macrocycle-forming linker;
each L₄ is independently alkylene, alkenylene, alkynylene, heteroalkylene, cycloalkylene, heterocycloalkylene, cycloarylene, heterocycloarylene, or [—R₄—K—R₄—]ₙ, any of which is unsubstituted or substituted with R₅;
each R₄ is independently alkylene, alkenylene, alkynylene, heteroalkylene, cycloalkylene, heterocycloalkylene, arylene, or heteroarylene, any of which is unsubstituted or substituted with R₅;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently H, alkyl, alkenyl, alkynyl, arylalkyl, cycloalkylalkyl, heterocycloalkyl, a fluorescent moiety, a radioisotope, or a therapeutic agent;
each Rₐ₇ is independently alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with a Dₐ amino acid;
R_{b7} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with a D_{b} amino acid;
each Rₐ₈ is independently alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with an Eₐ amino acid;
R_{b8} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or an amino acid sequence of 1-1000 amino acid residues;
each va and vb is independently an integer from 0-1000;
each wa and wb is independently an integer from 0-1000;
each ua and ub is independently 0, 1, 2, 3, 4, 5, 6, 7, 8, 9, or 10, wherein ua+ub is at least 1;
each xa and xb is independently 0, 1, 2, 3, 4, 5, 6, 7, 8, 9, or 10;
each ya and yb is independently 0, 1, 2, 3, 4, 5, 6, 7, 8, 9, or 10;
each za and zb is independently 0, 1, 2, 3, 4, 5, 6, 7, 8, 9, or 10; and
each n is independently 1, 2, 3, 4, or 5,
   or a pharmaceutically-acceptable salt thereof.

In some embodiments, the peptidomimetic macrocycle of the invention has the formula defined above, wherein:
each Lₐ is independently a macrocycle-forming linker of the formula -L₁-L₂-, and optionally forms a ring with Rₐ₁ or Rₐ₂ that is unsubstituted or substituted;
each L_{b} is independently a macrocycle-forming linker of the formula -L₁-L₂-, and optionally forms a ring with R_{b1} that is unsubstituted or substituted;
each L′ is independently a macrocycle-forming linker of the formula -L₁-L₂-;
each L₁ and L₂ is independently alkylene, alkenylene, alkynylene, heteroalkylene, cycloalkylene, heterocycloalkylene, cycloarylene, heterocycloarylene, or [—R₄—K—R₄—]ₙ, any of which is unsubstituted or substituted with R₅;
each R₄ is independently alkylene, alkenylene, alkynylene, heteroalkylene, cycloalkylene, heterocycloalkylene, arylene, or heteroarylene, any of which is unsubstituted or substituted with R₅;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent; and
each R₆ is independently H, alkyl, alkenyl, alkynyl, arylalkyl, cycloalkylalkyl, heterocycloalkyl, a fluorescent moiety, a radioisotope, or a therapeutic agent,
   or a pharmaceutically-acceptable salt thereof.

In some embodiments, the peptidomimetic macrocycle has the formula defined above wherein one of Lₐ and L_{b} is a bis-thioether-containing macrocycle-forming linker. In some embodiments, one of Lₐ and L_{b} is a macrocycle-forming linker of the formula -L₁-S-L₂-S-L₃-.

In some embodiments, the peptidomimetic macrocycle has the formula defined above wherein one of Lₐ and L_{b} is a bis-sulfone-containing macrocycle-forming linker. In some embodiments, one of Lₐ and L_{b} is a macrocycle-forming linker of the formula -L₁-SO₂-L₂-SO₂-L₃-.

In some embodiments, the peptidomimetic macrocycle has the formula defined above wherein one of Lₐ and L_{b} is a bis-sulfoxide-containing macrocycle-forming linker. In some embodiments, one of Lₐ and L_{b} is a macrocycle-forming linker of the formula -L₁-S(O)-L₂-S(O)-L₃-.

In some embodiments, a peptidomimetic macrocycle of the invention comprises one or more secondary structures. In some embodiments, the peptidomimetic macrocycle comprises a secondary structure that is an α-helix. In some embodiments, the peptidomimetic macrocycle comprises a secondary structure that is a β-hairpin turn.

In some embodiments, uₐ is 0. In some embodiments, uₐ is 0, and L_{b} is a macrocycle-forming linker that crosslinks an α-helical secondary structure. In some embodiments, uₐ is 0, and L_{b} is a macrocycle-forming linker that crosslinks a β-hairpin secondary structure. In some embodiments, uₐ is 0, and L_{b} is a hydrocarbon-containing macrocycle-forming linker that crosslinks an α-helical secondary structure. In some embodiments, uₐ is 0, and L_{b} is a hydrocarbon-containing macrocycle-forming linker that crosslinks a β-hairpin secondary structure.

In some embodiments, u_{b} is 0. In some embodiments, u_{b} is 0, and Lₐ is a macrocycle-forming linker that crosslinks an α-helical secondary structure. In some embodiments, u_{b} is 0, and Lₐ is a macrocycle-forming linker that crosslinks a β-hairpin secondary structure. In some embodiments, u_{b} is 0, and Lₐ is a hydrocarbon-containing macrocycle-forming linker that crosslinks an α-helical secondary structure. In some embodiments, u_{b} is 0, and Lₐ is a hydrocarbon-containing macrocycle-forming linker that crosslinks a β-hairpin secondary structure.

In some embodiments, the peptidomimetic macrocycle comprises only α-helical secondary structures. In other embodiments, the peptidomimetic macrocycle comprises only β-hairpin secondary structures.

In other embodiments, the peptidomimetic macrocycle comprises a combination of secondary structures, wherein the secondary structures are α-helical and β-hairpin structures. In some embodiments, Lₐ and L_{b} are a combination of hydrocarbon-, triazole, or sulfur-containing macrocycle-forming linkers. In some embodiments, the peptidomimetic macrocycle comprises Lₐ and L_{b}, wherein Lₐ is a hydrocarbon-containing macrocycle-forming linker that crosslinks a β-hairpin structure, and L_{b} is a triazole-containing macrocycle-forming linker that crosslinks an α-helical structure. In some embodiments, the peptidomimetic macrocycle comprises Lₐ and L_{b}, wherein Lₐ is a hydrocarbon-containing macrocycle-forming linker that crosslinks an α-helical structure, and L_{b} is a triazole-containing macrocycle-forming linker that crosslinks a β-hairpin structure. In some embodiments, the peptidomimetic macrocycle comprises Lₐ and L_{b}, wherein Lₐ is a triazole-containing macrocycle-forming linker that crosslinks an α-helical structure, and L_{b} is a hydrocarbon-containing macrocycle-forming linker that crosslinks a β-hairpin structure. In some embodiments, the peptidomimetic macrocycle comprises Lₐ and L_{b}, wherein Lₐ is a triazole-containing macrocycle-forming linker that crosslinks a β-hairpin structure, and L_{b} is a hydrocarbon-containing macrocycle-forming linker that crosslinks an α-helical structure.

In some embodiments, uₐ+u_{b} is at least 1. In some embodiments, uₐ+u_{b}=2.

In some embodiments, uₐ is 1, u_{b} is 1, Lₐ is a triazole-containing macrocycle-forming linker that crosslinks an α-helical secondary structure, and L_{b} is a hydrocarbon-containing macrocycle-forming linker that crosslinks an α-helical structure. In some embodiments, uₐ is 1, u_{b} is 1, Lₐ is a triazole-containing macrocycle-forming linker that crosslinks an α-helical secondary structure, and L_{b} is a hydrocarbon-containing macrocycle-forming linker that crosslinks a β-hairpin structure. In some embodiments, uₐ is 1, u_{b} is 1, Lₐ is a triazole-containing macrocycle-forming linker that crosslinks a β-hairpin secondary structure, and L_{b} is a hydrocarbon-containing macrocycle-forming linker that crosslinks an α-helical structure. In some embodiments, uₐ is 1, u_{b} is 1, Lₐ is a triazole-containing macrocycle-forming linker that crosslinks a β-hairpin secondary structure, and L_{b} is a hydrocarbon-containing macrocycle-forming linker that crosslinks a β-hairpin structure.

In some embodiments, uₐ is 1, u_{b} is 1, Lₐ is a hydrocarbon-containing macrocycle-forming linker that crosslinks an α-helical secondary structure, and L_{b} is a triazole-containing macrocycle-forming linker that crosslinks an α-helical secondary structure. In some embodiments, uₐ is 1, u_{b} is 1, Lₐ is a hydrocarbon-containing macrocycle-forming linker that crosslinks an α-helical secondary structure, and L_{b} is a triazole-containing macrocycle-forming linker that crosslinks a β-hairpin secondary structure. In some embodiments, uₐ is 1, u_{b} is 1, Lₐ is a hydrocarbon-containing macrocycle-forming linker that crosslinks a β-hairpin secondary structure, and L_{b} is a triazole-containing macrocycle-forming linker that crosslinks an α-helical secondary structure. In some embodiments, uₐ is 1, u_{b} is 1, Lₐ is a hydrocarbon-containing macrocycle-forming linker that crosslinks a β-hairpin secondary structure, and L_{b} is a triazole-containing macrocycle-forming linker that crosslinks a β-hairpin secondary structure.

In some embodiments, uₐ is 1, u_{b} is 1, Lₐ is a hydrocarbon-containing macrocycle-forming linker with an α-helical secondary structure, and L_{b} is a sulfur-containing macrocycle-forming linker. In some embodiments, uₐ is 1, u_{b} is 1, Lₐ is a hydrocarbon-containing macrocycle-forming linker with a β-hairpin secondary structure, and L_{b} is a sulfur-containing macrocycle-forming linker.

In some embodiments, uₐ is 1, u_{b} is 1, Lₐ is a sulfur-containing macrocycle-forming linker, and L_{b} is a hydrocarbon-containing macrocycle-forming linker with an a-helical secondary structure. In some embodiments, uₐ is 1, u_{b} is 1, Lₐ is a sulfur-containing macrocycle-forming linker, and L_{b} is a hydrocarbon-containing macrocycle-forming linker with a β-hairpin secondary structure.

In some embodiments, uₐ is 1, u_{b} is 1, Lₐ is a hydrocarbon-containing macrocycle-forming linker that crosslinks an α-helical structure, and L_{b} is a hydrocarbon-containing macrocycle-forming linker that crosslinks an α-helical structure. In some embodiments, uₐ is 1, u_{b} is 1, Lₐ is a hydrocarbon-containing macrocycle-forming linker that crosslinks an α-helical structure, and L_{b} is a hydrocarbon-containing macrocycle-forming linker that crosslinks a β-hairpin structure. In some embodiments, uₐ is 1, u_{b} is 1, Lₐ is a hydrocarbon-containing macrocycle-forming linker that crosslinks a β-hairpin structure, and L_{b} is a hydrocarbon-containing macrocycle-forming linker that crosslinks an α-helical structure. In some embodiments, uₐ is 1, u_{b} is 1, Lₐ is a hydrocarbon-containing macrocycle-forming linker that crosslinks a β-hairpin structure, and L_{b} is a hydrocarbon-containing macrocycle-forming linker that crosslinks a β-hairpin structure.

In some embodiments, R_{b1} is H.

Unless otherwise stated, any compounds (including peptidomimetic macrocycles, macrocycle precursors, and other compositions) are also meant to encompass compounds which differ only in the presence of one or more isotopically enriched atoms. For example, compounds having the described structures except for the replacement of a hydrogen atom by deuterium or tritium, or the replacement of a carbon atom by ¹³C or ¹⁴C are contemplated.

In some embodiments, the compounds disclosed herein can contain unnatural proportions of atomic isotopes at one or more of atoms that constitute such compounds. For example, the compounds can be radiolabeled with radioactive isotopes, such as for example tritium (³H), iodine-125 (¹²⁵I) or carbon-14 (¹⁴C). In other embodiments, one or more carbon atoms is replaced with a silicon atom. All isotopic variations of the compounds disclosed herein, whether radioactive or not, are contemplated herein.

In some embodiments, the peptidomimetic macrocycle comprises an amino acid sequence that is at least 60%, at least 65%, at least 70%, at least 75%, at least 80%, at least 85%, at least 90%, or at least 95% identical to an amino acid sequence listed in TABLE 1, TABLE 1a, TABLE 1b, TABLE 1c, TABLE 2a, or TABLE 2b. In some embodiments, the peptidomimetic macrocycle comprises an amino acid sequence that is at least 60% identical to an amino acid sequence listed in TABLE 1, TABLE 1a, TABLE 1b, TABLE 1c, TABLE 2a, or TABLE 2b. In some embodiments, the peptidomimetic macrocycle comprises an amino acid sequence that is at least 65% identical to an amino acid sequence listed in TABLE 1, TABLE 1a, TABLE 1b, TABLE 1c, TABLE 2a, or TABLE 2b. In some embodiments, the peptidomimetic macrocycle comprises an amino acid sequence that is at least 70% identical to an amino acid sequence listed in TABLE 1, TABLE 1a, TABLE 1b, TABLE 1c, TABLE 2a, or TABLE 2b. In some embodiments, the peptidomimetic macrocycle comprises an amino acid sequence that is at least 75% identical to an amino acid sequence listed in TABLE 1, TABLE 1a, TABLE 1b, TABLE 1c, TABLE 2a, or TABLE 2b.

In some embodiments, the peptidomimetic macrocycle is at least 60%, at least 65%, at least 70%, at least 75%, at least 80%, at least 85%, at least 90%, or at least 95% identical to an amino acid sequence listed in TABLE 1, TABLE 1a, TABLE 1b, TABLE 1c, TABLE 2a, or TABLE 2b. In some embodiments, the peptidomimetic macrocycle is at least 60% identical to an amino acid sequence listed in TABLE 1, TABLE 1a, TABLE 1b, TABLE 1c, TABLE 2a, or Table 2b. In some embodiments, the peptidomimetic macrocycle is at least 65% identical to an amino acid sequence listed in TABLE 1, TABLE 1a, TABLE 1b, TABLE 1c, TABLE 2a, or TABLE 2b. In some embodiments, the peptidomimetic macrocycle is at least 70% identical to an amino acid sequence listed in TABLE 1, TABLE 1a, TABLE 1b, TABLE 1c, TABLE 2a, or TABLE 2b. In some embodiments, the peptidomimetic macrocycle is at least 75% identical to an amino acid sequence listed in TABLE 1, TABLE 1a, TABLE 1b, TABLE 1c, TABLE 2a, or TABLE 2b.

### Preparation of Peptidomimetic Macrocycles

Peptidomimetic macrocycles can be prepared by any of a variety of methods known in the art. For example, any of the residues indicated by “$” or “$r8” in TABLE 1, TABLE 1a, TABLE 1b, TABLE 1c, TABLE 2a, or TABLE 2b can be substituted with a residue capable of forming a crosslinker with a second residue in the same molecule or a precursor of such a residue.

α,α-Disubstituted amino acids and amino acid precursors can be employed in synthesis of the peptidomimetic macrocycle precursor polypeptides. For example, the “S5-olefin amino acid” is (S)-α-(2′-pentenyl) alanine and the “R8 olefin amino acid” is (R)-α-(2′-octenyl) alanine. Following incorporation of such amino acids into precursor polypeptides, the terminal olefins are reacted with a metathesis catalyst, leading to the formation of the peptidomimetic macrocycle. In various embodiments, the following amino acids can be employed in the synthesis of the peptidomimetic macrocycle:

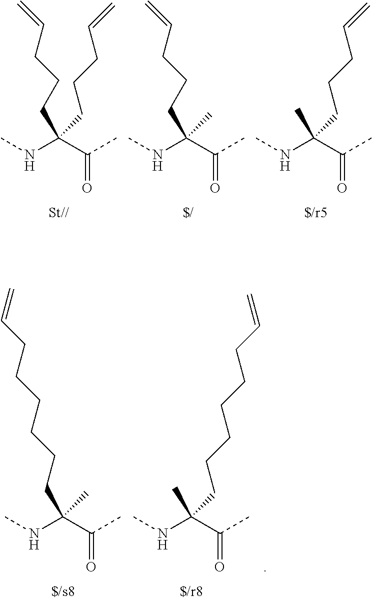

In other embodiments, the peptidomimetic macrocycles are of Formula IV or IVa. In such embodiments, amino acid precursors are used containing an additional substituent R— at the alpha position. Such amino acids are incorporated into the macrocycle precursor at the desired positions, which can be at the positions where the crosslinker is substituted or, alternatively, elsewhere in the sequence of the macrocycle precursor. Cyclization of the precursor is then effected according to the indicated method.

### Pharmaceutically-Acceptable Salts

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt , or a maleate salt.

### Purity of Compounds of the Invention

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Formulation and Administration

### Pharmaceutical Compositions

Pharmaceutical compositions disclosed herein include peptidomimetic macrocycles and pharmaceutically-acceptable derivatives or prodrugs thereof. A “pharmaceutically-acceptable derivative” means any pharmaceutically-acceptable salt, ester, salt of an ester, pro-drug or other derivative of a compound disclosed herein which, upon administration to a recipient, is capable of providing (directly or indirectly) a compound disclosed herein. Particularly favored pharmaceutically-acceptable derivatives are those that increase the bioavailability of the compounds when administered to a mammal (e.g., by increasing absorption into the blood of an orally administered compound) or which increases delivery of the active compound to a biological compartment (e.g., the brain or lymphatic system) relative to the parent species. Some pharmaceutically-acceptable derivatives include a chemical group which increases aqueous solubility or active transport across the gastrointestinal mucosa.

In some embodiments, peptidomimetic macrocycles are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow administration by injection, alter metabolism, and alter rate of excretion.

For preparing pharmaceutical compositions from the compounds disclosed herein, pharmaceutically-acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the crosslinked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation can be in unit dosage form. In such form the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

When one or more compositions disclosed herein comprise a combination of a peptidomimetic macrocycle and one or more additional therapeutic or prophylactic agents, both the compound and the additional agent are present at dosage levels of between about 1 to 100%, and more preferably between about 5 to 95% of the dosage normally administered in a monotherapy regimen. In some embodiments, the additional agents are administered separately, as part of a multiple dose regimen, from one or more compounds disclosed herein. Alternatively, those agents are part of a single dosage form, mixed together with the compounds disclosed herein in a single composition.

### Mode of Administration

An effective amount of a peptidomimetic macrocycles of the disclosure can be administered in either single or multiple doses by any of the accepted modes of administration. In some embodiments, the peptidomimetic macrocycles of the disclosure are administered parenterally, for example, by subcutaneous, intramuscular, intrathecal, intravenous or epidural injection. For example, the peptidomimetic macrocycle is administered intravenously, intra-arterially, subcutaneously or by infusion. In some examples, the peptidomimetic macrocycle is administered intravenously. In some examples, the peptidomimetic macrocycle is administered intra-arterially.

Regardless of the route of administration selected, the peptidomimetic macrocycles of the present disclosure, and/or the pharmaceutical compositions of the present disclosure, are formulated into pharmaceutically-acceptable dosage forms. The peptidomimetic macrocycles according to the disclosure can be formulated for administration in any convenient way for use in human or veterinary medicine, by analogy with other pharmaceuticals.

In one aspect, the disclosure provides pharmaceutical formulation comprising a therapeutically-effective amount of one or more of the peptidomimetic macrocycles described above, formulated together with one or more pharmaceutically-acceptable carriers (additives) and/or diluents. In one embodiment, one or more of the peptidomimetic macrocycles described herein are formulated for parenteral administration for parenteral administration, one or more peptidomimetic macrocycles disclosed herein can be formulated as aqueous or non-aqueous solutions, dispersions, suspensions or emulsions or sterile powders which can be reconstituted into sterile injectable solutions or dispersions just prior to use. Such formulations can comprise sugars, alcohols, antioxidants, buffers, bacteriostats, solutes which render the formulation isotonic with the blood of the intended recipient or suspending or thickening agents. These compositions can also contain adjuvants such as preservatives, wetting agents, emulsifying agents and dispersing agents. Prevention of the action of microorganisms upon the subject compounds can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin. If desired the formulation can be diluted prior to use with, for example, an isotonic saline solution or a dextrose solution. In some examples, the peptidomimetic macrocycle is formulated as an aqueous solution and is administered intravenously.

### Amount and Frequency of Administration

Dosing can be determined using various techniques. The selected dosage level can depend upon a variety of factors including the activity of the particular peptidomimetic macrocycle employed, the route of administration, the time of administration, the rate of excretion or metabolism of the particular peptidomimetic macrocycle being employed, the duration of the treatment, other drugs, compounds and/or materials used in combination with the particular peptidomimetic macrocycle employed, the age, sex, weight, condition, general health and prior medical history of the patient being treated, and like factors well known in the medical arts. The dosage values can also vary with the severity of the condition to be alleviated. For any particular subject, specific dosage regimens can be adjusted over time according to the individual need and the professional judgment of the person administering or supervising the administration of the compositions.

A physician or veterinarian can prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In some embodiments, a suitable daily dose of a peptidomimetic macrocycle of the disclosure can be that amount of the peptidomimetic macrocycle which is the lowest dose effective to produce a therapeutic effect. Such an effective dose will generally depend upon the factors described above. The precise time of administration and amount of any particular peptidomimetic macrocycle that will yield the most effective treatment in a given patient will depend upon the activity, pharmacokinetics, and bioavailability of a particular peptidomimetic macrocycle, physiological condition of the patient (including age, sex, disease type and stage, general physical condition, responsiveness to a given dosage and type of medication), route of administration, and the like.

Dosage can be based on the amount of the peptidomimetic macrocycle per kg body weight of the patient. Alternatively, the dosage of the subject disclosure can be determined by reference to the plasma concentrations of the peptidomimetic macrocycle. For example, the maximum plasma concentration (Cmax) and the area under the plasma concentration-time curve from time 0 to infinity (AUC) can be used.

The amount of the peptidomimetic macrocycle that is administered to a subject can be from about 1 μg/kg, 25 μg/kg, 50 μg/kg, 75 μg/kg, 100 μμg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg per body weight of the subject.

The amount of the peptidomimetic macrocycle that is administered to a subject can be from about 0.01 mg/kg to about 100 mg/kg body weight of the subject. In some embodiments, the amount of the peptidomimetic macrocycle administered is about 0.01-10 mg/kg, about 0.01-20 mg/kg, about 0.01-50 mg/kg, about 0.1-10 mg/kg, about 0.1-20 mg/kg, about 0.1-50 mg/kg, about 0.1-100 mg/kg, about 0.5-10 mg/kg, about 0.5-20 mg/kg, about 0.5-50 mg/kg, about 0.5-100 mg/kg, about 1-10 mg/kg, about 1-20 mg/kg, about 1-50 mg/kg, or about 1-100 mg/kg body weight of the human subject. In some embodiments, the amount of the peptidomimetic macrocycle administered is about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg body weight of the subject. In some embodiments, the amount of the peptidomimetic macrocycle administered is about 5 mg/kg. In some embodiments, the amount of the peptidomimetic macrocycle administered is about 10 mg/kg. In some embodiments, the amount of the peptidomimetic macrocycle administered is about 15 mg/kg.

In some embodiments, the amount of the peptidomimetic macrocycle administered is about 0.16 mg, about 0.32 mg, about 0.64 mg, about 1.28 mg, about 3.56 mg, about 7.12 mg, about 14.24 mg, or about 20 mg per kilogram body weight of the subject. In some examples the amount of the peptidomimetic macrocycle administered is about 0.16 mg per kilogram body weight of the subject. In some examples the amount of the peptidomimetic macrocycle administered is about 0.32 mg per kilogram body weight of the subject. In some examples the amount of the peptidomimetic macrocycle administered is about 0.64 mg per kilogram body weight of the subject. In some examples the amount of the peptidomimetic macrocycle administered is about 1.28 mg per kilogram body weight of the subject. In some examples the amount of the peptidomimetic macrocycle administered is about 3.56 mg per kilogram body weight of the subject. In some examples the amount of the peptidomimetic macrocycle administered is about 7.12 mg per kilogram body weight of the subject. In some examples the amount of the peptidomimetic macrocycle administered is about 14.24 mg per kilogram body weight of the subject.

In some embodiments, a pharmaceutically-acceptable amount of a peptidomimetic macrocycle is administered to a subject 1, 2, 3, 4, 5, 6, 7, 8, 9, 10, 11, 12, 13, or 14 times a week. In some embodiments about 0.5-about 20 mg or about 0.5-about 10 mg of the peptidomimetic macrocycle per kilogram body weight of the human subject is administered once a week. For example about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered once a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered once a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once a week. In some examples, the amount of the peptidomimetic macrocycle administered is about 1.25 mg, about 2.5 mg, about 5 mg or about 10 mg per kilogram body weight of the human subject and the peptidomimetic macrocycle is administered once a week.

In some embodiments about 0.5-about 20 mg or about 0.5-about 10 mg of the peptidomimetic macrocycle per kilogram body weight of the human subject is administered two times a week. For example about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered about twice a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered two times a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered two times a week. In some examples, the amount of the peptidomimetic macrocycle administered is about 1.25 mg, about 2.5 mg, about 5 mg or about 10 mg per kilogram body weight of the human subject and the peptidomimetic macrocycle is administered two times a week.

In some embodiments about 0.5-about 20 mg or about 0.5-about 10 mg of the peptidomimetic macrocycle per kilogram body weight of the human subject is administered 3, 4, 5, 6, or 7 times a week. For example, about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered 3, 4, 5, 6, or 7 times a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered 3, 4, 5, 6, or 7 times a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered 3, 4, 5, 6, or 7 times a week. In some examples, the amount of the peptidomimetic macrocycle administered is about 1.25 mg, about 2.5 mg, about 5 mg, or about 10 mg per kilogram body weight of the human subject and the peptidomimetic macrocycle is administered 3, 4, 5, 6, or 7 times a week.

In some embodiments, a pharmaceutically-acceptable amount of a peptidomimetic macrocycle is administered to a subject once every 1, 2, 3, 4, 5, 6, 7, or 8 weeks. In some embodiments, about 0.5-about 20 mg or about 0.5-about 10 mg of the peptidomimetic macrocycle per kilogram body weight of the human subject is administered once every 2, 3, or 4 weeks. For example, about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administrated 3, 4, 5, 6, or 7 once every 2 or 3 weeks. In some examples, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered once every 2 or 3 weeks. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once every 2 weeks. In some examples, the amount of the peptidomimetic macrocycle administered is about 1.25 mg, about 2.5 mg, about 5 mg or about 10 mg per kilogram body weight of the human subject and the peptidomimetic macrocycle is administered once every 2 weeks. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once every 3 weeks. In some examples, the amount of the peptidomimetic macrocycle administered is about 1.25 mg, about 2.5 mg, about 5 mg, or about 10 mg per kilogram body weight of the human subject and the peptidomimetic macrocycle is administered once every 3 weeks.

In some embodiments, a pharmaceutically-acceptable amount of a peptidomimetic macrocycle is administered to a subject gradually over a period of time. In some embodiments, an amount of a peptidomimetic macrocycle can be administered to a subject gradually over a period of from about 0.1 h to about 24 h. In some embodiments, an amount of a peptidomimetic macrocycle can be administered to a subject over a period of about 0.1 h, about 0.2 h, about 0.3 h, about 0.4 h, about 0.5 h, about 0.6 h, about 0.7 h, about 0.8 h, about 0.9 h, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h, about 10 h, about 10.5 h, about 11 h, about 11.5 h, about 12 h, about 12.5 h, about 13 h, about 13.5 h, about 14 h, about 14.5 h, about 15 h, about 15.5 h, about 16 h, about 16.5 h, about 17 h, about 17.5 h, about 18 h, about 18.5 h, about 19 h, about 19.5 h, about 20 h, about 20.5 h, about 21 h, about 21.5 h, about 22 h, about 22.5 h, about 23 h, about 23.5 h, or about 24 h. In some embodiments, a pharmaceutically-acceptable amount of a peptidomimetic macrocycle is administered gradually over a period of about 0.5 h. In some embodiments, a pharmaceutically-acceptable amount of a peptidomimetic macrocycle is administered gradually over a period of about 1 h. In some embodiments, a pharmaceutically-acceptable amount of a peptidomimetic macrocycle is administered gradually over a period of about 1.5 h.

Administration of the peptidomimetic macrocycles can continue for as long as clinically necessary. In some embodiments, a peptidomimetic macrocycle of the disclosure can be administered for more than 1 day, more than 1 week, more than 1 month, more than 2 months, more than 3 months, more than 4 months, more than 5 months, more than 6 months, more than 7 months, more than 8 months, more than 9 months, more than 10 months, more than 11 months, more than 12 months, more than 13 months, more than 14 months, more than 15 months, more than 16 months, more than 17 months, more than 18 months, more than 19 months, more than 20 months, more than 21 months, more than 22 months, more than 23 months, or more than 24 months. In some embodiments, one or more peptidomimetic macrocycle of the disclosure is administered for less than 1 week, less than 1 month, less than 2 months, less than 3 months, less than 4 months, less than 5 months, less than 6 months, less than 7 months, less than 8 months, less than 9 months, less than 10 months, less than 11 months, less than 12 months, less than 13 months, less than 14 months, less than 15 months, less than 16 months, less than 17 months, less than 18 months, less than 19 months, less than 20 months, less than 21 months, less than 22 months, less than 23 months, or less than 24 months.

In some embodiments, a peptidomimetic macrocycle can be administered to a subject 1, 2, 3, 4, 5, 6, 7, 8, 9, 10, 11, 12, 13, 14, 15, 16, 17, 18, 19, or 20 times over a treatment cycle. In some embodiments a peptidomimetic macrocycle can be administered to a subject 2, 4, 6, or 8 times over a treatment cycle. In some embodiments, a peptidomimetic macrocycle can be administered to a subject 4 times over a treatment cycle. In some embodiments, a treatment cycle is 7 days, 14 days, 21 days, or 28 days long. In some embodiments, a treatment cycle is 21 days long. In some embodiments, a treatment cycle is 28 days long.

In some embodiments, a peptidomimetic macrocycle is administered on day 1, 8, 15 and 28 of a 28-day cycle. In some embodiments, the peptidomimetic macrocycle is administered on day 1, 8, 15 and 28 of a 28-day cycle and administration is continued for two cycles. In some embodiments, the peptidomimetic macrocycle is administered on day 1, 8, 15 and 28 of a 28-day cycle and administration is continued for three cycles. In some embodiments, the peptidomimetic macrocycle is administered on day 1, 8, 15 and 28 of a 28-day cycle and administration is continued for 4, 5, 6, 7, 8, 9, 10, or more than 10 cycles.

In some embodiments, the peptidomimetic macrocycle is administered on day 1, 8, 11 and 21 of a 21-day cycle. In some embodiments, the peptidomimetic macrocycle is administered on day 1, 8, 11 and 21 of a 21-day cycle and administration is continued for two cycles. In some embodiments, the peptidomimetic macrocycle is administered on day 1, 8, 11 and 21 of a 21-day cycle and administration is continued for three cycles. In some embodiments, the peptidomimetic macrocycle is administered on day 1, 8, 11 and 21 of a 21-day cycle and administration is continued for 4, 5, 6, 7, 8, 9, 10, or more than 10 cycles.

In some embodiments, one or more peptidomimetic macrocycle of the disclosure is administered chronically on an ongoing basis. In some embodiments, administration of one or more peptidomimetic macrocycle of the disclosure is continued until documentation of disease progression, unacceptable toxicity, or patient or physician decision to discontinue administration.

In some embodiments, the compounds of the invention can be used to treat one condition. In some embodiments, the compounds of the invention can be used to treat two conditions. In some embodiments, the compounds of the invention can be used to treat three conditions. In some embodiments, the compounds of the invention can be used to treat four conditions. In some embodiments, the compounds of the invention can be used to treat five conditions.

### Methods of Use

In one aspect, provided herein are novel peptidomimetic macrocycles that are useful in competitive binding assays to identify agents which bind to the natural ligand(s) of the proteins or peptides upon which the peptidomimetic macrocycles are modeled. For example, in the p53/MDMX system, labeled peptidomimetic macrocycles based on p53 can be used in a MDMX binding assay along with small molecules that competitively bind to MDMX. Competitive binding studies allow for rapid in vitro evaluation and determination of drug candidates specific for the p53/MDMX system. Such binding studies can be performed with any of the peptidomimetic macrocycles disclosed herein and their binding partners. Further provided are methods for the generation of antibodies against the peptidomimetic macrocycles. In some embodiments, these antibodies specifically bind both the peptidomimetic macrocycle and the precursor peptides, such as p53, to which the peptidomimetic macrocycles are related. Such antibodies, for example, disrupt the native protein-protein interaction, for example, binding between p53 and MDMX.

In other aspects, provided herein are both prophylactic and therapeutic methods of treating a subject at risk of (or susceptible to) a disorder or having a disorder associated with aberrant (e.g., insufficient or excessive) expression or activity of the molecules including p53, MDM2 or MDMX.

In another embodiment, a disorder is caused, at least in part, by an abnormal level of p53 or MDM2 or MDMX, (e.g., over or under expression), or by the presence of p53 or MDM2 or MDMX exhibiting abnormal activity. As such, the reduction in the level and/or activity of p53 or MDM2 or MDMX, or the enhancement of the level and/or activity of p53 or MDM2 or MDMX, by peptidomimetic macrocycles derived from p53, is used, for example, to ameliorate or reduce the adverse symptoms of the disorder.

In another aspect, provided herein are methods for treating or preventing a disease including hyperproliferative disease and inflammatory disorder by interfering with the interaction or binding between binding partners, for example, between p53 and MDM2 or p53 and MDMX. These methods comprise administering an effective amount of a compound to a warm blooded animal, including a human. In some embodiments, the administration of one or more compounds disclosed herein induces cell growth arrest or apoptosis.

In some embodiments, the peptidomimetic macrocycles can be used to treat, prevent, and/or diagnose cancers and neoplastic conditions. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states can be categorized as pathologic, i.e., characterizing or constituting a disease state, or can be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiation disorders include cancer, e.g., carcinoma, sarcoma, or metastatic disorders. In some embodiments, the peptidomimetic macrocycles are novel therapeutic agents for controlling breast cancer, ovarian cancer, colon cancer, lung cancer, metastasis of such cancers and the like.

Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

In some embodiments, the cancer is head and neck cancer, melanoma, lung cancer, breast cancer, or glioma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. The diseases can arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CIVIL); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), periphieral T-cell lymphoma (PTCL), large granular lymphocytic leukemia (LGF), Hodgkin's disease and Reed-Stemberg disease.

Examples of cellular proliferative and/or differentiation disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the skin include, but are not limited to proliferative skin disease such as melanomas, including mucosal melanoma, superficial spreading melanoma, nodular melanoma, lentigo (e.g. lentigo maligna, lentigo maligna melanoma, or acral lentiginous melanoma), amelanotic melanoma, desmoplastic melanoma, melanoma with features of a Spitz nevus, melanoma with small nevus-like cells, polypoid melanoma, and soft-tissue melanoma; basal cell carcinomas including micronodular basal cell carcinoma, superficial basal cell carcinoma, nodular basal cell carcinoma (rodent ulcer), cystic basal cell carcinoma, cicatricial basal cell carcinoma, pigmented basal cell carcinoma, aberrant basal cell carcinoma, infiltrative basal cell carcinoma, nevoid basal cell carcinoma syndrome, polypoid basal cell carcinoma, pore-like basal cell carcinoma, and fibroepithelioma of Pinkus; squamus cell carcinomas including acanthoma (large cell acanthoma), adenoid squamous cell carcinoma, basaloid squamous cell carcinoma, clear cell squamous cell carcinoma, signet-ring cell squamous cell carcinoma, spindle cell squamous cell carcinoma, Marjolin's ulcer, erythroplasia of Queyrat, and Bowen's disease; or other skin or subcutaneous tumors.

Examples of cellular proliferative and/or differentiation disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

### Combination Treatment

Combination therapy with a peptidomimetic macrocycle of the disclosure and at least one additional therapeutic agent, for example, paclitaxel. In some embodiments, the combination therapy can produce a significantly better therapeutic result than the additive effects achieved by each individual constituent when administered alone at a therapeutic dose. In some embodiments, the dosage of the peptidomimetic macrocycle or additional therapeutic agent in combination therapy can be reduced as compared to monotherapy with each agent, while still achieving an overall therapeutic effect. In some embodiments, a peptidomimetic macrocycle and an additional therapeutic agent can exhibit a synergistic effect. In some embodiments, the synergistic effect of a peptidomimetic macrocycle and additional therapeutic agent can be used to reduce the total amount drugs administered to a subject, which decrease side effects experienced by the subject.

In some embodiments, the at least one additional pharmaceutically-active agent, for example, paclitaxel, can modulate the same or a different target as the peptidomimetic macrocycles of the disclosure. In some embodiments, the at least one additional pharmaceutically-active agent can modulate the same target as the peptidomimetic macrocycles of the disclosure, or other components of the same pathway, or overlapping sets of target enzymes. In some embodiments, the at least one additional pharmaceutically-active agent can modulate a different target from the peptidomimetic macrocycles of the disclosure.

Accordingly, in one aspect, the present disclosure provides a method for treating cancer, the method comprising administering to a subject in need thereof (a) an effective amount of a peptidomimetic macrocycle of the disclosure; and (b) an effective amount of at least one additional pharmaceutically active agent, for example, paclitaxel, to provide a combination therapy. In some embodiments, the combination therapy may have an enhanced therapeutic effect compared to the effect of the peptidomimetic macrocycle and paclitaxel each administered alone. According to certain exemplary embodiments, the combination therapy has a synergistic therapeutic effect. According to this embodiment, the combination therapy produces a significantly better therapeutic result (e.g., anti-cancer, cell growth arrest, apoptosis, induction of differentiation, cell death, etc.) than the additive effects achieved by each individual constituent when administered alone at a therapeutic dose.

Combination therapy includes but is not limited to the combination of peptidomimetic macrocycles of this disclosure with chemotherapeutic agents, therapeutic antibodies, and radiation treatment, to provide a synergistic therapeutic effect. In some embodiments, the peptidomimetic macrocycles of the disclosure are used in combination with one or more anti-cancer (antineoplastic or cytotoxic) chemotherapy drug. Suitable chemotherapeutic agents for use in the combinations of the present disclosure include, but are not limited to, alkylating agents, antibiotic agents, antimetabolic agents, hormonal agents, plant-derived agents, anti-angiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors, gene therapy agents, cell therapy, or any combination thereof.

Synergistic effects can be evaluated by a combination index (CI). CI can be calculated from an isobologram, a combination index plot, or a median-effect plot. Combination index plots show additive or increased complementarity (synergy) of combination treatments. The data can be expressed as log(CI). CI values can be defined as follows: 0-0.1, very strong synergism; 0.1-0.3, strong synergism; 0.3-0.7, synergism; 0.7-0.85, moderate synergism; 0.85-0.90, slight synergism; 0.90-1.10, nearly additive; 1.10-1.20, slight antagonism; 1.20-1.45, moderate antagonism; 1.45-3.3, antagonism; 3.3-10, strong antagonism; 10, very strong antagonism. In some embodiments, CI can be defined as follows: additive effect (CI=1), synergism (CI<1), and antagonism (CI>1).

In some embodiments, a combination therapy described herein has a combination index of less than 1, less than 0.9, less than 0.8, less than 0.7, less than 0.6, or less than 0.5. In some embodiments, a combination therapy described herein has a combination index of about 0.8 to about 0.9. In some embodiments, a combination therapy described herein has a combination index of about 0.9. In some embodiments, a combination therapy described herein has a combination index of about 0.8.

Combination index can be determined from a measure of therapeutic effect against a condition in a subject or inhibitory concentration in a cell proliferation assay. In some embodiments, combination index can be calculated from an in vitro cell proliferation assay. For example, combination index can be calculated from a half maximal inhibitory concentration (IC₅₀). In some embodiments, combination index can be calculated from an IC₇₅ value.

In some embodiments, combination index can be calculated from an in vivo animal study. A combination therapy described herein can be used for treatment of cancer in a subject in need thereof. For example, a combination therapy described herein can inhibit or delay tumor growth. A combination therapy described herein can delay tumor growth in a subject by at least 20 days, at least 21 days, at least 22 days, at least 23 days, at least 24 days, at least 25 days, at least 26 days, at least 27 days, at least 28 days, at least 29 days, or at least 30 days. A combination therapy described herein can result in a percentage tumor growth delay that is at least about 20%, at least about 30%, at least about 40%, at least about 50%, at least about 60%, at least about 70%, at least about 80%, at least about 90%, or at least about 100%.

In some embodiments, a method of treating cancer in a subject in need thereof can comprise administering to the subject a therapeutically effective amount of a p53 agent that inhibits the interaction between p53 and MDM2 and/or p53 and MDMX, and/or modulates the activity of p53 and/or MDM2 and/or MDMX; and at least one additional pharmaceutically-active agent. In some examples, the p53 agent is selected from the group consisting of a small organic or inorganic molecule; a saccharine; an oligosaccharide; a polysaccharide; a peptide, a protein, a peptide analog, a peptide derivative; an antibody, an antibody fragment, a peptidomimetic; a peptidomimetic macrocycle of the disclosure; a nucleic acid; a nucleic acid analog, a nucleic acid derivative; an extract made from biological materials; a naturally-occurring or synthetic composition; and any combination thereof

In some embodiments, the p53 agent is selected from the group consisting of RG7388 (RO5503781, idasanutlin), RG7112 (RO5045337), nutlin3a, nutlin3b, nutlin3, nutlin2, spirooxindole containing small molecules, 1,4-diazepines, 1,4-benzodiazepine-2,5-dione compounds, WK23, WK298, SJ172550, RO2443, RO5963, RO5353, RO2468, MK8242 (SCH900242), MI888, MI773 (SAR405838), NVPCGM097, DS3032b, AM8553, AMG232, NSC207895 (XI006), JNJ26854165 (serdemetan), RITA (NSC652287), YH239EE, or any combination thereof. In some examples, the at least one additional pharmaceutically-active agent is selected from the group consisting of palbociclib (PD0332991); abemaciclib (LY2835219); ribociclib (LEE 011); voruciclib (P1446A-05); fascaplysin; arcyriaflavin; 2-bromo-12,13-dihydro-5H-indolo[2,3-a]pyrrolo[3,4-c]carbazole-5,7(6H)-dione; 3-amino thioacridone (3-ATA), trans-4-((6-(ethylamino)-2-((1-(phenylmethyl)-1H-indol-5-yl)amino)-4-pyrimidinyl)amino)-cyclohexano (CINK4); 1,4-dimethoxyacridine-9(10H)-thione (NSC 625987); 2-methyl-5-(p-tolylamino)benzo[d]thiazole-4,7-dione (ryuvidine); and flavopiridol (alvocidib); and any combination thereof.

In some embodiments, the peptidomimetic macrocycles of the disclosure are used in combination with taxanes, such as paclitaxel (Abraxane® or Taxol®). In some embodiments, the peptidomimetic macrocycles of the instant disclosure are used in combination with paclitaxel.

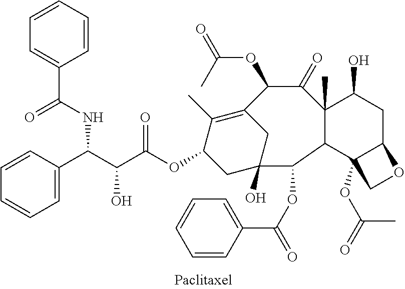

### Administration of Combination Treatment

The peptidomimetic macrocycles or a composition comprising same and the at least one additional pharmaceutically-active agent, for example, any additional therapeutic agent described herein, or a composition comprising same can be administered simultaneously (i.e., simultaneous administration) and/or sequentially (i.e., sequential administration).

According to certain embodiments, the peptidomimetic macrocycles and the at least one additional pharmaceutically-active agent, for example, paclitaxel, are administered simultaneously, either in the same composition or in separate compositions. The term “simultaneous administration,” as used herein, means that the peptidomimetic macrocycle and the at least one additional pharmaceutically-active agent, for example, paclitaxel, are administered with a time separation of no more than a few minutes, for example, less than about 15 minutes, less than about 10, less than about 5, or less than about 1 minute. When the drugs are administered simultaneously, the peptidomimetic macrocycle and the at least one additional pharmaceutically-active agent, for example, any additional therapeutic agent described herein, may be contained in the same composition (e.g., a composition comprising both the peptidomimetic macrocycle and the at least additional pharmaceutically-active agent) or in separate compositions (e.g., the peptidomimetic macrocycle is contained in one composition and the at least additional pharmaceutically-active agent is contained in another composition).

According to other embodiments, the peptidomimetic macrocycles and the at least one additional pharmaceutically-active agent, for example, any additional therapeutic agent described herein, are administered sequentially, i.e., the peptidomimetic macrocycle is administered either prior to or after the administration of the additional pharmaceutically-active agent. The term “sequential administration” as used herein means that the peptidomimetic macrocycle and the additional pharmaceutically-active agent, for example, any additional therapeutic agent described herein, are administered with a time separation of more than a few minutes, for example, more than about 15 minutes, more than about 20 or more minutes, more than about 30 or more minutes, more than about 40 or more minutes, more than about 50 or more minutes, or more than about 60 or more minutes. In some embodiments, the peptidomimetic macrocycle is administered before the additional pharmaceutically-active agent, for example, any additional therapeutic agent described herein. In some embodiments, the pharmaceutically-active agent, for example, any additional therapeutic agent described herein, is administered before the peptidomimetic macrocycle. The peptidomimetic macrocycle and the additional pharmaceutically-active agent, for example, any additional therapeutic agent described herein, are contained in separate compositions, which may be contained in the same or different packages.

In some embodiments, the administration of the peptidomimetic macrocycles and the additional pharmaceutically-active agent, for example, any additional therapeutic agent described herein, are concurrent, i.e., the administration period of the peptidomimetic macrocycles and that of the agent overlap with each other. In some embodiments, the administration of the peptidomimetic macrocycles and the additional pharmaceutically-active agent, for example, any additional therapeutic agent described herein, are non-concurrent. For example, in some embodiments, the administration of the peptidomimetic macrocycles is terminated before the additional pharmaceutically-active agent, for example, any additional therapeutic agent described herein, is administered. In some embodiments, the administration of the additional pharmaceutically-active agent, for example, any additional therapeutic agent described herein, is terminated before the peptidomimetic macrocycle is administered. The time period between these two non-concurrent administrations can range from being days apart to being weeks apart.

The dosing frequency of the peptidomimetic macrocycle and the at least one additional pharmaceutically-active agent, for example, any additional therapeutic agent described herein, may be adjusted over the course of the treatment, based on the judgment of the administering physician. When administered separately, the peptidomimetic macrocycle and the at least one additional pharmaceutically-active agent, for example, any additional therapeutic agent described herein, can be administered at different dosing frequency or intervals. For example, the peptidomimetic macrocycle can be administered weekly, while the at least one additional pharmaceutically-active agent, for example, any additional therapeutic agent described herein, can be administered more or less frequently. Or, the peptidomimetic macrocycle can be administered twice weekly, while the at least one additional pharmaceutically-active agent, for example, any additional therapeutic agent described herein, can be administered more or less frequently. In addition, the peptidomimetic macrocycle and the at least one additional pharmaceutically-active agent, for example, any additional therapeutic agent described herein, can be administered using the same route of administration or using different routes of administration.

A therapeutically effective amount of a peptidomimetic macrocycle and/or the additional pharmaceutically-active agent, for example, any additional therapeutic agent described herein, for use in therapy can vary with the nature of the condition being treated, the length of treatment time desired, the age and the condition of the patient, and can be determined by the attending physician. Doses employed for human treatment can be in the range of about 0.01 mg/kg to about 1000 mg/kg per day (e.g., about 0.01 mg/kg to about 100 mg/kg per day, about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day) of one or each component of the combinations described herein. In some embodiments, doses of a peptidomimetic macrocycle employed for human treatment are in the range of about 0.01 mg/kg to about 100 mg/kg per day (e.g., about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day, about 1 mg/kg per day). In some embodiments, doses of the additional pharmaceutically-active agent, for example, any additional therapeutic agent described herein, employed for human treatment can be in the range of about 0.01 mg/kg to about 100 mg/kg per day (e.g., about 0.1 mg/kg to about 100 mg/kg per day, about 0.1 mg/kg to about 50 mg/kg per day, about 10 mg/kg per day or about 30 mg/kg per day). The desired dose may be conveniently administered in a single dose, or as multiple doses administered at appropriate intervals, for example as two, three, four or more sub-doses per day.

In some embodiments, such as when given in combination with the at least one additional pharmaceutically active agent, for example, any additional therapeutic agent described herein, the dosage of a peptidomimetic macrocycle may be given at relatively lower dosages. In some embodiments, the dosage of a peptidomimetic macrocycle may be from about 1 ng/kg to about 100 mg/kg. The dosage of a peptidomimetic macrocycle may be at any dosage including, but not limited to, about 1 μg/kg, 25 μg/kg, 50 μg/kg, 75 μg/kg, 100 μ μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

In some embodiments, the dosage of the additional pharmaceutically-active agent, for example, any additional therapeutic agent described herein, may be from about 1 ng/kg to about 100 mg/kg. The dosage of the additional pharmaceutically-active agent may be at any dosage including, but not limited to, about 1μg/kg, 25 μg/kg, 50 μg/kg, 75 μg/kg, 100 μμg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

The peptidomimetic macrocycle and the additional pharmaceutically-active agent, for example, any additional therapeutic agent described herein, can be provided in a single unit dosage form for being taken together or as separate entities (e.g. in separate containers) to be administered simultaneously or with a certain time difference. This time difference may be between 1 hour and 1 month, e.g., between 1 day and 1 week, e.g., 48 hours and 3 days. In addition, it is possible to administer the peptidomimetic macrocycle via another administration way than the additional pharmaceutically-active agent, for example, paclitaxel. For example, it may be advantageous to administer either the peptidomimetic macrocycle or the additional pharmaceutically-active agent, for example, paclitaxel, intravenously and the other systemically or orally. For example, the peptidomimetic macrocycle is administered intravenously and the additional pharmaceutically-active agent orally.

In some embodiments, the peptidomimetic macrocycle is administered about 0.1 hour, 0.2 hour, 0.3 hour, 0.4 hour, 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or 12 months before the additional pharmaceutically-active agent, for example, paclitaxel, is administered. In some embodiments, the peptidomimetic macrocycle is administered about 6 hours before the additional pharmaceutically-active agent, for example, paclitaxel, is administered.

In some embodiments, the peptidomimetic macrocycle is administered about 0.1 hour, 0.2 hour, 0.3 hour, 0.4 hour, 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or 12 months after the additional pharmaceutically-active agent, for example, paclitaxel, is administered. In some embodiments, the peptidomimetic macrocycle is administered about 6 hours after the additional pharmaceutically-active agent, for example, paclitaxel, is administered.

In some embodiments, the peptidomimetic macrocycle is administered chronologically before the additional pharmaceutically-active agent, for example, paclitaxel. In some embodiments, the peptidomimetic macrocycle is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9,-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the additional pharmaceutically-active agent, for example, paclitaxel, is administered. In some embodiments, the peptidomimetic macrocycle is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the additional pharmaceutically-active agent, for example, paclitaxel, is administered.

In some embodiments, the peptidomimetic macrocycle is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before paclitaxelis administered.

In some embodiments, the peptidomimetic macrocycle is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the additional pharmaceutically-active agent, for example, paclitaxel, is administered.

In some embodiments, the peptidomimetic macrocycle is administered chronologically at the same time as paclitaxel.

In some embodiments, the peptidomimetic macrocycle is administered chronologically after the additional pharmaceutically-active agent, for example, paclitaxel. In some embodiments, the additional pharmaceutically-active agent, for example, paclitaxel, is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9,-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the peptidomimetic macrocycle is administered. In some embodiments, paclitaxelis administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered.

In some embodiments, paclitaxel at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the peptidomimetic macrocycle is administered.

Also, contemplated herein is a drug holiday utilized among the administration of a peptidomimetic macrocycle and an additional pharmaceutically-active agent, for example, paclitaxel. A drug holiday can be a period of days after the administration of the additional pharmaceutically-active agent, for example, any additional therapeutic agent described herein, and before the administration of a peptidomimetic macrocycle. A drug holiday can be a period of days after the administration of a peptidomimetic macrocycle and before the administration of the additional pharmaceutically-active agent, for example, paclitaxel. A drug holiday can be a period of days after the sequential administration of one or more of a peptidomimetic macrocycle and an additional pharmaceutically-active agent, for example, paclitaxel, and before the administration of the peptidomimetic macrocycle, the additional pharmaceutically-active agent or another therapeutic agent. For example, a drug holiday can be a period of days after the sequential administration of a peptidomimetic macrocycle first, followed administration of an additional pharmaceutically-active agent, for example, paclitaxel, and before the administration of the peptidomimetic macrocycle again. For example, a drug holiday can be a period of days after the sequential administration of an additional pharmaceutically-active agent first, followed administration of a peptidomimetic macrocycle and before the administration of the additional pharmaceutically-active agent, for example, paclitaxel.

Suitably the drug holiday will be a period of 1 day, 2 days, 3 days, 4 days, 5 days, 6 days, 7 days, 8 days, 9 days, 10 days, 11 days, 12 days, 13 days or 14 days; or from 1-24, 2-24, 3-24, 4-24, 5-24, 6-24, 7-24, 8-24, 9-24, 10-24, 11-24, or 12-24 hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 days, 1-4, 2-4,or 3-4 weeks; or from 1-12, 2-12, 3-12, 4-12, 5-12, 6-12, 7-12, 8-12, 9-12, 10-12, or 11-12 months.

In some embodiments, an additional pharmaceutically-active agent, for example, paclitaxel, will be administered first in the sequence, followed by an optional drug holiday, followed by administration of a peptidomimetic macrocycle. In some embodiments, an additional pharmaceutically-active agent, for example, paclitaxel, will be administered first in the sequence, followed by administration of a peptidomimetic macrocycle, followed by an optional drug holiday, followed by administration of an additional pharmaceutically-active agent.

In some embodiments, an additional pharmaceutically-active agent, for example, paclitaxel, is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months. In some embodiments, an additional pharmaceutically-active agent, for example, paclitaxel, is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of paclitaxel.

In some embodiments, a peptidomimetic macrocycle will be administered first in the sequence, followed by an optional drug holiday, followed by administration of an additional pharmaceutically-active agent, for example, paclitaxel. In some embodiments, a peptidomimetic macrocycle will be administered first in the sequence, followed by administration of an additional pharmaceutically-active agent, for example, paclitaxel, followed by an optional drug holiday, followed by administration of a peptidomimetic macrocycle.

In some embodiments, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of an additional pharmaceutically-active agent, for example, paclitaxel, for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months. In some embodiments, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by administration of an additional pharmaceutically-active agent, for example, paclitaxel, for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9,-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of a peptidomimetic macrocycle.

In some embodiments, an additional pharmaceutically-active agent, for example, paclitaxel, will be administered first in the sequence, followed by an optional drug holiday, followed by administration of a peptidomimetic macrocycle.

In some embodiments, an additional pharmaceutically-active agent, for example, paclitaxel, is administered for from 1 to 30 consecutive days, followed by an optional drug holiday, followed by administration of peptidomimetic macrocycle for from 1 to 30 consecutive days. In some embodiments, an additional pharmaceutically-active agent, for example, paclitaxel, is administered for from 1 to 21 consecutive days, followed by an optional drug holiday, followed by administration of a peptidomimetic macrocycle for from 1 to 21 consecutive days. In some embodiments, an additional pharmaceutically-active agent, for example, paclitaxel, is administered for from 1 to 14 consecutive days, followed by an optional drug holiday, followed by administration of a peptidomimetic macrocycle for from 1 to 14 consecutive days. In some embodiments, an additional pharmaceutically-active agent, for example, paclitaxel, is administered for 14 consecutive days, followed by an optional drug holiday, followed by administration of a peptidomimetic macrocycle for 7 consecutive days. In some embodiments, an additional pharmaceutically-active agent, for example, paclitaxel, is administered for 7 consecutive days, followed by an optional drug holiday, followed by administration of a peptidomimetic macrocycle for 7 consecutive days.

In some embodiments, a peptidomimetic macrocycle is administered for from 1 to 30 consecutive days, followed by an optional drug holiday, followed by administration of an additional pharmaceutically-active agent, for example, paclitaxel, for from 1 to 30 consecutive days. In some embodiments, a peptidomimetic macrocycle is administered for from 1 to 21 consecutive days, followed by an optional drug holiday, followed by administration of an additional pharmaceutically-active agent, for example, paclitaxel, for from 1 to 21 consecutive days. In some embodiments, a peptidomimetic macrocycle is administered for from 1 to 14 consecutive days, followed by an optional drug holiday, followed by administration of an additional pharmaceutically-active agent, for example, paclitaxel, for from 1 to 14 consecutive days. In some embodiments, a peptidomimetic macrocycle is administered for 14 consecutive days, followed by an optional drug holiday, followed by administration of an additional pharmaceutically-active agent, for example, paclitaxel, for 14 consecutive days. In some embodiments, a peptidomimetic macrocycle is administered for 7 consecutive days, followed by an optional drug holiday, followed by administration of an additional pharmaceutically-active agent, for example, paclitaxel, for 7 consecutive days.

In some embodiments, one of a peptidomimetic macrocycle and an additional pharmaceutically-active agent, for example, paclitaxel, is administered for from 2 to 30 consecutive days, followed by an optional drug holiday, followed by administration of the other of a peptidomimetic macrocycle and an additional pharmaceutically-active agent for from 2 to 30 consecutive days. In some embodiments, one of a peptidomimetic macrocycle and an additional pharmaceutically-active agent, for example, paclitaxel, is administered for from 2 to 21 consecutive days, followed by an optional drug holiday, followed by administration of the other of a peptidomimetic macrocycle and an additional pharmaceutically-active agent for from 2 to 21 consecutive days. In some embodiments, one of a peptidomimetic macrocycle and an additional pharmaceutically-active agent, for example, paclitaxel, is administered for from 2 to 14 consecutive days, followed by a drug holiday of from 1 to 14 days, followed by administration of the other of a peptidomimetic macrocycle and an additional pharmaceutically-active agent for from 2 to 14 consecutive days. In some embodiments, one of a peptidomimetic macrocycle and an additional pharmaceutically-active agent, for example, paclitaxel, is administered for from 3 to 7 consecutive days, followed by a drug holiday of from 3 to 10 days, followed by administration of the other of a peptidomimetic macrocycle and an additional pharmaceutically-active agent for from 3 to 7 consecutive days.

In some embodiments, paclitaxel is administered first in the sequence, followed by an optional drug holiday, followed by administration of a peptidomimetic macrocycle. In some embodiments, paclitaxel is administered for from 3 to 21 consecutive days, followed by an optional drug holiday, followed by administration of a peptidomimetic macrocycle for from 3 to 21 consecutive days. In some embodiments, paclitaxel is administered for from 3 to 21 consecutive days, followed by a drug holiday of from 1 to 14 days, followed by administration of a peptidomimetic macrocycle for from 3 to 21 consecutive days. In some embodiments, paclitaxel is administered for from 3 to 21 consecutive days, followed by a drug holiday of from 3 to 14 days, followed by administration of a peptidomimetic macrocycle for from 3 to 21 consecutive days. In some embodiments, paclitaxel is administered for 21 consecutive days, followed by an optional drug holiday, followed by administration of a peptidomimetic macrocycle for 14 consecutive days. In some embodiments, paclitaxel is administered for 14 consecutive days, followed by a drug holiday of from 1 to 14 days, followed by administration of a peptidomimetic macrocycle for 14 consecutive days. In some embodiments, paclitaxel is administered for 7 consecutive days, followed by a drug holiday of from 3 to 10 days, followed by administration of a peptidomimetic macrocycle for 7 consecutive days. In some embodiments, paclitaxel is administered for 3 consecutive days, followed by a drug holiday of from 3 to 14 days, followed by administration of a peptidomimetic macrocycle for 7 consecutive days. In some embodiments, paclitaxel is administered for 3 consecutive days, followed by a drug holiday of from 3 to 10 days, followed by administration of a peptidomimetic macrocycle for 3 consecutive days.

In some embodiments, a peptidomimetic macrocycle will be administered first in the sequence, followed by an optional drug holiday, followed by administration of paclitaxel. In some embodiments, a peptidomimetic macrocycle is administered for from 3 to 21 consecutive days, followed by an optional drug holiday, followed by administration of paclitaxel for from 3 to 21 consecutive days. In some embodiments, a peptidomimetic macrocycle is administered for from 3 to 21 consecutive days, followed by a drug holiday of from 1 to 14 days, followed by administration of paclitaxel for from 3 to 21 consecutive days. In some embodiments, a peptidomimetic macrocycle is administered for from 3 to 21 consecutive days, followed by a drug holiday of from 3 to 14 days, followed by administration of paclitaxel for from 3 to 21 consecutive days. In some embodiments, a peptidomimetic macrocycle is administered for 21 consecutive days, followed by an optional drug holiday, followed by administration of paclitaxel for 14 consecutive days. In some embodiments, a peptidomimetic macrocycle s administered for 14 consecutive days, followed by a drug holiday of from 1 to 14 days, followed by administration of paclitaxel for 14 consecutive days. In some embodiments, a peptidomimetic macrocycle is administered for 7 consecutive days, followed by a drug holiday of from 3 to 10 days, followed by administration of paclitaxel for 7 consecutive days. In some embodiments, a peptidomimetic macrocycle is administered for 3 consecutive days, followed by a drug holiday of from 3 to 14 days, followed by administration of paclitaxel for 7 consecutive days. In some embodiments, a peptidomimetic macrocycle is administered for 3 consecutive days, followed by a drug holiday of from 3 to 10 days, followed by administration of paclitaxel for 3 consecutive days.

In some embodiments, a peptidomimetic macrocycle is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the peptidomimetic macrocycle/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; and the additional pharmaceutically-active agent, for example, paclitaxel, is administered prior to, concomitantly with, or subsequent to administration of the peptidomimetic macrocycle on one or more days (e.g., on day 1 of cycle 1). In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In some embodiments, provided herein is a method of treating a condition or disease comprising administering to a patient in need thereof a therapeutically effective amount of a peptidomimetic macrocycle in combination with a therapeutically effective amount of an additional pharmaceutically-active agent, for example, paclitaxel, and a secondary active agent, such as a checkpoint inhibitor. In some embodiments, a peptidomimetic macrocycle is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the peptidomimetic macrocycle/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; the additional pharmaceutically-active agent, for example, paclitaxel, is administered prior to, concomitantly with, or subsequent to administration of the peptidomimetic macrocycle on one or more days (e.g., on day 1 of cycle 1), and the secondary agent is administered daily, weekly, or monthly. In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In some embodiments, the components of the combination therapies described herein (e.g., a peptidomimetic macrocycle and paclitaxel) are cyclically administered to a patient. In some embodiments, a secondary active agent is co-administered in a cyclic administration with the combination therapies provided herein. Cycling therapy involves the administration of an active agent for a period of time, followed by a rest for a period of time, and repeating this sequential administration. Cycling therapy can be performed independently for each active agent (e.g., a peptidomimetic macrocycle and paclitaxel, and/or a secondary agent) over a prescribed duration of time. In some embodiments, the cyclic administration of each active agent is dependent upon one or more of the active agents administered to the subject. In some embodiments, administration of a peptidomimetic macrocycle or paclitaxel fixes the day(s) or duration of administration of each agent. In some embodiments, administration of a peptidomimetic macrocycle or paclitaxel fixes the days(s) or duration of administration of a secondary active agent.

In some embodiments, a peptidomimetic macrocycle, paclitaxel, and/or a secondary active agent is administered continually (e.g., daily, weekly, monthly) without a rest period. Cycling therapy can reduce the development of resistance to one or more of the therapies, avoid, or reduce the side effects of one of the therapies, and/or improve the efficacy of the treatment or therapeutic agent.

In some embodiments, the frequency of administration is in the range of about a daily dose to about a monthly dose. In some embodiments, administration is once a day, twice a day, three times a day, four times a day, once every other day, twice a week, once every week, once every two weeks, once every three weeks, or once every four weeks. In some embodiments, a compound for use in combination therapies described herein is administered once a day. In some embodiments, a compound for use in combination therapies described herein is administered twice a day. In some embodiments, a compound for use in combination therapies described herein is administered three times a day. In some embodiments, a compound for use in combination therapies described herein is administered four times a day.

In some embodiments, the frequency of administration of a peptidomimetic macrocycle is in the range of about a daily dose to about a monthly dose. In some embodiments, administration of a peptidomimetic macrocycle is once a day, twice a day, three times a day, four times a day, once every other day, twice a week, once every week, once every two weeks, once every three weeks, or once every four weeks. In some embodiments, a peptidomimetic macrocycle for use in combination therapies described herein is administered once a day. In some embodiments, a peptidomimetic macrocycle for use in combination therapies described herein is administered twice a day. In some embodiments, a peptidomimetic macrocycle for use in combination therapies described herein is administered three times a day. In some embodiments, a peptidomimetic macrocycle for use in combination therapies described herein is administered four times a day.

In some embodiments, the frequency of administration of an additional pharmaceutically-active agent, for example, any additional therapeutic agent described herein, is in the range of about a daily dose to about a monthly dose. In some embodiments, administration of an additional pharmaceutically-active agent, for example, any additional therapeutic agent described herein, is once a day, twice a day, three times a day, four times a day, once every other day, twice a week, once every week, once every two weeks, once every three weeks, or once every four weeks. In some embodiments, an additional pharmaceutically-active agent, for example, any additional therapeutic agent described herein, for use in combination therapies described herein is administered once a day. In some embodiments, an additional pharmaceutically-active agent, for example, any additional therapeutic agent described herein, for use in combination therapies described herein is administered twice a day. In some embodiments, an additional pharmaceutically-active agent, for example, any additional therapeutic agent described herein, for use in combination therapies described herein is administered three times a day. In some embodiments, an additional pharmaceutically-active agent, for example, any additional therapeutic agent described herein, for use in combination therapies described herein is administered four times a day.

In some embodiments, a compound for use in combination therapies described herein is administered once per day from one day to six months, from one week to three months, from one week to four weeks, from one week to three weeks, or from one week to two weeks. In some embodiments, a compound for use in combination therapies described herein is administered once per day for one week, two weeks, three weeks, or four weeks. In some embodiments, a compound for use in combination therapies described herein is administered once per day for one week. In some embodiments, a compound for use in combination therapies described herein is administered once per day for two weeks. In some embodiments, a compound for use in combination therapies described herein is administered once per day for three weeks. In some embodiments, a compound for use in combination therapies described herein is administered once per day for four weeks.

Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

In some embodiments, the periodic administration of a peptidomimetic macrocycle and/or the additional pharmaceutically-active agent, for example, any additional therapeutic agent described herein, is effected daily. In some embodiments, the periodic administration of a peptidomimetic macrocycle and/or the additional pharmaceutically-active agent, for example, any additional therapeutic agent described herein, is effected twice daily at one half the amount.

In some embodiments, the periodic administration of a peptidomimetic macrocycle and/or the additional pharmaceutically-active agent, for example, any additional therapeutic agent described herein, is effected once every 3 to 11 days; or once every 5 to 9 days; or once every 7 days; or once every 24 hours. In some embodiments, the periodic administration of a peptidomimetic macrocycle and/or the additional pharmaceutically-active agent, for example, any additional therapeutic agent described herein, is effected once every 2 days, 3 days, 4 days, 5 days, 6 days, 7 days, 8 days, 9 days, 10 days, 11 days, 12 days, 13 days, 14 days, 15 days, 6 days, 16 days, 17 days, 18 days, 19 days, 20 days, 21 days, 22 days, 23 days, 24 days, 25 days, 26 days, 27 days, 28 days, 29 days, or 30 days.

In some embodiments, the periodic administration of a peptidomimetic macrocycle and/or additional pharmaceutically-active agentis effected one, twice, or thrice daily.

For each administration schedule of a peptidomimetic macrocycle, the periodic administration of the additional pharmaceutically-active agent, for example, any additional therapeutic agent described herein, may be effected once every 16-32 hours; or once every 18-30 hours; or once every 20-28 hours; or once every 22-26 hours. In some embodiments, the administration of a peptidomimetic macrocycle substantially precedes the additional pharmaceutically-active agent, for example, any additional therapeutic agent described herein. In some embodiments, the administration of the additional pharmaceutically-active agent, for example, any additional therapeutic agent described herein, substantially precedes the administration of a peptidomimetic macrocycle.

In some embodiments, a peptidomimetic macrocycle and the additional pharmaceutically-active agent, for example, any additional therapeutic agent described herein, may be administered for a period of time of at least 4 days. In some embodiments, the period of time may be 5 days to 5 years; or 10 days to 3 years; or 2 weeks to 1 year; or 1 month to 6 months; or 3 months to 4 months. In some embodiments, a peptidomimetic macrocycle and the additional pharmaceutically-active agent, for example, any additional therapeutic agent described herein, may be administered for the lifetime of the subject.

### Pharmaceutical Compositions for Combination Treatment

According to certain embodiments, the peptidomimetic macrocycles and the additional pharmaceutically-active agent, for example, any additional therapeutic agent described herein, are administered within a single pharmaceutical composition. In some embodiments, the peptidomimetic macrocycles of the invention and the additional pharmaceutically-active agent, for example, any additional therapeutic agent described herein, can be provided in a single unit dosage form for being taken together. According to some embodiments, the pharmaceutical composition further comprises pharmaceutically-acceptable diluents or carrier. According to certain embodiments, the peptidomimetic macrocycles and the additional pharmaceutically-active agent, for example, any additional therapeutic agent described herein, are administered within different pharmaceutical composition. In some embodiments, the peptidomimetic macrocycles of the invention and the additional pharmaceutically-active agent, for example, any additional therapeutic agent described herein, can be provided in a single unit dosage as separate entities (e.g., in separate containers) to be administered simultaneously or with a certain time difference. In some embodiments, the peptidomimetic macrocycles of the disclosure and the additional pharmaceutically-active agent, for example, any additional therapeutic agent described herein, can be administered via the same route of administration. In some embodiments, the peptidomimetic macrocycles of the disclosure and the additional pharmaceutically-active agent, for example, any additional therapeutic agent described herein, can be administered via the different route of administration.

In some embodiments, the at least one additional pharmaceutical agent, for example, any additional therapeutic agent described herein, is administered at the therapeutic amount known to be used for treating the specific type of cancer. In some embodiments, the at least one additional pharmaceutical agent, for example, any additional therapeutic agent described herein, is administered in an amount lower than the therapeutic amount known to be used for treating the disease, i.e. a sub-therapeutic amount of the at least one additional pharmaceutical agent is administered.

A peptidomimetic macrocycle of the disclosure and at least one additional pharmaceutical agent, for example, any additional therapeutic agent described herein, administered to the subject can each be from about 0.01 mg/kg to about 100 mg/kg per body weight of the subject. In some embodiments, a peptidomimetic macrocycle of the disclosure and the at least one additional pharmaceutical agent, for example, any additional therapeutic agent described herein, administered to the subject can each be from about 0.01 mg/kg to about 1 mg/kg, 0.01 mg/kg to about 10 mg/kg, 0.01 mg/kg to about 100 mg/kg, 0.1 mg to about 1 mg/kg, 0.1 mg/kg to about 10 mg/kg, or 0.1 mg/kg to about 100 mg/kg per body weight of the subject. In some embodiments, the doses of a peptidomimetic macrocycle and additional therapeutic agent, for example, any additional therapeutic agent described herein, can be administered as a single dose or as multiple doses.

### Sequence Homology

Two or more peptides can share a degree of homology. A pair of peptides can have, for example, up to about 20% pairwise homology, up to about 25% pairwise homology, up to about 30% pairwise homology, up to about 35% pairwise homology, up to about 40% pairwise homology, up to about 45% pairwise homology, up to about 50% pairwise homology, up to about 55% pairwise homology, up to about 60% pairwise homology, up to about 65% pairwise homology, up to about 70% pairwise homology, up to about 75% pairwise homology, up to about 80% pairwise homology, up to about 85% pairwise homology, up to about 90% pairwise homology, up to about 95% pairwise homology, up to about 96% pairwise homology, up to about 97% pairwise homology, up to about 98% pairwise homology, up to about 99% pairwise homology, up to about 99.5% pairwise homology, or up to about 99.9% pairwise homology. A pair of peptides can have, for example, at least about 20% pairwise homology, at least about 25% pairwise homology, at least about 30% pairwise homology, at least about 35% pairwise homology, at least about 40% pairwise homology, at least about 45% pairwise homology, at least about 50% pairwise homology, at least about 55% pairwise homology, at least about 60% pairwise homology, at least about 65% pairwise homology, at least about 70% pairwise homology, at least about 75% pairwise homology, at least about 80% pairwise homology, at least about 85% pairwise homology, at least about 90% pairwise homology, at least about 95% pairwise homology, at least about 96% pairwise homology, at least about 97% pairwise homology, at least about 98% pairwise homology, at least about 99% pairwise homology, at least about 99.5% pairwise homology, at least about 99.9% pairwise homology.

Methods of Detecting Wild Type p53 and/or p53 Mutations

In some embodiments, a subject lacking p53-deactivating mutations is a candidate for cancer treatment with a compound of the invention. Cancer cells from patient groups are assayed in order to determine p53-deactivating mutations and/or expression of wild type p53 prior to treatment with a compound of the invention.

The activity of the p53 pathway can be determined by the mutational status of genes involved in the p53 pathways, including, for example, AKT1, AKT2, AKT3, ALK, BRAF, CDK4, CDKN2A, DDR2, EGFR, ERBB2 (HER2), FGFR1, FGFR3, GNA11, GNQ, GNAS, KDR, KIT, KRAS, MAP2K1 (MEK1), MET, HRAS, NOTCH1, NRAS, NTRK2, PIK3CA, NF1, PTEN, RAC1, RB1, NTRK3, STK11, PIK3R1, TSC1, TSC2, RET, TP53, and VHL. Genes that modulate the activity of p53 can also be assessed, including, for example, kinases: ABL1, JAK1, JAAK2, JAK3; receptor tyrosine kinases: FLT3 and KIT; receptors: CSF3R, IL7R, MPL, and NOTCH1; transcription factors: BCOR, CEBPA, CREBBP, ETV6, GATA1, GATA2. MLL, KZF1, PAX5, RUNX1, STAT3, WT1, and TP53; epigenetic factors: ASXL1, DNMT3A, EZH2, KDM6A (UTX), SUZ12, TET2, PTPN11, SF3B1, SRSF2, U2AF35, ZRSR2; RAS proteins: HRAS, KRAS, and NRAS; adaptors CBL and CBL-B; FBXW7, IDH1, IDH2, and NPM1.

Cancer cell samples can be obtained, for example, from solid or liquid tumors via primary or metastatic tumor resection (e.g. pneumonectomy, lobetomy, wedge resection, and craniotomy) primary or metastatic disease biopsy (e.g. transbronchial or needle core), pleural or ascites fluid (e.g. FFPE cell pellet), bone marrow aspirate, bone marrow clot, and bone marrow biopsy, or macro-dissection of tumor rich areas (solid tumors).

To detect the p53 wild type gene and/or lack of p53 deactivation mutation in a tissue, cancerous tissue can be isolated from surrounding normal tissues. For example, the tissue can be isolated from paraffin or cryostat sections. Cancer cells can also be separated from normal cells by flow cytometry. If the cancer cells tissue is highly contaminated with normal cells, detection of mutations can be more difficult.

Various methods and assays for analyzing wild type p53 and/or p53 mutations are suitable for use in the invention. Non-limiting examples of assays include polymerase chain reaction (PCR), restriction fragment length polymorphism (RFLP), microarray, Southern Blot, Northern Blot, Western Blot, Eastern Blot, HandE staining, microscopic assessment of tumors, next-generation DNA sequencing (NGS) (e.g. extraction, purification, quantification, and amplification of DNA, library preparation) immunohistochemistry, and fluorescent in situ hybridization (FISH).

A microarray allows a researcher to investigate multiple DNA sequences attached to a surface, for example, a DNA chip made of glass or silicon, or a polymeric bead or resin. The DNA sequences are hybridized with fluorescent or luminescent probes. The microarray can indicate the presence of oligonucleotide sequences in a sample based on hybridization of sample sequences to the probes, followed by washing and subsequent detection of the probes. Quantification of the fluorescent or luminescent signal indicates the presence of known oligonucleotide sequences in the sample.

PCR allows amplification of DNA oligomers rapidly, and can be used to identify an oligonucleotide sequence in a sample. PCR experiments involve contacting an oligonucleotide sample with a PCR mixture containing primers complementary to a target sequence, one or more DNA polymerase enzymes, deoxnucleotide triphosphate (dNTP) building blocks, including dATP, dGTP, dTTP, and dCTP, and suitable buffers, salts, and additives. If a sample contains an oligonucleotide sequence complementary to a pair of primers, the experiment amplifies the sample sequence, which can be collected and identified.

In some embodiments, an assay comprises amplifying a biomolecule from the cancer sample. The biomolecule can be a nucleic acid molecule, such as DNA or RNA. In some embodiments, the assay comprises circularization of a nucleic acid molecule, followed by digestion of the circularized nucleic acid molecule.

In some embodiments, the assay comprises contacting an organism, or a biochemical sample collected from an organism, such as a nucleic acid sample, with a library of oligonucleotides, such as PCR primers. The library can contain any number of oligonucleotide molecules. The oligonucleotide molecules can bind individual DNA or RNA motifs, or any combination of motifs described herein. The motifs can be any distance apart, and the distance can be known or unknown. In some embodiments, two or more oligonucleotides in the same library bind motifs a known distance apart in a parent nucleic acid sequence. Binding of the primers to the parent sequence can take place based on the complementarity of the primers to the parent sequence. Binding can take place, for example, under annealing, or under stringent conditions.

In some embodiments, the results of an assay are used to design a new oligonucleotide sequence for future use. In some embodiments, the results of an assay are used to design a new oligonucleotide library for future use. In some embodiments, the results of an assay are used to revise, refine, or update an existing oligonucleotide library for future use. For example, an assay can reveal that a previously-undocumented nucleic acid sequence is associated with the presence of a target material. This information can be used to design or redesign nucleic acid molecules and libraries.

In some embodiments, one or more nucleic acid molecules in a library comprise a barcode tag. In some embodiments, one or more of the nucleic acid molecules in a library comprise type I or type II restriction sites suitable for circularization and cutting an amplified sample nucleic acid sequence. Such primers can be used to circularize a PCR product and cut the PCR product to provide a product nucleic acid sequence with a sequence that is organized differently from the nucleic acid sequence native to the sample organism.

After a PCR experiment, the presence of an amplified sequence can be verified. Non-limiting examples of methods for finding an amplified sequence include DNA sequencing, whole transcriptome shotgun sequencing (WTSS, or RNA-seq), mass spectrometry (MS), microarray, pyrosequencing, column purification analysis, polyacrylamide gel electrophoresis, and index tag sequencing of a PCR product generated from an index-tagged primer.

In some embodiments, more than one nucleic acid sequence in the sample organism is amplified. Non-limiting examples of methods of separating different nucleic acid sequences in a PCR product mixture include column purification, high performance liquid chromatography (HPLC), HPLC/MS, polyacrylamide gel electrophoresis, size exclusion chromatography.

The amplified nucleic acid molecules can be identified by sequencing. Nucleic acid sequencing can be done on automated instrumentation. Sequencing experiments can be done in parallel to analyze tens, hundreds, or thousands of sequences simultaneously. Non-limiting examples of sequencing techniques follow.

In pyrosequencing, DNA is amplified within a water droplet containing a single DNA template bound to a primer-coated bead in an oil solution. Nucleotides are added to a growing sequence, and the addition of each base is evidenced by visual light.

Ion semiconductor sequencing detects the addition of a nucleic acid residue as an electrical signal associated with a hydrogen ion liberated during synthesis. A reaction well containing a template is flooded with the four types of nucleotide building blocks, one at a time. The timing of the electrical signal identifies which building block was added, and identifies the corresponding residue in the template.

DNA nanoball uses rolling circle replication to amplify DNA into nanoballs. Unchained sequencing by ligation of the nanoballs reveals the DNA sequence.

In a reversible dyes approach, nucleic acid molecules are annealed to primers on a slide and amplified. Four types of fluorescent dye residues, each complementary to a native nucleobase, are added, the residue complementary to the next base in the nucleic acid sequence is added, and unincorporated dyes are rinsed from the slide. Four types of reversible terminator bases (RT-bases) are added, and non-incorporated nucleotides are washed away. Fluorescence indicates the addition of a dye residue, thus identifying the complementary base in the template sequence. The dye residue is chemically removed, and the cycle repeats.

Detection of point mutations can be accomplished by molecular cloning of the p53 allele(s) present in the cancer cell tissue and sequencing that allele(s). Alternatively, the polymerase chain reaction can be used to amplify p53 gene sequences directly from a genomic DNA preparation from the cancer cell tissue. The DNA sequence of the amplified sequences can then be determined. Specific deletions of p53 genes can also be detected. For example, restriction fragment length polymorphism (RFLP) probes for the p53 gene or surrounding marker genes can be used to score loss of a p53 allele.

Loss of wild type p53 genes can also be detected on the basis of the loss of a wild type expression product of the p53 gene. Such expression products include both the mRNA as well as the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via the polymerase chain reaction (PCR).

Alternatively, mismatch detection can be used to detect point mutations in the p53 gene or the mRNA product. The method can involve the use of a labeled riboprobe that is complementary to the human wild type p53 gene. The riboprobe and either mRNA or DNA isolated from the cancer cell tissue are annealed (hybridized) together and subsequently digested with the enzyme RNase A which is able to detect some mismatches in a duplex RNA structure. If a mismatch is detected by RNase A, the enzyme cleaves at the site of the mismatch. Thus, when the annealed RNA preparation is separated on an electrophoretic gel matrix, if a mismatch has been detected and cleaved by RNase A, an RNA product is seen that is smaller than the full-length duplex RNA for the riboprobe and the p53 mRNA or DNA. The riboprobe need not be the full length of the p53 mRNA or gene but can be a segment of either. If the riboprobe comprises only a segment of the p53 mRNA or gene it will be desirable to use a number of these probes to screen the whole mRNA sequence for mismatches.

In similar fashion, DNA probes can be used to detect mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA which might contain a mutation can be amplified using PCR before hybridization.

DNA sequences of the p53 gene from the cancer cell tissue which have been amplified by use of polymerase chain reaction can also be screened using allele-specific probes. These probes are nucleic acid oligomers, each of which contains a region of the p53 gene sequence harboring a known mutation. For example, one oligomer can be about 30 nucleotides in length, corresponding to a portion of the p53 gene sequence. At the position coding for the 175th codon of p53 gene the oligomer encodes an alanine, rather than the wild type codon valine. By use of a battery of such allele-specific probes, the PCR amplification products can be screened to identify the presence of a previously identified mutation in the p53 gene. Hybridization of allele-specific probes with amplified p53 sequences can be performed, for example, on a nylon filter. Hybridization to a particular probe indicates the presence of the same mutation in the cancer cell tissue as in the allele-specific probe.

The identification of p53 gene structural changes in cancer cells can be facilitated through the application of a diverse series of high resolution, high throughput microarray platforms. Essentially two types of array include those that carry PCR products from cloned nucleic acids (e.g. cDNA, BACs, cosmids) and those that use oligonucleotides. The methods can provide a way to survey genome wide DNA copy number abnormalities and expression levels to allow correlations between losses, gains and amplifications in cancer cells with genes that are over- and under-expressed in the same samples. The gene expression arrays that provide estimates of mRNA levels in cancer cells have given rise to exon-specific arrays that can identify both gene expression levels, alternative splicing events and mRNA processing alterations.

Oligonucleotide arrays can be used to interrogate single nucleotide polymorphisms (SNPs) throughout the genome for linkage and association studies and these have been adapted to quantify copy number abnormalities and loss of heterozygosity events. DNA sequencing arrays can allow resequencing of chromosome regions, exomes, and whole genomes.

SNP-based arrays or other gene arrays or chips can determine the presence of wild type p53 allele and the structure of mutations. A single nucleotide polymorphism (SNP), a variation at a single site in DNA, is the most frequent type of variation in the genome. For example, there are an estimated 5-10 million SNPs in the human genome. SNPs can be synonymous or nonsynonymous substitutions. Synonymous SNP substitutions do not result in a change of amino acid in the protein due to the degeneracy of the genetic code, but can affect function in other ways. For example, a seemingly silent mutation in a gene that codes for a membrane transport protein can slow down translation, allowing the peptide chain to misfold, and produce a less functional mutant membrane transport protein. Nonsynonymous SNP substitutions can be missense substitutions or nonsense substitutions. Missense substitutions occur when a single base change results in change in amino acid sequence of the protein and malfunction thereof leads to disease. Nonsense substitutions occur when a point mutation results in a premature stop codon, or a nonsense codon in the transcribed mRNA, which results in a truncated and usually, nonfunctional, protein product. As SNPs are highly conserved throughout evolution and within a population, the map of SNPs serves as an excellent genotypic marker for research. SNP array is a useful tool to study the whole genome.

In addition, SNP array can be used for studying the Loss Of Heterozygosity (LOH). LOH is a form of allelic imbalance that can result from the complete loss of an allele or from an increase in copy number of one allele relative to the other. While other chip-based methods (e.g., comparative genomic hybridization can detect only genomic gains or deletions), SNP array has the additional advantage of detecting copy number neutral LOH due to uniparental disomy (UPD). In UPD, one allele or whole chromosome from one parent are missing leading to reduplication of the other parental allele (uni-parental=from one parent, disomy=duplicated). In a disease setting this occurrence can be pathologic when the wild type allele (e.g., from the mother) is missing and instead two copies of the heterozygous allele (e.g., from the father) are present. This usage of SNP array has a huge potential in cancer diagnostics as LOH is a prominent characteristic of most human cancers. SNP array technology have shown that cancers (e.g. gastric cancer, liver cancer, etc.) and hematologic malignancies (ALL, MDS, CML etc) have a high rate of LOH due to genomic deletions or UPD and genomic gains. In the present disclosure, using high density SNP array to detect LOH allows identification of pattern of allelic imbalance to determine the presence of wild type p53 allele.

Mutations of wild type p53 genes can also be detected on the basis of the mutation of a wild type expression product of the p53 gene. Such expression products include both the mRNA as well as the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via the polymerase chain reaction (PCR). A panel of monoclonal antibodies could be used in which each of the epitopes involved in p53 functions are represented by a monoclonal antibody. Loss or perturbation of binding of a monoclonal antibody in the panel can indicate mutational alteration of the p53 protein and thus of the p53 gene itself. Mutant p53 genes or gene products can also be detected in body samples, including, for example, serum, stool, urine, and sputum. The same techniques discussed above for detection of mutant p53 genes or gene products in tissues can be applied to other body samples.

Loss of wild type p53 genes can also be detected by screening for loss of wild type p53 protein function. Although all of the functions which the p53 protein undoubtedly possesses have yet to be elucidated, at least two specific functions are known. Protein p53 binds to the SV40 large T antigen as well as to the adenovirus E1B antigen. Loss of the ability of the p53 protein to bind to either or both of these antigens indicates a mutational alteration in the protein which reflects a mutational alteration of the gene itself. Alternatively, a panel of monoclonal antibodies could be used in which each of the epitopes involved in p53 functions are represented by a monoclonal antibody. Loss or perturbation of binding of a monoclonal antibody in the panel would indicate mutational alteration of the p53 protein and thus of the p53 gene itself. Any method for detecting an altered p53 protein can be used to detect loss of wild type p53 genes.

### Assays

The properties of peptidomimetic macrocycles are assayed, for example, by using the methods described below. In some embodiments, a peptidomimetic macrocycle has improved biological properties relative to a corresponding polypeptide lacking the substituents described herein.

a. Assays to Determine α-Helicity

In solution, the secondary structure of polypeptides with α-helical domains will reach a dynamic equilibrium between random coil structures and α-helical structures, often expressed as a “percent helicity”. Thus, for example, alpha-helical domains are predominantly random coils in solution, with α-helical content usually under 25%. Peptidomimetic macrocycles with optimized linkers, on the other hand, possess, for example, an alpha-helicity that is at least two-fold greater than that of a corresponding uncrosslinked polypeptide. In some embodiments, macrocycles will possess an alpha-helicity of greater than 50%. To assay the helicity of peptidomimetic macrocycles, the compounds are dissolved in an aqueous solution (e.g. 50 mM potassium phosphate solution at pH 7, or distilled H₂O, to concentrations of 25-50 μM). Circular dichroism (CD) spectra are obtained on a spectropolarimeter using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g. [Φ]222obs) by the reported value for a model helical decapeptide.

b. Assay to Determine Melting Temperature (Tₘ)

A peptidomimetic macrocycle comprising a secondary structure such as an a-helix exhibits, for example, a higher melting temperature than a corresponding uncrosslinked polypeptide. Peptidomimetic macrocycles exhibit Tm of >60° C. representing a highly stable structure in aqueous solutions. To assay the effect of macrocycle formation on melting temperature, peptidomimetic macrocycles or unmodified peptides are dissolved in distilled H₂O (e.g. at a final concentration of 50 μM) and the Tm is determined by measuring the change in ellipticity over a temperature range (e.g. 4 to 95° C.) on a spectropolarimeter using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

c. Protease Resistance Assay

The amide bond of the peptide backbone is susceptible to hydrolysis by proteases, thereby rendering peptidic compounds vulnerable to rapid degradation in vivo. Peptide helix formation, however, buries the amide backbone and therefore can shield it from proteolytic cleavage. The peptidomimetic macrocycles can be subjected to in vitro trypsin proteolysis to assess for any change in degradation rate compared to a corresponding uncrosslinked polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (S/E˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time (k=−1Xslope).

d. Ex Vivo Stability Assay

Peptidomimetic macrocycles with optimized linkers possess, for example, an ex vivo half-life that is at least two-fold greater than that of a corresponding uncrosslinked polypeptide, and possess an ex vivo half-life of 12 hours or more. For ex vivo serum stability studies, a variety of assays can be used. For example, a peptidomimetic macrocycle and a corresponding uncrosslinked polypeptide (2 mcg) are incubated with fresh mouse, rat and/or human serum (2 mL) at 37° C. for 0, 1, 2, 4, 8, and 24 hours. To determine the level of intact compound, the following procedure can be used: The samples are extracted by transferring 100 μL of sera to 2 ml centrifuge tubes followed by the addition of 10 μL of 50% formic acid and 500 μL acetonitrile and centrifugation at 14,000 RPM for 10 min at 4±2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis.

e. In Vitro Binding Assays

To assess the binding and affinity of peptidomimetic macrocycles and peptidomimetic precursors to acceptor proteins, a fluorescence polarization assay (FPA) is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution).

For example, fluoresceinated peptidomimetic macrocycles (25 nM) are incubated with the acceptor protein (25-1000 nM) in binding buffer (140 mM NaCl, 50 mM Tris-HCL, pH 7.4) for 30 minutes at room temperature. Binding activity is measured, for example, by fluorescence polarization on a luminescence spectrophotometer. Kd values can be determined by nonlinear regression analysis using, for example, GraphPad Prism software. A peptidomimetic macrocycle shows, In some embodiments, similar or lower Kd than a corresponding uncrosslinked polypeptide.

f. In Vitro Displacement Assays to Characterize Antagonists of Peptide-Protein Interactions

To assess the binding and affinity of compounds that antagonize the interaction between a peptide and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution). A compound that antagonizes the interaction between the fluoresceinated peptidomimetic macrocycle and an acceptor protein will be detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCL, pH 7.4) for 30 minutes at room temperature. Antagonist binding activity is measured, for example, by fluorescence polarization on a luminescence spectrophotometer. Kd values can be determined by nonlinear regression analysis. Any class of molecule, such as small organic molecules, peptides, oligonucleotides or proteins can be examined as putative antagonists in this assay.

g. Assay for Protein-Ligand Binding by Affinity Selection-Mass Spectrometry

To assess the binding and affinity of test compounds for proteins, an affinity-selection mass spectrometry assay is used, for example. Protein-ligand binding experiments are conducted according to the following representative procedure outlined for a system-wide control experiment using 1 μM peptidomimetic macrocycle plus 5 μM hMDM2. A 1 μL DMSO aliquot of a 40 μM stock solution of peptidomimetic macrocycle is dissolved in 19 μL of PBS (50 mM, pH 7.5 Phosphate buffer containing 150 mM NaCl). The resulting solution is mixed by repeated pipetting and clarified by centrifugation at 10 000 g for 10 min. To a 4 μL aliquot of the resulting supernatant is added 4 μL of 10 μM hMDM2 in PBS. Each 8.0 μL experimental sample thus contains 40 pmol (1.5 μg) of protein at 5.0 μM concentration in PBS plus 1 μM peptidomimetic macrocycle and 2.5% DMSO. Duplicate samples thus prepared for each concentration point are incubated for 60 min at room temperature, and then chilled to 4° C. prior to size-exclusion chromatography-LC-MS analysis of 5.0 μL injections. Samples containing a target protein, protein-ligand complexes, and unbound compounds are injected onto an SEC column, where the complexes are separated from non-binding component by a rapid SEC step. The SEC column eluate is monitored using UV detectors to confirm that the early-eluting protein fraction, which elutes in the void volume of the SEC column, is well resolved from unbound components that are retained on the column. After the peak containing the protein and protein-ligand complexes elutes from the primary UV detector, it enters a sample loop where it is excised from the flow stream of the SEC stage and transferred directly to the LC-MS via a valving mechanism. The (M+3H)³⁺ ion of the peptidomimetic macrocycle is observed by ESI-MS at the expected m/z, confirming the detection of the protein-ligand complex.

h. Assay for Protein-Ligand K_{d} Titration Experiments

To assess the binding and affinity of test compounds for proteins, a protein-ligand Kd titration experiment is performed, for example. Protein-ligand K_{d} titrations experiments are conducted as follows: 2 μL DMSO aliquots of a serially diluted stock solution of titrant peptidomimetic macrocycle (5, 2.5, . . . , 0.098 mM) are prepared then dissolved in 38 μL of PBS. The resulting solutions are mixed by repeated pipetting and clarified by centrifugation at 10 000 g for 10 min. To 4.0 μL aliquots of the resulting supernatants is added 4.0 μL of 10 μM hMDM2 in PBS. Each 8.0 μL experimental sample thus contains 40 pmol (1.5 μg) of protein at 5.0 μM concentration in PBS, varying concentrations (125, 62.5, . . . , 0.24 μM) of the titrant peptide, and 2.5% DMSO. Duplicate samples thus prepared for each concentration point are incubated at room temperature for 30 min, then chilled to 4° C. prior to SEC-LC-MS analysis of 2.0 μL injections. The (M+H)¹⁺, (M+2H)²⁺, (M+3H)³⁺, and/or (M+Na)¹⁺ion is observed by ESI-MS; extracted ion chromatograms are quantified, then fit to equations to derive the binding affinity K_{d}.

i. Assay for Competitive Binding Experiments by Affinity Selection-Mass Spectrometry

To determine the ability of test compounds to bind competitively to proteins, an affinity selection mass spectrometry assay is performed, for example. A mixture of ligands at 40 μM per component is prepared by combining 2 μL aliquots of 400 μM stocks of each of the three compounds with 14 μL of DMSO. Then, 1 μL aliquots of this 40 μM per component mixture are combined with 1 μL DMSO aliquots of a serially diluted stock solution of titrant peptidomimetic macrocycle (10, 5, 2.5, . . . , 0.078 mM). These 2μL samples are dissolved in 38 μL of PBS. The resulting solutions were mixed by repeated pipetting and clarified by centrifugation at 10 000 g for 10 min. To 4.0 μL aliquots of the resulting supernatants is added 4.0 μL of 10 μM hMDM2 protein in PBS. Each 8.0 μL experimental sample thus contains 40 pmol (1.5 μg) of protein at 5.0 concentration in PBS plus 0.5 μM ligand, 2.5% DMSO, and varying concentrations (125, 62.5, . . . , 0.98 μM) of the titrant peptidomimetic macrocycle. Duplicate samples thus prepared for each concentration point are incubated at room temperature for 60 min, then chilled to 4° C. prior to SEC-LC-MS analysis of 2.0 μL injections.

j. Binding Assays in Intact Cells

It is possible to measure binding of peptides or peptidomimetic macrocycles to their natural acceptors in intact cells by immunoprecipitation experiments. For example, intact cells are incubated with fluoresceinated (FITC-labeled) compounds for 4 hrs in the absence of serum, followed by serum replacement and further incubation that ranges from 4-18 hrs. Cells are then pelleted and incubated in lysis buffer (50 mM Tris [pH 7.6], 150 mM NaCl, 1% CHAPS and protease inhibitor cocktail) for 10 minutes at 4° C. Extracts are centrifuged at 14,000 rpm for 15 minutes and supernatants collected and incubated with 10 μL goat anti-FITC antibody for 2 hrs, rotating at 4° C. followed by further 2 hrs incubation at 4° C. with protein A/G Sepharose (50 μL of 50% bead slurry). After quick centrifugation, the pellets are washed in lysis buffer containing increasing salt concentration (e.g., 150, 300, 500 mM). The beads are then re-equilibrated at 150 mM NaCl before addition of SDS-containing sample buffer and boiling. After centrifugation, the supernatants are optionally electrophoresed using 4%-12% gradient Bis-Tris gels followed by transfer into Immobilon-P membranes. After blocking, blots are optionally incubated with an antibody that detects FITC and also with one or more antibodies that detect proteins that bind to the peptidomimetic macrocycle.

k. Cellular Penetrability Assays

A peptidomimetic macrocycle is, for example, more cell penetrable compared to a corresponding uncrosslinked macrocycle. Peptidomimetic macrocycles with optimized linkers possess, for example, cell penetrability that is at least two-fold greater than a corresponding uncrosslinked macrocycle, and often 20% or more of the applied peptidomimetic macrocycle will be observed to have penetrated the cell after 4 hours. To measure the cell penetrability of peptidomimetic macrocycles and corresponding uncrosslinked macrocycle, intact cells are incubated with fluorescently-labeled (e.g. fluoresceinated) peptidomimetic macrocycles or corresponding uncrosslinked macrocycle (10 μM) for 4 hrs in serum free media at 37° C., washed twice with media and incubated with trypsin (0.25%) for 10 min at 37° C. The cells are washed again and resuspended in PBS. Cellular fluorescence is analyzed.

l. Cellular Efficacy Assays

The efficacy of certain peptidomimetic macrocycles is determined, for example, in cell-based killing assays using a variety of tumorigenic and non-tumorigenic cell lines and primary cells derived from human or mouse cell populations. Cell viability is monitored, for example, over 24-96 hrs of incubation with peptidomimetic macrocycles (0.5 to 50 μM) to identify those that kill at EC₅₀<10 μM. Several standard assays that measure cell viability are commercially available and are optionally used to assess the efficacy of the peptidomimetic macrocycles. In addition, assays that measure Annexin V and caspase activation are optionally used to assess whether the peptidomimetic macrocycles kill cells by activating the apoptotic machinery. For example, the Cell Titer-glo assay is used which determines cell viability as a function of intracellular ATP concentration.

m. In Vivo Stability Assay

To investigate the in vivo stability of the peptidomimetic macrocycles, the compounds are, for example, administered to mice and/or rats by IV, IP, PO or inhalation routes at concentrations ranging from 0.1 to 50 mg/kg and blood specimens withdrawn at 0′, 5′, 15′, 30′, 1 hr, 4 hrs, 8 hrs and 24 hours post-injection. Levels of intact compound in 25 μL of fresh serum are then measured by LC-MS/MS as above.

n. In Vivo Efficacy in Animal Models

To determine the anti-oncogenic activity of peptidomimetic macrocycles in vivo, the compounds are, for example, given alone (IP, IV, PO, by inhalation or nasal routes) or in combination with sub-optimal doses of relevant chemotherapy (e.g., cyclophosphamide, doxorubicin, etoposide). In one example, 5×10⁶ RS4; 11 cells (established from the bone marrow of a patient with acute lymphoblastic leukemia) that stably express luciferase are injected by tail vein in NOD-SCID mice 3 hrs after they have been subjected to total body irradiation. If left untreated, this form of leukemia is fatal in 3 weeks in this model. The leukemia is readily monitored, for example, by injecting the mice with D-luciferin (60 mg/kg) and imaging the anesthetized animals. Total body bioluminescence is quantified by integration of photonic flux (photons/sec) by Living Image Software. Peptidomimetic macrocycles alone or in combination with sub-optimal doses of relevant chemotherapeutics agents are, for example, administered to leukemic mice (10 days after injection/day 1 of experiment, in bioluminescence range of 14-16) by tail vein or IP routes at doses ranging from 0.1mg/kg to 50 mg/kg for 7 to 21 days. Optionally, the mice are imaged throughout the experiment every other day and survival monitored daily for the duration of the experiment. Expired mice are optionally subjected to necropsy at the end of the experiment. Another animal model is implantation into NOD-SCID mice of DoHH2, a cell line derived from human follicular lymphoma that stably expresses luciferase. These in vivo tests optionally generate preliminary pharmacokinetic, pharmacodynamic and toxicology data.

o. Clinical Trials

To determine the suitability of the peptidomimetic macrocycles for treatment of humans, clinical trials are performed. For example, patients diagnosed with cancer and in need of treatment can be selected and separated in treatment and one or more control groups, wherein the treatment group is administered a peptidomimetic macrocycle, while the control groups receive a placebo or a known anti-cancer drug. The treatment safety and efficacy of the peptidomimetic macrocycles can thus be evaluated by performing comparisons of the patient groups with respect to factors such as survival and quality-of-life. In this example, the patient group treated with a peptidomimetic macrocycle can show improved long-term survival compared to a patient control group treated with a placebo.

### EXAMPLES

### Example 1

### Peptidomimetic Macrocycles

Peptidomimetic macrocycles were designed by replacing two or more naturally-occurring amino acids with the corresponding synthetic amino acids. Substitutions were made at i and i+4, and i and i+7 positions. Peptide synthesis was performed manually or using an automated peptide synthesizer under solid phase conditions using rink amide AM resin and Fmoc main-chain protecting group chemistry. For the coupling of natural Fmoc-protected amino acids, 10 eq. of amino acid and a 1:1:2 molar ratio of coupling reagents HBTU/HOBt/DIEA were employed. Non-natural amino acids (4 eq.) were coupled with a 1:1:2 molar ratio of HATU/HOBt/DIEA. The N-termini of the synthetic peptides were acetylated, and the C-termini were amidated.

Purification of crosslinked compounds was achieved by HPLC on a reverse phase C18 column to yield the pure compounds. The chemical compositions of the pure products were confirmed by LC/MS mass spectrometry and amino acid analysis.

Synthesis of dialkyne-crosslinked peptidomimetic macrocycles, including SP662, SP663, and SP664. Fully protected resin-bound peptides were synthesized on a PEG-PS resin (loading 0.45 mmol/g) on a 0.2 mmol scale. Deprotection of the temporary Fmoc group was achieved by 3×10 min treatments of the resin bound peptide with 20% (v/v) piperidine in DMF. After washing with NMP (3×), dichloromethane (3×) and NMP (3×), coupling of each successive amino acid was achieved with 1×60 min incubation with the appropriate pre-activated Fmoc-amino acid derivative. All protected amino acids (0.4 mmol) were dissolved in NMP and activated with HCTU (0.4 mmol) and DIEA (0.8 mmol) prior to transfer of the coupling solution to the de-protected resin-bound peptide. After coupling was completed, the resin was washed in preparation for the next deprotection/coupling cycle.

Acetylation of the amino terminus was carried out in the presence of acetic anhydride/DIEA in NMP. The LC-MS analysis of a cleaved and de-protected sample obtained from an aliquot of the fully assembled resin-bound peptide was accomplished in order to verifying the completion of each coupling. In a typical example, tetrahydrofuran (4 ml) and triethylamine (2 ml) were added to the peptide resin (0.2 mmol) in a 40 ml glass vial and shaken for 10 minutes. Pd(PPh₃)₂Cl₂ (0.014 g, 0.02 mmol) and copper iodide (0.008 g, 0.04 mmol) were then added and the resulting reaction mixture was mechanically shaken 16 hours while open to atmosphere. The diyne-cyclized resin-bound peptides were de-protected and cleaved from the solid support by treatment with TFA/H₂O/TIS (95/5/5 v/v) for 2.5 h at room temperature. After filtration of the resin the TFA solution was precipitated in cold diethyl ether and centrifuged to yield the desired product as a solid. The crude product was purified by preparative HPLC.

Synthesis of single alkyne-crosslinked peptidomimetic macrocycles, including SP665. Fully protected resin-bound peptides were synthesized on a Rink amide MBHA resin (loading 0.62 mmol/g) on a 0.1 mmol scale. Deprotection of the temporary Fmoc group was achieved by 2×20 min treatments of the resin bound peptide with 25% (v/v) piperidine in NMP. After extensive flow washing with NMP and dichloromethane, coupling of each successive amino acid was achieved with 1×60 min incubation with the appropriate pre-activated Fmoc-amino acid derivative. All protected amino acids (1 mmol) were dissolved in NMP and activated with HCTU (1 mmol) and DIEA (1 mmol) prior to transfer of the coupling solution to the de-protected resin-bound peptide. After coupling was completed, the resin was extensively flow washed in preparation for the next deprotection/coupling cycle.

Acetylation of the amino terminus was carried out in the presence of acetic anhydride/DIEA in NMP/NMM. The LC-MS analysis of a cleaved and de-protected sample obtained from an aliquot of the fully assembled resin-bound peptide was accomplished to verify the completion of each coupling reaction. In a typical example, the peptide resin (0.1 mmol) was washed with DCM. Resin was loaded into a microwave vial. The vessel was evacuated and purged with nitrogen. Molybdenum hexacarbonyl (0.01 eq.) was added. Anhydrous chlorobenzene was added to the reaction vessel. Then 2-fluorophenol (1 eq.) was added. The reaction was then loaded into the microwave and held at 130° C. for 10 minutes. The reaction pushed for a longer period time when needed to complete the reaction. The alkyne-metathesized resin-bound peptides were de-protected and cleaved from the solid support by treating the solid support with TFA/H₂O/TIS (94/3/3 v/v) for 3 h at room temperature. After filtration of the resin, the TFA solution was precipitated in cold diethyl ether and centrifuged to yield the desired product as a solid. The crude product was purified by preparative HPLC.

TABLE 1 shows a list of peptidomimetic macrocycles prepared.

TABLE 1

| | | | Exact | Found | Calc | Calc | Calc |
|---|---|---|---|---|---|---|---|
| SP | Sequence | Isomer | Mass | Mass | (M + 1)/1 | (M + 2)/2 | (M + 3)/3 |
| 1 | Ac-F$r8AYWEAc3cL$AAA-NH₂ | | 1456.78 | 729.44 | 1457.79 | 729.4 | 486.6 |
| 2 | Ac-F$r8AYWEAc3cL$AAibA-NH₂ | | 1470.79 | 736.4 | 1471.8 | 736.4 | 491.27 |
| 3 | Ac-LTF$r8AYWAQL$SANle-NH₂ | | 1715.97 | 859.02 | 1716.98 | 858.99 | 573 |
| 4 | Ac-LTF$r8AYWAQL$SAL-NH₂ | | 1715.97 | 859.02 | 1716.98 | 858.99 | 573 |
| 5 | Ac-LTF$r8AYWAQL$SAM-NH₂ | | 1733.92 | 868.48 | 1734.93 | 867.97 | 578.98 |
| 6 | Ac-LTF$r8AYWAQL$SAhL-NH₂ | | 1729.98 | 865.98 | 1730.99 | 866 | 577.67 |
| 7 | Ac-LTF$r8AYWAQL$SAF-NH₂ | | 1749.95 | 876.36 | 1750.96 | 875.98 | 584.32 |
| 8 | Ac-LTF$r8AYWAQL$SAI-NH₂ | | 1715.97 | 859.02 | 1716.98 | 858.99 | 573 |
| 9 | Ac-LTF$r8AYWAQL$SAChg-NH₂ | | 1741.98 | 871.98 | 1742.99 | 872 | 581.67 |
| 10 | Ac-LTF$r8AYWAQL$SAAib-NH₂ | | 1687.93 | 845.36 | 1688.94 | 844.97 | 563.65 |
| 11 | Ac-LTF$r8AYWAQL$SAA-NH₂ | | 1673.92 | 838.01 | 1674.93 | 837.97 | 558.98 |
| 12 | Ac-LTF$r8AYWA$L$S$Nle-NH₂ | | 1767.04 | 884.77 | 1768.05 | 884.53 | 590.02 |
| 13 | Ac-LTF$r8AYWA$L$S$A-NH₂ | | 1724.99 | 864.23 | 1726 | 863.5 | 576 |
| 14 | Ac-F$r8AYWEAc3cL$AANle-NH₂ | | 1498.82 | 750.46 | 1499.83 | 750.42 | 500.61 |
| 15 | Ac-F$r8AYWEAc3cL$AAL-NH₂ | | 1498.82 | 750.46 | 1499.83 | 750.42 | 500.61 |
| 16 | Ac-F$r8AYWEAc3cL$AAM-NH₂ | | 1516.78 | 759.41 | 1517.79 | 759.4 | 506.6 |
| 17 | Ac-F$r8AYWEAc3cL$AAhL-NH₂ | | 1512.84 | 757.49 | 1513.85 | 757.43 | 505.29 |
| 18 | Ac-F$r8AYWEAc3cL$AAF-NH₂ | | 1532.81 | 767.48 | 1533.82 | 767.41 | 511.94 |
| 19 | Ac-F$r8AYWEAc3cL$AAI-NH₂ | | 1498.82 | 750.39 | 1499.83 | 750.42 | 500.61 |
| 20 | Ac-F$r8AYWEAc3cL$AAChg-NH₂ | | 1524.84 | 763.48 | 1525.85 | 763.43 | 509.29 |
| 21 | Ac-F$r8AYWEAc3cL$AACha-NH₂ | | 1538.85 | 770.44 | 1539.86 | 770.43 | 513.96 |
| 22 | Ac-F$r8AYWEAc3cL$AAAib-NH₂ | | 1470.79 | 736.84 | 1471.8 | 736.4 | 491.27 |
| 23 | Ac-LTF$r8AYWAQL$AAAibV-NH₂ | | 1771.01 | 885.81 | 1772.02 | 886.51 | 591.34 |
| 24 | Ac-LTF$r8AYWAQL$AAAibV-NH₂ | iso2 | 1771.01 | 886.26 | 1772.02 | 886.51 | 591.34 |
| 25 | Ac-LTF$r8AYWAQL$SAibAA-NH₂ | | 1758.97 | 879.89 | 1759.98 | 880.49 | 587.33 |
| 26 | Ac-LTF$r8AYWAQL$SAibAA-NH₂ | iso2 | 1758.97 | 880.34 | 1759.98 | 880.49 | 587.33 |
| 27 | Ac-HLTF$r8HHWHQL$AANleNle-NH₂ | | 2056.15 | 1028.86 | 2057.16 | 1029.08 | 686.39 |
| 28 | Ac-DLTF$r8HHWHQL$RRLV-NH₂ | | 2190.23 | 731.15 | 2191.24 | 1096.12 | 731.08 |
| 29 | Ac-HHTF$r8HHWHQL$AAML-NH₂ | | 2098.08 | 700.43 | 2099.09 | 1050.05 | 700.37 |
| 30 | Ac-F$r8HHWHQL$RRDCha-NH₂ | | 1917.06 | 959.96 | 1918.07 | 959.54 | 640.03 |
| 31 | Ac-F$r8HHWHQL$HRFV-NH₂ | | 1876.02 | 938.65 | 1877.03 | 939.02 | 626.35 |
| 32 | Ac-HLTF$r8HHWHQL$AAhLA-NH₂ | | 2028.12 | 677.2 | 2029.13 | 1015.07 | 677.05 |
| 33 | Ac-DLTF$r8HHWHQL$RRChgl-NH₂ | | 2230.26 | 1115.89 | 2231.27 | 1116.14 | 744.43 |
| 34 | Ac-DLTF$r8HHWHQL$RRChgl-NH₂ | iso2 | 2230.26 | 1115.96 | 2231.27 | 1116.14 | 744.43 |
| 35 | Ac-HHTF$r8HHWHQL$AAChav-NH₂ | | 2106.14 | 1053.95 | 2107.15 | 1054.08 | 703.05 |
| 36 | Ac-F$r8HHWHQL$RRDa-NH₂ | | 1834.99 | 918.3 | 1836 | 918.5 | 612.67 |
| 37 | Ac-F$r8HHWHQL$HRAibG-NH₂ | | 1771.95 | 886.77 | 1772.96 | 886.98 | 591.66 |
| 38 | Ac-F$r8AYWAQL$HHNleL-NH₂ | | 1730.97 | 866.57 | 1731.98 | 866.49 | 578 |
| 39 | Ac-F$r8AYWSAL$HQANle-NH₂ | | 1638.89 | 820.54 | 1639.9 | 820.45 | 547.3 |
| 40 | Ac-F$r8AYWVQL$QHChgl-NH₂ | | 1776.01 | 889.44 | 1777.02 | 889.01 | 593.01 |
| 41 | Ac-F$r8AYWTAL$QQNlev-NH₂ | | 1671.94 | 836.97 | 1672.95 | 836.98 | 558.32 |
| 42 | Ac-F$r8AYWYQL$HAibAa-NH₂ | | 1686.89 | 844.52 | 1687.9 | 844.45 | 563.3 |
| 43 | Ac-LTF$r8AYWAQL$HHLa-NH₂ | | 1903.05 | 952.27 | 1904.06 | 952.53 | 635.36 |
| 44 | Ac-LTF$r8AYWAQL$HHLa-NH₂ | iso2 | 1903.05 | 952.27 | 1904.06 | 952.53 | 635.36 |
| 45 | Ac-LTF$r8AYWAQL$HQNlev-NH₂ | | 1922.08 | 962.48 | 1923.09 | 962.05 | 641.7 |
| 46 | Ac-LTF$r8AYWAQL$HQNlev-NH₂ | iso2 | 1922.08 | 962.4 | 1923.09 | 962.05 | 641.7 |
| 47 | Ac-LTF$r8AYWAQL$QQMl-NH₂ | | 1945.05 | 973.95 | 1946.06 | 973.53 | 649.36 |
| 48 | Ac-LTF$r8AYWAQL$QQMl-NH₂ | iso2 | 1945.05 | 973.88 | 1946.06 | 973.53 | 649.36 |
| 49 | Ac-LTF$r8AYWAQL$HAibhLV-NH₂ | | 1893.09 | 948.31 | 1894.1 | 947.55 | 632.04 |
| 50 | Ac-LTF$r8AYWAQL$AHFA-NH₂ | | 1871.01 | 937.4 | 1872.02 | 936.51 | 624.68 |
| 51 | Ac-HLTF$r8HHWNQL$AANlel-NH₂ | | 2056.15 | 1028.79 | 2057.16 | 1029.08 | 686.39 |
| 52 | Ac-DLTF$r8HHWNQL$RRLa-NH₂ | | 2162.2 | 721.82 | 2163.21 | 1082.11 | 721.74 |
| 53 | Ac-HHTF$r8HHWHQL$AAMv-NH₂ | | 2084.07 | 1042.92 | 2085.08 | 1043.04 | 695.7 |
| 54 | Ac-F$r8HHWHQL$RRDA-NH₂ | | 1834.99 | 612.74 | 1836 | 918.5 | 612.67 |
| 55 | Ac-F$r8HHWHQL$HRFCha-NH₂ | | 1930.06 | 966.47 | 1931.07 | 966.04 | 644.36 |
| 56 | Ac-F$r8AYWEAL$AA-NHAm | | 1443.82 | 1445.71 | 1444.83 | 722.92 | 482.28 |
| 57 | Ac-F$r8AYWEAL$AA-NHiAm | | 1443.82 | 723.13 | 1444.83 | 722.92 | 482.28 |
| 58 | Ac-F$r8AYWEAL$AA-NHnPr3Ph | | 1491.82 | 747.3 | 1492.83 | 746.92 | 498.28 |
| 59 | Ac-F$r8AYWEAL$AA-NHnBu33Me | | 1457.83 | 1458.94 | 1458.84 | 729.92 | 486.95 |
| 60 | Ac-F$r8AYWEAL$AA-NHnPr | | 1415.79 | 709.28 | 1416.8 | 708.9 | 472.94 |
| 61 | Ac-F$r8AYWEAL$AA-NHnEt2Ch | | 1483.85 | 1485.77 | 1484.86 | 742.93 | 495.62 |
| 62 | Ac-F$r8AYWEAL$AA-NHnEt2Cp | | 1469.83 | 1470.78 | 1470.84 | 735.92 | 490.95 |
| 63 | Ac-F$r8AYWEAL$AA-NHHex | | 1457.83 | 730.19 | 1458.84 | 729.92 | 486.95 |
| 64 | Ac-LTF$r8AYWAQL$AAIA-NH₂ | | 1771.01 | 885.81 | 1772.02 | 886.51 | 591.34 |
| 65 | Ac-LTF$r8AYWAQL$AAIA-NH₂ | iso2 | 1771.01 | 866.8 | 1772.02 | 886.51 | 591.34 |
| 66 | Ac-LTF$r8AYWAAL$AAMA-NH₂ | | 1731.94 | 867.08 | 1732.95 | 866.98 | 578.32 |
| 67 | Ac-LTF$r8AYWAALSAAMA-NH₂ | iso2 | 1731.94 | 867.28 | 1732.95 | 866.98 | 578.32 |
| 68 | Ac-LTF$r8AYWAQL$AANleA-NH₂ | | 1771.01 | 867.1 | 1772.02 | 886.51 | 591.34 |
| 69 | Ac-LTF$r8AYWAQL$AANleA-NH₂ | iso2 | 1771.01 | 886.89 | 1772.02 | 886.51 | 591.34 |
| 70 | Ac-LTF$r8AYWAQL$AAIa-NH₂ | | 1771.01 | 886.8 | 1772.02 | 886.51 | 591.34 |
| 71 | Ac-LTF$r8AYWAQL$AAIa-NH₂ | iso2 | 1771.01 | 887.09 | 1772.02 | 886.51 | 591.34 |
| 72 | Ac-LTF$r8AYWAAL$AAMa-NH₂ | | 1731.94 | 867.17 | 1732.95 | 866.98 | 578.32 |
| 73 | Ac-LTF$r8AYWAALSAAMa-NH₂ | iso2 | 1731.94 | 867.37 | 1732.95 | 866.98 | 578.32 |
| 74 | Ac-LTF$r8AYWAQL$AANlea-NH₂ | | 1771.01 | 887.08 | 1772.02 | 886.51 | 591.34 |
| 75 | Ac-LTF$r8AYWAQL$AANlea-NH₂ | iso2 | 1771.01 | 887.08 | 1772.02 | 886.51 | 591.34 |
| 76 | Ac-LTF$r8AYWAAL$AAIv-NH₂ | | 1742.02 | 872.37 | 1743.03 | 872.02 | 581.68 |
| 77 | Ac-LTF$r8AYWAAL$AAIv-NH₂ | iso2 | 1742.02 | 872.74 | 1743.03 | 872.02 | 581.68 |
| 78 | Ac-LTF$r8AYWAQL$AAMv-NH₂ | | 1817 | 910.02 | 1818.01 | 909.51 | 606.67 |
| 79 | Ac-LTF$r8AYWAAL$AANlev-NH₂ | | 1742.02 | 872.37 | 1743.03 | 872.02 | 581.68 |
| 80 | Ac-LTF$r8AYWAAL$AANlev-NH₂ | iso2 | 1742.02 | 872.28 | 1743.03 | 872.02 | 581.68 |
| 81 | Ac-LTF$r8AYWAQL$AAIl-NH₂ | | 1813.05 | 907.81 | 1814.06 | 907.53 | 605.36 |
| 82 | Ac-LTF$r8AYWAQL$AAIl-NH₂ | iso2 | 1813.05 | 907.81 | 1814.06 | 907.53 | 605.36 |
| 83 | Ac-LTF$r8AYWAAL$AAMl-NH₂ | | 1773.99 | 887.37 | 1775 | 888 | 592.34 |
| 84 | Ac-LTF$r8AYWAQL$AANlel-NH₂ | | 1813.05 | 907.61 | 1814.06 | 907.53 | 605.36 |
| 85 | Ac-LTF$r8AYWAQL$AANlel-NH₂ | iso2 | 1813.05 | 907.71 | 1814.06 | 907.53 | 605.36 |
| 86 | Ac-F$r8AYWEAL$AAMA-NH₂ | | 1575.82 | 789.02 | 1576.83 | 788.92 | 526.28 |
| 87 | Ac-F$r8AYWEAL$AANleA-NH₂ | | 1557.86 | 780.14 | 1558.87 | 779.94 | 520.29 |
| 88 | Ac-F$r8AYWEAL$AAIa-NH₂ | | 1557.86 | 780.33 | 1558.87 | 779.94 | 520.29 |
| 89 | Ac-F$r8AYWEAL$AAMa-NH₂ | | 1575.82 | 789.3 | 1576.83 | 788.92 | 526.28 |
| 90 | Ac-F$r8AYWEAL$AANlea-NH₂ | | 1557.86 | 779.4 | 1558.87 | 779.94 | 520.29 |
| 91 | Ac-F$r8AYWEAL$AAIv-NH₂ | | 1585.89 | 794.29 | 1586.9 | 793.95 | 529.64 |
| 92 | Ac-F$r8AYWEAL$AAMv-NH₂ | | 1603.85 | 803.08 | 1604.86 | 802.93 | 535.62 |
| 93 | Ac-F$r8AYWEAL$AANlev-NH₂ | | 1585.89 | 793.46 | 1586.9 | 793.95 | 529.64 |
| 94 | Ac-F$r8AYWEAL$AAIl-NH₂ | | 1599.91 | 800.49 | 1600.92 | 800.96 | 534.31 |
| 95 | Ac-F$r8AYWEALSAAMl-NH₂ | | 1617.86 | 809.44 | 1618.87 | 809.94 | 540.29 |
| 96 | Ac-F$r8AYWEAL$AANlel-NH₂ | | 1599.91 | 801.7 | 1600.92 | 800.96 | 534.31 |
| 97 | Ac-F$r8AYWEAL$AANlel-NH₂ | iso2 | 1599.91 | 801.42 | 1600.92 | 800.96 | 534.31 |
| 98 | Ac-LTF$r8AY6clWAQL$SAA-NH₂ | | 1707.88 | 855.72 | 1708.89 | 854.95 | 570.3 |
| 99 | Ac-LTF$r8AY6clWAQL$SAA-NH₂ | iso2 | 1707.88 | 855.35 | 1708.89 | 854.95 | 570.3 |
| 100 | Ac-WTF$r8FYWSQL$AVAa-NH₂ | | 1922.01 | 962.21 | 1923.02 | 962.01 | 641.68 |
| 101 | Ac-WTF$r8FYWSQL$AVAa-NH₂ | iso2 | 1922.01 | 962.49 | 1923.02 | 962.01 | 641.68 |
| 102 | Ac-WTF$r8VYWSQL$AVA-NH₂ | | 1802.98 | 902.72 | 1803.99 | 902.5 | 602 |
| 103 | Ac-WTF$r8VYWSQL$AVA-NH₂ | iso2 | 1802.98 | 903 | 1803.99 | 902.5 | 602 |
| 104 | Ac-WTF$r8FYWSQL$SAAa-NH₂ | | 1909.98 | 956.47 | 1910.99 | 956 | 637.67 |
| 105 | Ac-WTF$r8FYWSQL$SAAa-NH₂ | iso2 | 1909.98 | 956.47 | 1910.99 | 956 | 637.67 |
| 106 | Ac-WTF$r8VYWSQL$AVAaa-NH₂ | | 1945.05 | 974.15 | 1946.06 | 973.53 | 649.36 |
| 107 | Ac-WTF$r8VYWSQL$AVAaa-NH₂ | iso2 | 1945.05 | 973.78 | 1946.06 | 973.53 | 649.36 |
| 108 | Ac-LTF$r8AYWAQL$AVG-NH₂ | | 1671.94 | 837.52 | 1672.95 | 836.98 | 558.32 |
| 109 | Ac-LTF$r8AYWAQL$AVG-NH₂ | iso2 | 1671.94 | 837.21 | 1672.95 | 836.98 | 558.32 |
| 110 | Ac-LTF$r8AYWAQL$AVQ-NH₂ | | 1742.98 | 872.74 | 1743.99 | 872.5 | 582 |
| 111 | Ac-LTF$r8AYWAQL$AVQ-NH₂ | iso2 | 1742.98 | 872.74 | 1743.99 | 872.5 | 582 |
| 112 | Ac-LTF$r8AYWAQL$SAa-NH₂ | | 1673.92 | 838.23 | 1674.93 | 837.97 | 558.98 |
| 113 | Ac-LTF$r8AYWAQL$SAa-NH₂ | iso2 | 1673.92 | 838.32 | 1674.93 | 837.97 | 558.98 |
| 114 | Ac-LTF$r8AYWAQhL$SAA-NH₂ | | 1687.93 | 844.37 | 1688.94 | 844.97 | 563.65 |
| 115 | Ac-LTF$r8AYWAQhL$SAA-NH₂ | iso2 | 1687.93 | 844.81 | 1688.94 | 844.97 | 563.65 |
| 116 | Ac-LTF$r8AYWEQLStSA$-NH₂ | | 1826 | 905.27 | 1827.01 | 914.01 | 609.67 |
| 117 | Ac-LTF$r8AYWAQL$SLA-NH₂ | | 1715.97 | 858.48 | 1716.98 | 858.99 | 573 |
| 118 | Ac-LTF$r8AYWAQL$SLA-NH₂ | iso2 | 1715.97 | 858.87 | 1716.98 | 858.99 | 573 |
| 119 | Ac-LTF$r8AYWAQL$SWA-NH₂ | | 1788.96 | 895.21 | 1789.97 | 895.49 | 597.33 |
| 120 | Ac-LTF$r8AYWAQL$SWA-NH₂ | iso2 | 1788.96 | 895.28 | 1789.97 | 895.49 | 597.33 |
| 121 | Ac-LTF$r8AYWAQL$SVS-NH₂ | | 1717.94 | 859.84 | 1718.95 | 859.98 | 573.65 |
| 122 | Ac-LTF$r8AYWAQL$SAS-NH₂ | | 1689.91 | 845.85 | 1690.92 | 845.96 | 564.31 |
| 123 | Ac-LTF$r8AYWAQL$SVG-NH₂ | | 1687.93 | 844.81 | 1688.94 | 844.97 | 563.65 |
| 124 | Ac-ETF$r8VYWAQL$SAa-NH₂ | | 1717.91 | 859.76 | 1718.92 | 859.96 | 573.64 |
| 125 | Ac-ETF$r8VYWAQL$SAA-NH₂ | | 1717.91 | 859.84 | 1718.92 | 859.96 | 573.64 |
| 126 | Ac-ETF$r8VYWAQL$SVA-NH₂ | | 1745.94 | 873.82 | 1746.95 | 873.98 | 582.99 |
| 127 | Ac-ETF$r8VYWAQL$SLA-NH₂ | | 1759.96 | 880.85 | 1760.97 | 880.99 | 587.66 |
| 128 | Ac-ETF$r8VYWAQL$SWA-NH₂ | | 1832.95 | 917.34 | 1833.96 | 917.48 | 611.99 |
| 129 | Ac-ETF$r8KYWAQL$SWA-NH₂ | | 1861.98 | 931.92 | 1862.99 | 932 | 621.67 |
| 130 | Ac-ETF$r8VYWAQL$SVS-NH₂ | | 1761.93 | 881.89 | 1762.94 | 881.97 | 588.32 |
| 131 | Ac-ETF$r8VYWAQL$SAS-NH₂ | | 1733.9 | 867.83 | 1734.91 | 867.96 | 578.97 |
| 132 | Ac-ETF$r8VYWAQL$SVG-NH₂ | | 1731.92 | 866.87 | 1732.93 | 866.97 | 578.31 |
| 133 | Ac-LTF$r8VYWAQL$SSa-NH₂ | | 1717.94 | 859.47 | 1718.95 | 859.98 | 573.65 |
| 134 | Ac-ETF$r8VYWAQL$SSa-NH₂ | | 1733.9 | 867.83 | 1734.91 | 867.96 | 578.97 |
| 135 | Ac-LTF$r8VYWAQL$SNa-NH₂ | | 1744.96 | 873.38 | 1745.97 | 873.49 | 582.66 |
| 136 | Ac-ETF$r8VYWAQL$SNa-NH₂ | | 1760.91 | 881.3 | 1761.92 | 881.46 | 587.98 |
| 137 | Ac-LTF$r8VYWAQL$SAa-NH₂ | | 1701.95 | 851.84 | 1702.96 | 851.98 | 568.32 |
| 138 | Ac-LTF$r8VYWAQL$SVA-NH₂ | | 1729.98 | 865.53 | 1730.99 | 866 | 577.67 |
| 139 | Ac-LTF$r8VYWAQL$SVA-NH₂ | iso2 | 1729.98 | 865.9 | 1730.99 | 866 | 577.67 |
| 140 | Ac-LTF$r8VYWAQL$SWA-NH₂ | | 1816.99 | 909.42 | 1818 | 909.5 | 606.67 |
| 141 | Ac-LTF$r8VYWAQL$SVS-NH₂ | | 1745.98 | 873.9 | 1746.99 | 874 | 583 |
| 142 | Ac-LTF$r8VYWAQL$SVS-NH₂ | iso2 | 1745.98 | 873.9 | 1746.99 | 874 | 583 |
| 143 | Ac-LTF$r8VYWAQL$SAS-NH₂ | | 1717.94 | 859.84 | 1718.95 | 859.98 | 573.65 |
| 144 | Ac-LTF$r8VYWAQL$SAS-NH₂ | iso2 | 1717.94 | 859.91 | 1718.95 | 859.98 | 573.65 |
| 145 | Ac-LTF$r8VYWAQL$SVG-NH₂ | | 1715.97 | 858.87 | 1716.98 | 858.99 | 573 |
| 146 | Ac-LTF$r8VYWAQL$SVG-NH₂ | iso2 | 1715.97 | 858.87 | 1716.98 | 858.99 | 573 |
| 147 | Ac-LTF$r8EYWAQCha$SAA-NH₂ | | 1771.96 | 886.85 | 1772.97 | 886.99 | 591.66 |
| 148 | Ac-LTF$r8EYWAQCha$SAA-NH₂ | iso2 | 1771.96 | 886.85 | 1772.97 | 886.99 | 591.66 |
| 149 | Ac-LTF$r8EYWAQCpg$SAA-NH₂ | | 1743.92 | 872.86 | 1744.93 | 872.97 | 582.31 |
| 150 | Ac-LTF$r8EYWAQCpg$SAA-NH₂ | iso2 | 1743.92 | 872.86 | 1744.93 | 872.97 | 582.31 |
| 151 | Ac-LTF$r8EYWAQF$SAA-NH₂ | | 1765.91 | 883.44 | 1766.92 | 883.96 | 589.64 |
| 152 | Ac-LTF$r8EYWAQF$SAA-NH₂ | iso2 | 1765.91 | 883.89 | 1766.92 | 883.96 | 589.64 |
| 153 | Ac-LTF$r8EYWAQCba$SAA-NH₂ | | 1743.92 | 872.42 | 1744.93 | 872.97 | 582.31 |
| 154 | Ac-LTF$r8EYWAQCba$SAA-NH₂ | iso2 | 1743.92 | 873.39 | 1744.93 | 872.97 | 582.31 |
| 155 | Ac-LTF3Cl$r8EYWAQL$SAA-NH₂ | | 1765.89 | 883.89 | 1766.9 | 883.95 | 589.64 |
| 156 | Ac-LTF3Cl$r8EYWAQL$SAA-NH₂ | iso2 | 1765.89 | 883.96 | 1766.9 | 883.95 | 589.64 |
| 157 | Ac-LTF34F2$r8EYWAQL$SAA-NH₂ | | 1767.91 | 884.48 | 1768.92 | 884.96 | 590.31 |
| 158 | Ac-LTF34F2$r8EYWAQL$SAA-NH₂ | iso2 | 1767.91 | 884.48 | 1768.92 | 884.96 | 590.31 |
| 159 | Ac-LTF34F2$r8EYWAQhL$SAA-NH₂ | | 1781.92 | 891.44 | 1782.93 | 891.97 | 594.98 |
| 160 | Ac-LTF34F2$r8EYWAQhL$SAA-NH₂ | iso2 | 1781.92 | 891.88 | 1782.93 | 891.97 | 594.98 |
| 161 | Ac-ETF$r8EYWAQL$SAA-NH₂ | | 1747.88 | 874.34 | 1748.89 | 874.95 | 583.63 |
| 162 | Ac-LTF$r8AYWVQL$SAA-NH₂ | | 1701.95 | 851.4 | 1702.96 | 851.98 | 568.32 |
| 163 | Ac-LTF$r8AHWAQL$SAA-NH₂ | | 1647.91 | 824.83 | 1648.92 | 824.96 | 550.31 |
| 164 | Ac-LTF$r8AEWAQL$SAA-NH₂ | | 1639.9 | 820.39 | 1640.91 | 820.96 | 547.64 |
| 165 | Ac-LTF$r8ASWAQL$SAA-NH₂ | | 1597.89 | 799.38 | 1598.9 | 799.95 | 533.64 |
| 166 | Ac-LTF$r8AEWAQL$SAA-NH₂ | iso2 | 1639.9 | 820.39 | 1640.91 | 820.96 | 547.64 |
| 167 | Ac-LTF$r8ASWAQL$SAA-NH₂ | iso2 | 1597.89 | 800.31 | 1598.9 | 799.95 | 533.64 |
| 168 | Ac-LTF$r8AF4coohWAQL$SAA-NH₂ | | 1701.91 | 851.4 | 1702.92 | 851.96 | 568.31 |
| 169 | Ac-LTF$r8AF4coohWAQL$SAA-NH₂ | iso2 | 1701.91 | 851.4 | 1702.92 | 851.96 | 568.31 |
| 170 | Ac-LTF$r8AHWAQL$AAIa-NH₂ | | 1745 | 874.13 | 1746.01 | 873.51 | 582.67 |
| 171 | Ac-ITF$r8FYWAQL$AAIa-NH₂ | | 1847.04 | 923.92 | 1848.05 | 924.53 | 616.69 |
| 172 | Ac-ITF$r8EHWAQL$AAIa-NH₂ | | 1803.01 | 903.17 | 1804.02 | 902.51 | 602.01 |
| 173 | Ac-ITF$r8EHWAQL$AAIa-NH₂ | iso2 | 1803.01 | 903.17 | 1804.02 | 902.51 | 602.01 |
| 174 | Ac-ETF$r8EHWAQL$AAIa-NH₂ | | 1818.97 | 910.76 | 1819.98 | 910.49 | 607.33 |
| 175 | Ac-ETF$r8EHWAQL$AAIa-NH₂ | iso2 | 1818.97 | 910.85 | 1819.98 | 910.49 | 607.33 |
| 176 | Ac-LTF$r8AHWVQL$AAIa-NH₂ | | 1773.03 | 888.09 | 1774.04 | 887.52 | 592.02 |
| 177 | Ac-ITF$r8FYWVQL$AAIa-NH₂ | | 1875.07 | 939.16 | 1876.08 | 938.54 | 626.03 |
| 178 | Ac-ITF$r8EYWVQL$AAIa-NH₂ | | 1857.04 | 929.83 | 1858.05 | 929.53 | 620.02 |
| 179 | Ac-ITF$r8EHWVQL$AAIa-NH₂ | | 1831.04 | 916.86 | 1832.05 | 916.53 | 611.35 |
| 180 | Ac-LTF$r8AEWAQL$AAIa-NH₂ | | 1736.99 | 869.87 | 1738 | 869.5 | 580 |
| 181 | Ac-LTF$r8AF4coohWAQL$AAIa-NH₂ | | 1799 | 900.17 | 1800.01 | 900.51 | 600.67 |
| 182 | Ac-LTF$r8AF4coohWAQL$AAIa-NH₂ | iso2 | 1799 | 900.24 | 1800.01 | 900.51 | 600.67 |
| 183 | Ac-LTF$r8AHWAQL$AHFA-NH₂ | | 1845.01 | 923.89 | 1846.02 | 923.51 | 616.01 |
| 184 | Ac-ITF$r8FYWAQL$AHFA-NH₂ | | 1947.05 | 975.05 | 1948.06 | 974.53 | 650.02 |
| 185 | Ac-ITF$r8FYWAQL$AHFA-NH₂ | iso2 | 1947.05 | 976.07 | 1948.06 | 974.53 | 650.02 |
| 186 | Ac-ITF$r8FHWAQL$AEFA-NH₂ | | 1913.02 | 958.12 | 1914.03 | 957.52 | 638.68 |
| 187 | Ac-ITF$r8FHWAQL$AEFA-NH₂ | iso2 | 1913.02 | 957.86 | 1914.03 | 957.52 | 638.68 |
| 188 | Ac-ITF$r8EHWAQL$AHFA-NH₂ | | 1903.01 | 952.94 | 1904.02 | 952.51 | 635.34 |
| 189 | Ac-ITF$r8EHWAQL$AHFA-NH₂ | iso2 | 1903.01 | 953.87 | 1904.02 | 952.51 | 635.34 |
| 190 | Ac-LTF$r8AHWVQL$AHFA-NH₂ | | 1873.04 | 937.86 | 1874.05 | 937.53 | 625.35 |
| 191 | Ac-ITF$r8FYWVQL$AHFA-NH₂ | | 1975.08 | 988.83 | 1976.09 | 988.55 | 659.37 |
| 192 | Ac-ITF$r8EYWVQL$AHFA-NH₂ | | 1957.05 | 979.35 | 1958.06 | 979.53 | 653.36 |
| 193 | Ac-ITF$r8EHWVQL$AHFA-NH₂ | | 1931.05 | 967 | 1932.06 | 966.53 | 644.69 |
| 194 | Ac-ITF$r8EHWVQL$AHFA-NH₂ | iso2 | 1931.05 | 967.93 | 1932.06 | 966.53 | 644.69 |
| 195 | Ac-ETF$r8EYWAAL$SAA-NH₂ | | 1690.86 | 845.85 | 1691.87 | 846.44 | 564.63 |
| 196 | Ac-LTF$r8AYWVAL$SAA-NH₂ | | 1644.93 | 824.08 | 1645.94 | 823.47 | 549.32 |
| 197 | Ac-LTF$r8AHWAAL$SAA-NH₂ | | 1590.89 | 796.88 | 1591.9 | 796.45 | 531.3 |
| 198 | Ac-LTF$r8AEWAAL$SAA-NH₂ | | 1582.88 | 791.9 | 1583.89 | 792.45 | 528.63 |
| 199 | Ac-LTF$r8AEWAAL$SAA-NH₂ | iso2 | 1582.88 | 791.9 | 1583.89 | 792.45 | 528.63 |
| 200 | Ac-LTF$r8ASWAAL$SAA-NH₂ | | 1540.87 | 770.74 | 1541.88 | 771.44 | 514.63 |
| 201 | Ac-LTF$r8ASWAAL$SAA-NH₂ | iso2 | 1540.87 | 770.88 | 1541.88 | 771.44 | 514.63 |
| 202 | Ac-LTF$r8AYWAAL$AAIa-NH₂ | | 1713.99 | 857.39 | 1715 | 858 | 572.34 |
| 203 | Ac-LTF$r8AYWAAL$AAIa-NH₂ | iso2 | 1713.99 | 857.84 | 1715 | 858 | 572.34 |
| 204 | Ac-LTF$r8AYWAALSAHFA-NH₂ | | 1813.99 | 907.86 | 1815 | 908 | 605.67 |
| 205 | Ac-LTF$r8EHWAQL$AHIa-NH₂ | | 1869.03 | 936.1 | 1870.04 | 935.52 | 624.02 |
| 206 | Ac-LTF$r8EHWAQL$AHIa-NH₂ | iso2 | 1869.03 | 937.03 | 1870.04 | 935.52 | 624.02 |
| 207 | Ac-LTF$r8AHWAQL$AHIa-NH₂ | | 1811.03 | 906.87 | 1812.04 | 906.52 | 604.68 |
| 208 | Ac-LTF$r8EYWAQL$AHIa-NH₂ | | 1895.04 | 949.15 | 1896.05 | 948.53 | 632.69 |
| 209 | Ac-LTF$r8AYWAQL$AAFa-NH₂ | | 1804.99 | 903.2 | 1806 | 903.5 | 602.67 |
| 210 | Ac-LTF$r8AYWAQL$AAFa-NH₂ | iso2 | 1804.99 | 903.28 | 1806 | 903.5 | 602.67 |
| 211 | Ac-LTF$r8AYWAQL$AAWa-NH₂ | | 1844 | 922.81 | 1845.01 | 923.01 | 615.67 |
| 212 | Ac-LTF$r8AYWAQL$AAVa-NH₂ | | 1756.99 | 878.86 | 1758 | 879.5 | 586.67 |
| 213 | Ac-LTF$r8AYWAQL$AAVa-NH₂ | iso2 | 1756.99 | 879.3 | 1758 | 879.5 | 586.67 |
| 214 | Ac-LTF$r8AYWAQL$AALa-NH₂ | | 1771.01 | 886.26 | 1772.02 | 886.51 | 591.34 |
| 215 | Ac-LTF$r8AYWAQL$AALa-NH₂ | iso2 | 1771.01 | 886.33 | 1772.02 | 886.51 | 591.34 |
| 216 | Ac-LTF$r8EYWAQL$AAIa-NH₂ | | 1829.01 | 914.89 | 1830.02 | 915.51 | 610.68 |
| 217 | Ac-LTF$r8EYWAQL$AAIa-NH₂ | iso2 | 1829.01 | 915.34 | 1830.02 | 915.51 | 610.68 |
| 218 | Ac-LTF$r8EYWAQL$AAFa-NH₂ | | 1863 | 932.87 | 1864.01 | 932.51 | 622.01 |
| 219 | Ac-LTF$r8EYWAQL$AAFa-NH₂ | iso2 | 1863 | 932.87 | 1864.01 | 932.51 | 622.01 |
| 220 | Ac-LTF$r8EYWAQL$AAVa-NH₂ | | 1815 | 908.23 | 1816.01 | 908.51 | 606.01 |
| 221 | Ac-LTF$r8EYWAQL$AAVa-NH₂ | iso2 | 1815 | 908.31 | 1816.01 | 908.51 | 606.01 |
| 222 | Ac-LTF$r8EHWAQL$AAIa-NH₂ | | 1803.01 | 903.17 | 1804.02 | 902.51 | 602.01 |
| 223 | Ac-LTF$r8EHWAQL$AAIa-NH₂ | iso2 | 1803.01 | 902.8 | 1804.02 | 902.51 | 602.01 |
| 224 | Ac-LTF$r8EHWAQL$AAWa-NH₂ | | 1876 | 939.34 | 1877.01 | 939.01 | 626.34 |
| 225 | Ac-LTF$r8EHWAQL$AAWa-NH₂ | iso2 | 1876 | 939.62 | 1877.01 | 939.01 | 626.34 |
| 226 | Ac-LTF$r8EHWAQL$AALa-NH₂ | | 1803.01 | 902.8 | 1804.02 | 902.51 | 602.01 |
| 227 | Ac-LTF$r8EHWAQL$AALa-NH₂ | iso2 | 1803.01 | 902.9 | 1804.02 | 902.51 | 602.01 |
| 228 | Ac-ETF$r8EHWVQL$AALa-NH₂ | | 1847 | 924.82 | 1848.01 | 924.51 | 616.67 |
| 229 | Ac-LTF$r8AYWAQL$AAAa-NH₂ | | 1728.96 | 865.89 | 1729.97 | 865.49 | 577.33 |
| 230 | Ac-LTF$r8AYWAQL$AAAa-NH₂ | iso2 | 1728.96 | 865.89 | 1729.97 | 865.49 | 577.33 |
| 231 | Ac-LTF$r8AYWAQL$AAAibA-NH₂ | | 1742.98 | 872.83 | 1743.99 | 872.5 | 582 |
| 232 | Ac-LTF$r8AYWAQL$AAAibA-NH₂ | iso2 | 1742.98 | 872.92 | 1743.99 | 872.5 | 582 |
| 233 | Ac-LTF$r8AYWAQL$AAAAa-NH₂ | | 1800 | 901.42 | 1801.01 | 901.01 | 601.01 |
| 234 | Ac-LTF$r5AYWAQL$s8AAIa-NH₂ | | 1771.01 | 887.17 | 1772.02 | 886.51 | 591.34 |
| 235 | Ac-LTF$r5AYWAQL$s8SAA-NH₂ | | 1673.92 | 838.33 | 1674.93 | 837.97 | 558.98 |
| 236 | Ac-LTF$r8AYWAQCba$AANleA-NH₂ | | 1783.01 | 892.64 | 1784.02 | 892.51 | 595.34 |
| 237 | Ac-ETF$r8AYWAQCba$AANleA-NH₂ | | 1798.97 | 900.59 | 1799.98 | 900.49 | 600.66 |
| 238 | Ac-LTF$r8EYWAQCba$AANleA-NH₂ | | 1841.01 | 922.05 | 1842.02 | 921.51 | 614.68 |
| 239 | Ac-LTF$r8AYWAQCba$AWNleA-NH₂ | | 1898.05 | 950.46 | 1899.06 | 950.03 | 633.69 |
| 240 | Ac-ETF$r8AYWAQCba$AWNleA-NH₂ | | 1914.01 | 958.11 | 1915.02 | 958.01 | 639.01 |
| 241 | Ac-LTF$r8EYWAQCba$AWNleA-NH₂ | | 1956.06 | 950.62 | 1957.07 | 979.04 | 653.03 |
| 242 | Ac-LTF$r8EYWAQCba$SAFA-NH₂ | | 1890.99 | 946.55 | 1892 | 946.5 | 631.34 |
| 243 | Ac-LTF34F2$r8EYWAQCba$SANleA-NH₂ | | 1892.99 | 947.57 | 1894 | 947.5 | 632 |
| 244 | Ac-LTF$r8EF4coohWAQCba$SANleA-NH₂ | | 1885 | 943.59 | 1886.01 | 943.51 | 629.34 |
| 245 | Ac-LTF$r8EYWSQCba$SANleA-NH₂ | | 1873 | 937.58 | 1874.01 | 937.51 | 625.34 |
| 246 | Ac-LTF$r8EYWWQCba$SANleA-NH₂ | | 1972.05 | 987.61 | 1973.06 | 987.03 | 658.36 |
| 247 | Ac-LTF$r8EYWAQCba$AAIa-NH₂ | | 1841.01 | 922.05 | 1842.02 | 921.51 | 614.68 |
| 248 | Ac-LTF34F2$r8EYWAQCba$AAIa-NH₂ | | 1876.99 | 939.99 | 1878 | 939.5 | 626.67 |
| 249 | Ac-LTF$r8EF4coohWAQCba$AAIa-NH₂ | | 1869.01 | 935.64 | 1870.02 | 935.51 | 624.01 |
| 250 | Pam-ETF$r8EYWAQCba$SAA-NH₂ | | 1956.1 | 979.57 | 1957.11 | 979.06 | 653.04 |
| 251 | Ac-LThF$r8EFWAQCba$SAA-NH₂ | | 1741.94 | 872.11 | 1742.95 | 871.98 | 581.65 |
| 252 | Ac-LTA$r8EYWAQCba$SAA-NH₂ | | 1667.89 | 835.4 | 1668.9 | 834.95 | 556.97 |
| 253 | Ac-LTF$r8EYAAQCba$SAA-NH₂ | | 1628.88 | 815.61 | 1629.89 | 815.45 | 543.97 |
| 254 | Ac-LTF$r8EY2NalAQCba$SAA-NH₂ | | 1754.93 | 879.04 | 1755.94 | 878.47 | 585.98 |
| 255 | Ac-LTF$r8AYWAQCba$SAA-NH₂ | | 1685.92 | 844.71 | 1686.93 | 843.97 | 562.98 |
| 256 | Ac-LTF$r8EYWAQCba$SAF-NH₂ | | 1819.96 | 911.41 | 1820.97 | 910.99 | 607.66 |
| 257 | Ac-LTF$r8EYWAQCba$SAFa-NH₂ | | 1890.99 | 947.41 | 1892 | 946.5 | 631.34 |
| 258 | Ac-LTF$r8AYWAQCba$SAF-NH₂ | | 1761.95 | 882.73 | 1762.96 | 881.98 | 588.32 |
| 259 | Ac-LTF34F2$r8AYWAQCba$SAF-NH₂ | | 1797.93 | 900.87 | 1798.94 | 899.97 | 600.32 |
| 260 | Ac-LTF$r8AF4coohWAQCba$SAF-NH₂ | | 1789.94 | 896.43 | 1790.95 | 895.98 | 597.65 |
| 261 | Ac-LTF$r8EY6clWAQCba$SAF-NH₂ | | 1853.92 | 929.27 | 1854.93 | 927.97 | 618.98 |
| 262 | Ac-LTF$r8AYWSQCba$SAF-NH₂ | | 1777.94 | 890.87 | 1778.95 | 889.98 | 593.65 |
| 263 | Ac-LTF$r8AYWWQCba$SAF-NH₂ | | 1876.99 | 939.91 | 1878 | 939.5 | 626.67 |
| 264 | Ac-LTF$r8AYWAQCba$AAIa-NH₂ | | 1783.01 | 893.19 | 1784.02 | 892.51 | 595.34 |
| 265 | Ac-LTF34F2$r8AYWAQCba$AAIa-NH₂ | | 1818.99 | 911.23 | 1820 | 910.5 | 607.34 |
| 266 | Ac-LTF$r8AY6clWAQCba$AAIa-NH₂ | | 1816.97 | 909.84 | 1817.98 | 909.49 | 606.66 |
| 267 | Ac-LTF$r8AF4coohWAQCba$AAIa-NH₂ | | 1811 | 906.88 | 1812.01 | 906.51 | 604.67 |
| 268 | Ac-LTF$r8EYWAQCba$AAFa-NH₂ | | 1875 | 938.6 | 1876.01 | 938.51 | 626.01 |
| 269 | Ac-LTF$r8EYWAQCba$AAFa-NH₂ | iso2 | 1875 | 938.6 | 1876.01 | 938.51 | 626.01 |
| 270 | Ac-ETF$r8AYWAQCba$AWNlea-NH₂ | | 1914.01 | 958.42 | 1915.02 | 958.01 | 639.01 |
| 271 | Ac-LTF$r8EYWAQCba$AWNlea-NH₂ | | 1956.06 | 979.42 | 1957.07 | 979.04 | 653.03 |
| 272 | Ac-ETF$r8EYWAQCba$AWNlea-NH₂ | | 1972.01 | 987.06 | 1973.02 | 987.01 | 658.34 |
| 273 | Ac-ETF$r8EYWAQCba$AWNlea-NH₂ | iso2 | 1972.01 | 987.06 | 1973.02 | 987.01 | 658.34 |
| 274 | Ac-LTF$r8AYWAQCba$SAFa-NH₂ | | 1832.99 | 917.89 | 1834 | 917.5 | 612 |
| 275 | Ac-LTF$r8AYWAQCba$SAFa-NH₂ | iso2 | 1832.99 | 918.07 | 1834 | 917.5 | 612 |
| 276 | Ac-ETF$r8AYWAQL$AWNlea-NH₂ | | 1902.01 | 952.22 | 1903.02 | 952.01 | 635.01 |
| 277 | Ac-LTF$r8EYWAQL$AWNlea-NH₂ | | 1944.06 | 973.5 | 1945.07 | 973.04 | 649.03 |
| 278 | Ac-ETF$r8EYWAQL$AWNlea-NH₂ | | 1960.01 | 981.46 | 1961.02 | 981.01 | 654.34 |
| 279 | Dmaac-LTF$r8EYWAQhL$SAA-NH₂ | | 1788.98 | 896.06 | 1789.99 | 895.5 | 597.33 |
| 280 | Hexac-LTF$r8EYWAQhL$SAA-NH₂ | | 1802 | 902.9 | 1803.01 | 902.01 | 601.67 |
| 281 | Napac-LTF$r8EYWAQhL$SAA-NH₂ | | 1871.99 | 937.58 | 1873 | 937 | 625 |
| 282 | Decac-LTF$r8EYWAQhL$SAA-NH₂ | | 1858.06 | 930.55 | 1859.07 | 930.04 | 620.36 |
| 283 | Admac-LTF$r8EYWAQhL$SAA-NH₂ | | 1866.03 | 934.07 | 1867.04 | 934.02 | 623.02 |
| 284 | Tmac-LTF$r8EYWAQhL$SAA-NH₂ | | 1787.99 | 895.41 | 1789 | 895 | 597 |
| 285 | Pam-LTF$r8EYWAQhL$SAA-NH₂ | | 1942.16 | 972.08 | 1943.17 | 972.09 | 648.39 |
| 286 | Ac-LTF$r8AYWAQCba$AANleA-NH₂ | iso2 | 1783.01 | 892.64 | 1784.02 | 892.51 | 595.34 |
| 287 | Ac-LTF34F2$r8EYWAQCba$AAIa-NH₂ | iso2 | 1876.99 | 939.62 | 1878 | 939.5 | 626.67 |
| 288 | Ac-LTF34F2$r8EYWAQCba$SAA-NH₂ | | 1779.91 | 892.07 | 1780.92 | 890.96 | 594.31 |
| 289 | Ac-LTF34F2$r8EYWAQCba$SAA-NH₂ | iso2 | 1779.91 | 891.61 | 1780.92 | 890.96 | 594.31 |
| 290 | Ac-LTF$r8EF4coohWAQCba$SAA-NH₂ | | 1771.92 | 887.54 | 1772.93 | 886.97 | 591.65 |
| 291 | Ac-LTF$r8EF4coohWAQCba$SAA-NH₂ | iso2 | 1771.92 | 887.63 | 1772.93 | 886.97 | 591.65 |
| 292 | Ac-LTF$r8EYWSQCba$SAA-NH₂ | | 1759.92 | 881.9 | 1760.93 | 880.97 | 587.65 |
| 293 | Ac-LTF$r8EYWSQCba$SAA-NH₂ | iso2 | 1759.92 | 881.9 | 1760.93 | 880.97 | 587.65 |
| 294 | Ac-LTF$r8EYWAQhL$SAA-NH₂ | | 1745.94 | 875.05 | 1746.95 | 873.98 | 582.99 |
| 295 | Ac-LTF$r8AYWAQhL$SAF-NH₂ | | 1763.97 | 884.02 | 1764.98 | 882.99 | 589 |
| 296 | Ac-LTF$r8AYWAQhL$SAF-NH₂ | iso2 | 1763.97 | 883.56 | 1764.98 | 882.99 | 589 |
| 297 | Ac-LTF34F2$r8AYWAQhL$SAA-NH₂ | | 1723.92 | 863.67 | 1724.93 | 862.97 | 575.65 |
| 298 | Ac-LTF34F2$r8AYWAQhL$SAA-NH₂ | iso2 | 1723.92 | 864.04 | 1724.93 | 862.97 | 575.65 |
| 299 | Ac-LTF$r8AF4coohWAQhL$SAA-NH₂ | | 1715.93 | 859.44 | 1716.94 | 858.97 | 572.98 |
| 300 | Ac-LTF$r8AF4coohWAQhL$SAA-NH₂ | iso2 | 1715.93 | 859.6 | 1716.94 | 858.97 | 572.98 |
| 301 | Ac-LTF$r8AYWSQhL$SAA-NH₂ | | 1703.93 | 853.96 | 1704.94 | 852.97 | 568.98 |
| 302 | Ac-LTF$r8AYWSQhL$SAA-NH₂ | iso2 | 1703.93 | 853.59 | 1704.94 | 852.97 | 568.98 |
| 303 | Ac-LTF$r8EYWAQL$AANleA-NH₂ | | 1829.01 | 915.45 | 1830.02 | 915.51 | 610.68 |
| 304 | Ac-LTF34F2$r8AYWAQL$AANleA-NH₂ | | 1806.99 | 904.58 | 1808 | 904.5 | 603.34 |
| 305 | Ac-LTF$r8AF4coohWAQL$AANleA-NH₂ | | 1799 | 901.6 | 1800.01 | 900.51 | 600.67 |
| 306 | Ac-LTF$r8AYWSQL$AANleA-NH₂ | | 1787 | 894.75 | 1788.01 | 894.51 | 596.67 |
| 307 | Ac-LTF34F2$r8AYWAQhL$AANleA-NH₂ | | 1821 | 911.79 | 1822.01 | 911.51 | 608.01 |
| 308 | Ac-LTF34F2$r8AYWAQhL$AANleA-NH₂ | iso2 | 1821 | 912.61 | 1822.01 | 911.51 | 608.01 |
| 309 | Ac-LTF$r8AF4coohWAQhL$AANleA-NH₂ | | 1813.02 | 907.95 | 1814.03 | 907.52 | 605.35 |
| 310 | Ac-LTF$r8AF4coohWAQhL$AANleA-NH₂ | iso2 | 1813.02 | 908.54 | 1814.03 | 907.52 | 605.35 |
| 311 | Ac-LTF$r8AYWSQhL$AANleA-NH₂ | | 1801.02 | 901.84 | 1802.03 | 901.52 | 601.35 |
| 312 | Ac-LTF$r8AYWSQhL$AANleA-NH₂ | iso2 | 1801.02 | 902.62 | 1802.03 | 901.52 | 601.35 |
| 313 | Ac-LTF$r8AYWAQhL$AAAAa-NH₂ | | 1814.01 | 908.63 | 1815.02 | 908.01 | 605.68 |
| 314 | Ac-LTF$r8AYWAQhL$AAAAa-NH₂ | iso2 | 1814.01 | 908.34 | 1815.02 | 908.01 | 605.68 |
| 315 | Ac-LTF$r8AYWAQL$AAAAAa-NH₂ | | 1871.04 | 936.94 | 1872.05 | 936.53 | 624.69 |
| 316 | Ac-LTF$r8AYWAQL$AAAAAAa-NH₂ | iso2 | 1942.07 | 972.5 | 1943.08 | 972.04 | 648.37 |
| 317 | Ac-LTF$r8AYWAQL$AAAAAAa-NH₂ | iso1 | 1942.07 | 972.5 | 1943.08 | 972.04 | 648.37 |
| 318 | Ac-LTF$r8EYWAQhL$AANleA-NH₂ | | 1843.03 | 922.54 | 1844.04 | 922.52 | 615.35 |
| 319 | Ac-AATF$r8AYWAQL$AANleA-NH₂ | | 1800 | 901.39 | 1801.01 | 901.01 | 601.01 |
| 320 | Ac-LTF$r8AYWAQL$AANleAA-NH₂ | | 1842.04 | 922.45 | 1843.05 | 922.03 | 615.02 |
| 321 | Ac-ALTF$r8AYWAQL$AANleAA-NH₂ | | 1913.08 | 957.94 | 1914.09 | 957.55 | 638.7 |
| 322 | Ac-LTF$r8AYWAQCba$AANleAA-NH₂ | | 1854.04 | 928.43 | 1855.05 | 928.03 | 619.02 |
| 323 | Ac-LTF$r8AYWAQhL$AANleAA-NH₂ | | 1856.06 | 929.4 | 1857.07 | 929.04 | 619.69 |
| 324 | Ac-LTF$r8EYWAQCba$SAAA-NH₂ | | 1814.96 | 909.37 | 1815.97 | 908.49 | 605.99 |
| 325 | Ac-LTF$r8EYWAQCba$SAAA-NH₂ | iso2 | 1814.96 | 909.37 | 1815.97 | 908.49 | 605.99 |
| 326 | Ac-LTF$r8EYWAQCba$SAAAA-NH₂ | | 1886 | 944.61 | 1887.01 | 944.01 | 629.67 |
| 327 | Ac-LTF$r8EYWAQCba$SAAAA-NH₂ | iso2 | 1886 | 944.61 | 1887.01 | 944.01 | 629.67 |
| 328 | Ac-ALTF$r8EYWAQCba$SAA-NH₂ | | 1814.96 | 909.09 | 1815.97 | 908.49 | 605.99 |
| 329 | Ac-ALTF$r8EYWAQCba$SAAA-NH₂ | | 1886 | 944.61 | 1887.01 | 944.01 | 629.67 |
| 330 | Ac-ALTF$r8EYWAQCba$SAA-NH₂ | iso2 | 1814.96 | 909.09 | 1815.97 | 908.49 | 605.99 |
| 331 | Ac-LTF$r8EYWAQL$AAAAAa-NH₂ | iso2 | 1929.04 | 966.08 | 1930.05 | 965.53 | 644.02 |
| 332 | Ac-LTF$r8EY6clWAQCba$SAA-NH₂ | | 1777.89 | 890.78 | 1778.9 | 889.95 | 593.64 |
| 333 | Ac-LTF$r8EF4cooh6clWAQCba$SANleA-NH₂ | | 1918.96 | 961.27 | 1919.97 | 960.49 | 640.66 |
| 334 | Ac-LTF$r8EF4cooh6clWAQCba$SANleA-NH₂ | iso2 | 1918.96 | 961.27 | 1919.97 | 960.49 | 640.66 |
| 335 | Ac-LTF$r8EF4cooh6clWAQCba$AAIa-NH₂ | | 1902.97 | 953.03 | 1903.98 | 952.49 | 635.33 |
| 336 | Ac-LTF$r8EF4cooh6clWAQCba$AAIa-NH₂ | iso2 | 1902.97 | 953.13 | 1903.98 | 952.49 | 635.33 |
| 337 | Ac-LTF$r8AY6clWAQL$AAAAAa-NH₂ | | 1905 | 954.61 | 1906.01 | 953.51 | 636.01 |
| 338 | Ac-LTF$r8AY6clWAQL$AAAAAa-NH₂ | iso2 | 1905 | 954.9 | 1906.01 | 953.51 | 636.01 |
| 339 | Ac-F$r8AY6clWEAL$AAAAAAa-NH₂ | | 1762.89 | 883.01 | 1763.9 | 882.45 | 588.64 |
| 340 | Ac-ETF$r8EYWAQL$AAAAAa-NH₂ | | 1945 | 974.31 | 1946.01 | 973.51 | 649.34 |
| 341 | Ac-ETF$r8EYWAQL$AAAAAa-NH₂ | iso2 | 1945 | 974.49 | 1946.01 | 973.51 | 649.34 |
| 342 | Ac-LTF$r8EYWAQL$AAAAAAa-NH₂ | | 2000.08 | 1001.6 | 2001.09 | 1001.05 | 667.7 |
| 343 | Ac-LTF$r8EYWAQL$AAAAAAa-NH₂ | iso2 | 2000.08 | 1001.6 | 2001.09 | 1001.05 | 667.7 |
| 344 | Ac-LTF$r8AYWAQL$AANleAAa-NH₂ | | 1913.08 | 958.58 | 1914.09 | 957.55 | 638.7 |
| 345 | Ac-LTF$r8AYWAQL$AANleAAa-NH₂ | iso2 | 1913.08 | 958.58 | 1914.09 | 957.55 | 638.7 |
| 346 | Ac-LTF$r8EYWAQCba$AAAAAa-NH₂ | | 1941.04 | 972.55 | 1942.05 | 971.53 | 648.02 |
| 347 | Ac-LTF$r8EYWAQCba$AAAAAa-NH₂ | iso2 | 1941.04 | 972.55 | 1942.05 | 971.53 | 648.02 |
| 348 | Ac-LTF$r8EF4coohWAQCba$AAAAAa-NH₂ | | 1969.04 | 986.33 | 1970.05 | 985.53 | 657.35 |
| 349 | Ac-LTF$r8EF4coohWAQCba$AAAAAa-NH₂ | iso2 | 1969.04 | 986.06 | 1970.05 | 985.53 | 657.35 |
| 350 | Ac-LTF$r8EYWSQCba$AAAAAa-NH₂ | | 1957.04 | 980.04 | 1958.05 | 979.53 | 653.35 |
| 351 | Ac-LTF$r8EYWSQCba$AAAAAa-NH₂ | iso2 | 1957.04 | 980.04 | 1958.05 | 979.53 | 653.35 |
| 352 | Ac-LTF$r8EYWAQCba$SAAa-NH₂ | | 1814.96 | 909 | 1815.97 | 908.49 | 605.99 |
| 353 | Ac-LTF$r8EYWAQCba$SAAa-NH₂ | iso2 | 1814.96 | 909 | 1815.97 | 908.49 | 605.99 |
| 354 | Ac-ALTF$r8EYWAQCba$SAAa-NH₂ | | 1886 | 944.52 | 1887.01 | 944.01 | 629.67 |
| 355 | Ac-ALTF$r8EYWAQCba$SAAa-NH₂ | iso2 | 1886 | 944.98 | 1887.01 | 944.01 | 629.67 |
| 356 | Ac-ALTF$r8EYWAQCba$SAAAa-NH₂ | | 1957.04 | 980.04 | 1958.05 | 979.53 | 653.35 |
| 357 | Ac-ALTF$r8EYWAQCba$SAAAa-NH₂ | iso2 | 1957.04 | 980.04 | 1958.05 | 979.53 | 653.35 |
| 358 | Ac-AALTF$r8EYWAQCba$SAAAa-NH₂ | | 2028.07 | 1016.1 | 2029.08 | 1015.04 | 677.03 |
| 359 | Ac-AALTF$r8EYWAQCba$SAAAa-NH₂ | iso2 | 2028.07 | 1015.57 | 2029.08 | 1015.04 | 677.03 |
| 360 | Ac-RTF$r8EYWAQCba$SAA-NH₂ | | 1786.94 | 895.03 | 1787.95 | 894.48 | 596.65 |
| 361 | Ac-LRF$r8EYWAQCba$SAA-NH₂ | | 1798.98 | 901.51 | 1799.99 | 900.5 | 600.67 |
| 362 | Ac-LTF$r8EYWRQCba$SAA-NH₂ | | 1828.99 | 916.4 | 1830 | 915.5 | 610.67 |
| 363 | Ac-LTF$r8EYWARCba$SAA-NH₂ | | 1771.97 | 887.63 | 1772.98 | 886.99 | 591.66 |
| 364 | Ac-LTF$r8EYWAQCba$RAA-NH₂ | | 1812.99 | 908.08 | 1814 | 907.5 | 605.34 |
| 365 | Ac-LTF$r8EYWAQCba$SRA-NH₂ | | 1828.99 | 916.12 | 1830 | 915.5 | 610.67 |
| 366 | Ac-LTF$r8EYWAQCba$SAR-NH₂ | | 1828.99 | 916.12 | 1830 | 915.5 | 610.67 |
| 367 | 5-FAM-BaLTF$r8EYWAQCba$SAA-NH₂ | | 2131 | 1067.09 | 2132.01 | 1066.51 | 711.34 |
| 368 | 5-FAM-BaLTF$r8AYWAQL$AANleA-NH₂ | | 2158.08 | 1080.6 | 2159.09 | 1080.05 | 720.37 |
| 369 | Ac-LAF$r8EYWAQL$AANleA-NH₂ | | 1799 | 901.05 | 1800.01 | 900.51 | 600.67 |
| 370 | Ac-ATF$r8EYWAQL$AANleA-NH₂ | | 1786.97 | 895.03 | 1787.98 | 894.49 | 596.66 |
| 371 | Ac-AAF$r8EYWAQL$AANleA-NH₂ | | 1756.96 | 880.05 | 1757.97 | 879.49 | 586.66 |
| 372 | Ac-AAAF$r8EYWAQL$AANleA-NH₂ | | 1827.99 | 915.57 | 1829 | 915 | 610.34 |
| 373 | Ac-AAAAF$r8EYWAQL$AANleA-NH₂ | | 1899.03 | 951.09 | 1900.04 | 950.52 | 634.02 |
| 374 | Ac-AATF$r8EYWAQL$AANleA-NH₂ | | 1858 | 930.92 | 1859.01 | 930.01 | 620.34 |
| 375 | Ac-AALTF$r8EYWAQL$AANleA-NH₂ | | 1971.09 | 987.17 | 1972.1 | 986.55 | 658.04 |
| 376 | Ac-AAALTF$r8EYWAQL$AANleA-NH₂ | | 2042.12 | 1023.15 | 2043.13 | 1022.07 | 681.71 |
| 377 | Ac-LTF$r8EYWAQL$AANleAA-NH₂ | | 1900.05 | 952.02 | 1901.06 | 951.03 | 634.36 |
| 378 | Ac-ALTF$r8EYWAQL$AANleAA-NH₂ | | 1971.09 | 987.63 | 1972.1 | 986.55 | 658.04 |
| 379 | Ac-AALTF$r8EYWAQL$AANleAA-NH₂ | | 2042.12 | 1022.69 | 2043.13 | 1022.07 | 681.71 |
| 380 | Ac-LTF$r8EYWAQCba$AANleAA-NH₂ | | 1912.05 | 958.03 | 1913.06 | 957.03 | 638.36 |
| 381 | Ac-LTF$r8EYWAQhL$AANleAA-NH₂ | | 1914.07 | 958.68 | 1915.08 | 958.04 | 639.03 |
| 382 | Ac-ALTF$r8EYWAQhL$AANleAA-NH₂ | | 1985.1 | 994.1 | 1986.11 | 993.56 | 662.71 |
| 383 | Ac-LTF$r8ANmYWAQL$AANleA-NH₂ | | 1785.02 | 894.11 | 1786.03 | 893.52 | 596.01 |
| 384 | Ac-LTF$r8ANmYWAQL$AANleA-NH₂ | iso2 | 1785.02 | 894.11 | 1786.03 | 893.52 | 596.01 |
| 385 | Ac-LTF$r8AYNmWAQL$AANleA-NH₂ | | 1785.02 | 894.11 | 1786.03 | 893.52 | 596.01 |
| 386 | Ac-LTF$r8AYNmWAQL$AANleA-NH₂ | iso2 | 1785.02 | 894.11 | 1786.03 | 893.52 | 596.01 |
| 387 | Ac-LTF$r8AYAmwAQL$AANleA-NH₂ | | 1785.02 | 894.01 | 1786.03 | 893.52 | 596.01 |
| 388 | Ac-LTF$r8AYAmwAQL$AANleA-NH₂ | iso2 | 1785.02 | 894.01 | 1786.03 | 893.52 | 596.01 |
| 389 | Ac-LTF$r8AYWAibQL$AANleA-NH₂ | | 1785.02 | 894.01 | 1786.03 | 893.52 | 596.01 |
| 390 | Ac-LTF$r8AYWAibQL$AANleA-NH₂ | iso2 | 1785.02 | 894.01 | 1786.03 | 893.52 | 596.01 |
| 391 | Ac-LTF$r8AYWAQL$AAibNleA-NH₂ | | 1785.02 | 894.38 | 1786.03 | 893.52 | 596.01 |
| 392 | Ac-LTF$r8AYWAQL$AAibNleA-NH₂ | iso2 | 1785.02 | 894.38 | 1786.03 | 893.52 | 596.01 |
| 393 | Ac-LTF$r8AYWAQL$AaNleA-NH₂ | | 1771.01 | 887.54 | 1772.02 | 886.51 | 591.34 |
| 394 | Ac-LTF$r8AYWAQL$AaNleA-NH₂ | iso2 | 1771.01 | 887.54 | 1772.02 | 886.51 | 591.34 |
| 395 | Ac-LTF$r8AYWAQL$ASarNleA-NH₂ | | 1771.01 | 887.35 | 1772.02 | 886.51 | 591.34 |
| 396 | Ac-LTF$r8AYWAQL$ASarNleA-NH₂ | iso2 | 1771.01 | 887.35 | 1772.02 | 886.51 | 591.34 |
| 397 | Ac-LTF$r8AYWAQL$AANleAib-NH₂ | | 1785.02 | 894.75 | 1786.03 | 893.52 | 596.01 |
| 398 | Ac-LTF$r8AYWAQL$AANleAib-NH₂ | iso2 | 1785.02 | 894.75 | 1786.03 | 893.52 | 596.01 |
| 399 | Ac-LTF$r8AYWAQL$AANleNmA-NH₂ | | 1785.02 | 894.6 | 1786.03 | 893.52 | 596.01 |
| 400 | Ac-LTF$r8AYWAQL$AANleNmA-NH₂ | iso2 | 1785.02 | 894.6 | 1786.03 | 893.52 | 596.01 |
| 401 | Ac-LTF$r8AYWAQL$AANleSar-NH₂ | | 1771.01 | 886.98 | 1772.02 | 886.51 | 591.34 |
| 402 | Ac-LTF$r8AYWAQL$AANleSar-NH₂ | iso2 | 1771.01 | 886.98 | 1772.02 | 886.51 | 591.34 |
| 403 | Ac-LTF$r8AYWAQL$AANleAAib-NH₂ | | 1856.06 | | 1857.07 | 929.04 | 619.69 |
| 404 | Ac-LTF$r8AYWAQL$AANleAAib-NH₂ | iso2 | 1856.06 | | 1857.07 | 929.04 | 619.69 |
| 405 | Ac-LTF$r8AYWAQL$AANleANmA-NH₂ | | 1856.06 | 930.37 | 1857.07 | 929.04 | 619.69 |
| 406 | Ac-LTF$r8AYWAQL$AANleANmA-NH₂ | iso2 | 1856.06 | 930.37 | 1857.07 | 929.04 | 619.69 |
| 407 | Ac-LTF$r8AYWAQL$AANleAa-NH₂ | | 1842.04 | 922.69 | 1843.05 | 922.03 | 615.02 |
| 408 | Ac-LTF$r8AYWAQL$AANleAa-NH₂ | iso2 | 1842.04 | 922.69 | 1843.05 | 922.03 | 615.02 |
| 409 | Ac-LTF$r8AYWAQL$AANleASar-NH₂ | | 1842.04 | 922.6 | 1843.05 | 922.03 | 615.02 |
| 410 | Ac-LTF$r8AYWAQL$AANleASar-NH₂ | iso2 | 1842.04 | 922.6 | 1843.05 | 922.03 | 615.02 |
| 411 | Ac-LTF$/r8AYWAQLS/AANleA-NH₂ | | 1799.04 | 901.14 | 1800.05 | 900.53 | 600.69 |
| 412 | Ac-LTFAibAYWAQLAibAANleA-NH₂ | | 1648.9 | 826.02 | 1649.91 | 825.46 | 550.64 |
| 413 | Ac-LTF$r8Cou4YWAQL$AANleA-NH₂ | | 1975.05 | 989.11 | 1976.06 | 988.53 | 659.36 |
| 414 | Ac-LTF$r8Cou4YWAQL$AANleA-NH₂ | iso2 | 1975.05 | 989.11 | 1976.06 | 988.53 | 659.36 |
| 415 | Ac-LTF$r8AYWCou4QL$AANleA-NH₂ | | 1975.05 | 989.11 | 1976.06 | 988.53 | 659.36 |
| 416 | Ac-LTF$r8AYWAQL$Cou4ANleA-NH₂ | | 1975.05 | 989.57 | 1976.06 | 988.53 | 659.36 |
| 417 | Ac-LTF$r8AYWAQL$Cou4ANleA-NH₂ | iso2 | 1975.05 | 989.57 | 1976.06 | 988.53 | 659.36 |
| 418 | Ac-LTF$r8AYWAQL$ACou4NleA-NH₂ | | 1975.05 | 989.57 | 1976.06 | 988.53 | 659.36 |
| 419 | Ac-LTF$r8AYWAQL$ACou4NleA-NH₂ | iso2 | 1975.05 | 989.57 | 1976.06 | 988.53 | 659.36 |
| 420 | Ac-LTF$r8AYWAQL$AANleA-OH | | 1771.99 | 887.63 | 1773 | 887 | 591.67 |
| 421 | Ac-LTF$r8AYWAQL$AANleA-OH | iso2 | 1771.99 | 887.63 | 1773 | 887 | 591.67 |
| 422 | Ac-LTF$r8AYWAQL$AANleA-NHnPr | | 1813.05 | 908.08 | 1814.06 | 907.53 | 605.36 |
| 423 | Ac-LTF$r8AYWAQL$AANleA-NHnPr | iso2 | 1813.05 | 908.08 | 1814.06 | 907.53 | 605.36 |
| 424 | Ac-LTF$r8AYWAQL$AANleA-NHnBu33Me | | 1855.1 | 929.17 | 1856.11 | 928.56 | 619.37 |
| 425 | Ac-LTF$r8AYWAQL$AANleA-NHnBu33Me | iso2 | 1855.1 | 929.17 | 1856.11 | 928.56 | 619.37 |
| 426 | Ac-LTF$r8AYWAQL$AANleA-NHHex | | 1855.1 | 929.17 | 1856.11 | 928.56 | 619.37 |
| 427 | Ac-LTF$r8AYWAQL$AANleA-NHHex | iso2 | 1855.1 | 929.17 | 1856.11 | 928.56 | 619.37 |
| 428 | Ac-LTA$r8AYWAQL$AANleA-NH₂ | | 1694.98 | 849.33 | 1695.99 | 848.5 | 566 |
| 429 | Ac-LThL$r8AYWAQL$AANleA-NH₂ | | 1751.04 | 877.09 | 1752.05 | 876.53 | 584.69 |
| 430 | Ac-LTF$r8AYAAQL$AANleA-NH₂ | | 1655.97 | 829.54 | 1656.98 | 828.99 | 553 |
| 431 | Ac-LTF$r8AY2NalAQL$AANleA-NH₂ | | 1782.01 | 892.63 | 1783.02 | 892.01 | 595.01 |
| 432 | Ac-LTF$r8EYWCou4QCba$SAA-NH₂ | | 1947.97 | 975.8 | 1948.98 | 974.99 | 650.33 |
| 433 | Ac-LTF$r8EYWCou7QCba$SAA-NH₂ | | 16.03 | 974.9 | 17.04 | 9.02 | 6.35 |
| 434 | Ac-LTF%r8EYWAQCba%SAA-NH₂ | | 1745.94 | 874.8 | 1746.95 | 873.98 | 582.99 |
| 435 | Dmaac-LTF$r8EYWAQCba$SAA-NH₂ | | 1786.97 | 894.8 | 1787.98 | 894.49 | 596.66 |
| 436 | Dmaac-LTF$r8AYWAQL$AAAAAa-NH₂ | | 1914.08 | 958.2 | 1915.09 | 958.05 | 639.03 |
| 437 | Dmaac-LTF$r8AYWAQL$AAAAAa-NH₂ | iso2 | 1914.08 | 958.2 | 1915.09 | 958.05 | 639.03 |
| 438 | Dmaac-LTF$r8EYWAQL$AAAAAa-NH₂ | | 1972.08 | 987.3 | 1973.09 | 987.05 | 658.37 |
| 439 | Dmaac-LTF$r8EYWAQL$AAAAAa-NH₂ | iso2 | 1972.08 | 987.3 | 1973.09 | 987.05 | 658.37 |
| 440 | Dmaac-LTF$r8EF4coohWAQCba$AAIa-NH₂ | | 1912.05 | 957.4 | 1913.06 | 957.03 | 638.36 |
| 441 | Dmaac-LTF$r8EF4coohWAQCba$AAIa-NH₂ | iso2 | 1912.05 | 957.4 | 1913.06 | 957.03 | 638.36 |
| 442 | Dmaac-LTF$r8AYWAQL$AANleA-NH₂ | | 1814.05 | 908.3 | 1815.06 | 908.03 | 605.69 |
| 443 | Dmaac-LTF$r8AYWAQL$AANleA-NH₂ | iso2 | 1814.05 | 908.3 | 1815.06 | 908.03 | 605.69 |
| 444 | Ac-LTF%r8AYWAQL%AANleA-NH₂ | | 1773.02 | 888.37 | 1774.03 | 887.52 | 592.01 |
| 445 | Ac-LTF%r8EYWAQL%AAAAAa-NH₂ | | 1931.06 | 966.4 | 1932.07 | 966.54 | 644.69 |
| 446 | Cou6BaLTF$r8EYWAQhL$SAA-NH₂ | | 2018.05 | 1009.9 | 2019.06 | 1010.03 | 673.69 |
| 447 | Cou8BaLTF$r8EYWAQhL$SAA-NH₂ | | 1962.96 | 982.34 | 1963.97 | 982.49 | 655.32 |
| 448 | Ac-LTF4I$r8EYWAQL$AAAAAa-NH₂ | | 2054.93 | 1028.68 | 2055.94 | 1028.47 | 685.98 |
| 449 | Ac-LTF$r8EYWAQL$AAAAAa-NH₂ | | 1929.04 | 966.17 | 1930.05 | 965.53 | 644.02 |
| 550 | Ac-LTF$r8EYWAQL$AAAAAa-OH | | 1930.02 | 966.54 | 1931.03 | 966.02 | 644.35 |
| 551 | Ac-LTF$r8EYWAQL$AAAAAa-OH | iso2 | 1930.02 | 965.89 | 1931.03 | 966.02 | 644.35 |
| 552 | Ac-LTF$r8EYWAELSAAAAAa-NH₂ | | 1930.02 | 966.82 | 1931.03 | 966.02 | 644.35 |
| 553 | Ac-LTF$r8EYWAELSAAAAAa-NH₂ | iso2 | 1930.02 | 966.91 | 1931.03 | 966.02 | 644.35 |
| 554 | Ac-LTF$r8EYWAEL$AAAAAa-OH | | 1931.01 | 967.28 | 1932.02 | 966.51 | 644.68 |
| 555 | Ac-LTF$r8EY6clWAQL$AAAAAa-NH₂ | | 1963 | 983.28 | 1964.01 | 982.51 | 655.34 |
| 556 | Ac-LTF$r8EF4bOH2WAQL$AAAAAa-NH₂ | | 1957.05 | 980.04 | 1958.06 | 979.53 | 653.36 |
| 557 | Ac-AAALTF$r8EYWAQL$AAAAAa-NH₂ | | 2142.15 | 1072.83 | 2143.16 | 1072.08 | 715.06 |
| 558 | Ac-LTF34F2$r8EYWAQL$AAAAAa-NH₂ | | 1965.02 | 984.3 | 1966.03 | 983.52 | 656.01 |
| 559 | Ac-RTF$r8EYWAQL$AAAAAa-NH₂ | | 1972.06 | 987.81 | 1973.07 | 987.04 | 658.36 |
| 560 | Ac-LTA$r8EYWAQL$AAAAAa-NH₂ | | 1853.01 | 928.33 | 1854.02 | 927.51 | 618.68 |
| 561 | Ac-LTF$r8EYWAibQL$AAAAAa-NH₂ | | 1943.06 | 973.48 | 1944.07 | 972.54 | 648.69 |
| 562 | Ac-LTF$r8EYWAQL$AAibAAAa-NH₂ | | 1943.06 | 973.11 | 1944.07 | 972.54 | 648.69 |
| 563 | Ac-LTF$r8EYWAQL$AAAibAAa-NH₂ | | 1943.06 | 973.48 | 1944.07 | 972.54 | 648.69 |
| 564 | Ac-LTF$r8EYWAQL$AAAAibAa-NH₂ | | 1943.06 | 973.48 | 1944.07 | 972.54 | 648.69 |
| 565 | Ac-LTF$r8EYWAQL$AAAAAiba-NH₂ | | 1943.06 | 973.38 | 1944.07 | 972.54 | 648.69 |
| 566 | Ac-LTF$r8EYWAQL$AAAAAiba-NH₂ | iso2 | 1943.06 | 973.38 | 1944.07 | 972.54 | 648.69 |
| 567 | Ac-LTF$r8EYWAQL$AAAAAAib-NH₂ | | 1943.06 | 973.01 | 1944.07 | 972.54 | 648.69 |
| 568 | Ac-LTF$r8EYWAQL$AaAAAa-NH₂ | | 1929.04 | 966.54 | 1930.05 | 965.53 | 644.02 |
| 569 | Ac-LTF$r8EYWAQL$AAaAAa-NH₂ | | 1929.04 | 966.35 | 1930.05 | 965.53 | 644.02 |
| 570 | Ac-LTF$r8EYWAQL$AAAaAa-NH₂ | | 1929.04 | 966.54 | 1930.05 | 965.53 | 644.02 |
| 571 | Ac-LTF$r8EYWAQL$AAAaAa-NH₂ | iso2 | 1929.04 | 966.35 | 1930.05 | 965.53 | 644.02 |
| 572 | Ac-LTF$r8EYWAQL$AAAAaa-NH₂ | | 1929.04 | 966.35 | 1930.05 | 965.53 | 644.02 |
| 573 | Ac-LTF$r8EYWAQL$AAAAAA-NH₂ | | 1929.04 | 966.35 | 1930.05 | 965.53 | 644.02 |
| 574 | Ac-LTF$r8EYWAQL$ASarAAAa-NH₂ | | 1929.04 | 966.54 | 1930.05 | 965.53 | 644.02 |
| 575 | Ac-LTF$r8EYWAQL$AASarAAa-NH₂ | | 1929.04 | 966.35 | 1930.05 | 965.53 | 644.02 |
| 576 | Ac-LTF$r8EYWAQL$AAASarAa-NH₂ | | 1929.04 | 966.35 | 1930.05 | 965.53 | 644.02 |
| 577 | Ac-LTF$r8EYWAQL$AAAASara-NH₂ | | 1929.04 | 966.35 | 1930.05 | 965.53 | 644.02 |
| 578 | Ac-LTF$r8EYWAQL$AAAAASar-NH₂ | | 1929.04 | 966.08 | 1930.05 | 965.53 | 644.02 |
| 579 | Ac-7LTF$r8EYWAQL$AAAAAa-NH₂ | | 1918.07 | 951.99 | 1919.08 | 960.04 | 640.37 |
| 581 | Ac-TF$r8EYWAQL$AAAAAa-NH₂ | | 1815.96 | 929.85 | 1816.97 | 908.99 | 606.33 |
| 582 | Ac-F$r8EYWAQL$AAAAAa-NH₂ | | 1714.91 | 930.92 | 1715.92 | 858.46 | 572.64 |
| 583 | Ac-LVF$r8EYWAQL$AAAAAa-NH₂ | | 1927.06 | 895.12 | 1928.07 | 964.54 | 643.36 |
| 584 | Ac-AAF$r8EYWAQL$AAAAAa-NH₂ | | 1856.98 | 859.51 | 1857.99 | 929.5 | 620 |
| 585 | Ac-LTF$r8EYWAQL$AAAAa-NH₂ | | 1858 | 824.08 | 1859.01 | 930.01 | 620.34 |
| 586 | Ac-LTF$r8EYWAQL$AAAa-NH₂ | | 1786.97 | 788.56 | 1787.98 | 894.49 | 596.66 |
| 587 | Ac-LTF$r8EYWAQL$AAa-NH₂ | | 1715.93 | 1138.57 | 1716.94 | 858.97 | 572.98 |
| 588 | Ac-LTF$r8EYWAQL$Aa-NH₂ | | 1644.89 | 1144.98 | 1645.9 | 823.45 | 549.3 |
| 589 | Ac-LTF$r8EYWAQL$a-NH₂ | | 1573.85 | 1113.71 | 1574.86 | 787.93 | 525.62 |
| 590 | Ac-LTF$r8EYWAQL$AAA-OH | | 1716.91 | 859.55 | 1717.92 | 859.46 | 573.31 |
| 591 | Ac-LTF$r8EYWAQL$A-OH | | 1574.84 | 975.14 | 1575.85 | 788.43 | 525.95 |
| 592 | Ac-LTF$r8EYWAQL$AAA-NH₂ | | 1715.93 | 904.75 | 1716.94 | 858.97 | 572.98 |
| 593 | Ac-LTF$r8EYWAQCba$SAA-OH | | 1744.91 | 802.49 | 1745.92 | 873.46 | 582.64 |
| 594 | Ac-LTF$r8EYWAQCba$S-OH | | 1602.83 | 913.53 | 1603.84 | 802.42 | 535.28 |
| 595 | Ac-LTF$r8EYWAQCba$S-NH₂ | | 1601.85 | 979.58 | 1602.86 | 801.93 | 534.96 |
| 596 | 4-FBzl-LTF$r8EYWAQL$AAAAAa-NH₂ | | 2009.05 | 970.52 | 2010.06 | 1005.53 | 670.69 |
| 597 | 4-FBzl-LTF$r8EYWAQCba$SAA-NH₂ | | 1823.93 | 965.8 | 1824.94 | 912.97 | 608.98 |
| 598 | Ac-LTF$r8RYWAQL$AAAAAa-NH₂ | | 1956.1 | 988.28 | 1957.11 | 979.06 | 653.04 |
| 599 | Ac-LTF$r8HYWAQL$AAAAAa-NH₂ | | 1937.06 | 1003.54 | 1938.07 | 969.54 | 646.69 |
| 600 | Ac-LTF$r8QYWAQL$AAAAAa-NH₂ | | 1928.06 | 993.92 | 1929.07 | 965.04 | 643.69 |
| 601 | Ac-LTF$r8CitYWAQL$AAAAAa-NH₂ | | 1957.08 | 987 | 1958.09 | 979.55 | 653.37 |
| 602 | Ac-LTF$r8GlaYWAQL$AAAAAa-NH₂ | | 1973.03 | 983 | 1974.04 | 987.52 | 658.68 |
| 603 | Ac-LTF$r8F4gYWAQL$AAAAAa-NH₂ | | 2004.1 | 937.86 | 2005.11 | 1003.06 | 669.04 |
| 604 | Ac-LTF$r82mRYWAQL$AAAAAa-NH₂ | | 1984.13 | 958.58 | 1985.14 | 993.07 | 662.38 |
| 605 | Ac-LTF$r8ipKYWAQL$AAAAAa-NH₂ | | 1970.14 | 944.52 | 1971.15 | 986.08 | 657.72 |
| 606 | Ac-LTF$r8F4NH₂YWAQL$AAAAAa-NH₂ | | 1962.08 | 946 | 1963.09 | 982.05 | 655.03 |
| 607 | Ac-LTF$r8EYWAAL$AAAAAa-NH₂ | | 1872.02 | 959.32 | 1873.03 | 937.02 | 625.01 |
| 608 | Ac-LTF$r8EYWALL$AAAAAa-NH₂ | | 1914.07 | 980.88 | 1915.08 | 958.04 | 639.03 |
| 609 | Ac-LTF$r8EYWAAibL$AAAAAa-NH₂ | | 1886.03 | 970.61 | 1887.04 | 944.02 | 629.68 |
| 610 | Ac-LTF$r8EYWASL$AAAAAa-NH₂ | | 1888.01 | 980.51 | 1889.02 | 945.01 | 630.34 |
| 611 | Ac-LTF$r8EYWANL$AAAAAa-NH₂ | | 1915.02 | 1006.41 | 1916.03 | 958.52 | 639.35 |
| 612 | Ac-LTF$r8EYWACitL$AAAAAa-NH₂ | | 1958.07 | | 1959.08 | 980.04 | 653.7 |
| 613 | Ac-LTF$r8EYWAHL$AAAAAa-NH₂ | | 1938.04 | 966.24 | 1939.05 | 970.03 | 647.02 |
| 614 | Ac-LTF$r8EYWARL$AAAAAa-NH₂ | | 1957.08 | | 1958.09 | 979.55 | 653.37 |
| 615 | Ac-LTF$r8EpYWAQL$AAAAAa-NH₂ | | 2009.01 | | 2010.02 | 1005.51 | 670.68 |
| 616 | Cbm-LTF$r8EYWAQCba$SAA-NH₂ | | 1590.85 | | 1591.86 | 796.43 | 531.29 |
| 617 | Cbm-LTF$r8EYWAQL$AAAAAa-NH₂ | | 1930.04 | | 1931.05 | 966.03 | 644.35 |
| 618 | Ac-LTF$r8EYWAQL$SAAAAa-NH₂ | | 1945.04 | 1005.11 | 1946.05 | 973.53 | 649.35 |
| 619 | Ac-LTF$r8EYWAQL$AAAASa-NH₂ | | 1945.04 | 986.52 | 1946.05 | 973.53 | 649.35 |
| 620 | Ac-LTF$r8EYWAQL$SAAASa-NH₂ | | 1961.03 | 993.27 | 1962.04 | 981.52 | 654.68 |
| 621 | Ac-LTF$r8EYWAQTba$AAAAAa-NH₂ | | 1943.06 | 983.1 | 1944.07 | 972.54 | 648.69 |
| 622 | Ac-LTF$r8EYWAQAdm$AAAAAa-NH₂ | | 2007.09 | 990.31 | 2008.1 | 1004.55 | 670.04 |
| 623 | Ac-LTF$r8EYWAQCha$AAAAAa-NH₂ | | 1969.07 | 987.17 | 1970.08 | 985.54 | 657.36 |
| 624 | Ac-LTF$r8EYWAQhCha$AAAAAa-NH₂ | | 1983.09 | 1026.11 | 1984.1 | 992.55 | 662.04 |
| 625 | Ac-LTF$r8EYWAQF$AAAAAa-NH₂ | | 1963.02 | 957.01 | 1964.03 | 982.52 | 655.35 |
| 626 | Ac-LTF$r8EYWAQhF$AAAAAa-NH₂ | | 1977.04 | 1087.81 | 1978.05 | 989.53 | 660.02 |
| 627 | Ac-LTF$r8EYWAQL$AANleAAa-NH₂ | | 1971.09 | 933.45 | 1972.1 | 986.55 | 658.04 |
| 628 | Ac-LTF$r8EYWAQAdm$AANleAAa-NH₂ | | 2049.13 | 1017.97 | 2050.14 | 1025.57 | 684.05 |
| 629 | 4-FBz-BaLTF$r8EYWAQL$AAAAAa-NH₂ | | 2080.08 | | 2081.09 | 1041.05 | 694.37 |
| 630 | 4-FBz-BaLTF$r8EYWAQCba$SAA-NH₂ | | 1894.97 | | 1895.98 | 948.49 | 632.66 |
| 631 | Ac-LTF$r5EYWAQL$s8AAAAAa-NH₂ | | 1929.04 | 1072.68 | 1930.05 | 965.53 | 644.02 |
| 632 | Ac-LTF$r5EYWAQCba$s8SAA-NH₂ | | 1743.92 | 1107.79 | 1744.93 | 872.97 | 582.31 |
| 633 | Ac-LTF$r8EYWAQL$AAhhLAAa-NH₂ | | 1999.12 | | 2000.13 | 1000.57 | 667.38 |
| 634 | Ac-LTF$r8EYWAQL$AAAAAAAa-NH₂ | | 2071.11 | | 2072.12 | 1036.56 | 691.38 |
| 635 | Ac-LTF$r8EYWAQL$AAAAAAAAa-NH₂ | | 2142.15 | 778.1 | 2143.16 | 1072.08 | 715.06 |
| 636 | Ac-LTF$r8EYWAQL$AAAAAAAAAa-NH₂ | | 2213.19 | 870.53 | 2214.2 | 1107.6 | 738.74 |
| 637 | Ac-LTA$r8EYAAQCba$SAA-NH₂ | | 1552.85 | | 1553.86 | 777.43 | 518.62 |
| 638 | Ac-LTA$r8EYAAQL$AAAAAa-NH₂ | | 1737.97 | 779.45 | 1738.98 | 869.99 | 580.33 |
| 639 | Ac-LTF$r8EPmpWAQL$AAAAAa-NH₂ | | 2007.03 | 779.54 | 2008.04 | 1004.52 | 670.02 |
| 640 | Ac-LTF$r8EPmpWAQCba$SAA-NH₂ | | 1821.91 | 838.04 | 1822.92 | 911.96 | 608.31 |
| 641 | Ac-ATF$r8HYWAQL$S-NH₂ | | 1555.82 | 867.83 | 1556.83 | 778.92 | 519.61 |
| 642 | Ac-LTF$r8HAWAQL$S-NH₂ | | 1505.84 | 877.91 | 1506.85 | 753.93 | 502.95 |
| 643 | Ac-LTF$r8HYWAQA$S-NH₂ | | 1555.82 | 852.52 | 1556.83 | 778.92 | 519.61 |
| 644 | Ac-LTF$r8EYWAQCba$SA-NH₂ | | 1672.89 | 887.18 | 1673.9 | 837.45 | 558.64 |
| 645 | Ac-LTF$r8EYWAQL$SAA-NH₂ | | 1731.92 | 873.32 | 1732.93 | 866.97 | 578.31 |
| 646 | Ac-LTF$r8HYWAQCba$SAA-NH₂ | | 1751.94 | 873.05 | 1752.95 | 876.98 | 584.99 |
| 647 | Ac-LTF$r8SYWAQCba$SAA-NH₂ | | 1701.91 | 844.88 | 1702.92 | 851.96 | 568.31 |
| 648 | Ac-LTF$r8RYWAQCba$SAA-NH₂ | | 1770.98 | 865.58 | 1771.99 | 886.5 | 591.33 |
| 649 | Ac-LTF$r8KYWAQCba$SAA-NH₂ | | 1742.98 | 936.57 | 1743.99 | 872.5 | 582 |
| 650 | Ac-LTF$r8QYWAQCba$SAA-NH₂ | | 1742.94 | 930.93 | 1743.95 | 872.48 | 581.99 |
| 651 | Ac-LTF$r8EYWAACba$SAA-NH₂ | | 1686.9 | 1032.45 | 1687.91 | 844.46 | 563.31 |
| 652 | Ac-LTF$r8EYWAQCba$AAA-NH₂ | | 1727.93 | 895.46 | 1728.94 | 864.97 | 576.98 |
| 653 | Ac-LTF$r8EYWAQL$AAAAA-OH | | 1858.99 | 824.54 | 1860 | 930.5 | 620.67 |
| 654 | Ac-LTF$r8EYWAQL$AAAA-OH | | 1787.95 | 894.48 | 1788.96 | 894.98 | 596.99 |
| 655 | Ac-LTF$r8EYWAQL$AA-OH | | 1645.88 | 856 | 1646.89 | 823.95 | 549.63 |
| 656 | Ac-LTF$r8AF4bOH2WAQL$AAAAAa-NH₂ | | | | | | |
| 657 | Ac-LTF$r8AF4bOH2WAAL$AAAAAa-NH₂ | | | | | | |
| 658 | Ac-LTF$r8EF4bOH2WAQCba$SAA-NH₂ | | | | | | |
| 659 | Ac-LTF$r8ApYWAQL$AAAAAa-NH₂ | | | | | | |
| 660 | Ac-LTF$r8ApYWAAL$AAAAAa-NH₂ | | | | | | |
| 661 | Ac-LTF$r8EpYWAQCba$SAA-NH₂ | | | | | | |
| 662 | Ac-LTF$rda6AYWAQL$da5AAAAAa-NH₂ | | 1974.06 | 934.44 | | | |
| 663 | Ac-LTF$rda6EYWAQCba$da5SAA-NH₂ | | 1846.95 | 870.52 | | 869.94 | |
| 664 | Ac-LTF$rda6EYWAQL$da5AAAAAa-NH₂ | | | | | | |
| 665 | Ac-LTF$ra9EYWAQL$a6AAAAAa-NH₂ | | | 936.57 | | 935.51 | |
| 666 | Ac-LTF$ra9EYWAQL$a6AAAAAa-NH₂ | | | | | | |
| 667 | Ac-LTF$ra9EYWAQCba$a6SAA-NH₂ | | | | | | |
| 668 | Ac-LTA$ra9EYWAQCba$a6SAA-NH₂ | | | | | | |
| 669 | 5-FAM-BaLTF$ra9EYWAQCba$a6SAA-NH₂ | | | | | | |
| 670 | 5-FAM-BaLTF$r8EYWAQL$AAAAAa-NH₂ | | 2316.11 | | | | |
| 671 | 5-FAM-BaLTF$/r8EYWAQL$/AAAAAa-NH₂ | | 2344.15 | | | | |
| 672 | 5-FAM-BaLTA$r8EYWAQL$AAAAAa-NH₂ | | 2240.08 | | | | |
| 673 | 5-FAM-BaLTF$r8AYWAQL$AAAAAa-NH₂ | | 2258.11 | | | | |
| 674 | 5-FAM-BaATF$r8EYWAQL$AAAAAa-NH₂ | | 2274.07 | | | | |
| 675 | 5-FAM-BaLAF$r8EYWAQL$AAAAAa-NH₂ | | 2286.1 | | | | |
| 676 | 5-FAM-BaLTF$r8EAWAQL$AAAAAa-NH₂ | | 2224.09 | | | | |
| 677 | 5-FAM-BaLTF$r8EYAAQL$AAAAAa-NH₂ | | 2201.07 | | | | |
| 678 | 5-FAM-BaLTA$r8EYAAQL$AAAAAa-NH₂ | | 2125.04 | | | | |
| 679 | 5-FAM-BaLTF$r8EYWAAL$AAAAAa-NH₂ | | 2259.09 | | | | |
| 680 | 5-FAM-BaLTF$r8EYWAQA$AAAAAa-NH₂ | | 2274.07 | | | | |
| 681 | 5-FAM-BaLTF$/r8EYWAQCba$/SAA-NH₂ | | 2159.03 | | | | |
| 682 | 5-FAM-BaLTA$r8EYWAQCba$SAA-NH₂ | | 2054.97 | | | | |
| 683 | 5-FAM-BaLTF$r8EYAAQCba$SAA-NH₂ | | 2015.96 | | | | |
| 684 | 5-FAM-BaLTA$r8EYAAQCba$SAA-NH₂ | | 1939.92 | | | | |
| 685 | 5-FAM-BaQSQQTF$r8NLWRLL$QN-NH₂ | | 2495.23 | | | | |
| 686 | 5-TAMRA-BaLTF$r8EYWAQCba$SAA-NH₂ | | 2186.1 | | | | |
| 687 | 5-TAMRA-BaLTA$r8EYWAQCba$SAA-NH₂ | | 2110.07 | | | | |
| 688 | 5-TAMRA-BaLTF$r8EYAAQCba$SAA-NH₂ | | 2071.06 | | | | |
| 689 | 5-TAMRA-BaLTA$r8EYAAQCba$SAA-NH₂ | | 1995.03 | | | | |
| 690 | 5-TAMRA-BaLTF$/r8EYWAQCba$/SAA-NH₂ | | 2214.13 | | | | |
| 691 | 5-TAMRA-BaLTF$r8EYWAQL$AAAAAa-NH₂ | | 2371.22 | | | | |
| 692 | 5-TAMRA-BaLTA$r8EYWAQL$AAAAAa-NH₂ | | 2295.19 | | | | |
| 693 | 5-TAMRA-BaLTF$/r8EYWAQL$/AAAAAa-NH₂ | | 2399.25 | | | | |
| 694 | Ac-LTF$r8EYWCou7QCba$SAA-OH | | 1947.93 | | | | |
| 695 | Ac-LTF$r8EYWCou7QCba$S-OH | | 1805.86 | | | | |
| 696 | Ac-LTA$r8EYWCou7QCba$SAA-NH₂ | | 1870.91 | | | | |
| 697 | Ac-LTF$r8EYACou7QCba$SAA-NH₂ | | 1831.9 | | | | |
| 698 | Ac-LTA$r8EYACou7QCba$SAA-NH₂ | | 1755.87 | | | | |
| 699 | Ac-LTF$/r8EYWCou7QCba$/SAA-NH₂ | | 1974.98 | | | | |
| 700 | Ac-LTF$r8EYWCou7QL$AAAAAa-NH₂ | | 2132.06 | | | | |
| 701 | Ac-LTF$/r8EYWCou7QL$/AAAAAa-NH₂ | | 2160.09 | | | | |
| 702 | Ac-LTF$r8EYWCou7QL$AAAAA-OH | | 2062.01 | | | | |
| 703 | Ac-LTF$r8EYWCou7QL$AAAA-OH | | 1990.97 | | | | |
| 704 | Ac-LTF$r8EYWCou7QL$AAA-OH | | 1919.94 | | | | |
| 705 | Ac-LTF$r8EYWCou7QL$AA-OH | | 1848.9 | | | | |
| 706 | Ac-LTF$r8EYWCou7QL$A-OH | | 1777.86 | | | | |
| 707 | Ac-LTF$r8EYWAQL$AAAASa-NH₂ | iso2 | | 974.4 | | 973.53 | |
| 708 | Ac-LTF$r8AYWAAL$AAAAAa-NH₂ | iso2 | 1814.01 | 908.82 | 1815.02 | 908.01 | 605.68 |
| 709 | Biotin-BaLTF$r8EYWAQL$AAAAAa-NH₂ | | 2184.14 | 1093.64 | 2185.15 | 1093.08 | 729.05 |
| 710 | Ac-LTF$r8NAWAQL$S-NN₂ | iso2 | 1505.84 | 754.43 | 1506.85 | 753.93 | 502.95 |
| 711 | Ac-LTF$r8EYWAQCba$SA-NH₂ | iso2 | 1672.89 | 838.05 | 1673.9 | 837.45 | 558.64 |
| 712 | Ac-LTF$r8HYWAQCba$SAA-NH₂ | iso2 | 1751.94 | 877.55 | 1752.95 | 876.98 | 584.99 |
| 713 | Ac-LTF$r8SYWAQCba$SAA-NH₂ | iso2 | 1701.91 | 852.48 | 1702.92 | 851.96 | 568.31 |
| 714 | Ac-LTF$r8RYWAQCba$SAA-NH₂ | iso2 | 1770.98 | 887.45 | 1771.99 | 886.5 | 591.33 |
| 715 | Ac-LTF$r8KYWAQCba$SAA-NH₂ | iso2 | 1742.98 | 872.92 | 1743.99 | 872.5 | 582 |
| 716 | Ac-LTF$r8EYWAQCba$AAA-NH₂ | iso2 | 1727.93 | 865.71 | 1728.94 | 864.97 | 576.98 |
| 717 | Ac-LTF$r8EYWAQL$AAAAAaBaC-NH₂ | | 2103.09 | 1053.12 | 2104.1 | 1052.55 | 702.04 |
| 718 | Ac-LTF$r8EYWAQL$AAAAAadPeg4C-NH₂ | | 2279.19 | 1141.46 | 2280.2 | 1140.6 | 760.74 |
| 719 | Ac-LTA$r8AYWAAL$AAAAAa-NH₂ | | 1737.98 | 870.43 | 1738.99 | 870 | 580.33 |
| 720 | Ac-LTF$r8AYAAAL$AAAAAa-NH₂ | | 1698.97 | 851 | 1699.98 | 850.49 | 567.33 |
| 721 | 5-FAM-BaLTF$r8AYWAAL$AAAAAa-NH₂ | | 2201.09 | 1101.87 | 2202.1 | 1101.55 | 734.7 |
| 722 | Ac-LTA$r8AYWAQL$AAAAAa-NH₂ | | 1795 | 898.92 | 1796.01 | 898.51 | 599.34 |
| 723 | Ac-LTF$r8AYAAQL$AAAAAa-NH₂ | | 1755.99 | 879.49 | 1757 | 879 | 586.34 |
| 724 | Ac-LTF$rda6AYWAAL$da5AAAAAa-NH₂ | | 1807.97 | | 1808.98 | 904.99 | 603.66 |
| 725 | FITC-BaLTF$r8EYWAQL$AAAAAa-NH₂ | | 2347.1 | 1174.49 | 2348.11 | 1174.56 | 783.37 |
| 726 | FITC-BaLTF$r8EYWAQCba$SAA-NH₂ | | 2161.99 | 1082.35 | 2163 | 1082 | 721.67 |
| 733 | Ac-LTF$r8EYWAQL$EAAAAa-NH₂ | | 1987.05 | 995.03 | 1988.06 | 994.53 | 663.36 |
| 734 | Ac-LTF$r8AYWAQL$EAAAAa-NH₂ | | 1929.04 | 966.35 | 1930.05 | 965.53 | 644.02 |
| 735 | Ac-LTF$r8EYWAQL$AAAAAaBaKbio-NH₂ | | 2354.25 | 1178.47 | 2355.26 | 1178.13 | 785.76 |
| 736 | Ac-LTF$r8AYWAAL$AAAAAa-NH₂ | | 1814.01 | 908.45 | 1815.02 | 908.01 | 605.68 |
| 737 | Ac-LTF$r8AYAAAL$AAAAAa-NH₂ | iso2 | 1698.97 | 850.91 | 1699.98 | 850.49 | 567.33 |
| 738 | Ac-LTF$r8AYAAQL$AAAAAa-NH₂ | iso2 | 1755.99 | 879.4 | 1757 | 879 | 586.34 |
| 739 | Ac-LTF$r8EYWAQL$EAAAAa-NH₂ | iso2 | 1987.05 | 995.21 | 1988.06 | 994.53 | 663.36 |
| 740 | Ac-LTF$r8AYWAQL$EAAAAa-NH₂ | iso2 | 1929.04 | 966.08 | 1930.05 | 965.53 | 644.02 |
| 741 | Ac-LTF$r8EYWAQCba$SAAAAa-NH₂ | | 1957.04 | 980.04 | 1958.05 | 979.53 | 653.35 |
| 742 | Ac-LTF$r8EYWAQLStAAA$r5AA-NH₂ | | 2023.12 | 1012.83 | 2024.13 | 1012.57 | 675.38 |
| 743 | Ac-LTF$r8EYWAQL$A$AAA$A-NH₂ | | 2108.17 | 1055.44 | 2109.18 | 1055.09 | 703.73 |
| 744 | Ac-LTF$r8EYWAQL$AA$AAA$A-NH₂ | | 2179.21 | 1090.77 | 2180.22 | 1090.61 | 727.41 |
| 745 | Ac-LTF$r8EYWAQL$AAA$AAA$A-NH₂ | | 2250.25 | 1126.69 | 2251.26 | 1126.13 | 751.09 |
| 746 | Ac-AAALTF$r8EYWAQL$AAA-OH | | 1930.02 | | 1931.03 | 966.02 | 644.35 |
| 747 | Ac-AAALTF$r8EYWAQL$AAA-NH₂ | | 1929.04 | 965.85 | 1930.05 | 965.53 | 644.02 |
| 748 | Ac-AAAALTF$r8EYWAQL$AAA-NH₂ | | 2000.08 | 1001.4 | 2001.09 | 1001.05 | 667.7 |
| 749 | Ac-AAAAALTF$r8EYWAQL$AAA-NH₂ | | 2071.11 | 1037.13 | 2072.12 | 1036.56 | 691.38 |
| 750 | Ac-AAAAAALTF$r8EYWAQL$AAA-NH₂ | | 2142.15 | | 2143.16 | 1072.08 | 715.06 |
| 751 | Ac-LTF$rda6EYWAQCba$da6SAA-NH₂ | iso2 | 1751.89 | 877.36 | 1752.9 | 876.95 | 584.97 |
| 752 | Ac-t$r5wya$r5f4CF3ekllr-NH₂ | | | 844.25 | | | |
| 753 | Ac-tawy$r5nf4CF3e$r5llr-NH₂ | | | 837.03 | | | |
| 754 | Ac-tawya$r5f4CF3ek$r5lr-NH₂ | | | 822.97 | | | |
| 755 | Ac-tawyanf4CF3e$r5llr$r5a-NH₂ | | | 908.35 | | | |
| 756 | Ac-t$s8wyanf4CF3e$r5llr-NH₂ | | | 858.03 | | | |
| 757 | Ac-tawy$s8nf4CF3ekll$r5a-NH₂ | | | 879.86 | | | |
| 758 | Ac-tawya$s8f4CF3ekllr$r5a-NH₂ | | | 936.38 | | | |
| 759 | Ac-tawy$s8naekll$r5a-NH₂ | | | 844.25 | | | |
| 760 | 5-FAM-Batawy$s8nf4CF3ekll$r5a-NH₂ | | | | | | |
| 761 | 5-FAM-Batawy$s8naekll$r5a-NH₂ | | | | | | |
| 762 | Ac-tawy$s8nf4CF3eall$r5a-NH₂ | | | | | | |
| 763 | Ac-tawy$s8nf4CF3ekll$r5aaaaa-NH₂ | | | | | | |
| 764 | Ac-tawy$s8nf4CF3eall$r5aaaaa-NH₂ | | | | | | |

TABLE 1a shows a selection of peptidomimetic macrocycles.

TABLE 1a

| | | | Exact | Found | Calc | Calc | Calc |
|---|---|---|---|---|---|---|---|
| SP | Sequence | Isomer | Mass | Mass | (M + 1)/1 | (M + 2)/2 | (M + 3)/3 |
| 244 | Ac-LTF$r8EF4coohWAQCba$SANleA-NH₂ | | 1885 | 943.59 | 1886.01 | 943.51 | 629.34 |
| 331 | Ac-LTF$r8EYWAQL$AAAAAa-NH₂ | iso2 | 1929.04 | 966.08 | 1930.05 | 965.53 | 644.02 |
| 555 | Ac-LTF$r8EY6clWAQL$AAAAAa-NH₂ | | 1963 | 983.28 | 1964.01 | 982.51 | 655.34 |
| 557 | Ac-AAALTF$r8EYWAQL$AAAAAa-NH₂ | | 2142.15 | 1072.83 | 2143.16 | 1072.08 | 715.06 |
| 558 | Ac-LTF34F2$r8EYWAQL$AAAAAa-NH₂ | | 1965.02 | 984.3 | 1966.03 | 983.52 | 656.01 |
| 562 | Ac-LTF$r8EYWAQL$AAibAAAa-NH₂ | | 1943.06 | 973.11 | 1944.07 | 972.54 | 648.69 |
| 564 | Ac-LTF$r8EYWAQL$AAAAibAa-NH₂ | | 1943.06 | 973.48 | 1944.07 | 972.54 | 648.69 |
| 566 | Ac-LTF$r8EYWAQL$AAAAAiba-NH₂ | iso2 | 1943.06 | 973.38 | 1944.07 | 972.54 | 648.69 |
| 567 | Ac-LTF$r8EYWAQL$AAAAAAib-NH₂ | | 1943.06 | 973.01 | 1944.07 | 972.54 | 648.69 |
| 572 | Ac-LTF$r8EYWAQL$AAAAaa-NH₂ | | 1929.04 | 966.35 | 1930.05 | 965.53 | 644.02 |
| 573 | Ac-LTF$r8EYWAQL$AAAAAA-NH₂ | | 1929.04 | 966.35 | 1930.05 | 965.53 | 644.02 |
| 578 | Ac-LTF$r8EYWAQL$AAAAASar-NH₂ | | 1929.04 | 966.08 | 1930.05 | 965.53 | 644.02 |
| 551 | Ac-LTF$r8EYWAQL$AAAAAa-OH | iso2 | 1930.02 | 965.89 | 1931.03 | 966.02 | 644.35 |
| 662 | Ac-LTF$rda6AYWAQL$da5AAAAAa-NH₂ | | 1974.06 | 934.44 | | 933.49 | |
| 367 | 5-FAM-BaLTF$r8EYWAQCba$SAA-NH₂ | | 2131 | 1067.09 | 2132.01 | 1066.51 | 711.34 |
| 349 | Ac-LTF$r8EF4coohWAQCba$AAAAAa-NH₂ | iso2 | 1969.04 | 986.06 | 1970.05 | 985.53 | 657.35 |
| 347 | Ac-LTF$r8EYWAQCba$AAAAAa-NH₂ | iso2 | 1941.04 | 972.55 | 1942.05 | 971.53 | 648.02 |

TABLE 1b shows a further selection of peptidomimetic macrocycles.

TABLE 1b

| | | | | | Calc | Calc | Calc |
|---|---|---|---|---|---|---|---|
| | | Iso- | Exact | Found | (M + | (M + | (M + |
| Sp | Sequence | mer | Mass | Mass | 1)/1 | 2)/2 | 3)/3 |
| 581 | Ac-TF$r8EYWAQL$AAAAAa-NH₂ | | 1815.96 | 929.85 | 1816.97 | 908.99 | 606.33 |
| 582 | Ac-F$r8EYWAQL$AAAAAa-NH₂ | | 1714.91 | 930.92 | 1715.92 | 858.46 | 572.64 |
| 583 | Ac-LVF$r8EYWAQL$AAAAAa-NH₂ | | 1927.06 | 895.12 | 1928.07 | 964.54 | 643.36 |
| 584 | Ac-AAF$r8EYWAQL$AAAAAa-NH₂ | | 1856.98 | 859.51 | 1857.99 | 929.5 | 620 |
| 585 | Ac-LTF$r8EYWAQL$AAAAa-NH₂ | | 1858 | 824.08 | 1859.01 | 930.01 | 620.34 |
| 586 | Ac-LTF$r8EYWAQL$AAAa-NH₂ | | 1786.97 | 788.56 | 1787.98 | 894.49 | 596.66 |
| 587 | Ac-LTF$r8EYWAQL$AAa-NH₂ | | 1715.93 | 1138.57 | 1716.94 | 858.97 | 572.98 |
| 588 | Ac-LTF$r8EYWAQL$Aa-NH₂ | | 1644.89 | 1144.98 | 1645.9 | 823.45 | 549.3 |
| 589 | Ac-LTF$r8EYWAQL$a-NH₂ | | 1573.85 | 1113.71 | 1574.86 | 787.93 | 525.62 |

In the sequences shown above and elsewhere, the following abbreviations are used: “Nle” represents norleucine, “Aib” represents 2-aminoisobutyric acid, “Ac” represents acetyl, and “Pr” represents propionyl. Amino acids represented as “$” are alpha-Me S5-pentenyl-alanine olefin amino acids connected by an all-carbon crosslinker comprising one double bond. Amino acids represented as “$r5” are alpha-Me R5-pentenyl-alanine olefin amino acids connected by an all-carbon comprising one double bond. Amino acids represented as “$s8” are alpha-Me S8-octenyl-alanine olefin amino acids connected by an all-carbon crosslinker comprising one double bond. Amino acids represented as “$r8” are alpha-Me R8-octenyl-alanine olefin amino acids connected by an all-carbon crosslinker comprising one double bond. “Ahx” represents an aminocyclohexyl linker.

The crosslinkers are linear all-carbon crosslinker comprising eight or eleven carbon atoms between the alpha carbons of each amino acid. Amino acids represented as “$1” are alpha-Me S5-pentenyl-alanine olefin amino acids that are not connected by any crosslinker. Amino acids represented as “$/r5” are alpha-Me R5-pentenyl-alanine olefin amino acids that are not connected by any crosslinker. Amino acids represented as “$/s8” are alpha-Me S8-octenyl-alanine olefin amino acids that are not connected by any crosslinker. Amino acids represented as “$/r8” are alpha-Me R8-octenyl-alanine olefin amino acids that are not connected by any crosslinker.

Amino acids represented as “Amw” are alpha-Me tryptophan amino acids. Amino acids represented as “Aml” are alpha-Me leucine amino acids. Amino acids represented as “Amf” are alpha-Me phenylalanine amino acids. Amino acids represented as “2ff” are 2-fluoro-phenylalanine amino acids. Amino acids represented as “3ff” are 3-fluoro-phenylalanine amino acids. Amino acids represented as “St” are amino acids comprising two pentenyl-alanine olefin side chains, each of which is crosslinked to another amino acid as indicated. Amino acids represented as “St//” are amino acids comprising two pentenyl-alanine olefin side chains that are not crosslinked. Amino acids represented as “%St” are amino acids comprising two pentenyl-alanine olefin side chains, each of which is crosslinked to another amino acid as indicated via fully saturated hydrocarbon crosslinks. Amino acids represented as “Ba” are beta-alanine. The lower-case character “e” or “z” within the designation of a crosslinked amino acid (e.g. “$er8” or “$zr8”) represents the configuration of the double bond (E or Z, respectively). In other contexts, lower-case letters such as “a” or “f” represent D amino acids (e.g. D-alanine, or D-phenylalanine, respectively).

Amino acids designated as “NmW” represent N-methyltryptophan. Amino acids designated as “NmY” represent N-methyltyrosine. Amino acids designated as “NmA” represent N-methylalanine. “Kbio” represents a biotin group attached to the side chain amino group of a lysine residue. Amino acids designated as “Sar” represent sarcosine. Amino acids designated as “Cha” represent cyclohexyl alanine. Amino acids designated as “Cpg” represent cyclopentyl glycine. Amino acids designated as “Chg” represent cyclohexyl glycine. Amino acids designated as “Cba” represent cyclobutyl alanine. Amino acids designated as “F4I” represent 4-iodo phenylalanine. “7L” represents N15 isotopic leucine. Amino acids designated as “F3Cl” represent 3-chloro phenylalanine. Amino acids designated as “F4cooh” represent 4-carboxy phenylalanine. Amino acids designated as “F34F2” represent 3,4-difluoro phenylalanine. Amino acids designated as “6clW” represent 6-chloro tryptophan. Amino acids designated as “$rda6” represent alpha-Me R6-hexynyl-alanine alkynyl amino acids, crosslinked via a dialkyne bond to a second alkynyl amino acid.

Amino acids designated as “$da5” represent alpha-Me S5-pentynyl-alanine alkynyl amino acids, wherein the alkyne forms one half of a dialkyne bond with a second alkynyl amino acid. Amino acids designated as “$ra9” represent alpha-Me R9-nonynyl-alanine alkynyl amino acids, crosslinked via an alkyne metathesis reaction with a second alkynyl amino acid. Amino acids designated as “$a6” represent alpha-Me S6-hexynyl-alanine alkynyl amino acids, crosslinked via an alkyne metathesis reaction with a second alkynyl amino acid. The designation “iso1” or “iso2” indicates that the peptidomimetic macrocycle is a single isomer.

Amino acids designated as “Cit” represent citrulline. Amino acids designated as “Cou4”, “Cou6”, “Cou7” and “Cou8”, respectively, represent the following structures:

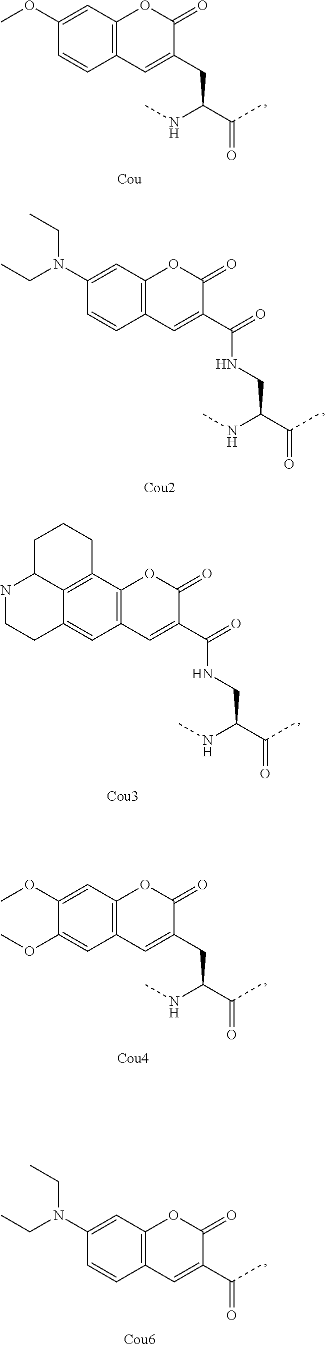

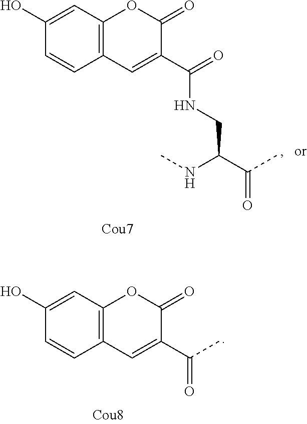

In some embodiments, a peptidomimetic macrocycle is obtained in more than one isomer, for example due to the configuration of a double bond within the structure of the crosslinker (E vs Z). Such isomers can or cannot be separable by conventional chromatographic methods. In some embodiments, one isomer has improved biological properties relative to the other isomer. In one embodiment, an E crosslinker olefin isomer of a peptidomimetic macrocycle has better solubility, better target affinity, better in vivo or in vitro efficacy, higher helicity, or improved cell permeability relative to its Z counterpart. In another embodiment, a Z crosslinker olefin isomer of a peptidomimetic macrocycle has better solubility, better target affinity, better in vivo or in vitro efficacy, higher helicity, or improved cell permeability relative to its E counterpart.

TABLE 1c shows exemplary peptidomimetic macrocycles.

TABLE 1c

| SP# | Structure |
|---|---|
| 154 | 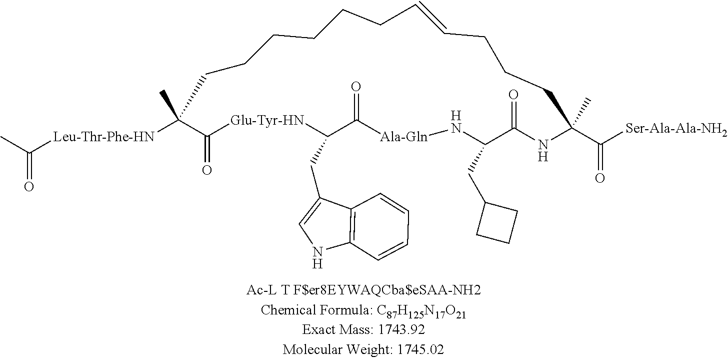 |
| 115 | 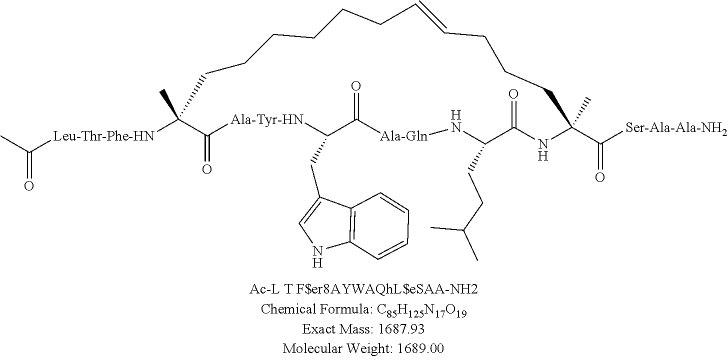 |
| 114 | 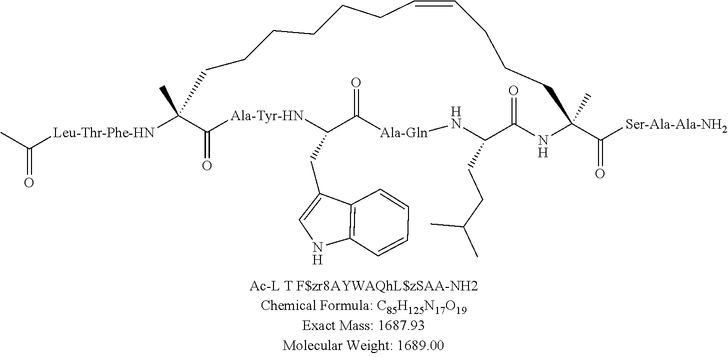 |
| 99 | 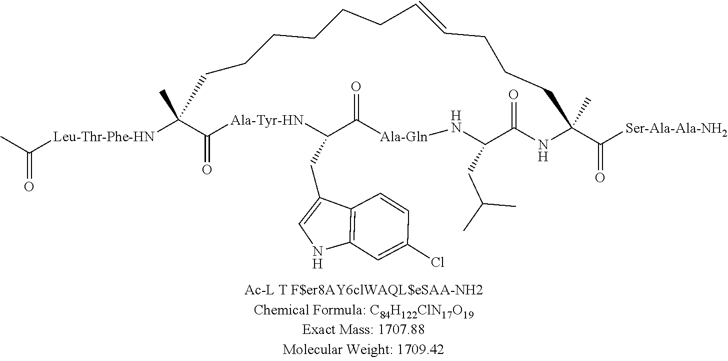 |
| 388 | 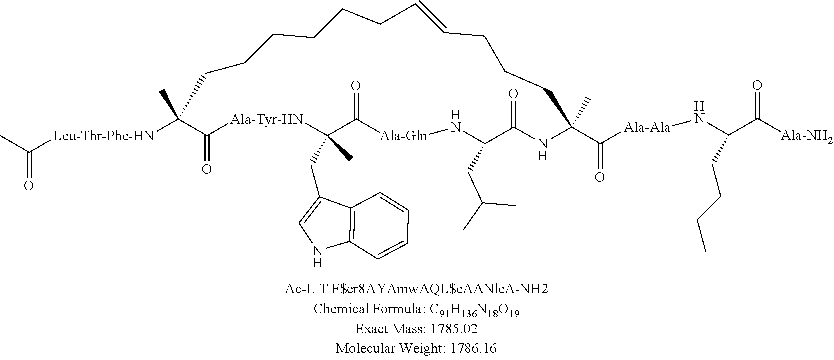 |
| 331 | 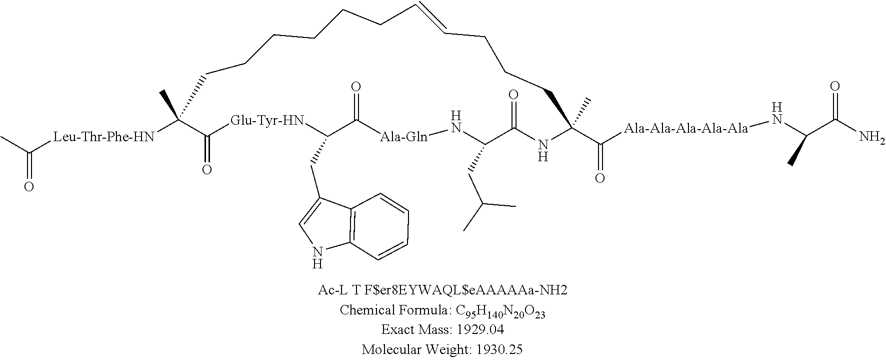 |
| 445 | 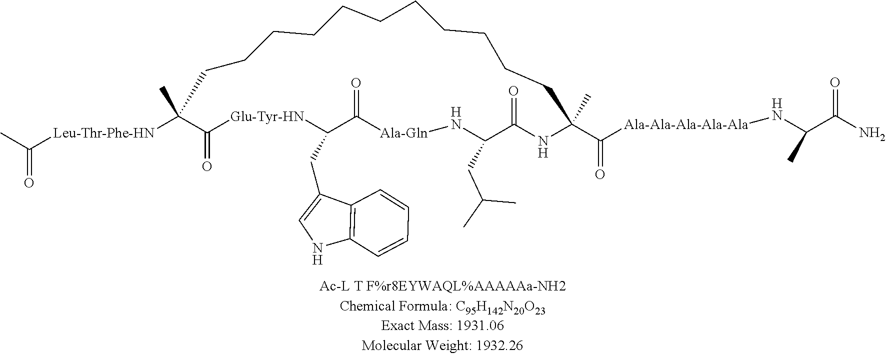 |
| 351 | 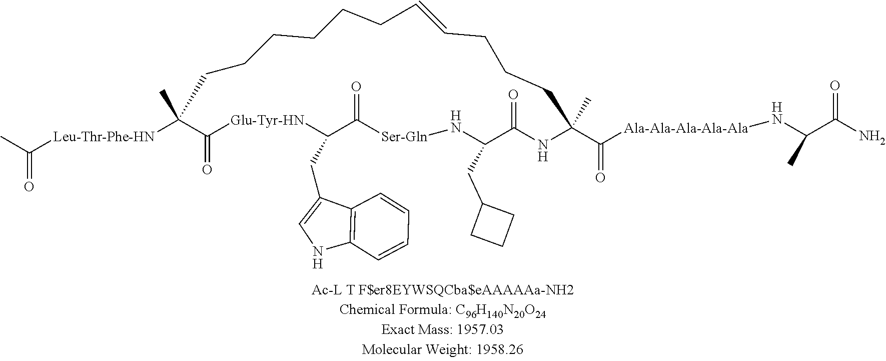 |
| 71 | 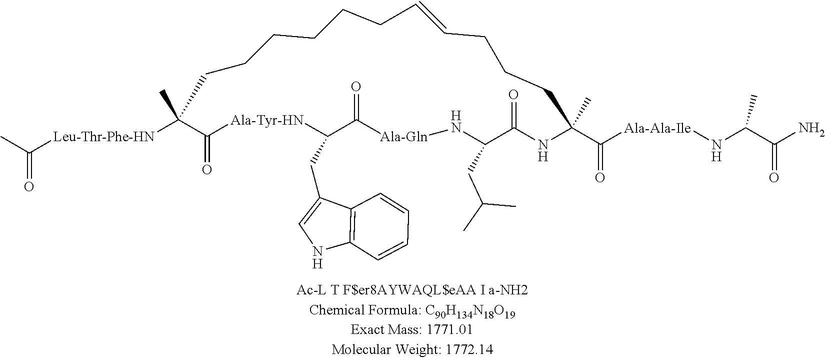 |
| 69 | 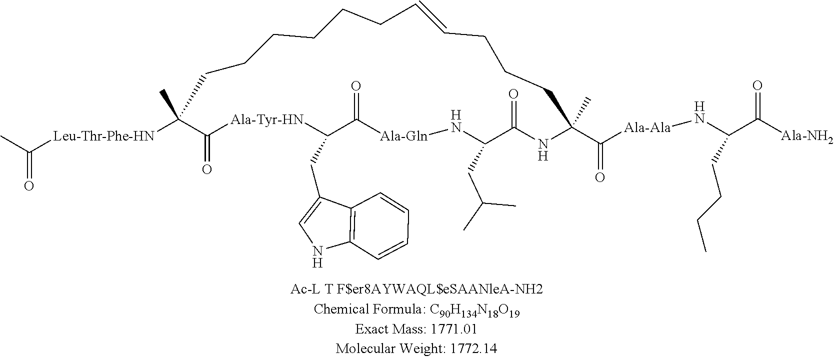 |
| 7 | 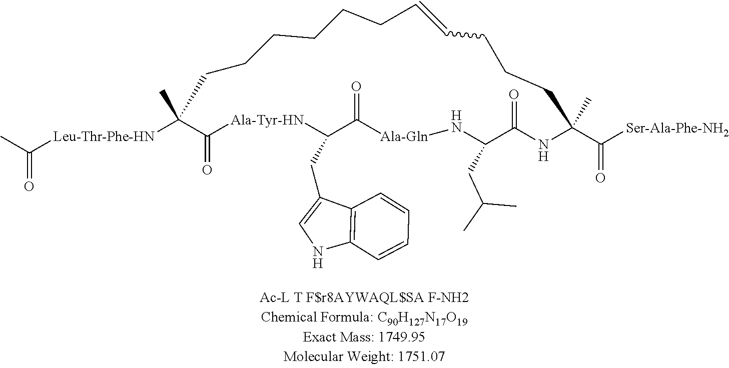 |
| 160 | 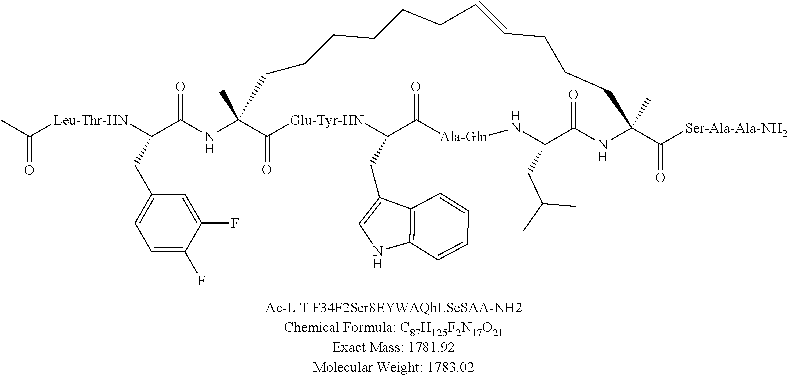 |
| 315 | 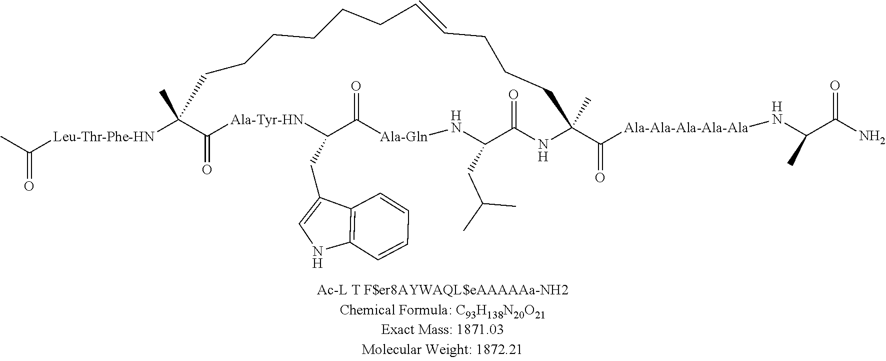 |
| 249 | 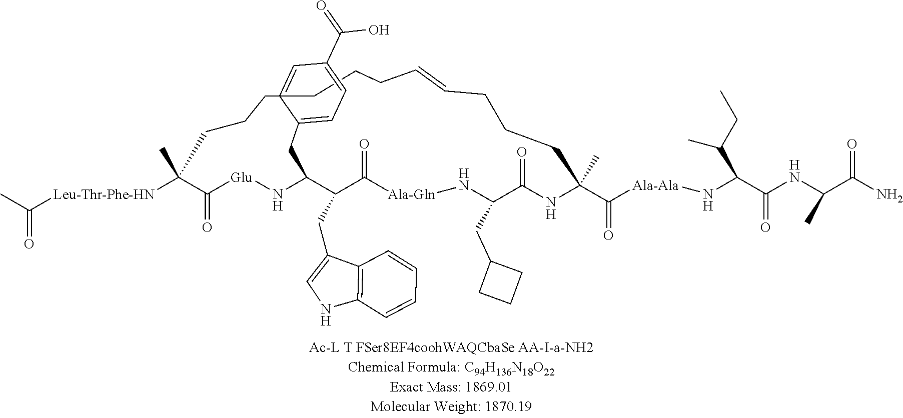 |
| 437 | 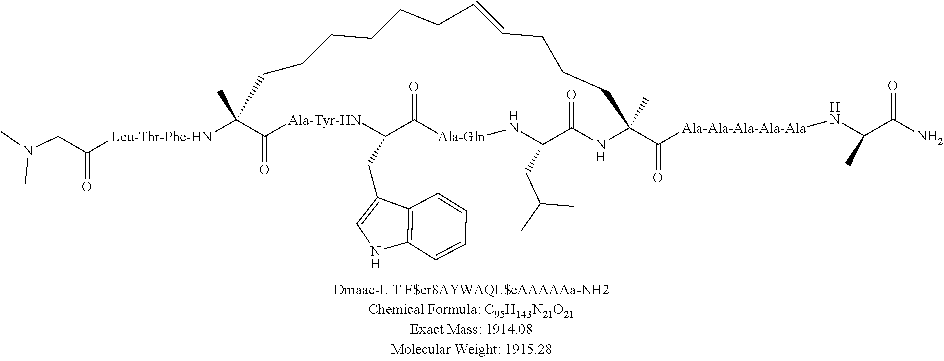 |
| 349 | 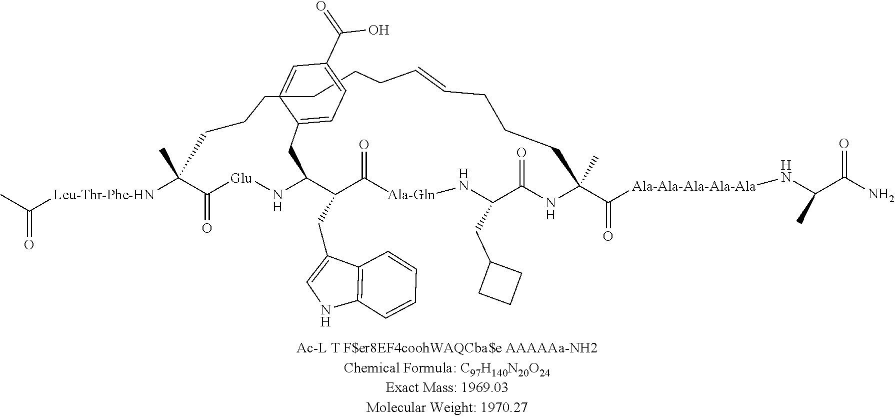 |
| 555 | 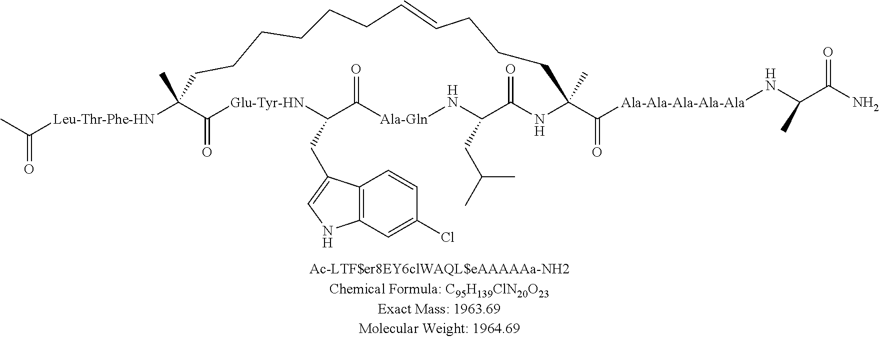 |
| 557 | 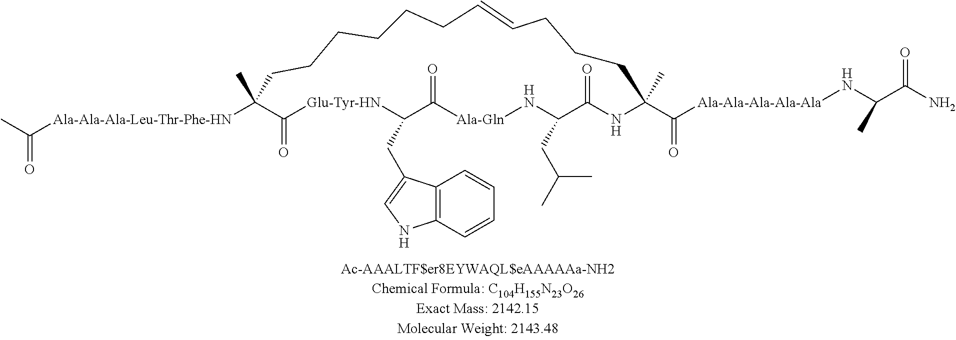 |
| 558 | 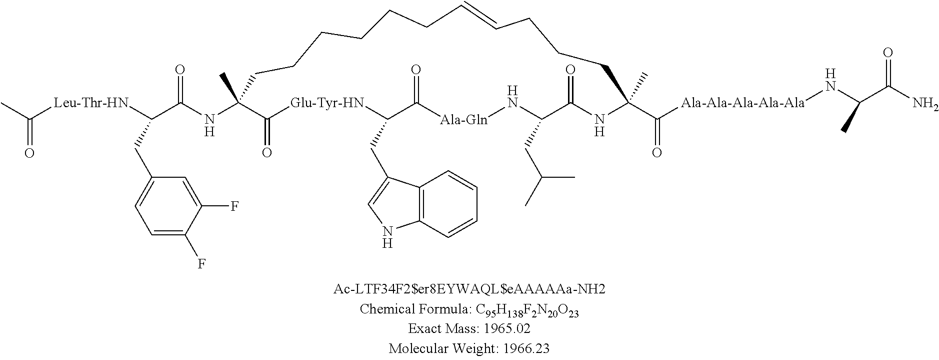 |
| 367 | 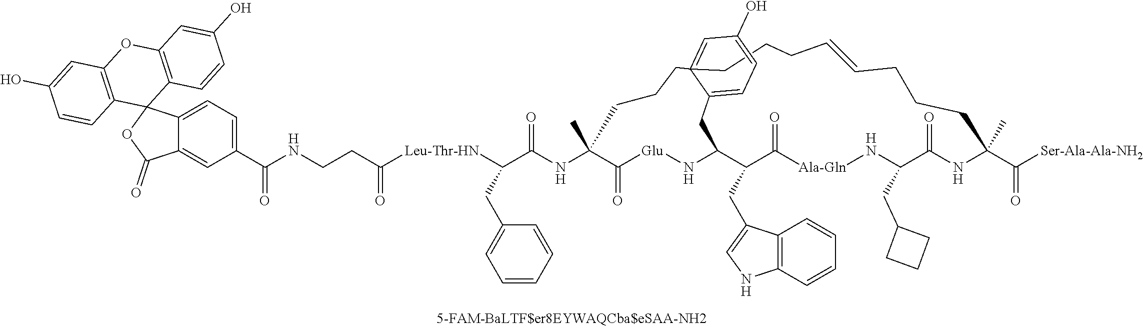 |
| 562 | 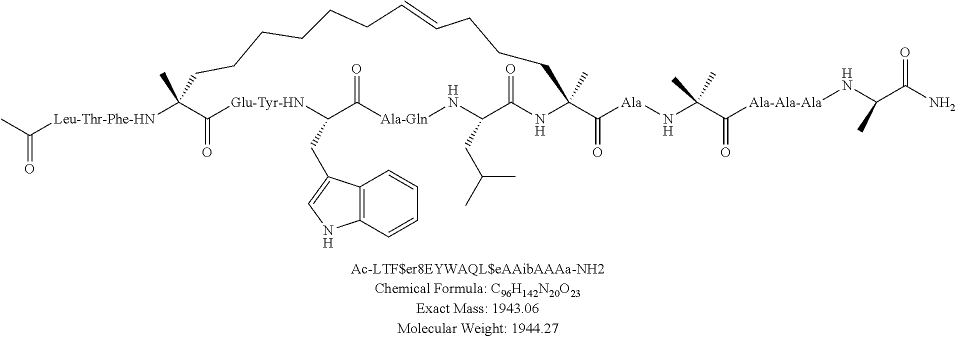 |
| 564 | 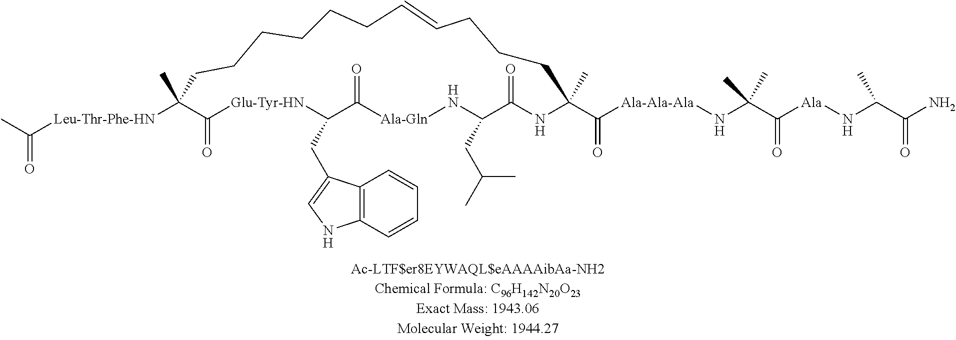 |
| 566 | 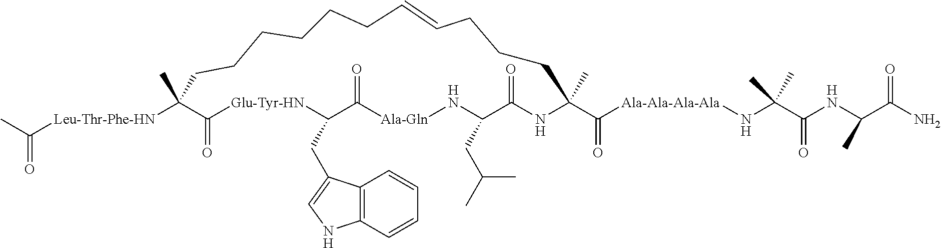 |
| 567 | 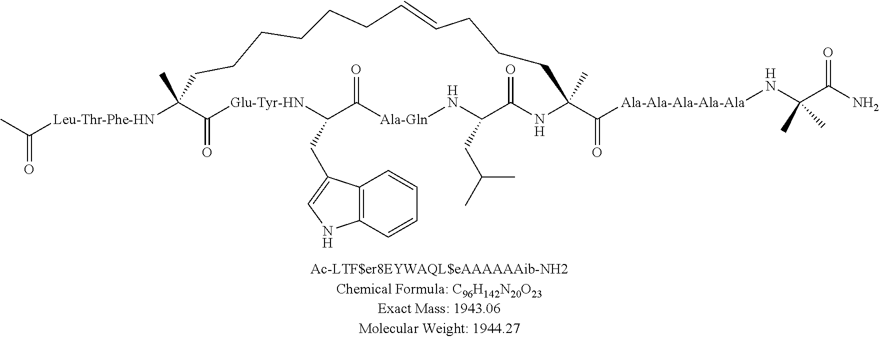 |
| 572 | 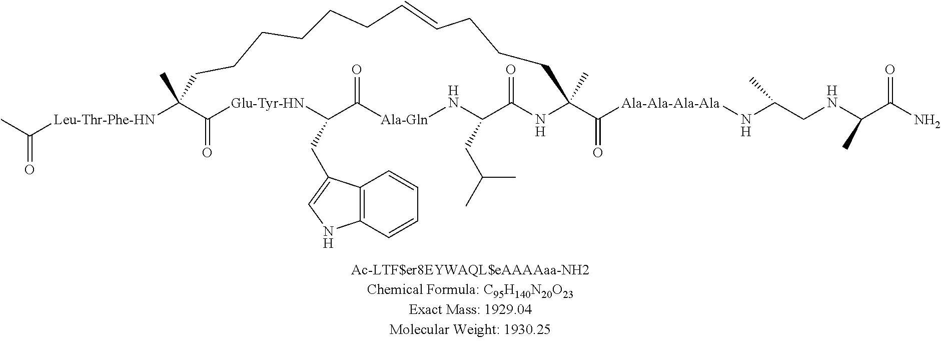 |
| 573 | 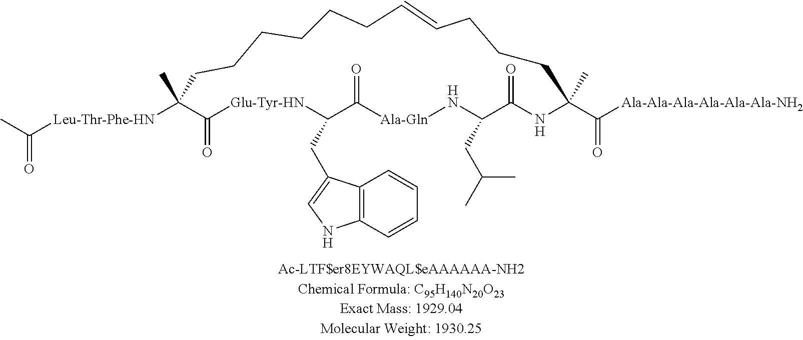 |
| 578 | 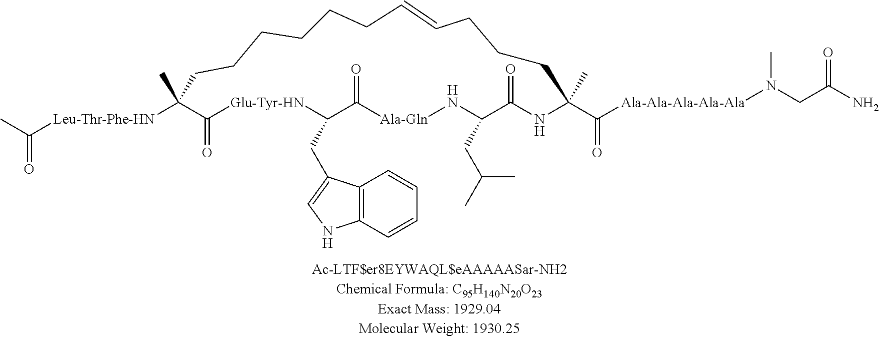 |
| 664 | 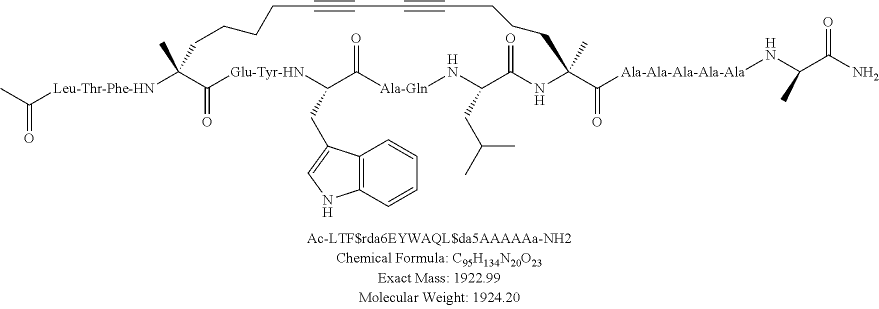 |
| 662 | 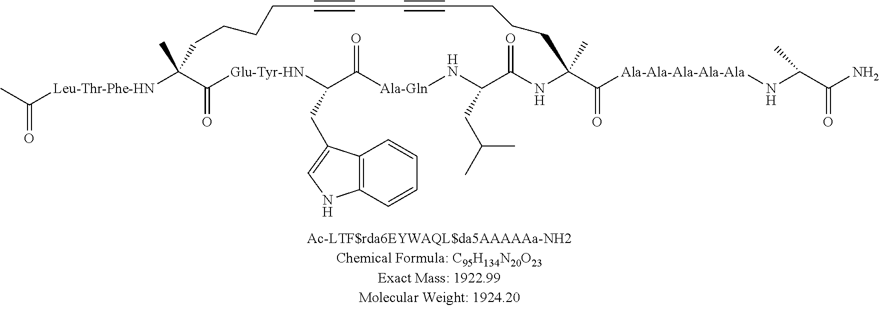 |
| | 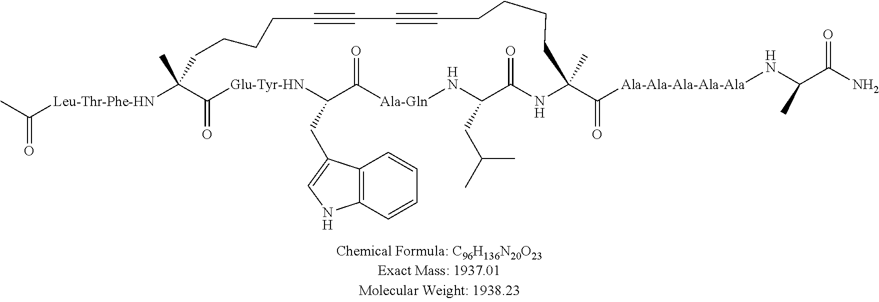 |

In some embodiments, peptidomimetic macrocycles exclude peptidomimetic macrocycles shown in TABLE 2a:

TABLE 2a

| SP | Sequence |
|---|---|
| 765 | L$r5QETFSD$s8WKLLPEN |
| 766 | LSQ$r5TFSDLW$s8LLPEN |
| 767 | LSQE$r5FSDLWK$s8LPEN |
| 768 | LSQET$r5SDLWKL$s8PEN |
| 769 | LSQETF$r5DLWKLL$s8EN |
| 770 | LXQETFS$r5LWKLLP$s8N |
| 771 | LSQETFSD$r5WKLLPE$s8 |
| 772 | LSQQTF$r5DLWKLL$s8EN |
| 773 | LSQETF$r5DLWKLL$s8QN |
| 774 | LSQQTF$r5DLWKLL$s8QN |
| 775 | LSQETF$r5NLWKLL$s8QN |
| 776 | LSQQTF$r5NLWKLL$s8QN |
| 777 | LSQQTF$r5NLWRLL$s8QN |
| 778 | QSQQTF$r5NLWKLL$s8QN |
| 779 | QSQQTF$r5NLWRLL$s8QN |
| 780 | QSQQTA$r5NLWRLL$s8QN |
| 781 | L$r8QETFSD$WKLLPEN |
| 782 | LSQ$r8TFSDLW$LLPEN |
| 783 | LSQE$r8FSDLWK$LPEN |
| 784 | LSQET$r8SDLWKL$PEN |
| 785 | LSQETF$r8DLWKLL$EN |
| 786 | LXQETFS$r8LWKLLP$N |
| 787 | LSQETFSD$r8WKLLPE$ |
| 788 | LSQQTF$r8DLWKLL$EN |
| 789 | LSQETF$r8DLWKLL$QN |
| 790 | LSQQTF$r8DLWKLL$QN |
| 791 | LSQETF$r8NLWKLL$QN |
| 792 | LSQQTF$r8NLWKLL$QN |
| 793 | LSQQTF$r8NLWRLL$QN |
| 794 | QSQQTF$r8NLWKLL$QN |
| 795 | QSQQTF$r8NLWRLL$QN |
| 796 | QSQQTA$r8NLWRLL$QN |
| 797 | QSQQTF$r8NLWRKK$QN |
| 798 | QQTF$r8DLWRLL$EN |
| 799 | QQTF$r8DLWRLL$ |
| 800 | LSQQTF$DLW$LL |
| 801 | QQTF$DLW$LL |
| 802 | QQTA$r8DLWRLL$EN |
| 803 | QSQQTF$r5NLWRLL$s8QN |
| | (dihydroxylated olefin) |
| 804 | QSQQTA$r5NLWRLL$s8QN |
| | (dihydroxylated olefin) |
| 805 | QSQQTF$r8DLWRLL$QN |
| 806 | QTF$r8NLWRLL$ |
| 807 | QSQQTF$NLW$LLPQN |
| 808 | QS$QTF$NLWRLLPQN |
| 809 | $TFS$LWKLL |
| 810 | ETF$DLW$LL |
| 811 | QTF$NLW$LL |
| 812 | $SQE$FSNLWKLL |

In TABLE 2a, the peptides can comprise an N-terminal capping group such as acetyl or an additional linker such as beta-alanine between the capping group and the start of the peptide sequence.

In some embodiments, peptidomimetic macrocycles do not comprise a peptidomimetic macrocycle structure as shown in TABLE 2a.

In some embodiments, peptidomimetic macrocycles exclude those shown in TABLE 2b:

TABLE 2b

| | | | | Observed |
|---|---|---|---|---|
| | | Exact | | mass |
| SP | Sequence | Mass | M + 2 | (m/e) |
| 813 | Ac-LSQETF$r8DLWKLL$EN-NH₂ | 2068.13 | 1035.07 | 1035.36 |
| 814 | Ac-LSQETF$r8NLWKLL$QN-NH₂ | 2066.16 | 1034.08 | 1034.31 |
| 815 | Ac-LSQQTF$r8NLWRLL$QN-NH₂ | 2093.18 | 1047.59 | 1047.73 |
| 816 | Ac-QSQQTF$r8NLWKLL$QN-NH₂ | 2080.15 | 1041.08 | 1041.31 |
| 817 | Ac-QSQQTF$r8NLWRLL$QN-NH₂ | 2108.15 | 1055.08 | 1055.32 |
| 818 | Ac-QSQQTA$r8NLWRLL$QN-NH₂ | 2032.12 | 1017.06 | 1017.24 |
| 819 | Ac-QAibQQTF$r8NLWRLL$QN-NH₂ | 2106.17 | 1054.09 | 1054.34 |
| 820 | Ac-QSQQTFSNLWRLLPQN-NH₂ | 2000.02 | 1001.01 | 1001.26 |
| 821 | Ac-QSQQTF$/r8NLWRLL$/QN-NH₂ | 2136.18 | 1069.09 | 1069.37 |
| 822 | Ac-QSQAibTF$r8NLWRLL$QN-NH₂ | 2065.15 | 1033.58 | 1033.71 |
| 823 | Ac-QSQQTF$r8NLWRLL$AN-NH₂ | 2051.13 | 1026.57 | 1026.70 |
| 824 | Ac-ASQQTF$r8NLWRLL$QN-NH₂ | 2051.13 | 1026.57 | 1026.90 |
| 825 | Ac-QSQQTF$r8ALWRLL$QN-NH₂ | 2065.15 | 1033.58 | 1033.41 |
| 826 | Ac-QSQETF$r8NLWRLL$QN-NH₂ | 2109.14 | 1055.57 | 1055.70 |
| 827 | Ac-RSQQTF$r8NLWRLL$QN-NH₂ | 2136.20 | 1069.10 | 1069.17 |
| 828 | Ac-RSQQTF$r8NLWRLL$EN-NH₂ | 2137.18 | 1069.59 | 1069.75 |
| 829 | Ac-LSQETFSDLWKLLPEN-NH₂ | 1959.99 | 981.00 | 981.24 |
| 830 | Ac-QSQ$TFS$LWRLLPQN-NH₂ | 2008.09 | 1005.05 | 1004.97 |
| 831 | Ac-QSQQ$FSN$WRLLPQN-NH₂ | 2036.06 | 1019.03 | 1018.86 |
| 832 | Ac-QSQQT$SNL$RLLPQN-NH₂ | 1917.04 | 959.52 | 959.32 |
| 833 | Ac-QsQQTF$NLW$LLPQN-NH₂ | 2007.06 | 1004.53 | 1004.97 |
| 834 | Ac-RTQATF$r8NQWAibANle$TNAibTR-NH₂ | 2310.26 | 1156.13 | 1156.52 |
| 835 | Ac-QSQQTF$r8NLWRLL$RN-NH₂ | 2136.20 | 1069.10 | 1068.94 |
| 836 | Ac-QSQRTF$r8NLWRLL$QN-NH₂ | 2136.20 | 1069.10 | 1068.94 |
| 837 | Ac-QSQQTF$r8NNleWRLL$QN-NH₂ | 2108.15 | 1055.08 | 1055.44 |
| 838 | Ac-QSQQTF$r8NLWRNleL$QN-NH₂ | 2108.15 | 1055.08 | 1055.84 |
| 839 | Ac-QSQQTF$r8NLWRLNle$QN-NH₂ | 2108.15 | 1055.08 | 1055.12 |
| 840 | Ac-QSQQTY$r8NLWRLL$QN-NH₂ | 2124.15 | 1063.08 | 1062.92 |
| 841 | Ac-RAibQQTF$r8NLWRLL$QN-NH₂ | 2134.22 | 1068.11 | 1068.65 |
| 842 | Ac-MPRFMDYWEGLN-NH₂ | 1598.70 | 800.35 | 800.45 |
| 843 | Ac-RSQQRF$r8NLWRLL$QN-NH₂ | 2191.25 | 1096.63 | 1096.83 |
| 844 | Ac-QSQQRF$r8NLWRLL$QN-NH₂ | 2163.21 | 1082.61 | 1082.87 |
| 845 | Ac-RAibQQRF$r8NLWRLL$QN-NH₂ | 2189.27 | 1095.64 | 1096.37 |
| 846 | Ac-RSQQRF$r8NFWRLL$QN-NH₂ | 2225.23 | 1113.62 | 1114.37 |
| 847 | Ac-RSQQRF$r8NYWRLL$QN-NH₂ | 2241.23 | 1121.62 | 1122.37 |
| 848 | Ac-RSQQTF$r8NLWQLL$QN-NH₂ | 2108.15 | 1055.08 | 1055.29 |
| 849 | Ac-QSQQTF$r8NLWQAmlL$QN-NH₂ | 2094.13 | 1048.07 | 1048.32 |
| 850 | Ac-QSQQTF$r8NAmlWRLL$QN-NH₂ | 2122.17 | 1062.09 | 1062.35 |
| 851 | Ac-NlePRF$r8DYWEGL$QN-NH₂ | 1869.98 | 935.99 | 936.20 |
| 852 | Ac-NlePRF$r8NYWRLL$QN-NH₂ | 1952.12 | 977.06 | 977.35 |
| 853 | Ac-RF$r8NLWRLL$Q-NH₂ | 1577.96 | 789.98 | 790.18 |
| 854 | Ac-QSQQTF$r8N2ffWRLL$QN-NH₂ | 2160.13 | 1081.07 | 1081.40 |
| 855 | Ac-QSQQTF$r8N3ffWRLL$QN-NH₂ | 2160.13 | 1081.07 | 1081.34 |
| 856 | Ac-QSQQTF#r8NLWRLL#QN-NH₂ | 2080.12 | 1041.06 | 1041.34 |
| 857 | Ac-RSQQTA$r8NLWRLL$QN-NH₂ | 2060.16 | 1031.08 | 1031.38 |
| 858 | Ac-QSQQTF%r8NLWRLL%QN-NH₂ | 2110.17 | 1056.09 | 1056.55 |
| 859 | HepQSQ$TFSNLWRLLPQN-NH₂ | 2051.10 | 1026.55 | 1026.82 |
| 860 | HepQSQ$TF$r8NLWRLL$QN-NH₂ | 2159.23 | 1080.62 | 1080.89 |
| 861 | Ac-QSQQTF$r8NL6clWRLL$QN-NH₂ | 2142.11 | 1072.06 | 1072.35 |
| 862 | Ac-QSQQTF$r8NLMe6clwRLL$QN-NH₂ | 2156.13 | 1079.07 | 1079.27 |
| 863 | Ac-LTFEHYWAQLTS-NH₂ | 1535.74 | 768.87 | 768.91 |
| 864 | Ac-LTF$HYW$QLTS-NH₂ | 1585.83 | 793.92 | 794.17 |
| 865 | Ac-LTFE$YWA$LTS-NH₂ | 1520.79 | 761.40 | 761.67 |
| 866 | Ac-LTF$zr8HYWAQL$zS-NH₂ | 1597.87 | 799.94 | 800.06 |
| 867 | Ac-LTF$r8HYWRQL$S-NH₂ | 1682.93 | 842.47 | 842.72 |
| 868 | Ac-QS$QTFStNLWRLL$s8QN-NH₂ | 2145.21 | 1073.61 | 1073.90 |
| 869 | Ac-QSQQTASNLWRLLPQN-NH₂ | 1923.99 | 963.00 | 963.26 |
| 870 | Ac-QSQQTA$/r8NLWRLL$/QN-NH₂ | 2060.15 | 1031.08 | 1031.24 |
| 871 | Ac-ASQQTF$/r8NLWRLL$/QN-NH₂ | 2079.16 | 1040.58 | 1040.89 |
| 872 | Ac-$SQQ$FSNLWRLLAibQN-NH₂ | 2009.09 | 1005.55 | 1005.86 |
| 873 | Ac-QS$QTF$NLWRLLAibQN-NH₂ | 2023.10 | 1012.55 | 1012.79 |
| 874 | Ac-QSQQ$FSN$WRLLAibQN-NH₂ | 2024.06 | 1013.03 | 1013.31 |
| 875 | Ac-QSQQTF$NLW$LLAibQN-NH₂ | 1995.06 | 998.53 | 998.87 |
| 876 | Ac-QSQQTFS$LWR$LAibQN-NH₂ | 2011.06 | 1006.53 | 1006.83 |
| 877 | Ac-QSQQTFSNLW$LLA$N-NH₂ | 1940.02 | 971.01 | 971.29 |
| 878 | Ac-$/SQQ$/FSNLWRLLAibQN-NH₂ | 2037.12 | 1019.56 | 1019.78 |
| 879 | Ac-QS$/QTF$/NLWRLLAibQN-NH₂ | 2051.13 | 1026.57 | 1026.90 |
| 880 | Ac-QSQQ$/FSN$/WRLLAibQN-NH₂ | 2052.09 | 1027.05 | 1027.36 |
| 881 | Ac-QSQQTF$/NLW$/LLAibQN-NH₂ | 2023.09 | 1012.55 | 1013.82 |
| 882 | Ac-QSQ$TFS$LWRLLAibQN-NH₂ | 1996.09 | 999.05 | 999.39 |
| 883 | Ac-QSQ$/TFS$/LWRLLAibQN-NH₂ | 2024.12 | 1013.06 | 1013.37 |
| 884 | Ac-QS$/QTFSt//NLWRLL$/s8QN-NH₂ | 2201.27 | 1101.64 | 1102.00 |
| 885 | Ac-$r8SQQTFS$LWRLLAibQN-NH₂ | 2038.14 | 1020.07 | 1020.23 |
| 886 | Ac-QSQ$r8TFSNLW$LLAibQN-NH₂ | 1996.08 | 999.04 | 999.32 |
| 887 | Ac-QSQQTFS$r8LWRLLA$N-NH₂ | 2024.12 | 1013.06 | 1013.37 |
| 888 | Ac-QS$r5QTFStNLW$LLAibQN-NH₂ | 2032.12 | 1017.06 | 1017.39 |
| 889 | Ac-$/r8SQQTFS$/LWRLLAibQN-NH₂ | 2066.17 | 1034.09 | 1034.80 |
| 890 | Ac-QSQ$/r8TFSNLW$/LLAibQN-NH₂ | 2024.11 | 1013.06 | 1014.34 |
| 891 | Ac-QSQQTFS$/r8LWRLLA$/N-NH₂ | 2052.15 | 1027.08 | 1027.16 |
| 892 | Ac-QS$/r5QTFSt//NLW$/LLAibQN-NH₂ | 2088.18 | 1045.09 | 1047.10 |
| 893 | Ac-QSQQTFSNLWRLLAibQN-NH₂ | 1988.02 | 995.01 | 995.31 |
| 894 | Hep/QSQ$/TF$/r8NLWRLL$/QN-NH₂ | 2215.29 | 1108.65 | 1108.93 |
| 895 | Ac-ASQQTF$r8NLRWLL$QN-NH₂ | 2051.13 | 1026.57 | 1026.90 |
| 896 | Ac-QSQQTF$/r8NLWRLL$/Q-NH₂ | 2022.14 | 1012.07 | 1012.66 |
| 897 | Ac-QSQQTF$r8NLWRLL$Q-NH₂ | 1994.11 | 998.06 | 998.42 |
| 898 | Ac-AAARAA$r8AAARAA$AA-NH₂ | 1515.90 | 758.95 | 759.21 |
| 899 | Ac-LTFEHYWAQLTSA-NH₂ | 1606.78 | 804.39 | 804.59 |
| 900 | Ac-LTF$r8HYWAQL$SA-NH₂ | 1668.90 | 835.45 | 835.67 |
| 901 | Ac-ASQQTFSNLWRLLPQN-NH₂ | 1943.00 | 972.50 | 973.27 |
| 902 | Ac-QS$QTFStNLW$r5LLAibQN-NH₂ | 2032.12 | 1017.06 | 1017.30 |
| 903 | Ac-QSQQTFAibNLWRLLAibQN-NH₂ | 1986.04 | 994.02 | 994.19 |
| 904 | Ac-QSQQTFNleNLWRLLNleQN-NH₂ | 2042.11 | 1022.06 | 1022.23 |
| 905 | Ac-QSQQTF$/r8NLWRLLAibQN-NH₂ | 2082.14 | 1042.07 | 1042.23 |
| 906 | Ac-QSQQTF$/r8NLWRLLNleQN-NH₂ | 2110.17 | 1056.09 | 1056.29 |
| 907 | Ac-QSQQTFAibNLWRLL$/QN-NH₂ | 2040.09 | 1021.05 | 1021.25 |
| 908 | Ac-QSQQTFNleNLWRLL$/QN-NH₂ | 2068.12 | 1035.06 | 1035.31 |
| 909 | Ac-QSQQTF%r8NL6clWRNleL%QN-NH₂ | 2144.13 | 1073.07 | 1073.32 |
| 910 | Ac-QSQQTF%r8NLMe6clWRLL%QN-NH₂ | 2158.15 | 1080.08 | 1080.31 |
| 911 | Ac-FNle$YWE$L-NH₂ | 1160.63 | — | 1161.70 |
| 912 | Ac-F$r8AYWELL$A-NH₂ | 1344.75 | — | 1345.90 |
| 913 | Ac-F$r8AYWQLL$A-NH₂ | 1343.76 | — | 1344.83 |
| 914 | Ac-NlePRF$r8NYWELL$QN-NH₂ | 1925.06 | 963.53 | 963.69 |
| 915 | Ac-NlePRF$r8DYWRLL$QN-NH₂ | 1953.10 | 977.55 | 977.68 |
| 916 | Ac-NlePRF$r8NYWRLL$Q-NH₂ | 1838.07 | 920.04 | 920.18 |
| 917 | Ac-NlePRF$r8NYWRLL$-NH₂ | 1710.01 | 856.01 | 856.13 |
| 918 | Ac-QSQQTF$r8DLWRLL$QN-NH₂ | 2109.14 | 1055.57 | 1055.64 |
| 919 | Ac-QSQQTF$r8NLWRLL$EN-NH₂ | 2109.14 | 1055.57 | 1055.70 |
| 920 | Ac-QSQQTF$r8NLWRLL$QD-NH₂ | 2109.14 | 1055.57 | 1055.64 |
| 921 | Ac-QSQQTF$r8NLWRLL$S-NH₂ | 1953.08 | 977.54 | 977.60 |
| 922 | Ac-ESQQTF$r8NLWRLL$QN-NH₂ | 2109.14 | 1055.57 | 1055.70 |
| 923 | Ac-LTF$r8NLWRNleL$Q-NH₂ | 1635.99 | 819.00 | 819.10 |
| 924 | Ac-LRF$r8NLWRNleL$Q-NH₂ | 1691.04 | 846.52 | 846.68 |
| 925 | Ac-QSQQTF$r8NWWRNleL$QN-NH₂ | 2181.15 | 1091.58 | 1091.64 |
| 926 | Ac-QSQQTF$r8NLWRNleL$Q-NH₂ | 1994.11 | 998.06 | 998.07 |
| 927 | Ac-QTF$r8NLWRNleL$QN-NH₂ | 1765.00 | 883.50 | 883.59 |
| 928 | Ac-NlePRF$r8NWWRLL$QN-NH₂ | 1975.13 | 988.57 | 988.75 |
| 929 | Ac-NlePRF$r8NWWRLL$A-NH₂ | 1804.07 | 903.04 | 903.08 |
| 930 | Ac-TSFAEYWNLLNH₂ | 1467.70 | 734.85 | 734.90 |
| 931 | Ac-QTF$r8HWWSQL$S-NH₂ | 1651.85 | 826.93 | 827.12 |
| 932 | Ac-FM$YWE$L-NH₂ | 1178.58 | — | 1179.64 |
| 933 | Ac-QTFEHWWSQLLS-NH₂ | 1601.76 | 801.88 | 801.94 |
| 934 | Ac-QSQQTF$r8NLAmwRLNle$QN-NH₂ | 2122.17 | 1062.09 | 1062.24 |
| 935 | Ac-FMAibY6clWEAc3cL-NH₂ | 1130.47 | — | 1131.53 |
| 936 | Ac-FNle$Y6clWE$L-NH₂ | 1194.59 | — | 1195.64 |
| 937 | Ac-F$zr8AY6clWEAc3cL$z-NH₂ | 1277.63 | 639.82 | 1278.71 |
| 938 | Ac-F$r8AY6clWEAc3cL$A-NH₂ | 1348.66 | — | 1350.72 |
| 939 | Ac-NlePRF$r8NY6clWRLL$QN-NH₂ | 1986.08 | 994.04 | 994.64 |
| 940 | Ac-AF$r8AAWALA$A-NH₂ | 1223.71 | — | 1224.71 |
| 941 | Ac-TF$r8AAWRLA$Q-NH₂ | 1395.80 | 698.90 | 399.04 |
| 942 | Pr-TF$r8AAWRLA$Q-NH₂ | 1409.82 | 705.91 | 706.04 |
| 943 | Ac-QSQQTF%r8NLWRNleL%QN-NH₂ | 2110.17 | 1056.09 | 1056.22 |
| 944 | Ac-LTF%r8HYWAQL%SA-NH₂ | 1670.92 | 836.46 | 836.58 |
| 945 | Ac-NlePRF%r8NYWRLL%QN-NH₂ | 1954.13 | 978.07 | 978.19 |
| 946 | Ac-NlePRF%r8NY6clWRLL%QN-NH₂ | 1988.09 | 995.05 | 995.68 |
| 947 | Ac-LTF%r8HY6clWAQL%S-NH₂ | 1633.84 | 817.92 | 817.93 |
| 948 | Ac-QS%QTF%StNLWRLL%s8QN-NH₂ | 2149.24 | 1075.62 | 1075.65 |
| 949 | Ac-LTF%r8HY6clWRQL%S-NH₂ | 1718.91 | 860.46 | 860.54 |
| 950 | Ac-QSQQTF%r8NL6clWRLL%QN-NH₂ | 2144.13 | 1073.07 | 1073.64 |
| 951 | Ac-%r8SQQTFS%LWRLLAibQN-NH₂ | 2040.15 | 1021.08 | 1021.13 |
| 952 | Ac-LTF%r8HYWAQL%S-NH₂ | 1599.88 | 800.94 | 801.09 |
| 953 | Ac-TSF%r8QYWNLL%P-NH₂ | 1602.88 | 802.44 | 802.58 |
| 954 | Ac-LTFEHYWAQLTS-NH₂ | 1535.74 | 768.87 | 769.5 |
| 955 | Ac-F$er8AY6clWEAc3cL$e-NH₂ | 1277.63 | 639.82 | 1278.71 |
| 956 | Ac-AF$r8AAWALA$A-NH₂ | 1277.63 | 639.82 | 1277.84 |
| 957 | Ac-TF$r8AAWRLA$Q-NH₂ | 1395.80 | 698.90 | 699.04 |
| 958 | Pr-TF$r8AAWRLA$Q-NH₂ | 1409.82 | 705.91 | 706.04 |
| 959 | Ac-LTF$er8HYWAQL$eS-NH₂ | 1597.87 | 799.94 | 800.44 |
| 960 | Ac-CCPGCCBaQSQQTF$r8NLWRLL$QN-NH₂ | 2745.30 | 1373.65 | 1372.99 |
| 961 | Ac-CCPGCCBaQSQQTA$r8NLWRLL$QN-NH₂ | 2669.27 | 1335.64 | 1336.09 |
| 962 | Ac-CCPGCCBaNlePRF$r8NYWRLL$QN-NH₂ | 2589.26 | 1295.63 | 1296.2 |
| 963 | Ac-LTF$/r8HYWAQL$/S-NH₂ | 1625.90 | 813.95 | 814.18 |
| 964 | Ac-F%r8HY6clWRAc3cL%-NH₂ | 1372.72 | 687.36 | 687.59 |
| 965 | Ac-QTF%r8HWWSQL%S-NH₂ | 1653.87 | 827.94 | 827.94 |
| 966 | Ac-LTA$r8HYWRQL$S-NH₂ | 1606.90 | 804.45 | 804.66 |
| 967 | Ac-Q$r8QQTFSN$WRLLAibQN-NH₂ | 2080.12 | 1041.06 | 1041.61 |
| 968 | Ac-QSQQ$r8FSNLWR$LAibQN-NH₂ | 2066.11 | 1034.06 | 1034.58 |
| 969 | Ac-F$r8AYWEAc3cL$A-NH₂ | 1314.70 | 658.35 | 1315.88 |
| 970 | Ac-F$r8AYWEAc3cL$S-NH₂ | 1330.70 | 666.35 | 1331.87 |
| 971 | Ac-F$r8AYWEAc3cL$Q-NH₂ | 1371.72 | 686.86 | 1372.72 |
| 972 | Ac-F$r8AYWEAibL$S-NH₂ | 1332.71 | 667.36 | 1334.83 |
| 973 | Ac-F$r8AYWEAL$S-NH₂ | 1318.70 | 660.35 | 1319.73 |
| 974 | Ac-F$r8AYWEQL$S-NH₂ | 1375.72 | 688.86 | 1377.53 |
| 975 | Ac-F$r8HYWEQL$S-NH₂ | 1441.74 | 721.87 | 1443.48 |
| 976 | Ac-F$r8HYWAQL$S-NH₂ | 1383.73 | 692.87 | 1385.38 |
| 977 | Ac-F$r8HYWAAc3cL$S-NH₂ | 1338.71 | 670.36 | 1340.82 |
| 978 | Ac-F$r8HYWRAc3cL$S-NH₂ | 1423.78 | 712.89 | 713.04 |
| 979 | Ac-F$r8AYWEAc3cL#A-NH₂ | 1300.69 | 651.35 | 1302.78 |
| 980 | Ac-NlePTF%r8NYWRLL%QN-NH₂ | 1899.08 | 950.54 | 950.56 |
| 981 | Ac-TF$r8AAWRAL$Q-NH₂ | 1395.80 | 698.90 | 699.13 |
| 982 | Ac-TSF%r8HYWAQL%S-NH₂ | 1573.83 | 787.92 | 787.98 |
| 983 | Ac-F%r8AY6clWEAc3cL%A-NH₂ | 1350.68 | 676.34 | 676.91 |
| 984 | Ac-LTF$r8HYWAQI$S-NH₂ | 1597.87 | 799.94 | 800.07 |
| 985 | Ac-LTF$r8HYWAQNle$S-NH₂ | 1597.87 | 799.94 | 800.07 |
| 986 | Ac-LTF$r8HYWAQL$A-NH₂ | 1581.87 | 791.94 | 792.45 |
| 987 | Ac-LTF$r8HYWAQL$Abu-NH₂ | 1595.89 | 798.95 | 799.03 |
| 988 | Ac-LTF$r8HYWAbuQL$S-NH₂ | 1611.88 | 806.94 | 807.47 |
| 989 | Ac-LTF$er8AYWAQL$eS-NH₂ | 1531.84 | 766.92 | 766.96 |
| 990 | Ac-LAF$r8HYWAQL$S-NH₂ | 1567.86 | 784.93 | 785.49 |
| 991 | Ac-LAF$r8AYWAQL$S-NH₂ | 1501.83 | 751.92 | 752.01 |
| 992 | Ac-LTF$er8AYWAQL$eA-NH₂ | 1515.85 | 758.93 | 758.97 |
| 993 | Ac-LAF$r8AYWAQL$A-NH₂ | 1485.84 | 743.92 | 744.05 |
| 994 | Ac-LTF$r8NLWANleL$Q-NH₂ | 1550.92 | 776.46 | 776.61 |
| 995 | Ac-LTF$r8NLWANleL$A-NH₂ | 1493.90 | 747.95 | 1495.6 |
| 996 | Ac-LTF$r8ALWANleL$Q-NH₂ | 1507.92 | 754.96 | 755 |
| 997 | Ac-LAF$r8NLWANleL$Q-NH₂ | 1520.91 | 761.46 | 761.96 |
| 998 | Ac-LAF$r8ALWANleL$A-NH₂ | 1420.89 | 711.45 | 1421.74 |
| 999 | Ac-A$r8AYWEAc3cL$A-NH₂ | 1238.67 | 620.34 | 1239.65 |
| 1000 | Ac-F$r8AYWEAc3cL$AA-NH₂ | 1385.74 | 693.87 | 1386.64 |
| 1001 | Ac-F$r8AYWEAc3cL$Abu-NH₂ | 1328.72 | 665.36 | 1330.17 |
| 1002 | Ac-F$r8AYWEAc3cL$Nle-NH₂ | 1356.75 | 679.38 | 1358.22 |
| 1003 | Ac-F$r5AYWEAc3cL$s8A-NH₂ | 1314.70 | 658.35 | 1315.51 |
| 1004 | Ac-F$AYWEAc3cL$r8A-NH₂ | 1314.70 | 658.35 | 1315.66 |
| 1005 | Ac-F$r8AYWEAc3cI$A-NH₂ | 1314.70 | 658.35 | 1316.18 |
| 1006 | Ac-F$r8AYWEAc3cNle$A-NH₂ | 1314.70 | 658.35 | 1315.66 |
| 1007 | Ac-F$r8AYWEAmlL$A-NH₂ | 1358.76 | 680.38 | 1360.21 |
| 1008 | Ac-F$r8AYWENleL$A-NH₂ | 1344.75 | 673.38 | 1345.71 |
| 1009 | Ac-F$r8AYWQAc3cL$A-NH₂ | 1313.72 | 657.86 | 1314.7 |
| 1010 | Ac-F$r8AYWAAc3cL$A-NH₂ | 1256.70 | 629.35 | 1257.56 |
| 1011 | Ac-F$r8AYWAbuAc3cL$A-NH₂ | 1270.71 | 636.36 | 1272.14 |
| 1012 | Ac-F$r8AYWNleAc3cL$A-NH₂ | 1298.74 | 650.37 | 1299.67 |
| 1013 | Ac-F$r8AbuYWEAc3cL$A-NH₂ | 1328.72 | 665.36 | 1329.65 |
| 1014 | Ac-F$r8NleYWEAc3cL$A-NH₂ | 1356.75 | 679.38 | 1358.66 |
| 1015 | 5-FAM-BaLTFEHYWAQLTS-NH₂ | 1922.82 | 962.41 | 962.87 |
| 1016 | 5-FAM-BaLTF%r8HYWAQL%S-NH₂ | 1986.96 | 994.48 | 994.97 |
| 1017 | Ac-LTF$r8HYWAQhL$S-NH₂ | 1611.88 | 806.94 | 807 |
| 1018 | Ac-LTF$r8HYWAQTle$S-NH₂ | 1597.87 | 799.94 | 799.97 |
| 1019 | Ac-LTF$r8HYWAQAdm$S-NH₂ | 1675.91 | 838.96 | 839.09 |
| 1020 | Ac-LTF$r8HYWAQhCha$S-NH₂ | 1651.91 | 826.96 | 826.98 |
| 1021 | Ac-LTF$r8HYWAQCha$S-NH₂ | 1637.90 | 819.95 | 820.02 |
| 1022 | Ac-LTF$r8HYWAc6cQL$S-NH₂ | 1651.91 | 826.96 | 826.98 |
| 1023 | Ac-LTF$r8HYWAc5cQL$S-NH₂ | 1637.90 | 819.95 | 820.02 |
| 1024 | Ac-LThF$r8HYWAQL$S-NH₂ | 1611.88 | 806.94 | 807 |
| 1025 | Ac-LTIgl$r8HYWAQL$S-NH₂ | 1625.90 | 813.95 | 812.99 |
| 1026 | Ac-LTF$r8HYWAQChg$S-NH₂ | 1623.88 | 812.94 | 812.99 |
| 1027 | Ac-LTF$r8HYWAQF$S-NH₂ | 1631.85 | 816.93 | 816.99 |
| 1028 | Ac-LTF$r8HYWAQIgl$S-NH₂ | 1659.88 | 830.94 | 829.94 |
| 1029 | Ac-LTF$r8HYWAQCba$S-NH₂ | 1609.87 | 805.94 | 805.96 |
| 1030 | Ac-LTF$r8HYWAQCpg$S-NH₂ | 1609.87 | 805.94 | 805.96 |
| 1031 | Ac-LTF$r8HhYWAQL$S-NH₂ | 1611.88 | 806.94 | 807 |
| 1032 | Ac-F$r8AYWEAc3chL$A-NH₂ | 1328.72 | 665.36 | 665.43 |
| 1033 | Ac-F$r8AYWEAc3cTle$A-NH₂ | 1314.70 | 658.35 | 1315.62 |
| 1034 | Ac-F$r8AYWEAc3cAdm$A-NH₂ | 1392.75 | 697.38 | 697.47 |
| 1035 | Ac-F$r8AYWEAc3chCha$A-NH₂ | 1368.75 | 685.38 | 685.34 |
| 1036 | Ac-F$r8AYWEAc3cCha$A-NH₂ | 1354.73 | 678.37 | 678.38 |
| 1037 | Ac-F$r8AYWEAc6cL$A-NH₂ | 1356.75 | 679.38 | 679.42 |
| 1038 | Ac-F$r8AYWEAc5cL$A-NH₂ | 1342.73 | 672.37 | 672.46 |
| 1039 | Ac-hF$r8AYWEAc3cL$A-NH₂ | 1328.72 | 665.36 | 665.43 |
| 1040 | Ac-Igl$r8AYWEAc3cL$A-NH₂ | 1342.73 | 672.37 | 671.5 |
| 1041 | Ac-F$r8AYWEAc3cF$A-NH₂ | 1348.69 | 675.35 | 675.35 |
| 1042 | Ac-F$r8AYWEAc3cIg1$A-NH₂ | 1376.72 | 689.36 | 688.37 |
| 1043 | Ac-F$r8AYWEAc3cCba$A-NH₂ | 1326.70 | 664.35 | 664.47 |
| 1044 | Ac-F$r8AYWEAc3cCpg$A-NH₂ | 1326.70 | 664.35 | 664.39 |
| 1045 | Ac-F$r8AhYWEAc3cL$A-NH₂ | 1328.72 | 665.36 | 665.43 |
| 1046 | Ac-F$r8AYWEAc3cL$Q-NH₂ | 1371.72 | 686.86 | 1372.87 |
| 1047 | Ac-F$r8AYWEAibL$A-NH₂ | 1316.72 | 659.36 | 1318.18 |
| 1048 | Ac-F$r8AYWEAL$A-NH₂ | 1302.70 | 652.35 | 1303.75 |
| 1049 | Ac-LAF$r8AYWAAL$A-NH₂ | 1428.82 | 715.41 | 715.49 |
| 1050 | Ac-LTF$r8HYWAAc3cL$S-NH₂ | 1552.84 | 777.42 | 777.5 |
| 1051 | Ac-NleTF$r8HYWAQL$S-NH₂ | 1597.87 | 799.94 | 800.04 |
| 1052 | Ac-VTF$r8HYWAQL$S-NH₂ | 1583.85 | 792.93 | 793.04 |
| 1053 | Ac-FTF$r8HYWAQL$S-NH₂ | 1631.85 | 816.93 | 817.02 |
| 1054 | Ac-WTF$r8HYWAQL$S-NH₂ | 1670.86 | 836.43 | 836.85 |
| 1055 | Ac-RTF$r8HYWAQL$S-NH₂ | 1640.88 | 821.44 | 821.9 |
| 1056 | Ac-KTF$r8HYWAQL$S-NH₂ | 1612.88 | 807.44 | 807.91 |
| 1057 | Ac-LNleF$r8HYWAQL$S-NH₂ | 1609.90 | 805.95 | 806.43 |
| 1058 | Ac-LVF$r8HYWAQL$S-NH₂ | 1595.89 | 798.95 | 798.93 |
| 1059 | Ac-LFF$r8HYWAQL$S-NH₂ | 1643.89 | 822.95 | 823.38 |
| 1060 | Ac-LWF$r8HYWAQL$S-NH₂ | 1682.90 | 842.45 | 842.55 |
| 1061 | Ac-LRF$r8HYWAQL$S-NH₂ | 1652.92 | 827.46 | 827.52 |
| 1062 | Ac-LKF$r8HYWAQL$S-NH₂ | 1624.91 | 813.46 | 813.51 |
| 1063 | Ac-LTF$r8NleYWAQL$S-NH₂ | 1573.89 | 787.95 | 788.05 |
| 1064 | Ac-LTF$r8VYWAQL$S-NH₂ | 1559.88 | 780.94 | 780.98 |
| 1065 | Ac-LTF$r8FYWAQL$S-NH₂ | 1607.88 | 804.94 | 805.32 |
| 1066 | Ac-LTF$r8WYWAQL$S-NH₂ | 1646.89 | 824.45 | 824.86 |
| 1067 | Ac-LTF$r8RYWAQL$S-NH₂ | 1616.91 | 809.46 | 809.51 |
| 1068 | Ac-LTF$r8KYWAQL$S-NH₂ | 1588.90 | 795.45 | 795.48 |
| 1069 | Ac-LTF$r8HNleWAQL$S-NH₂ | 1547.89 | 774.95 | 774.98 |
| 1070 | Ac-LTF$r8HVWAQL$S-NH₂ | 1533.87 | 767.94 | 767.95 |
| 1071 | Ac-LTF$r8HFWAQL$S-NH₂ | 1581.87 | 791.94 | 792.3 |
| 1072 | Ac-LTF$r8HWWAQL$S-NH₂ | 1620.88 | 811.44 | 811.54 |
| 1073 | Ac-LTF$r8HRWAQL$S-NH₂ | 1590.90 | 796.45 | 796.52 |
| 1074 | Ac-LTF$r8HKWAQL$S-NH₂ | 1562.90 | 782.45 | 782.53 |
| 1075 | Ac-LTF$r8HYWNleQL$S-NH₂ | 1639.91 | 820.96 | 820.98 |
| 1076 | Ac-LTF$r8HYWVQL$S-NH₂ | 1625.90 | 813.95 | 814.03 |
| 1077 | Ac-LTF$r8HYWFQL$S-NH₂ | 1673.90 | 837.95 | 838.03 |
| 1078 | Ac-LTF$r8HYWWQL$S-NH₂ | 1712.91 | 857.46 | 857.5 |
| 1079 | Ac-LTF$r8HYWKQL$S-NH₂ | 1654.92 | 828.46 | 828.49 |
| 1080 | Ac-LTF$r8HYWANleL$S-NH₂ | 1582.89 | 792.45 | 792.52 |
| 1081 | Ac-LTF$r8HYWAVL$S-NH₂ | 1568.88 | 785.44 | 785.49 |
| 1082 | Ac-LTF$r8HYWAFL$S-NH₂ | 1616.88 | 809.44 | 809.47 |
| 1083 | Ac-LTF$r8HYWAWL$S-NH₂ | 1655.89 | 828.95 | 829 |
| 1084 | Ac-LTF$r8HYWARL$S-NH₂ | 1625.91 | 813.96 | 813.98 |
| 1085 | Ac-LTF$r8HYWAQL$Nle-NH₂ | 1623.92 | 812.96 | 813.39 |
| 1086 | Ac-LTF$r8HYWAQL$V-NH₂ | 1609.90 | 805.95 | 805.99 |
| 1087 | Ac-LTF$r8HYWAQL$F-NH₂ | 1657.90 | 829.95 | 830.26 |
| 1088 | Ac-LTF$r8HYWAQL$W-NH₂ | 1696.91 | 849.46 | 849.5 |
| 1089 | Ac-LTF$r8HYWAQL$R-NH₂ | 1666.94 | 834.47 | 834.56 |
| 1090 | Ac-LTF$r8HYWAQL$K-NH₂ | 1638.93 | 820.47 | 820.49 |
| 1091 | Ac-Q$r8QQTFSN$WRLLAibQN-NH₂ | 2080.12 | 1041.06 | 1041.54 |
| 1092 | Ac-QSQQ$r8FSNLWR$LAibQN-NH₂ | 2066.11 | 1034.06 | 1034.58 |
| 1093 | Ac-LT2Pal$r8HYWAQL$S-NH₂ | 1598.86 | 800.43 | 800.49 |
| 1094 | Ac-LT3Pal$r8HYWAQL$S-NH₂ | 1598.86 | 800.43 | 800.49 |
| 1095 | Ac-LT4Pal$r8HYWAQL$S-NH₂ | 1598.86 | 800.43 | 800.49 |
| 1096 | Ac-LTF2CF3$r8HYWAQL$S-NH₂ | 1665.85 | 833.93 | 834.01 |
| 1097 | Ac-LTF2CN$r8HYWAQL$S-NH₂ | 1622.86 | 812.43 | 812.47 |
| 1098 | Ac-LTF2Me$r8HYWAQL$S-NH₂ | 1611.88 | 806.94 | 807 |
| 1099 | Ac-LTF3Cl$r8HYWAQL$S-NH₂ | 1631.83 | 816.92 | 816.99 |
| 1100 | Ac-LTF4CF3$r8HYWAQL$S-NH₂ | 1665.85 | 833.93 | 833.94 |
| 1101 | Ac-LTF4tBu$r8HYWAQL$S-NH₂ | 1653.93 | 827.97 | 828.02 |
| 1102 | Ac-LTF5F$r8HYWAQL$S-NH₂ | 1687.82 | 844.91 | 844.96 |
| 1103 | Ac-LTF$r8HY3BthAAQL$S-NH₂ | 1614.83 | 808.42 | 808.48 |
| 1104 | Ac-LTF2Br$r8HYWAQL$S-NH₂ | 1675.78 | 838.89 | 838.97 |
| 1105 | Ac-LTF4Br$r8HYWAQL$S-NH₂ | 1675.78 | 838.89 | 839.86 |
| 1106 | Ac-LTF2Cl$r8HYWAQL$S-NH₂ | 1631.83 | 816.92 | 816.99 |
| 1107 | Ac-LTF4Cl$r8HYWAQL$S-NH₂ | 1631.83 | 816.92 | 817.36 |
| 1108 | Ac-LTF3CN$r8HYWAQL$S-NH₂ | 1622.86 | 812.43 | 812.47 |
| 1109 | Ac-LTF4CN$r8HYWAQL$S-NH₂ | 1622.86 | 812.43 | 812.47 |
| 1110 | Ac-LTF34Cl2$r8HYWAQL$S-NH₂ | 1665.79 | 833.90 | 833.94 |
| 1111 | Ac-LTF34F2$r8HYWAQL$S-NH₂ | 1633.85 | 817.93 | 817.95 |
| 1112 | Ac-LTF35F2$r8HYWAQL$S-NH₂ | 1633.85 | 817.93 | 817.95 |
| 1113 | Ac-LTDip$r8HYWAQL$S-NH₂ | 1673.90 | 837.95 | 838.01 |
| 1114 | Ac-LTF2F$r8HYWAQL$S-NH₂ | 1615.86 | 808.93 | 809 |
| 1115 | Ac-LTF3F$r8HYWAQL$S-NH₂ | 1615.86 | 808.93 | 809 |
| 1116 | Ac-LTF4F$r8HYWAQL$S-NH₂ | 1615.86 | 808.93 | 809 |
| 1117 | Ac-LTF4I$r8HYWAQL$S-NH₂ | 1723.76 | 862.88 | 862.94 |
| 1118 | Ac-LTF3Me$r8HYWAQL$S-NH₂ | 1611.88 | 806.94 | 807.07 |
| 1119 | Ac-LTF4Me$r8HYWAQL$S-NH₂ | 1611.88 | 806.94 | 807 |
| 1120 | Ac-LT1Nal$r8HYWAQL$S-NH₂ | 1647.88 | 824.94 | 824.98 |
| 1121 | Ac-LT2Nal$r8HYWAQL$S-NH₂ | 1647.88 | 824.94 | 825.06 |
| 1122 | Ac-LTF3CF3$r8HYWAQL$S-NH₂ | 1665.85 | 833.93 | 834.01 |
| 1123 | Ac-LTF4NO2$r8HYWAQL$S-NH₂ | 1642.85 | 822.43 | 822.46 |
| 1124 | Ac-LTF3NO2$r8HYWAQL$S-NH₂ | 1642.85 | 822.43 | 822.46 |
| 1125 | Ac-LTF$r82ThiYWAQL$S-NH₂ | 1613.83 | 807.92 | 807.96 |
| 1126 | Ac-LTF$r8HBibWAQL$S-NH₂ | 1657.90 | 829.95 | 830.01 |
| 1127 | Ac-LTF$r8HF4tBuWAQL$S-NH₂ | 1637.93 | 819.97 | 820.02 |
| 1128 | Ac-LTF$r8HF4CF3WAQL$S-NH₂ | 1649.86 | 825.93 | 826.02 |
| 1129 | Ac-LTF$r8HF4ClWAQL$S-NH₂ | 1615.83 | 808.92 | 809.37 |
| 1130 | Ac-LTF$r8HF4MeWAQL$S-NH₂ | 1595.89 | 798.95 | 799.01 |
| 1131 | Ac-LTF$r8HF4BrWAQL$S-NH₂ | 1659.78 | 830.89 | 830.98 |
| 1132 | Ac-LTF$r8HF4CNWAQL$S-NH₂ | 1606.87 | 804.44 | 804.56 |
| 1133 | Ac-LTF$r8HF4NO2WAQL$S-NH₂ | 1626.86 | 814.43 | 814.55 |
| 1134 | Ac-LTF$r8H1NalWAQL$S-NH₂ | 1631.89 | 816.95 | 817.06 |
| 1135 | Ac-LTF$r8H2NalWAQL$S-NH₂ | 1631.89 | 816.95 | 816.99 |
| 1136 | Ac-LTF$r8HWAQL$S-NH₂ | 1434.80 | 718.40 | 718.49 |
| 1137 | Ac-LTF$r8HYlNalAQL$S-NH₂ | 1608.87 | 805.44 | 805.52 |
| 1138 | Ac-LTF$r8HY2NalAQL$S-NH₂ | 1608.87 | 805.44 | 805.52 |
| 1139 | Ac-LTF$r8HYWAQI$S-NH₂ | 1597.87 | 799.94 | 800.07 |
| 1140 | Ac-LTF$r8HYWAQNle$S-NH₂ | 1597.87 | 799.94 | 800.44 |
| 1141 | Ac-LTF$er8HYWAQL$eA-NH₂ | 1581.87 | 791.94 | 791.98 |
| 1142 | Ac-LTF$r8HYWAQL$Abu-NH₂ | 1595.89 | 798.95 | 799.03 |
| 1143 | Ac-LTF$r8HYWAbuQL$S-NH₂ | 1611.88 | 806.94 | 804.47 |
| 1144 | Ac-LAF$r8HYWAQL$S-NH₂ | 1567.86 | 784.93 | 785.49 |
| 1145 | Ac-LTF$r8NLWANleL$Q-NH₂ | 1550.92 | 776.46 | 777.5 |
| 1146 | Ac-LTF$r8ALWANleL$Q-NH₂ | 1507.92 | 754.96 | 755.52 |
| 1147 | Ac-LAF$r8NLWANleL$Q-NH₂ | 1520.91 | 761.46 | 762.48 |
| 1148 | Ac-F$r8AYWAAc3cL$A-NH₂ | 1256.70 | 629.35 | 1257.56 |
| 1149 | Ac-LTF$r8AYWAAL$S-NH₂ | 1474.82 | 738.41 | 738.55 |
| 1150 | Ac-LVF$r8AYWAQL$S-NH₂ | 1529.87 | 765.94 | 766 |
| 1151 | Ac-LTF$r8AYWAbuQL$S-NH₂ | 1545.86 | 773.93 | 773.92 |
| 1152 | Ac-LTF$r8AYWNleQL$S-NH₂ | 1573.89 | 787.95 | 788.17 |
| 1153 | Ac-LTF$r8AbuYWAQL$S-NH₂ | 1545.86 | 773.93 | 773.99 |
| 1154 | Ac-LTF$r8AYWHQL$S-NH₂ | 1597.87 | 799.94 | 799.97 |
| 1155 | Ac-LTF$r8AYWKQL$S-NH₂ | 1588.90 | 795.45 | 795.53 |
| 1156 | Ac-LTF$r8AYWOQL$S-NH₂ | 1574.89 | 788.45 | 788.5 |
| 1157 | Ac-LTF$r8AYWRQL$S-NH₂ | 1616.91 | 809.46 | 809.51 |
| 1158 | Ac-LTF$r8AYWSQL$S-NH₂ | 1547.84 | 774.92 | 774.96 |
| 1159 | Ac-LTF$r8AYWRAL$S-NH₂ | 1559.89 | 780.95 | 780.95 |
| 1160 | Ac-LTF$r8AYWRQL$A-NH₂ | 1600.91 | 801.46 | 801.52 |
| 1161 | Ac-LTF$r8AYWRAL$A-NH₂ | 1543.89 | 772.95 | 773.03 |
| 1162 | Ac-LTF$r5HYWAQL$s8S-NH₂ | 1597.87 | 799.94 | 799.97 |
| 1163 | Ac-LTF$HYWAQL$r8S-NH₂ | 1597.87 | 799.94 | 799.97 |
| 1164 | Ac-LTF$r8HYWAAL$S-NH₂ | 1540.84 | 771.42 | 771.48 |
| 1165 | Ac-LTF$r8HYWAAbuL$S-NH₂ | 1554.86 | 778.43 | 778.51 |
| 1166 | Ac-LTF$r8HYWALL$S-NH₂ | 1582.89 | 792.45 | 792.49 |
| 1167 | Ac-F$r8AYWHAL$A-NH₂ | 1310.72 | 656.36 | 656.4 |
| 1168 | Ac-F$r8AYWAAL$A-NH₂ | 1244.70 | 623.35 | 1245.61 |
| 1169 | Ac-F$r8AYWSAL$A-NH₂ | 1260.69 | 631.35 | 1261.6 |
| 1170 | Ac-F$r8AYWRAL$A-NH₂ | 1329.76 | 665.88 | 1330.72 |
| 1171 | Ac-F$r8AYWKAL$A-NH₂ | 1301.75 | 651.88 | 1302.67 |
| 1172 | Ac-F$r8AYWOAL$A-NH₂ | 1287.74 | 644.87 | 1289.13 |
| 1173 | Ac-F$r8VYWEAc3cL$A-NH₂ | 1342.73 | 672.37 | 1343.67 |
| 1174 | Ac-F$r8FYWEAc3cL$A-NH₂ | 1390.73 | 696.37 | 1392.14 |
| 1175 | Ac-F$r8WYWEAc3cL$A-NH₂ | 1429.74 | 715.87 | 1431.44 |
| 1176 | Ac-F$r8RYWEAc3cL$A-NH₂ | 1399.77 | 700.89 | 700.95 |
| 1177 | Ac-F$r8KYWEAc3cL$A-NH₂ | 1371.76 | 686.88 | 686.97 |
| 1178 | Ac-F$r8ANleWEAc3cL$A-NH₂ | 1264.72 | 633.36 | 1265.59 |
| 1179 | Ac-F$r8AVWEAc3cL$A-NH₂ | 1250.71 | 626.36 | 1252.2 |
| 1180 | Ac-F$r8AFWEAc3cL$A-NH₂ | 1298.71 | 650.36 | 1299.64 |
| 1181 | Ac-F$r8AWWEAc3cL$A-NH₂ | 1337.72 | 669.86 | 1338.64 |
| 1182 | Ac-F$r8ARWEAc3cL$A-NH₂ | 1307.74 | 654.87 | 655 |
| 1183 | Ac-F$r8AKWEAc3cL$A-NH₂ | 1279.73 | 640.87 | 641.01 |
| 1184 | Ac-F$r8AYWVAc3cL$A-NH₂ | 1284.73 | 643.37 | 643.38 |
| 1185 | Ac-F$r8AYWFAc3cL$A-NH₂ | 1332.73 | 667.37 | 667.43 |
| 1186 | Ac-F$r8AYWWAc3cL$A-NH₂ | 1371.74 | 686.87 | 686.97 |
| 1187 | Ac-F$r8AYWRAc3cL$A-NH₂ | 1341.76 | 671.88 | 671.94 |
| 1188 | Ac-F$r8AYWKAc3cL$A-NH₂ | 1313.75 | 657.88 | 657.88 |
| 1189 | Ac-F$r8AYWEVL$A-NH₂ | 1330.73 | 666.37 | 666.47 |
| 1190 | Ac-F$r8AYWEFL$A-NH₂ | 1378.73 | 690.37 | 690.44 |
| 1191 | Ac-F$r8AYWEWL$A-NH₂ | 1417.74 | 709.87 | 709.91 |
| 1192 | Ac-F$r8AYWERL$A-NH₂ | 1387.77 | 694.89 | 1388.66 |
| 1193 | Ac-F$r8AYWEKL$A-NH₂ | 1359.76 | 680.88 | 1361.21 |
| 1194 | Ac-F$r8AYWEAc3cL$V-NH₂ | 1342.73 | 672.37 | 1343.59 |
| 1195 | Ac-F$r8AYWEAc3cL$F-NH₂ | 1390.73 | 696.37 | 1392.58 |
| 1196 | Ac-F$r8AYWEAc3cL$W-NH₂ | 1429.74 | 715.87 | 1431.29 |
| 1197 | Ac-F$r8AYWEAc3cL$R-NH₂ | 1399.77 | 700.89 | 700.95 |
| 1198 | Ac-F$r8AYWEAc3cL$K-NH₂ | 1371.76 | 686.88 | 686.97 |
| 1199 | Ac-F$r8AYWEAc3cL$AV-NH₂ | 1413.77 | 707.89 | 707.91 |
| 1200 | Ac-F$r8AYWEAc3cL$AF-NH₂ | 1461.77 | 731.89 | 731.96 |
| 1201 | Ac-F$r8AYWEAc3cL$AW-NH₂ | 1500.78 | 751.39 | 751.5 |
| 1202 | Ac-F$r8AYWEAc3cL$AR-NH₂ | 1470.80 | 736.40 | 736.47 |
| 1203 | Ac-F$r8AYWEAc3cL$AK-NH₂ | 1442.80 | 722.40 | 722.41 |
| 1204 | Ac-F$r8AYWEAc3cL$AH-NH₂ | 1451.76 | 726.88 | 726.93 |
| 1205 | Ac-LTF2NO2$r8HYWAQL$S-NH₂ | 1642.85 | 822.43 | 822.54 |
| 1206 | Ac-LTA$r8HYAAQL$S-NH₂ | 1406.79 | 704.40 | 704.5 |
| 1207 | Ac-LTF$r8HYAAQL$S-NH₂ | 1482.82 | 742.41 | 742.47 |
| 1208 | Ac-QSQQTF$r8NLWALL$AN-NH₂ | 1966.07 | 984.04 | 984.38 |
| 1209 | Ac-QAibQQTF$r8NLWALL$AN-NH₂ | 1964.09 | 983.05 | 983.42 |
| 1210 | Ac-QAibQQTF$r8ALWALL$AN-NH₂ | 1921.08 | 961.54 | 961.59 |
| 1211 | Ac-AAAATF$r8AAWAAL$AA-NH₂ | 1608.90 | 805.45 | 805.52 |
| 1212 | Ac-F$r8AAWRAL$Q-NH₂ | 1294.76 | 648.38 | 648.48 |
| 1213 | Ac-TF$r8AAWAAL$Q-NH₂ | 1310.74 | 656.37 | 1311.62 |
| 1214 | Ac-TF$r8AAWRAL$A-NH₂ | 1338.78 | 670.39 | 670.46 |
| 1215 | Ac-VF$r8AAWRAL$Q-NH₂ | 1393.82 | 697.91 | 697.99 |
| 1216 | Ac-AF$r8AAWAAL$A-NH₂ | 1223.71 | 612.86 | 1224.67 |
| 1217 | Ac-TF$r8AAWKAL$Q-NH₂ | 1367.80 | 684.90 | 684.97 |
| 1218 | Ac-TF$r8AAWOAL$Q-NH₂ | 1353.78 | 677.89 | 678.01 |
| 1219 | Ac-TF$r8AAWSAL$Q-NH₂ | 1326.73 | 664.37 | 664.47 |
| 1220 | Ac-LTF$r8AAWRAL$Q-NH₂ | 1508.89 | 755.45 | 755.49 |
| 1221 | Ac-F$r8AYWAQL$A-NH₂ | 1301.72 | 651.86 | 651.96 |
| 1222 | Ac-F$r8AWWAAL$A-NH₂ | 1267.71 | 634.86 | 634.87 |
| 1223 | Ac-F$r8AWWAQL$A-NH₂ | 1324.73 | 663.37 | 663.43 |
| 1224 | Ac-F$r8AYWEAL$-NH₂ | 1231.66 | 616.83 | 1232.93 |
| 1225 | Ac-F$r8AYWAAL$-NH₂ | 1173.66 | 587.83 | 1175.09 |
| 1226 | Ac-F$r8AYWKAL$-NH₂ | 1230.72 | 616.36 | 616.44 |
| 1227 | Ac-F$r8AYWOAL$-NH₂ | 1216.70 | 609.35 | 609.48 |
| 1228 | Ac-F$r8AYWQAL$-NH₂ | 1230.68 | 616.34 | 616.44 |
| 1229 | Ac-F$r8AYWAQL$-NH₂ | 1230.68 | 616.34 | 616.37 |
| 1230 | Ac-F$r8HYWDQL$S-NH₂ | 1427.72 | 714.86 | 714.86 |
| 1231 | Ac-F$r8HFWEQL$S-NH₂ | 1425.74 | 713.87 | 713.98 |
| 1232 | Ac-F$r8AYWHQL$S-NH₂ | 1383.73 | 692.87 | 692.96 |
| 1233 | Ac-F$r8AYWKQL$S-NH₂ | 1374.77 | 688.39 | 688.45 |
| 1234 | Ac-F$r8AYWOQL$S-NH₂ | 1360.75 | 681.38 | 681.49 |
| 1235 | Ac-F$r8HYWSQL$S-NH₂ | 1399.73 | 700.87 | 700.95 |
| 1236 | Ac-F$r8HWWEQL$S-NH₂ | 1464.76 | 733.38 | 733.44 |
| 1237 | Ac-F$r8HWWAQL$S-NH₂ | 1406.75 | 704.38 | 704.43 |
| 1238 | Ac-F$r8AWWHQL$S-NH₂ | 1406.75 | 704.38 | 704.43 |
| 1239 | Ac-F$r8AWWKQL$S-NH₂ | 1397.79 | 699.90 | 699.92 |
| 1240 | Ac-F$r8AWWOQL$S-NH₂ | 1383.77 | 692.89 | 692.96 |
| 1241 | Ac-F$r8HWWSQL$S-NH₂ | 1422.75 | 712.38 | 712.42 |
| 1242 | Ac-LTF$r8NYWANleL$Q-NH₂ | 1600.90 | 801.45 | 801.52 |
| 1243 | Ac-LTF$r8NLWAQL$Q-NH₂ | 1565.90 | 783.95 | 784.06 |
| 1244 | Ac-LTF$r8NYWANleL$A-NH₂ | 1543.88 | 772.94 | 773.03 |
| 1245 | Ac-LTF$r8NLWAQL$A-NH₂ | 1508.88 | 755.44 | 755.49 |
| 1246 | Ac-LTF$r8AYWANleL$Q-NH₂ | 1557.90 | 779.95 | 780.06 |
| 1247 | Ac-LTF$r8ALWAQL$Q-NH₂ | 1522.89 | 762.45 | 762.45 |
| 1248 | Ac-LAF$r8NYWANleL$Q-NH₂ | 1570.89 | 786.45 | 786.5 |
| 1249 | Ac-LAF$r8NLWAQL$Q-NH₂ | 1535.89 | 768.95 | 769.03 |
| 1250 | Ac-LAF$r8AYWANleL$A-NH₂ | 1470.86 | 736.43 | 736.47 |
| 1251 | Ac-LAF$r8ALWAQL$A-NH₂ | 1435.86 | 718.93 | 719.01 |
| 1252 | Ac-LAF$r8AYWAAL$A-NH₂ | 1428.82 | 715.41 | 715.41 |
| 1253 | Ac-F$r8AYWEAc3cL$AAib-NH₂ | 1399.75 | 700.88 | 700.95 |
| 1254 | Ac-F$r8AYWAQL$AA-NH₂ | 1372.75 | 687.38 | 687.78 |
| 1255 | Ac-F$r8AYWAAc3cL$AA-NH₂ | 1327.73 | 664.87 | 664.84 |
| 1256 | Ac-F$r8AYWSAc3cL$AA-NH₂ | 1343.73 | 672.87 | 672.9 |
| 1257 | Ac-F$r8AYWEAc3cL$AS-NH₂ | 1401.73 | 701.87 | 701.84 |
| 1258 | Ac-F$r8AYWEAc3cL$AT-NH₂ | 1415.75 | 708.88 | 708.87 |
| 1259 | Ac-F$r8AYWEAc3cL$AL-NH₂ | 1427.79 | 714.90 | 714.94 |
| 1260 | Ac-F$r8AYWEAc3cL$AQ-NH₂ | 1442.76 | 722.38 | 722.41 |
| 1261 | Ac-F$r8AFWEAc3cL$AA-NH₂ | 1369.74 | 685.87 | 685.93 |
| 1262 | Ac-F$r8AWWEAc3cL$AA-NH₂ | 1408.75 | 705.38 | 705.39 |
| 1263 | Ac-F$r8AYWEAc3cL$SA-NH₂ | 1401.73 | 701.87 | 701.99 |
| 1264 | Ac-F$r8AYWEAL$AA-NH₂ | 1373.74 | 687.87 | 687.93 |
| 1265 | Ac-F$r8AYWENleL$AA-NH₂ | 1415.79 | 708.90 | 708.94 |
| 1266 | Ac-F$r8AYWEAc3cL$AbuA-NH₂ | 1399.75 | 700.88 | 700.95 |
| 1267 | Ac-F$r8AYWEAc3cL$NleA-NH₂ | 1427.79 | 714.90 | 714.86 |
| 1268 | Ac-F$r8AYWEAibL$NleA-NH₂ | 1429.80 | 715.90 | 715.97 |
| 1269 | Ac-F$r8AYWEAL$NleA-NH₂ | 1415.79 | 708.90 | 708.94 |
| 1270 | Ac-F$r8AYWENleL$NleA-NH₂ | 1457.83 | 729.92 | 729.96 |
| 1271 | Ac-F$r8AYWEAibL$Abu-NH₂ | 1330.73 | 666.37 | 666.39 |
| 1272 | Ac-F$r8AYWENleL$Abu-NH₂ | 1358.76 | 680.38 | 680.39 |
| 1273 | Ac-F$r8AYWEAL$Abu-NH₂ | 1316.72 | 659.36 | 659.36 |
| 1274 | Ac-LTF$r8AFWAQL$S-NH₂ | 1515.85 | 758.93 | 759.12 |
| 1275 | Ac-LTF$r8AWWAQL$S-NH₂ | 1554.86 | 778.43 | 778.51 |
| 1276 | Ac-LTF$r8AYWAQI$S-NH₂ | 1531.84 | 766.92 | 766.96 |
| 1277 | Ac-LTF$r8AYWAQNle$S-NH₂ | 1531.84 | 766.92 | 766.96 |
| 1278 | Ac-LTF$r8AYWAQL$SA-NH₂ | 1602.88 | 802.44 | 802.48 |
| 1279 | Ac-LTF$r8AWWAQL$A-NH₂ | 1538.87 | 770.44 | 770.89 |
| 1280 | Ac-LTF$r8AYWAQI$A-NH₂ | 1515.85 | 758.93 | 759.42 |
| 1281 | Ac-LTF$r8AYWAQNle$A-NH₂ | 1515.85 | 758.93 | 759.42 |
| 1282 | Ac-LTF$r8AYWAQL$AA-NH₂ | 1586.89 | 794.45 | 794.94 |
| 1283 | Ac-LTF$r8HWWAQL$S-NH₂ | 1620.88 | 811.44 | 811.47 |
| 1284 | Ac-LTF$r8HRWAQL$S-NH₂ | 1590.90 | 796.45 | 796.52 |
| 1285 | Ac-LTF$r8HKWAQL$S-NH₂ | 1562.90 | 782.45 | 782.53 |
| 1286 | Ac-LTF$r8HYWAQL$W-NH₂ | 1696.91 | 849.46 | 849.5 |
| 1287 | Ac-F$r8AYWAbuAL$A-NH₂ | 1258.71 | 630.36 | 630.5 |
| 1288 | Ac-F$r8AbuYWEAL$A-NH₂ | 1316.72 | 659.36 | 659.51 |
| 1289 | Ac-NlePRF%r8NYWRLL%QN-NH₂ | 1954.13 | 978.07 | 978.54 |
| 1290 | Ac-TSF%r8HYWAQL%S-NH₂ | 1573.83 | 787.92 | 787.98 |
| 1291 | Ac-LTF%r8AYWAQL%S-NH₂ | 1533.86 | 767.93 | 768 |
| 1292 | Ac-HTF$r8HYWAQL$S-NH₂ | 1621.84 | 811.92 | 811.96 |
| 1293 | Ac-LHF$r8HYWAQL$S-NH₂ | 1633.88 | 817.94 | 818.02 |
| 1294 | Ac-LTF$r8HHWAQL$S-NH₂ | 1571.86 | 786.93 | 786.94 |
| 1295 | Ac-LTF$r8HYWHQL$S-NH₂ | 1663.89 | 832.95 | 832.38 |
| 1296 | Ac-LTF$r8HYWAHL$S-NH₂ | 1606.87 | 804.44 | 804.48 |
| 1297 | Ac-LTF$r8HYWAQL$H-NH₂ | 1647.89 | 824.95 | 824.98 |
| 1298 | Ac-LTF$r8HYWAQL$S-NHPr | 1639.91 | 820.96 | 820.98 |
| 1299 | Ac-LTF$r8HYWAQL$S-NHsBu | 1653.93 | 827.97 | 828.02 |
| 1300 | Ac-LTF$r8HYWAQL$S-NHiBu | 1653.93 | 827.97 | 828.02 |
| 1301 | Ac-LTF$r8HYWAQL$S-NHBn | 1687.91 | 844.96 | 844.44 |
| 1302 | Ac-LTF$r8HYWAQL$S-NHPe | 1700.92 | 851.46 | 851.99 |
| 1303 | Ac-LTF$r8HYWAQL$S-NHChx | 1679.94 | 840.97 | 841.04 |
| 1304 | Ac-ETF$r8AYWAQL$S-NH₂ | 1547.80 | 774.90 | 774.96 |
| 1305 | Ac-STF$r8AYWAQL$S-NH₂ | 1505.79 | 753.90 | 753.94 |
| 1306 | Ac-LEF$r8AYWAQL$S-NH₂ | 1559.84 | 780.92 | 781.25 |
| 1307 | Ac-LSF$r8AYWAQL$S-NH₂ | 1517.83 | 759.92 | 759.93 |
| 1308 | Ac-LTF$r8EYWAQL$S-NH₂ | 1589.85 | 795.93 | 795.97 |
| 1309 | Ac-LTF$r8SYWAQL$S-NH₂ | 1547.84 | 774.92 | 774.96 |
| 1310 | Ac-LTF$r8AYWEQL$S-NH₂ | 1589.85 | 795.93 | 795.9 |
| 1311 | Ac-LTF$r8AYWAEL$S-NH₂ | 1532.83 | 767.42 | 766.96 |
| 1312 | Ac-LTF$r8AYWASL$S-NH₂ | 1490.82 | 746.41 | 746.46 |
| 1313 | Ac-LTF$r8AYWAQL$E-NH₂ | 1573.85 | 787.93 | 787.98 |
| 1314 | Ac-LTF2CN$r8HYWAQL$S-NH₂ | 1622.86 | 812.43 | 812.47 |
| 1315 | Ac-LTF3Cl$r8HYWAQL$S-NH₂ | 1631.83 | 816.92 | 816.99 |
| 1316 | Ac-LTDip$r8HYWAQL$S-NH₂ | 1673.90 | 837.95 | 838.01 |
| 1317 | Ac-LTF$r8HYWAQTle$S-NH₂ | 1597.87 | 799.94 | 800.04 |
| 1318 | Ac-F$r8AY6clWEAL$A-NH₂ | 1336.66 | 669.33 | 1338.56 |
| 1319 | Ac-F$r8AYdl6brWEAL$A-NH₂ | 1380.61 | 691.31 | 692.2 |
| 1320 | Ac-F$r8AYdl6fWEAL$A-NH₂ | 1320.69 | 661.35 | 1321.61 |
| 1321 | Ac-F$r8AYdl4mWEAL$A-NH₂ | 1316.72 | 659.36 | 659.36 |
| 1322 | Ac-F$r8AYdl5clWEAL$A-NH₂ | 1336.66 | 669.33 | 669.35 |
| 1323 | Ac-F$r8AYdl7mWEAL$A-NH₂ | 1316.72 | 659.36 | 659.36 |
| 1324 | Ac-LTF%r8HYWAQL%A-NH₂ | 1583.89 | 792.95 | 793.01 |
| 1325 | Ac-LTF$r8HCouWAQL$S-NH₂ | 1679.87 | 840.94 | 841.38 |
| 1326 | Ac-LTFEHC0uWAQLTS-NH₂ | 1617.75 | 809.88 | 809.96 |
| 1327 | Ac-LTA$r8HCouWAQL$S-NH₂ | 1603.84 | 802.92 | 803.36 |
| 1328 | Ac-F$r8AYWEAL$AbuA-NH₂ | 1387.75 | 694.88 | 694.88 |
| 1329 | Ac-F$r8AYWEAI$AA-NH₂ | 1373.74 | 687.87 | 687.93 |
| 1330 | Ac-F$r8AYWEANle$AA-NH₂ | 1373.74 | 687.87 | 687.93 |
| 1331 | Ac-F$r8AYWEAmlL$AA-NH₂ | 1429.80 | 715.90 | 715.97 |
| 1332 | Ac-F$r8AYWQAL$AA-NH₂ | 1372.75 | 687.38 | 687.48 |
| 1333 | Ac-F$r8AYWAAL$AA-NH₂ | 1315.73 | 658.87 | 658.92 |
| 1334 | Ac-F$r8AYWAbuAL$AA-NH₂ | 1329.75 | 665.88 | 665.95 |
| 1335 | Ac-F$r8AYWNleAL$AA-NH₂ | 1357.78 | 679.89 | 679.94 |
| 1336 | Ac-F$r8AbuYWEAL$AA-NH₂ | 1387.75 | 694.88 | 694.96 |
| 1337 | Ac-F$r8NleYWEAL$AA-NH₂ | 1415.79 | 708.90 | 708.94 |
| 1338 | Ac-F$r8FYWEAL$AA-NH₂ | 1449.77 | 725.89 | 725.97 |
| 1339 | Ac-LTF$r8HYWAQhL$S-NH₂ | 1611.88 | 806.94 | 807 |
| 1340 | Ac-LTF$r8HYWAQAdm$S-NH₂ | 1675.91 | 838.96 | 839.04 |
| 1341 | Ac-LTF$r8HYWAQIgl$S-NH₂ | 1659.88 | 830.94 | 829.94 |
| 1342 | Ac-F$r8AYWAQL$AA-NH₂ | 1372.75 | 687.38 | 687.48 |
| 1343 | Ac-LTF$r8ALWAQL$Q-NH₂ | 1522.89 | 762.45 | 762.52 |
| 1344 | Ac-F$r8AYWEAL$AA-NH₂ | 1373.74 | 687.87 | 687.93 |
| 1345 | Ac-F$r8AYWENleL$AA-NH₂ | 1415.79 | 708.90 | 708.94 |
| 1346 | Ac-F$r8AYWEAibL$Abu-NH₂ | 1330.73 | 666.37 | 666.39 |
| 1347 | Ac-F$r8AYWENleL$Abu-NH₂ | 1358.76 | 680.38 | 680.38 |
| 1348 | Ac-F$r8AYWEAL$Abu-NH₂ | 1316.72 | 659.36 | 659.36 |
| 1349 | Ac-F$r8AYWEAc3cL$AbuA-NH₂ | 1399.75 | 700.88 | 700.95 |
| 1350 | Ac-F$r8AYWEAc3cL$NleA-NH₂ | 1427.79 | 714.90 | 715.01 |
| 1351 | H-LTF$r8AYWAQL$S-NH₂ | 1489.83 | 745.92 | 745.95 |
| 1352 | mdPEG3-LTF$r8AYWAQL$S-NH₂ | 1679.92 | 840.96 | 840.97 |
| 1353 | mdPEG7-LTF$r8AYWAQL$S-NH₂ | 1856.02 | 929.01 | 929.03 |
| 1354 | Ac-F$r8ApmpEt6clWEAL$A-NH₂ | 1470.71 | 736.36 | 788.17 |
| 1355 | Ac-LTF3Cl$r8AYWAQL$S-NH₂ | 1565.81 | 783.91 | 809.18 |
| 1356 | Ac-LTF3Cl$r8HYWAQL$A-NH₂ | 1615.83 | 808.92 | 875.24 |
| 1357 | Ac-LTF3Cl$r8HYWWQL$S-NH₂ | 1746.87 | 874.44 | 841.65 |
| 1358 | Ac-LTF3Cl$r8AYWWQL$S-NH₂ | 1680.85 | 841.43 | 824.63 |
| 1359 | Ac-LTF$r8AYWWQL$S-NH₂ | 1646.89 | 824.45 | 849.98 |
| 1360 | Ac-LTF$r8HYWWQL$A-NH₂ | 1696.91 | 849.46 | 816.67 |
| 1361 | Ac-LTF$r8AYWWQL$A-NH₂ | 1630.89 | 816.45 | 776.15 |
| 1362 | Ac-LTF4F$r8AYWAQL$S-NH₂ | 1549.83 | 775.92 | 776.15 |
| 1363 | Ac-LTF2F$r8AYWAQL$S-NH₂ | 1549.83 | 775.92 | 776.15 |
| 1364 | Ac-LTF3F$r8AYWAQL$S-NH₂ | 1549.83 | 775.92 | 785.12 |
| 1365 | Ac-LTF34F2$r8AYWAQL$S-NH₂ | 1567.83 | 784.92 | 785.12 |
| 1366 | Ac-LTF35F2$r8AYWAQL$S-NH₂ | 1567.83 | 784.92 | 1338.74 |
| 1367 | Ac-F3Cl$r8AYWEAL$A-NH₂ | 1336.66 | 669.33 | 705.28 |
| 1368 | Ac-F3Cl$r8AYWEAL$AA-NH₂ | 1407.70 | 704.85 | 680.11 |
| 1369 | Ac-F$r8AY6clWEAL$AA-NH₂ | 1407.70 | 704.85 | 736.83 |
| 1370 | Ac-F$r8AY6clWEAL$-NH₂ | 1265.63 | 633.82 | 784.1 |
| 1371 | Ac-LTF$r8HYWAQLSt/S-NH₂ | 16.03 | 9.02 | 826.98 |
| 1372 | Ac-LTF$r8HYWAQL$S-NHsBu | 1653.93 | 827.97 | 828.02 |
| 1373 | Ac-STF$r8AYWAQL$S-NH₂ | 1505.79 | 753.90 | 753.94 |
| 1374 | Ac-LTF$r8AYWAEL$S-NH₂ | 1532.83 | 767.42 | 767.41 |
| 1375 | Ac-LTF$r8AYWAQL$E-NH₂ | 1573.85 | 787.93 | 787.98 |
| 1376 | mdPEG3-LTF$r8AYWAQL$S-NH₂ | 1679.92 | 840.96 | 840.97 |
| 1377 | Ac-LTF$r8AYWAQhL$S-NH₂ | 1545.86 | 773.93 | 774.31 |
| 1378 | Ac-LTF$r8AYWAQCha$S-NH₂ | 1571.88 | 786.94 | 787.3 |
| 1379 | Ac-LTF$r8AYWAQChg$S-NH₂ | 1557.86 | 779.93 | 780.4 |
| 1380 | Ac-LTF$r8AYWAQCba$S-NH₂ | 1543.84 | 772.92 | 780.13 |
| 1381 | Ac-LTF$r8AYWAQF$S-NH₂ | 1565.83 | 783.92 | 784.2 |
| 1382 | Ac-LTF4F$r8HYWAQhL$S-NH₂ | 1629.87 | 815.94 | 815.36 |
| 1383 | Ac-LTF4F$r8HYWAQCha$S-NH₂ | 1655.89 | 828.95 | 828.39 |
| 1384 | Ac-LTF4F$r8HYWAQChg$S-NH₂ | 1641.87 | 821.94 | 821.35 |
| 1385 | Ac-LTF4F$r8HYWAQCba$S-NH₂ | 1627.86 | 814.93 | 814.32 |
| 1386 | Ac-LTF4F$r8AYWAQhL$S-NH₂ | 1563.85 | 782.93 | 782.36 |
| 1387 | Ac-LTF4F$r8AYWAQCha$S-NH₂ | 1589.87 | 795.94 | 795.38 |
| 1388 | Ac-LTF4F$r8AYWAQChg$S-NH₂ | 1575.85 | 788.93 | 788.35 |
| 1389 | Ac-LTF4F$r8AYWAQCba$S-NH₂ | 1561.83 | 781.92 | 781.39 |
| 1390 | Ac-LTF3Cl$r8AYWAQhL$S-NH₂ | 1579.82 | 790.91 | 790.35 |
| 1391 | Ac-LTF3Cl$r8AYWAQCha$S-NH₂ | 1605.84 | 803.92 | 803.67 |
| 1392 | Ac-LTF3Cl$r8AYWAQChg$S-NH₂ | 1591.82 | 796.91 | 796.34 |
| 1393 | Ac-LTF3Cl$r8AYWAQCba$S-NH₂ | 1577.81 | 789.91 | 789.39 |
| 1394 | Ac-LTF$r8AYWAQhF$S-NH₂ | 1579.84 | 790.92 | 791.14 |
| 1395 | Ac-LTF$r8AYWAQF3CF3$S-NH₂ | 1633.82 | 817.91 | 818.15 |
| 1396 | Ac-LTF$r8AYWAQF3Me$S-NH₂ | 1581.86 | 791.93 | 791.32 |
| 1397 | Ac-LTF$r8AYWAQ1Nal$S-NH₂ | 1615.84 | 808.92 | 809.18 |
| 1398 | Ac-LTF$r8AYWAQBip$S-NH₂ | 1641.86 | 821.93 | 822.13 |
| 1399 | Ac-LTF$r8FYWAQL$A-NH₂ | 1591.88 | 796.94 | 797.33 |
| 1400 | Ac-LTF$r8HYWAQL$S-NHAm | 1667.94 | 834.97 | 835.92 |
| 1401 | Ac-LTF$r8HYWAQL$S-NHiAm | 1667.94 | 834.97 | 835.55 |
| 1402 | Ac-LTF$r8HYWAQL$S-NHnPr3Ph | 1715.94 | 858.97 | 859.79 |
| 1403 | Ac-LTF$r8HYWAQL$S-NHnBu3,3Me | 1681.96 | 841.98 | 842.49 |
| 1404 | Ac-LTF$r8HYWAQL$S-NHnPr | 1639.91 | 820.96 | 821.58 |
| 1405 | Ac-LTF$r8HYWAQL$S-NHnEt2Ch | 1707.98 | 854.99 | 855.35 |
| 1406 | Ac-LTF$r8HYWAQL$S-NHHex | 1681.96 | 841.98 | 842.4 |
| 1407 | Ac-LTF$r8AYWAQL$S-NHmdPeg2 | 1633.91 | 817.96 | 818.35 |
| 1408 | Ac-LTF$r8AYWAQL$A-NHmdPeg2 | 1617.92 | 809.96 | 810.3 |
| 1409 | Ac-LTF$r8AYWAQL$A-NHmdPeg4 | 1705.97 | 853.99 | 854.33 |
| 1410 | Ac-F$r8AYdl4mWEAL$A-NH₂ | 1316.72 | 659.36 | 659.44 |
| 1411 | Ac-F$r8AYdl5clWEAL$A-NH₂ | 1336.66 | 669.33 | 669.43 |
| 1412 | Ac-LThF$r8AYWAQL$S-NH₂ | 1545.86 | 773.93 | 774.11 |
| 1413 | Ac-LT2Nal$r8AYWAQL$S-NH₂ | 1581.86 | 791.93 | 792.43 |
| 1414 | Ac-LTA$r8AYWAQL$S-NH₂ | 1455.81 | 728.91 | 729.15 |
| 1415 | Ac-LTF$r8AYWVQL$S-NH₂ | 1559.88 | 780.94 | 781.24 |
| 1416 | Ac-LTF$r8HYWAAL$A-NH₂ | 1524.85 | 763.43 | 763.86 |
| 1417 | Ac-LTF$r8VYWAQL$A-NH₂ | 1543.88 | 772.94 | 773.37 |
| 1418 | Ac-LTF$r8IYWAQL$S-NH₂ | 1573.89 | 787.95 | 788.17 |
| 1419 | Ac-FTF$r8VYWSQL$S-NH₂ | 1609.85 | 805.93 | 806.22 |
| 1420 | Ac-ITF$r8FYWAQL$S-NH₂ | 1607.88 | 804.94 | 805.2 |
| 1421 | Ac-2NalTF$r8VYWSQL$S-NH₂ | 1659.87 | 830.94 | 831.2 |
| 1422 | Ac-ITF$r8LYWSQL$S-NH₂ | 1589.89 | 795.95 | 796.13 |
| 1423 | Ac-FTF$r8FYWAQL$S-NH₂ | 1641.86 | 821.93 | 822.13 |
| 1424 | Ac-WTF$r8VYWAQL$S-NH₂ | 1632.87 | 817.44 | 817.69 |
| 1425 | Ac-WTF$r8WYWAQL$S-NH₂ | 1719.88 | 860.94 | 861.36 |
| 1426 | Ac-VTF$r8AYWSQL$S-NH₂ | 1533.82 | 767.91 | 768.19 |
| 1427 | Ac-WTF$r8FYWSQL$S-NH₂ | 1696.87 | 849.44 | 849.7 |
| 1428 | Ac-FTF$r8IYWAQL$S-NH₂ | 1607.88 | 804.94 | 805.2 |
| 1429 | Ac-WTF$r8VYWSQL$S-NH₂ | 1648.87 | 825.44 | 824.8 |
| 1430 | Ac-FTF$r8LYWSQL$S-NH₂ | 1623.87 | 812.94 | 812.8 |
| 1431 | Ac-YTF$r8FYWSQL$S-NH₂ | 1673.85 | 837.93 | 837.8 |
| 1432 | Ac-LTF$r8AY6clWEAL$A-NH₂ | 1550.79 | 776.40 | 776.14 |
| 1433 | Ac-LTF$r8AY6clWSQL$S-NH₂ | 1581.80 | 791.90 | 791.68 |
| 1434 | Ac-F$r8AY6clWSAL$A-NH₂ | 1294.65 | 648.33 | 647.67 |
| 1435 | Ac-F$r8AY6clWQAL$AA-NH₂ | 1406.72 | 704.36 | 703.84 |
| 1436 | Ac-LHF$r8AYWAQL$S-NH₂ | 1567.86 | 784.93 | 785.21 |
| 1437 | Ac-LTF$r8AYWAQL$S-NH₂ | 1531.84 | 766.92 | 767.17 |
| 1438 | Ac-LTF$r8AHWAQL$S-NH₂ | 1505.84 | 753.92 | 754.13 |
| 1439 | Ac-LTF$r8AYWAHL$S-NH₂ | 1540.84 | 771.42 | 771.61 |
| 1440 | Ac-LTF$r8AYWAQL$H-NH₂ | 1581.87 | 791.94 | 792.15 |
| 1441 | H-LTF$r8AYWAQL$A-NH₂ | 1473.84 | 737.92 | 737.29 |
| 1442 | Ac-HHF$r8AYWAQL$S-NH₂ | 1591.83 | 796.92 | 797.35 |
| 1443 | Ac-aAibWTF$r8VYWSQL$S-NH₂ | 1804.96 | 903.48 | 903.64 |
| 1444 | Ac-AibWTF$r8HYWAQL$S-NH₂ | 1755.91 | 878.96 | 879.4 |
| 1445 | Ac-AibAWTF$r8HYWAQL$S-NH₂ | 1826.95 | 914.48 | 914.7 |
| 1446 | Ac-fWTF$r8HYWAQL$S-NH₂ | 1817.93 | 909.97 | 910.1 |
| 1447 | Ac-AibWWTF$r8HYWAQL$S-NH₂ | 1941.99 | 972.00 | 972.2 |
| 1448 | Ac-WTF$r8LYWSQL$S-NH₂ | 1662.88 | 832.44 | 832.8 |
| 1449 | Ac-WTF$r8NleYWSQL$S-NH₂ | 1662.88 | 832.44 | 832.6 |
| 1450 | Ac-LTF$r8AYWSQL$a-NH₂ | 1531.84 | 766.92 | 767.2 |
| 1451 | Ac-LTF$r8EYWARL$A-NH₂ | 1601.90 | 801.95 | 802.1 |
| 1452 | Ac-LTF$r8EYWAHL$A-NH₂ | 1582.86 | 792.43 | 792.6 |
| 1453 | Ac-aTF$r8AYWAQL$S-NH₂ | 1489.80 | 745.90 | 746.08 |
| 1454 | Ac-AibTF$r8AYWAQL$S-NH₂ | 1503.81 | 752.91 | 753.11 |
| 1455 | Ac-AmfTF$r8AYWAQL$S-NH₂ | 1579.84 | 790.92 | 791.14 |
| 1456 | Ac-AmwTF$r8AYWAQL$S-NH₂ | 1618.86 | 810.43 | 810.66 |
| 1457 | Ac-NmLTF$r8AYWAQL$S-NH₂ | 1545.86 | 773.93 | 774.11 |
| 1458 | Ac-LNmTF$r8AYWAQL$S-NH₂ | 1545.86 | 773.93 | 774.11 |
| 1459 | Ac-LSarF$r8AYWAQL$S-NH₂ | 1501.83 | 751.92 | 752.18 |
| 1460 | Ac-LGF$r8AYWAQL$S-NH₂ | 1487.82 | 744.91 | 745.15 |
| 1461 | Ac-LTNmF$r8AYWAQL$S-NH₂ | 1545.86 | 773.93 | 774.2 |
| 1462 | Ac-TF$r8AYWAQL$S-NH₂ | 1418.76 | 710.38 | 710.64 |
| 1463 | Ac-ETF$r8AYWAQL$A-NH₂ | 1531.81 | 766.91 | 767.2 |
| 1464 | Ac-LTF$r8EYWAQL$A-NH₂ | 1573.85 | 787.93 | 788.1 |
| 1465 | Ac-LT2Nal$r8AYWSQL$S-NH₂ | 1597.85 | 799.93 | 800.4 |
| 1466 | Ac-LTF$r8AYWAAL$S-NH₂ | 1474.82 | 738.41 | 738.68 |
| 1467 | Ac-LTF$r8AYWAQhCha$S-NH₂ | 1585.89 | 793.95 | 794.19 |
| 1468 | Ac-LTF$r8AYWAQChg$S-NH₂ | 1557.86 | 779.93 | 780.97 |
| 1469 | Ac-LTF$r8AYWAQCba$S-NH₂ | 1543.84 | 772.92 | 773.19 |
| 1470 | Ac-LTF$r8AYWAQF3CF3$S-NH₂ | 1633.82 | 817.91 | 818.15 |
| 1471 | Ac-LTF$r8AYWAQ1Nal$S-NH₂ | 1615.84 | 808.92 | 809.18 |
| 1472 | Ac-LTF$r8AYWAQBip$S-NH₂ | 1641.86 | 821.93 | 822.32 |
| 1473 | Ac-LT2Nal$r8AYWAQL$S-NH₂ | 1581.86 | 791.93 | 792.15 |
| 1474 | Ac-LTF$r8AYWVQL$S-NH₂ | 1559.88 | 780.94 | 781.62 |
| 1475 | Ac-LTF$r8AWWAQL$S-NH₂ | 1554.86 | 778.43 | 778.65 |
| 1476 | Ac-FTF$r8VYWSQL$S-NH₂ | 1609.85 | 805.93 | 806.12 |
| 1477 | Ac-ITF$r8FYWAQL$S-NH₂ | 1607.88 | 804.94 | 805.2 |
| 1478 | Ac-ITF$r8LYWSQL$S-NH₂ | 1589.89 | 795.95 | 796.22 |
| 1479 | Ac-FTF$r8FYWAQL$S-NH₂ | 1641.86 | 821.93 | 822.41 |
| 1480 | Ac-VTF$r8AYWSQL$S-NH₂ | 1533.82 | 767.91 | 768.19 |
| 1481 | Ac-LTF$r8AHWAQL$S-NH₂ | 1505.84 | 753.92 | 754.31 |
| 1482 | Ac-LTF$r8AYWAQL$H-NH₂ | 1581.87 | 791.94 | 791.94 |
| 1483 | Ac-LTF$r8AYWAHL$S-NH₂ | 1540.84 | 771.42 | 771.61 |
| 1484 | Ac-aAibWTF$r8VYWSQL$S-NH₂ | 1804.96 | 903.48 | 903.9 |
| 1485 | Ac-AibWTF$r8HYWAQL$S-NH₂ | 1755.91 | 878.96 | 879.5 |
| 1486 | Ac-AibAWTF$r8HYWAQL$S-NH₂ | 1826.95 | 914.48 | 914.7 |
| 1487 | Ac-fWTF$r8HYWAQL$S-NH₂ | 1817.93 | 909.97 | 910.2 |
| 1488 | Ac-AibWWTF$r8HYWAQL$S-NH₂ | 1941.99 | 972.00 | 972.7 |
| 1489 | Ac-WTF$r8LYWSQL$S-NH₂ | 1662.88 | 832.44 | 832.7 |
| 1490 | Ac-WTF$r8NleYWSQL$S-NH₂ | 1662.88 | 832.44 | 832.7 |
| 1491 | Ac-LTF$r8AYWSQL$a-NH₂ | 1531.84 | 766.92 | 767.2 |
| 1492 | Ac-LTF$r8EYWARL$A-NH₂ | 1601.90 | 801.95 | 802.2 |
| 1493 | Ac-LTF$r8EYWAHL$A-NH₂ | 1582.86 | 792.43 | 792.6 |
| 1494 | Ac-aTF$r8AYWAQL$S-NH₂ | 1489.80 | 745.90 | 746.1 |
| 1495 | Ac-AibTF$r8AYWAQL$S-NH₂ | 1503.81 | 752.91 | 753.2 |
| 1496 | Ac-AmfTF$r8AYWAQL$S-NH₂ | 1579.84 | 790.92 | 791.2 |
| 1497 | Ac-AmwTF$r8AYWAQL$S-NH₂ | 1618.86 | 810.43 | 810.7 |
| 1498 | Ac-NmLTF$r8AYWAQL$S-NH₂ | 1545.86 | 773.93 | 774.1 |
| 1499 | Ac-LNmTF$r8AYWAQL$S-NH₂ | 1545.86 | 773.93 | 774.4 |
| 1500 | Ac-LSarF$r8AYWAQL$S-NH₂ | 1501.83 | 751.92 | 752.1 |
| 1501 | Ac-TF$r8AYWAQL$S-NH₂ | 1418.76 | 710.38 | 710.8 |
| 1502 | Ac-ETF$r8AYWAQL$A-NH₂ | 1531.81 | 766.91 | 767.4 |
| 1503 | Ac-LTF$r8EYWAQL$A-NH₂ | 1573.85 | 787.93 | 788.2 |
| 1504 | Ac-WTF$r8VYWSQL$S-NH₂ | 1648.87 | 825.44 | 825.2 |
| 1505 | Ac-YTF$r8FYWSQL$S-NH₂ | 1673.85 | 837.93 | 837.3 |
| 1506 | Ac-F$r8AY6clWSAL$A-NH₂ | 1294.65 | 648.33 | 647.74 |
| 1507 | Ac-ETF$r8EYWVQL$S-NH₂ | 1633.84 | 817.92 | 817.36 |
| 1508 | Ac-ETF$r8EHWAQL$A-NH₂ | 1563.81 | 782.91 | 782.36 |
| 1509 | Ac-ITF$r8EYWAQL$S-NH₂ | 1589.85 | 795.93 | 795.38 |
| 1510 | Ac-ITF$r8EHWVQL$A-NH₂ | 1575.88 | 788.94 | 788.42 |
| 1511 | Ac-ITF$r8EHWAQL$S-NH₂ | 1563.85 | 782.93 | 782.43 |
| 1512 | Ac-LTF4F$r8AYWAQCba$S-NH₂ | 1561.83 | 781.92 | 781.32 |
| 1513 | Ac-LTF3Cl$r8AYWAQhL$S-NH₂ | 1579.82 | 790.91 | 790.64 |
| 1514 | Ac-LTF3Cl$r8AYWAQCha$S-NH₂ | 1605.84 | 803.92 | 803.37 |
| 1515 | Ac-LTF3Cl$r8AYWAQChg$S-NH₂ | 1591.82 | 796.91 | 796.27 |
| 1516 | Ac-LTF3Cl$r8AYWAQCba$S-NH₂ | 1577.81 | 789.91 | 789.83 |
| 1517 | Ac-LTF$r8AY6clWSQL$S-NH₂ | 1581.80 | 791.90 | 791.75 |
| 1518 | Ac-LTF4F$r8HYWAQhL$S-NH₂ | 1629.87 | 815.94 | 815.36 |
| 1519 | Ac-LTF4F$r8HYWAQCba$S-NH₂ | 1627.86 | 814.93 | 814.32 |
| 1520 | Ac-LTF4F$r8AYWAQhL$S-NH₂ | 1563.85 | 782.93 | 782.36 |
| 1521 | Ac-LTF4F$r8AYWAQChg$S-NH₂ | 1575.85 | 788.93 | 788.35 |
| 1522 | Ac-ETF$r8EYWVAL$S-NH₂ | 1576.82 | 789.41 | 788.79 |
| 1523 | Ac-ETF$r8EHWAAL$A-NH₂ | 1506.79 | 754.40 | 754.8 |
| 1524 | Ac-ITF$r8EYWAAL$S-NH₂ | 1532.83 | 767.42 | 767.75 |
| 1525 | Ac-ITF$r8EHWVAL$A-NH₂ | 1518.86 | 760.43 | 760.81 |
| 1526 | Ac-ITF$r8EHWAAL$S-NH₂ | 1506.82 | 754.41 | 754.8 |
| 1527 | Pam-LTF$r8EYWAQL$S-NH₂ | 1786.07 | 894.04 | 894.48 |
| 1528 | Pam-ETF$r8EYWAQL$S-NH₂ | 1802.03 | 902.02 | 902.34 |
| 1529 | Ac-LTF$r8AYWLQL$S-NH₂ | 1573.89 | 787.95 | 787.39 |
| 1530 | Ac-LTF$r8EYWLQL$S-NH₂ | 1631.90 | 816.95 | 817.33 |
| 1531 | Ac-LTF$r8EHWLQL$S-NH₂ | 1605.89 | 803.95 | 804.29 |
| 1532 | Ac-LTF$r8VYWAQL$S-NH₂ | 1559.88 | 780.94 | 781.34 |
| 1533 | Ac-LTF$r8AYWSQL$S-NH₂ | 1547.84 | 774.92 | 775.33 |
| 1534 | Ac-ETF$r8AYWAQL$S-NH₂ | 1547.80 | 774.90 | 775.7 |
| 1535 | Ac-LTF$r8EYWAQL$S-NH₂ | 1589.85 | 795.93 | 796.33 |
| 1536 | Ac-LTF$r8HYWAQL$S-NHAm | 1667.94 | 834.97 | 835.37 |
| 1537 | Ac-LTF$r8HYWAQL$S-NHiAm | 1667.94 | 834.97 | 835.27 |
| 1538 | Ac-LTF$r8HYWAQL$S-NHnPr3Ph | 1715.94 | 858.97 | 859.42 |
| 1539 | Ac-LTF$r8HYWAQL$S-NHnBu3,3Me | 1681.96 | 841.98 | 842.67 |
| 1540 | Ac-LTF$r8HYWAQL$S-NHnBu | 1653.93 | 827.97 | 828.24 |
| 1541 | Ac-LTF$r8HYWAQL$S-NHnPr | 1639.91 | 820.96 | 821.31 |
| 1542 | Ac-LTF$r8HYWAQL$S-NHnEt2Ch | 1707.98 | 854.99 | 855.35 |
| 1543 | Ac-LTF$r8HYWAQL$S-NHHex | 1681.96 | 841.98 | 842.4 |
| 1544 | Ac-LTF$r8AYWAQL$S-NHmdPeg2 | 1633.91 | 817.96 | 855.35 |
| 1545 | Ac-LTF$r8AYWAQL$A-NHmdPeg2 | 1617.92 | 809.96 | 810.58 |
| 1546 | Ac-LTF$r5AYWAAL$s8S-NH₂ | 1474.82 | 738.41 | 738.79 |
| 1547 | Ac-LTF$r8AYWCouQL$S-NH₂ | 1705.88 | 853.94 | 854.61 |
| 1548 | Ac-LTF$r8CouYWAQL$S-NH₂ | 1705.88 | 853.94 | 854.7 |
| 1549 | Ac-CouTF$r8AYWAQL$S-NH₂ | 1663.83 | 832.92 | 833.33 |
| 1550 | H-LTF$r8AYWAQL$A-NH₂ | 1473.84 | 737.92 | 737.29 |
| 1551 | Ac-HHF$r8AYWAQL$S-NH₂ | 1591.83 | 796.92 | 797.72 |
| 1552 | Ac-LT2NalSr8AYWSQL$S-NH₂ | 1597.85 | 799.93 | 800.68 |
| 1553 | Ac-LTF$r8HCouWAQL$S-NH₂ | 1679.87 | 840.94 | 841.38 |
| 1554 | Ac-LTF$r8AYWCou2QL$S-NH₂ | 1789.94 | 895.97 | 896.51 |
| 1555 | Ac-LTF$r8Cou2YWAQL$S-NH₂ | 1789.94 | 895.97 | 896.5 |
| 1556 | Ac-Cou2TF$r8AYWAQL$S-NH₂ | 1747.90 | 874.95 | 875.42 |
| 1557 | Ac-LTF$r8ACou2WAQL$S-NH₂ | 1697.92 | 849.96 | 850.82 |
| 1558 | Dmaac-LTF$r8AYWAQL$S-NH₂ | 1574.89 | 788.45 | 788.82 |
| 1559 | Hexac-LTF$r8AYWAQL$S-NH₂ | 1587.91 | 794.96 | 795.11 |
| 1560 | Napac-LTF$r8AYWAQL$S-NH₂ | 1657.89 | 829.95 | 830.36 |
| 1561 | Pam-LTF$r8AYWAQL$S-NH₂ | 1728.06 | 865.03 | 865.45 |
| 1562 | Ac-LT2Nal$r8HYAAQL$S-NH₂ | 1532.84 | 767.42 | 767.61 |
| 1563 | Ac-LT2NalS/r8HYWAQL$/S-NH₂ | 1675.91 | 838.96 | 839.1 |
| 1564 | Ac-LT2Nal$r8HYFAQL$S-NH₂ | 1608.87 | 805.44 | 805.9 |
| 1565 | Ac-LT2Nal$r8HWAAQL$S-NH₂ | 1555.86 | 778.93 | 779.08 |
| 1566 | Ac-LT2Nal$r8HYAWQL$S-NH₂ | 1647.88 | 824.94 | 825.04 |
| 1567 | Ac-LT2Nal$r8HYAAQW$S-NH₂ | 1605.83 | 803.92 | 804.05 |
| 1568 | Ac-LTW$r8HYWAQL$S-NH₂ | 1636.88 | 819.44 | 819.95 |
| 1569 | Ac-LT1NalSr8HYWAQL$S-NH₂ | 1647.88 | 824.94 | 825.41 |

In some embodiments, a peptidomimetic macrocycle disclosed herein does not comprise a peptidomimetic macrocycle structure as shown in TABLE 2b.

TABLE 2c shows examples of non-crosslinked polypeptides comprising D-amino acids.

TABLE 2c

| | | | Exact | Found | Calc | Calc | Calc |
|---|---|---|---|---|---|---|---|
| SP | Sequence | Isomer | Mass | Mass | (M + 1)/1 | (M + 2)/2 | (M + 3)/3 |
| 1570 | Ac-tawyanfekllr-NH₂ | | | 777.46 | | | |
| 1571 | Ac-tawyanf4CF3ekllr-NH₂ | | | 811.41 | | | |

### Example 2

### In Vitro and In Vivo Effects of Combination Therapy Using AP1 and Paclitaxel

Paclitaxel is one of the most widely used chemotherapeutic agents that promotes the assembly of microtubules from tubulin dimers. Paclitaxel stabilizes microtubules by preventing depolymerization, which results in the inhibition of the normal dynamic reorganization of the microtubule network that is essential for vital interphase and mitotic cellular functions.

Following inhibition of mitotic spindle disassembly in G2/M by paclitaxel, aberrant mitosis (with improper chromosome segregation) or mitotic slippage (an improper exit from mitosis in the absence of chromosome segregation and cytokinesis producing tetraploid cells) may occur, both of which result in apoptosis in the presence of an activated p53 signaling by AP1.

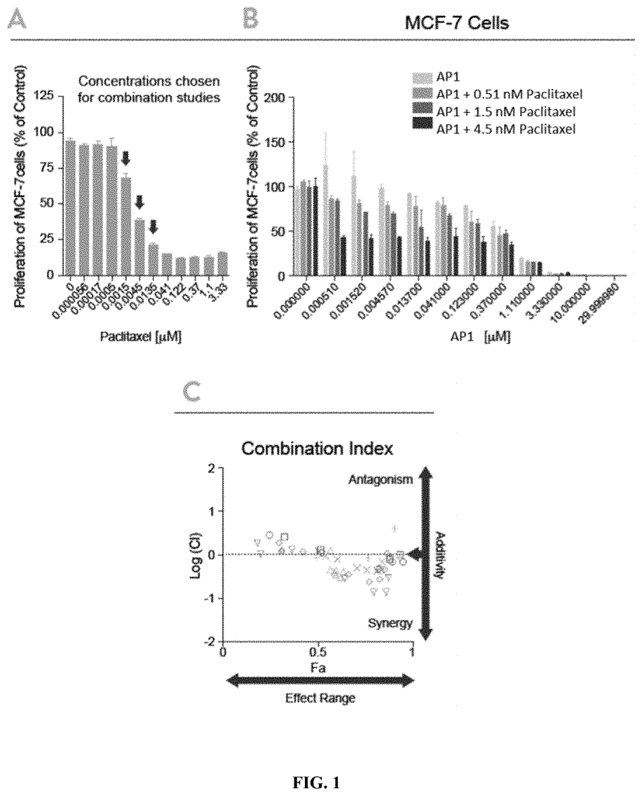

FIG. 1 shows results obtained from in vitro cell proliferation assays performed in estrogen receptor-positive (ER-positive) TP53 wild-type MCF-7 breast cancer cell lines, to determine the IC50 of AP1 and paclitaxel, using isobologram curves compared to paclitaxel and compared to AP1. FIG. 1 PANEL A shows cell viability data in response to varying concentrations of paclitaxel (arrows denote concentrations chosen for combination studies). PANEL B shows cell proliferation data of MCF-7 cells treated with the indicated dose of paclitaxel and varying concentrations of AP1. PANEL C shows combination indices for the drug combination of AP1 and paclitaxel. The results showed additive to synergistic cytotoxic activity of the combination of AP1 with paclitaxel in vitro in MCF-7 breast cancer cells. TABLE 3 shows the combination index of AP1 with paclitaxel in MCF-7 breast cancer cell lines.

TABLE 3

| | CI of IC₅₀ | CI of IC₇₅ | |
|---|---|---|---|
| Paclitaxel and AP1 | 0.874 | 0.834 | |
| Drug interaction | Synergistic | Additive | Antagonistic |
| CI value | <0.9 | 0.9-1.1 | >1.1 |

To further characterize the effects of AP1 in combination with paclitaxel, a mouse xenograft experiment was conducted. WT TP53 ER-positive MCF-7 breast cancer cells were implanted on the mammary fat-pad into nude mice. The mice received estrogen via a slow release subcutaneous implant. Mice were then treated with different dose levels of AP1 and paclitaxel and tumor volume and body weights were measured by caliper twice weekly for 28 days. No significant weight loss was observed in mice. In the group of mice treated with 15 mg/kg paclitaxel and 10 mg/kg AP1, a 38% animal loss was observed (2 had tail vein necrosis at site of injection).

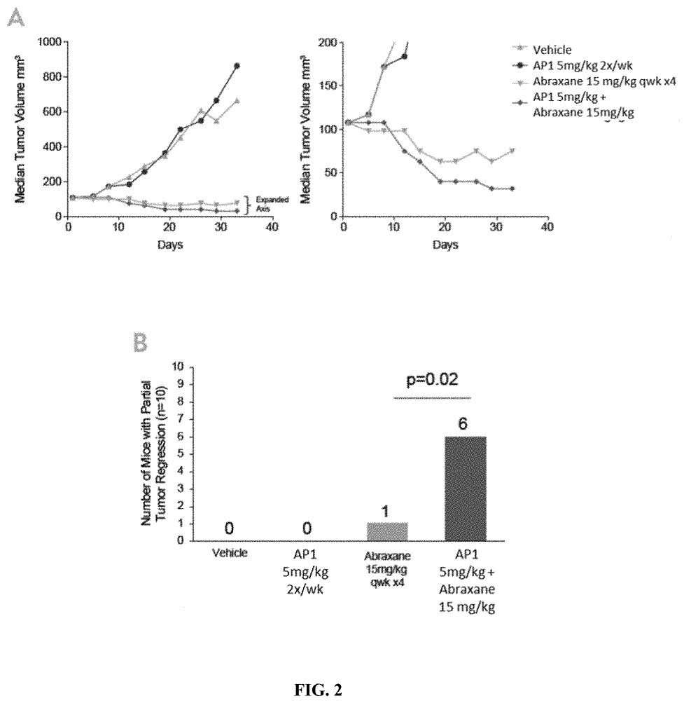

FIG. 2 PANEL A shows data collected from athymic nude mice with established tumors (n=10 per group) that were treated for 4 weeks with AP1 twice-weekly alone or in combination with weekly doses of nab-paclitaxel. Compounds were co-administered intravenously at the indicated doses. PANEL B shows objective tumor responses on d32 (partial regression=3 consecutive measurements<50% of starting volume).

Overall, the combination of AP1 with paclitaxel had greater anti-tumor efficacy than either agent alone. Results of statistical comparisons are presented in TABLE 4.

TABLE 4
2-Way ANOVA (Tukey's multiple comparisons test 95%)

| | | 95.00% | Adjusted |
|---|---|---|---|
| | Mean | CI of | P |
| Day28 | Difference | Difference | Value |
| Control vs. AP1 10 mg/kg + | 1.722 | 0.8772 to | <0.0001 |
| Paclitaxel 15 mg/kg | | 2.567 | |
| Control vs. AP1 10 mg/kg + | 1.5 | 0.7591 to | <0.0001 |
| Paclitaxel 10 mg/kg | | 2.241 | |
| AP1 10 mg/kg vs. AP1 10 mg/kg + | 1.489 | 0.6444 to | <0.0001 |
| Paclitaxel 15 mg/kg | | 2.334 | |
| AP1 10 mg/kg vs. AP1 10 mg/kg + | 1.267 | 0.5263 to | <0.0001 |
| Paclitaxel 10mg/kg | | 2.008 | |
| Paclitaxel 15 mg/kg vs. AP1 | 1.032 | 0.1874 to | 0.0049 |
| 10 mg/kg + Paclitaxel 15 mg/kg | | 1.877 | |
| Paclitaxel 10 mg/kg vs. AP1 | 2.257 | 1.49 to | <0.0001 |
| 10 mg/kg + Paclitaxel 10 mg/kg | | 3.024 | |
| Control vs. AP1 5 mg/kg + | 2.257 | 1.517 to | <0.0001 |
| Paclitaxel 15 mg/kg | | 2.998 | |
| Control vs. AP1 5 mg/kg + | 1.838 | 1.037 to | <0.0001 |
| Paclitaxel 10 mg/kg | | 2.638 | |
| AP1 5 mg/kg vs. AP1 5 mg/kg + | 1.497 | 0.7299 to | <0.0001 |
| Paclitaxel 15 mg/kg | | 2.263 | |
| AP1 5 mg/kg vs. AP1 5 mg/kg + | 1.077 | 0.2526 to | 0.0018 |
| Paclitaxel 10 mg/kg | | 1.901 | |
| Paclitaxel 15 mg/kg vs. AP1 | 1.568 | 0.8268 to | <0.0001 |
| 5 mg/kg + Paclitaxel 15 mg/kg | | 2.308 | |
| Paclitaxel 10 mg/kg vs. AP1 | 2.594 | 1.77 to | <0.0001 |
| 5 mg/kg + Paclitaxel 10 mg/kg | | 3.419 | |

### Example 3

### Phase 1b Study of AP1 in Combination with Paclitaxel in Wild-Type TP53 Advanced or Metastatic Solid Tumors Including ER-Positive Breast Cancer

A phase 1b study of AP1 in combination with paclitaxel in wild-type TP53 advanced or metastatic solid tumors including ER-positive breast cancer is conducted. The study is an open-label, single center, dose-escalation and dose expansion study, and is used to evaluate the safety, tolerability, PK, PD, and anti-tumor effects of AP1 in combination with paclitaxel for the treatment of adults with solid tumors and WT TP53. Patients receive AP1 plus paclitaxel on Days 1, 8, and 15 of consecutive 28-day cycles until they experience disease progression, unacceptable toxicity, or another criterion for treatment withdrawal. In case of clinical benefits, the patients continue treatment beyond first tumor progression as defined by RECIST 1.1.

The study enrolls patients over a period of 18 months. Each individual patient is expected to participate in the study for approximately 4 months, excluding survival follow-up.

a. Study Objectives

Primary objectives: The primary objectives of the study are to 1) determine the dose-limiting toxicities (DLT) and the maximum tolerated dose (MTD) of AP1 in combination with paclitaxel in adult patients with advanced or metastatic solid tumors with wild-type (WT) TP53; and 2) evaluate the safety and tolerability of AP1 in combination with paclitaxel in patients with advanced or metastatic WT TP53 solid tumors.

Key secondary objective: The key secondary objective of the study is to evaluate the anti-tumor activity of AP1 in combination with paclitaxel in solid tumors (in dose escalation) and hormone-receptor positive breast cancer (in expansion).

Other secondary objective: The other secondary objective of the study is to describe the pharmacokinetics (PK) of AP1 and paclitaxel in plasma following single and multiple intravenous (IV) infusions (Cycle 1 D1, D2, D15, and Cycle 2 D1).

Exploratory objectives: Additional exploratory objectives of the study are to 1) assess predictive and pharmacodynamic (PD) markers of response; 2) assess the effects of AP1 and paclitaxel on cell proliferation and apoptosis; and 3) assess the effects of AP1 and paclitaxel on cell-free DNA (cfDNA) dynamics and macrophage inhibitory cytokine-1 (MIC-1).

b. Study Endpoints

Primary endpoints: The primary endpoint of the study are: 1) the MTD of the combination of AP1 and paclitaxel, defined as the isotonic estimate of the toxicity rate closest to 0.30; and 2) adverse events (AEs), serious adverse events (SAEs), and changes from baseline in physical examination findings, vital signs, clinical laboratory parameters and electrocardiogram (ECG) parameters.

Key secondary endpoints: The key secondary endpoints of the phase 1b study are 1) objective response rate (ORR) defined as the proportion of patients with complete response (CR) or partial response (PR), as determined by investigator assessment using Response Evaluation Criteria in Solid Tumors (RECIST v1.1); 2) duration of response (DoR) defined as the time from documentation of tumor response to disease progression; 3) progression-free survival (PFS) defined as the time from the start of treatment to disease progression or death, whichever occurs first; 4) clinical benefit rate at 24 weeks defined as the proportion of patients with CR, PR, or stable disease (SD); and 5) overall survival (OS) defined as the time from the start of treatment to death from any cause.

Other secondary endpoint: The other secondary endpoint of the study includes PK parameters, including area under the curve (AUC), maximum concentration (Cₘₐₓ), and time to Cₘₐₓ (Tₘₐₓ), and half-life (t_{1/2}) for AP1 and paclitaxel.

Exploratory endpoints: Exploratory endpoints of the study include 1) correlation of response with p53 status, p21 status, murine double minute 2 (MDM2) and murine double minute X (MDMX) expression by immunohistochemistry (IHC) and by reverse phase proteomic array (RPPA) in pre- and on-treatment tumor biopsy samples; 2) whole exome sequencing on pre-treatment biopsy and at progression for TP53 mutations, MDM2 and MDMX copy number and other genomic alterations; 3) RNAseq for gene expression profiling pre-treatment, on-treatment and at progression; 4) cell proliferation and apoptosis assays (Ki67, cleaved caspase3) on pre- and on-treatment tumor biopsy samples; and 5) cell-free DNA (cfDNA) in blood, and serum concentrations of MIC-1.

c. Study Design and Description

The phase lb study is conducted in two stages: 1) dose escalation stage; and 2) expansion stage. During the dose escalation stage of study, the Bayesian Optimal Interval Design is implemented to establish the MTD of AP1 and paclitaxel administered in combination. Patients are enrolled and treated in cohorts of 3. In the expansion stage, 15 additional patients with ER positive (ER+) WT TP53 metastatic breast cancers are treated at the MTD to evaluate preliminary activity of AP1 and paclitaxel combination and identification of biomarkers of response. Tumor biopsies are performed pre-treatment and after start of treatment (Day 8-10 of Cycle 1) for identification of predictive and pharmacodynamic markers of response. Tumor biopsies are optional for patients in dose escalation; however, tumor biopsies are mandatory in the dose expansion cohort in patients in whom biopsies can be safely performed.

Safety assessments: Safety assessments include AEs/SAEs, physical examinations, collection of vital signs, clinical laboratory parameters, and ECG parameters. Clinically significant changes in physical examinations findings are reported as AEs. Adverse events are monitored from the start of study treatment until 30 days after the last dose or start of subsequent therapy, whichever occurs first.

Definition of dose-limiting toxicities: Toxicities are graded according to the National Cancer Institute (NCI) Common Toxicity Criteria for Adverse Events version 5.0 (CTCAE v5.0). Dose-limiting toxicity (DLT) is a toxicity that occurs during Cycle 1 and is felt to be possibly, probably, or definitely related to the study treatment as follows:

Hematologic toxicity: Hematologic toxicity is graded using the following criteria: 1) Grade 3 or 4 neutropenia complicated by fever>38.5° C. or infection; 2) Grade 4 neutropenia of at least 7 days duration; 3) Grade 3 or 4 thrombocytopenia complicated by clinically significant hemorrhage; or 4) Grade 4 thrombocytopenia of at least 7 days duration.

Non-hematologic toxicity: Non-hematologic toxicity includes 1) any non-hematologic AE of Grade 3-4 or higher except a) nausea, vomiting or diarrhea that can be controlled by appropriate medical intervention or prophylaxis and that resolves to Grade 1 within 48 hours with medical intervention; b) clinically significant electrolyte toxicities able to be corrected to ≤Grade 1 or baseline within 3 days; c) fatigue that resolves to ≤Grade 1 or baseline within 7 days; d) elevations of lipase and/or amylase in the absence of clinical pancreatitis; e) asymptomatic transient hyperbilirubinemia; or f) infusion related reactions; 2) allergic reaction/hypersensitivity are not considered to be dose-limiting; 3) alopecia is not be considered to be dose-limiting. Delays in starting Cycle 2 by ≥2 weeks due to treatment-related toxicity constitute a DLT.

Efficacy assessments: Tumor assessment is perfomed using computed tomography (CT), and magnetic resonance imaging (MRI) as needed, approximately every 8 weeks during treatment. Following the discontinuation of study treatment, patients continue to be followed for survival.

Pharmacokinetic, pharmacodynamic, and other assessments: Whole exome sequencing is performed on a tissue sample obtained from the pre-treatment biopsy to evaluate the association of response with any particular genomic alterations (e.g., MDM2/MDMX amplification). RNA sequencing is performed to assess association with baseline gene expression (e.g., expression of MDM2/4 and relative expression of MDM4 splicing isoforms) and modulation of gene expression with therapy, including p53 target gene PHLDA3. Cell proliferation and apoptosis assays (Ki67, cleaved caspase3) are performed to test the hypothesis that AP1 in addition to paclitaxel induces apoptosis in cancer cells with WT TP53. Expression of p53, p21, and MDM2 is also be assessed by IHC. Cell-free circulating DNA (cfDNA) is performed using Guardant or alternate technology. Samples for cfDNA are obtained prior to the start of each cycle and at the end of treatment. The serum concentration of MIC-1 is assessed as an additional pharmacodynamic marker.

Formulation: The AP1 drug product is a frozen or refrigerated liquid product supplied in single-use glass vials in a single dose strength of 75 mg in 5.0 mL, dissolved in 20 mM sodium phosphate, 240 mM trehalose, and 300 ppm Polysorbate 20 at pH 7.5. Each vial contains a recoverable volume of 5.0 mL and is filled with formulated AP1 to 5.5±0.2 mL. AP1 for injection is stored as a refrigerated product at 2° to 8° C. or frozen product at −15° to −25° C.

Preparation: AP1 is introduced into an IV infusion bag containing D5W, which is known as AP1 Dosing Solution and is provided by the site pharmacy for administration to the patient. AP1 Dosing Solution is labeled with the Patient Identification Number. The investigative staff confirms the Patient Identification Number and the relevancy of the Patient Identification Number to the intended patient. The start of the AP1 infusion begins within 6 hours of dilution into 250 mL D5W, and the infusion bag is kept at room temperature until use.

d. Study Population

Patients are required to meet all of the following criteria before the patients are eligible to enter the study. Approximately 30-45 patients are enrolled in the phase 1b study. 15-30 patients are assigned to the dose escalation stage of the phase 1b study, and 15 patients are assigned to the expansion stage of the phase 1b study.

Inclusion Criteria
1. 18 years of age or older
2. Histologically- or cytologically-confirmed solid tumors (excluding lymphomas) that are metastatic or unresectable and that meet the following criteria:
   a. Escalation and expansion cohorts: wild type (WT) TP53 status defined as no mutation on a Clinical Laboratory Improvement Amendments (CLIA)-certified next-generation sequencing (NGS) assay that has sequenced the full length TP53 gene. Patients can be enrolled based on tissue testing or liquid biopsies. If enrolled based on liquid biopsies, testing is conducted to detect other somatic mutations.
   b. Expansion cohort only: estrogen receptor (ER) positive (>1%), human epidermal growth factor 2 (HER2) negative, WT TP53 metastatic or inoperable locally advanced or locally recurrent breast cancer. Patients can be HER2 0+ or 1+, 2+ or fluorescent in situ hybridization (FISH) non-amplified to be considered HER2 negative.
3. Standard treatment with therapies known to confer a survival benefit does not exist, is no longer effective or tolerated, or the patient declines standard treatment.
4. Measurable disease by Response Evaluation Criteria in Solid Tumors (RECIST) 1.1. In the dose escalation stage, patients without measurable disease by RECIST 1.1, but evaluable disease are also eligible.
5. Eastern Cooperative Oncology Group (ECOG) performance status of 0-1.
6. Demonstrate adequate organ function as defined by the parameters listed below:
   a. Serum creatinine≤1.5×upper limit of normal (ULN) or ≥45 mL/min/1.73 m² by CKD-EPI equation for subjects with creatinine levels>1.5×institutional ULN.
   b. Total bilirubin≤1.5×ULN, or direct bilirubin≤ULN for subjects with total bilirubin levels>1.5×ULN, or unless due to Gilbert's Syndrome.
   c. Alanine aminotransferase (ALT)/aspartate aminotransferase (AST)≤2.5×ULN or ≤5×ULN if hepatic abnormalities are related to underlying liver metastases or liver/biliary primary.
   d. Absolute neutrophil count (ANC)≥1500/mm³ (without granulocyte-colony stimulating factor [GCSF] in the 2 weeks prior to treatment start)
   e. Platelet count≥100,000/mm³
   f. Hemoglobin≥9 g/dL (without blood transfusion in the 2 weeks prior to treatment start)
   g. International normalized ratio (INR) and activated partial thromboplastin time (aPTT)≤1.5×ULN.
7. All patients (males and females) of childbearing potential agree to use medically effective contraception during the study and for 6 months after the last dose of study drugs. Females have a negative serum pregnancy test during screening and a negative urine pregnancy test at study day 1 prior to initiation of treatment.
8. Have no concomitant medical condition that in the judgment of the investigator will interfere with the patient's ability to participate in the study or render such participation medically inappropriate.
9. No medical history of another cancer (except basal or squamous cell skin cancer or in situ cervical cancer, or carcinomas in situ or other malignancies with a ≥95% 5-year survival) within 2 years of the start of study treatment.
10. No investigational drug or other anticancer treatments (including chemotherapy or radiation therapy) within 21 days or at least 5 half-lives, whichever is shorter, of the start of the study treatment.
11. No major surgery within 1 month of treatment and fully recovered.
12. Willing and able to provide informed consent.

Exclusion Criteria
1. Previous treatment with investigational agents that inhibit MDM2 or MDMX activity.
2. Known active hepatitis B, hepatitis C, and/or human immunodeficiency virus (HIV)-positive patients who have a cluster of differentiation 4 (CD4) count<200. No antiretroviral medications that are CYP3A4 substrates will be allowed.
3. Requirement for therapeutic anticoagulation
4. Pre-existing history of or known cardiovascular risk:
   a. History of acute coronary syndromes within 6 months prior to the first dose of AP1 (including myocardial infarction, unstable angina, coronary artery bypass graft, angioplasty, or stenting).
   b. Uncontrolled hypertension.
   c. Pre-existing cardiac failure (New York Heart Association class III-IV).
   d. Atrial fibrillation on anti-coagulants.
   e. Clinically significant uncontrolled arrhythmias.
   f. Corrected QT (QTcF) interval on screening ECG≥450 msec for males and ≥470 msec for females (QTcF>480 msec for any patient with a bundle branch block).
5. Clinically significant gastrointestinal bleeding within 6 months prior to the start of study treatment.
6. Females who are pregnant or nursing.
7. Symptomatic central nervous system (CNS) metastases by history, clinical signs or radiologic findings. Stable brain metastases (1 month after completion of treatment) confirmed by imaging are allowed.
8. Known hypersensitivity to any study drug component.
9. The required use of any concomitant medications that are predominantly cleared by hepatobiliary transporters, OATP members OATP1B1 and OATP1B3, on the day of the AP1 infusion or within 48 hours after an AP1 infusion.
10. Patients with Grade≥2 neuropathy will be excluded.

Replacement of patients: Any patient who completes screening and does not receive at least one dose each of AP1 and paclitaxel is replaced. A patient in the dose escalation portion of the study who discontinues the study prior to completion of the first cycle for reasons other than toxicity, and who does not receive at least 2 doses in the first cycle (C1D1 and C1D8 or C1D15) of AP1 and paclitaxel), is considered unevaluable for DLT assessment and is replaced.

A patient in the dose expansion portion of the study who discontinues study participation prior to the completion of the first cycle of treatment for any reason or who does not receive all required doses in the first cycle is replaced. Patients who are determined be TP53 mutant on pre-treatment biopsy is excluded from response assessment.

e. Treatment Regimen

Paclitaxel is administered by IV infusion over 1 hour on Days 1, 8, and 15 of every 28-day cycle. AP1 is administered by IV infusion over 1 hour on Days 1, 8, and 15 of every 28-day cycle beginning 2 hours after the end of paclitaxel infusion. The patients do not receive treatment on Day 21.

The dose levels of paclitaxel and AP1 to be evaluated during dose escalation are shown in TABLE 5. If toxicity is observed at Level 1 (L1), two dose de-escalation levels are possible.

TABLE 5

| | AP1 (mg/kg) IV | Paclitaxel (mg/m²) IV |
|---|---|---|
| Dose Level | D1, D8, D15 | D1, D8, D15 |
| L-2 | 0.64 | 60 |
| L-1 | 1.25 | 60 |
| L1 | 1.25 | 80 |
| L2 | 2.1 | 80 |
| L3 | 3.1 | 80 |

During the expansion stage, patients are treated at the MTD identified during the dose escalation stage.

Treatment continues until disease progression, unacceptable toxicity, or other criteria for treatment withdrawal are met. However, in case of clinical benefit, treatment beyond first radiologic disease progression is allowed.

TABLE 6 shows the schedule of study activities presented for cycle 1, and for cycles 2 and beyond in TABLE 7.

TABLE 6

| | | D1 | | | D8 ± 1 d | | | D15 ± 1 d | | |
|---|---|---|---|---|---|---|---|---|---|---|
| | Screening | Pre- | Post- | | Pre- | Post- | | Pre- | | |
| Procedure | −21 days | dose | dose | D2 | dose | dose | D10 | dose | Post-dose | D22 ± 1 d |
| Written informed consent | X | | | | | | | | | |
| Medical and disease | X | | | | | | | | | |
| history | | | | | | | | | | |
| Demographics | X | | | | | | | | | |
| Archive tissue sample¹ | X | | | | | | | | | |
| Tumor biopsy² | X | | | | X² | | | | | |
| Eligibility | X | | | | | | | | | |
| Blood test for CD4 count | X | | | | | | | | | |
| and | | | | | | | | | | |
| hepatitis B and C viral | | | | | | | | | | |
| load (if history of | | | | | | | | | | |
| hepatitis B or C and/or | | | | | | | | | | |
| HIV infection)³ | | | | | | | | | | |
| Pregnancy test⁴ | X | X | | | | | | | | |
| Vital signs⁵ | Within 7 | X | X | X | X | X | | X | X | X |
| | days prior | | | | | | | | | |
| | to Day 1 | | | | | | | | | |
| Physical exam⁶ | X | X | | | X | | | X | | X |
| 12-lead ECG⁷ | X | X | | | | | | | | |
| Laboratory assessments - | Within 7 | X | | X | X | | | X | | X |
| chemistry | days prior | | | | | | | | | |
| | to Day 1 | | | | | | | | | |
| Laboratory assessments - | Within 7 | X | | X | X | | | X | | X |
| hematology | days prior | | | | | | | | | |
| | to Day 1 | | | | | | | | | |
| Laboratory assessments - | Within 7 | X | | X | X | | | X | | X |
| coagulation | days prior | | | | | | | | | |
| | to Day 1 | | | | | | | | | |
| Laboratory assessments - | Within 7 | | | | | | | | | |
| urinalysis | days prior | Perform as clinically indicated | | | | | | | | |
| | to Day 1 | | | | | | | | | |
| Laboratory assessments - | X | | | | | | | | | |
| tumor markers (as | | | | | | | | | | |
| appropriate) | | | | | | | | | | |
| Blood Collection - | | X | | | | | | | | |
| normal control for NGS | | | | | | | | | | |
| Blood Collection-PD- | | X⁸ | X⁸ | X⁹ | | | | | | |
| MIC-1 | | | | | | | | | | |
| Blood Collection-PD- | X | | | | | | | | | |
| cfDNA | | | | | | | | | | |
| Blood Collection-PK | | X¹⁰ | | X¹⁰ | | | | X¹⁰ | | |
| assessments | | | | | | | | | | |
| ECOG Performance | Within 7 | X | | X | | | X | | | |
| Status | days prior | | | | | | | | | |
| | to Day 1 | | | | | | | | | |
| Tumor | Within 28 | | | | | | | | | |
| Assessment/Imaging | days prior | | | | | | | | | |
| | to Day 1 | | | | | | | | | |

| | | | |
|---|---|---|---|
| Paclitaxel dosing¹¹ | X | X | X |
| AP1 dosing¹² | X | X | X |

| Concomitant medications | Within 28 days prior to C1D1 until 30 days after last infusion or start of | |
|---|---|---|
| | subsequent therapy | |
| AE assessment | | AE collection period begins with first dose of study treatment |
| | | until 30 days post last dose or start of subsequent therapy |

AE = adverse event;
ECG = electrocardiogram;
NGS = next-generation sequencing;
PD = pharmacodynamics;
PK = pharmacokinetics
¹All patients are required to submit an archived tissue sample (if no archived tissue is available, pre-treatment tumor biopsy is required).
²Pre-treatment tumor biopsies are optional for patients enrolled in the dose escalation stange and required for patients enrolled in the expansion stage. Pre-treatment biopsies are collected within 15 days prior to the start of Cycle 1. On-treatment biopsies are collected on Days 8-10 of Cycle 1 (after the second dose of paclitaxel and AP1).
³For HIV-positive patients CD4 counts are obtained for confirmation of eligibllity; for patients with Hepatitis B oor C, viral loads are determined via PCR testing.
⁴Females of child-bearing potential have negative serum pregnancy test during screening and a negative urine pregnancy test on Day 1 prior to treatment.
⁵Blood pressure, pulse, respiration rate, body temperature.
Cycle 1, Days 1, 8, 15: On the days of drug administration vital signs are recorded pre-dose (within 30 minutes prior to SOI) and at the following time points:
During infusion: 15 min ( ±3 min) and 30 min (±3 min)
Post-infusion: At EOI ( ±5 min), 1 hr ( ±5 min) and 2 hr ( ±10 min), 4 hrs ( ±10 min) following EOI. On Cycle 1 Day 1 additional time points include 6 hrs ( ±10 min) and 8 hrs (±10 min) following EOI.
Additional vital signs are collected ar the discretion of the investigator.
⁶Full physical examinations are performed at Scrrening (including height), pre-dose on Days 1, 8 and 15 of Cycle 1, Day 22 of Cycle 1, and End of Treatment; all other physical examinations are symptom directed. weight to be collected on Day 1.
⁷ECGs are performed after the patient has been supine for at least 10 minutes. Readings are performed with the patient in the same physical position. ECG recordings are taken in triplicate with 5-10 minutes between readings.
⁸PD (MIC-1): 1 hour prior to the start of AP1 infusion and 3 ( ±10 min) hours after the end of AP1 infusion.
⁹PD (MIC-1): Blood should be collected 21 21 hours ( ±4 hours) after the end of AP1 infusion.
¹⁰PK sampling time points are on Days 1 and 2 and Day 15 as follows:
Paclitaxel—pre-dose, end of infusion, 1 h, 2 h, 3 h, 4 h, 6 h after end of infusion (Day 1); 24 h after end of infusion (Day 2)
AP1—pre-dose (prior to start of paclitaxel infusion), end of infusion, 1 h, 3 h after end of infusion (Day 1); 21 h after end of infusion (Day 2)
Paclitaxel—pre-dose, end of infusion, 1 h, 3 h, 4 h after end of infusion (Day 15)
AP1—pre-dose (prior to start of paclitaxel infusion), end of infusion, 1 h after end of infusion (Day 15)
¹¹Paclitaxel is infused over 1 hour (±15 min).
¹²AP1 is infused over 1 hour (±15 min) beginning 2 hours after the end of paclitaxel infusion. At the end of AP1 infusion, IV fluids (saline) or oral (500-1000 mL) are administered unless clinically contraindicated.

TABLE 7

| | | | | | | | End-of- | |
|---|---|---|---|---|---|---|---|---|
| | | | | | | | Treatment | |
| | | | | | | | 30 ± 5 d after | Long- |
| | D1¹ ± 3 d | | D8 ± 1 d | | D15 ± 1 d | | last dose or at | Term |
| | Pre- | Post- | Pre- | Post- | Pre- | Post- | study | Follow |
| Procedure | dose | dose | dose | dose | dose | dose | withdrawal | Up¹⁰ |
| Serum or urine | X | | | | | | X | |
| pregnancy test | | | | | | | | |
| Vital signs² | X | X | X | X | X | X | X | |
| Physical exam³ | X³ | | | | | | X | |
| 12-lead ECG (single) | X | | | | | | X | |
| Biopsy (optional)⁴ | | | | | | | X⁴ | |
| Laboratory assessments- | X | | | X | | | X | |
| chemistry⁵ | | | | | | | | |
| Laboratory assessments- | X | | X | | X | | X | |
| hematology⁵ | | | | | | | | |
| Laboratory assessments- | X | | | | X | | X | |
| coagulation⁵ | | | | | | | | |
| Laboratory assessments- | Perform as clinically indicated. | | | | | | | |
| urianalysis | | | | | | | | |
| Laboratory assessments- | To be performed approximately every 8 weeks during | | | | | | X⁶ | |
| tumor markers (as | treatment. Coinciding with tumor | | | | | | | |
| appropriate | assessment/imaging. | | | | | | | |
| Blood Collection-PK | Cycle 2 only⁷ | | | | | | | |
| assessments⁷ | | | | | | | | |
| ECOG Performance | X | | | | | | X | |
| status | | | | | | | | |
| Blood Collection-PD- | X | | | | | | X | |
| cfDNA | | | | | | | | |
| Tumor | To be performed approximately every 8 weeks during treatment. | | | | | | X⁸ | |
| Assessment/Imaging | Breast cancer patients will also undergo bone scans at | | | | | | | |
| | reimaging if bone metastases were present at baseline and | | | | | | | |
| | baseline bone scan was positive. | | | | | | | |
| Paclitaxel dosing⁹ | X | | X | | X | | | |
| AP1 dosing¹⁰ | X | | X | | X | | | |
| Concomitant | Within 28 days prior to C1D1 until 30 days after last | | | | | | X | |
| medications | infusion or start of subsequent therapy | | | | | | | |
| AE assessment | AE collection period begins with first dose of study | | | | | | X | |
| | treatment until 30 days post last dose or start of | | | | | | | |
| | susequent therapy | | | | | | | |
| Phone calls or other contact | | | | | | | | X¹¹ |

ECG = electrocardiogram;
PD = pharmacodynamics;
PK = pharmacokinetics
^{1“}Day 29” = Day 1 of next cycle for patients continuing treatment. Day 1 pre-dose evaluations for Cycle 2 and susequent cycles are completed within 3 days prior to next cycle drug administration.
²Blood pressure, pulse, respiration rate, body temperature. For patients on > 1 year, measurements are not a mandatory study procedure.
On the days of drug administration (Days 1, 8, 15 of each cycle), vital signs are recorded pre-dose (within 30 minutes prior to SOI) and at the following time points:
During infusion: 15 min (±3 min) and 30 min (±3 min)
Post-infusion: At EOI (±5 min) and as clinically following EOI.
Additional vital signs are collected at the discretion of the investigator.
³Weight is collected at Day 1 (or up to 3 days prior) of each cycle. A full physical examination is performed at End of Treatment.
⁴Biopsies (for TP53 sequencing) are collected at time of progression for patients who progress after response or clinical benefit are optional in both dose escalation and dose expansion.
⁵For patients on > 1 year, the required labs are: full labs to be collected on Day 1, and hematology only at Day 15.
⁶Upon discontinuation, a tumor marker assessment is collected coinciding with end of treatment tumor assessment/imaging if required.
⁷For Cycle 2 only, PK sampling time points on Day 1 are as follows:
Paclitaxel—pre-dose, end of infusion, 1 h, 3 h, 4 h after end of infusion
AP1—pre-dose (prior to start of paclitaxel infusion), end of infusion, 1 h after end of infusion
⁸Perform only if no tumor assessment was performed within 6-8 weeks prior.
⁹Paclitaxel is infused over 1 hour (±15 min).
¹⁰AP1 is infused over 1 hour (±15 min) beginning 2 hours after the end of paclitaxel infusion. At the end of AP1 infusion, IV fluids (saline) or oral fluids (500mL-1000 mL) are administered unless clinically contraindicated.
¹¹Phone calls or other contact are made approximately every 2 months for 1 year following end of treatment visit, and every 3 months thereafter, to assess survival status and collect information on subsequent therapies.

f. Statistical Methods

Tabulations are produced for appropriate demographic and baseline clinical characteristics, efficacy, pharmacokinetic/pharmacodynamic, and safety parameters. Results are summarized by dose levels and overall. For categorical variables, summary tabulations of the number and percentage of patients within each category of the parameter are presented. For continuous variables, the number of patients, mean, median, standard deviation, minimum, and maximum values are presented. Time-to-event data (PFS and DoR) are summarized using Kaplan-Meier methodology.

g. Determination of Maximum Tolerated Dose

Dose escalation phase: During the dose escalation phase, the BOIN design is employed to find the MTD. The MTD is considered the dose for which the isotonic estimate of the toxicity rate is closest to 0.30. The maximum sample size is 30. Patients are enrolled and treated in cohorts of 3. At the discretion of the Principal Investigator (PI), a 4^{th} patient is enrolled in a given cohort if operationally indicated, e.g., if 2 patients have signed the ICF simultaneously.

The BOIN design is described as follows:
1. Patients in the first cohort are treated at dose level 1 (L1)
2. To assign a dose to the next cohort of patients, dose escalation/de-escalation is conducted according to the rule displayed in TABLE 8, which minimizes the probability of incorrect dose assignment with the toxicity rate of ϕ₁=0.18 and ϕ₂=0.42 designated as underdosing and overdosing, respectively. When using TABLE 8, the following is noted:
   a. “Eliminate” means that the current and higher doses are eliminated from the trial to prevent treating any future patients at the current and higher doses because the doses are overly toxic.
   b. When a dose is eliminated, the patient is automatically de-escalated to the next lower level dose. When the lowest dose is eliminated, the trial is stopped for safety. In this case, no dose is selected as the MTD.
   c. If none of the actions (i.e., escalation, de-escalation or elimination) are triggered, new patients are treated at the current dose.
   d. If the current dose is the lowest dose and the rule indicates dose de-escalation, the new patients are treated at the lowest dose unless the number of DLTs reaches the elimination boundary, at which point the trial is terminated for safety.
   e. If the current dose is the highest dose and the rule indicates dose escalation, the new patients are treated at the highest dose.
3. Step 2 is repeated until the maximum sample size of 30 is reached or stop the trial if the number of patients treated at the current dose reaches 15.

TABLE 8 shows dose escalation/de-escalation rules for the BOIN design

TABLE 8

| Number of patients treated at the | | | | | | | | | | | | | | | |
|---|---|---|---|---|---|---|---|---|---|---|---|---|---|---|---|
| current dose | 1 | 2 | 3 | 4 | 5 | 6 | 7 | 8 | 9 | 10 | 11 | 12 | 13 | 14 | 15 |
| Escalate if # of DLT ≤ | NA | NA | 0 | 0 | 1 | 1 | 1 | 1 | 2 | 2 | 2 | 2 | 3 | 3 | 3 |
| Deescalate if # of DLT ≥ | NA | NA | 2 | 2 | 2 | 3 | 3 | 3 | 4 | 4 | 4 | 5 | 5 | 6 | 6 |
| Eliminate if # of DLT ≥ | NA | NA | 3 | 3 | 4 | 4 | 5 | 5 | 5 | 6 | 6 | 7 | 7 | 8 | 8 |

Dose expansion phase: Once the MTD is determined, an additional 15 patients are enrolled for additional experience with safety and efficacy. The BOIN design allows for the toxicity to be monitored in the expansion phase, therefore the MTD can be redesigned as needed. The dose is modified if toxicity is seen using TABLE 8.

TABLE 9 shows dose escalation/de-escalation rules for the BOIN design after treating 15 patients

TABLE 9

| | The number of patients treated at the current dose | | | | | | | | | | | | | | |
|---|---|---|---|---|---|---|---|---|---|---|---|---|---|---|---|
| Actions | 16 | 17 | 18 | 19 | 20 | 21 | 22 | 23 | 24 | 25 | 26 | 27 | 28 | 29 | 30 |
| Escalate if # of DLT <= | 3 | 4 | 4 | 4 | 4 | 4 | 5 | 5 | 5 | 5 | 6 | 6 | 6 | 6 | 7 |
| De-escalate if # of DLT >= | 6 | 7 | 7 | 7 | 8 | 8 | 8 | 9 | 9 | 9 | 10 | 10 | 11 | 11 | 11 |
| Eliminate if # of DLT >= | 8 | 9 | 9 | 9 | 10 | 10 | 11 | 11 | 11 | 12 | 12 | 12 | 13 | 13 | 14 |

h. Dosing and Administration

Treatment administration: Paclitaxel and AP1 are each be administered on Days 1, 8, and 15 of every 28-day cycle. The patients do not receive treatment on Day 21.

Administration of paclitaxel: Paclitaxel is infused intravenously over 1 hour on Days 1, 8, and 15 of every 28-day cycle. Paclitaxel is administered according to the current approved US prescribing information including pretreatment with corticosteroids, diphenhydramine, and H2 antagonists.

Administration of AP1: AP1 is infused intravenously over 1 hour, beginning 2 hours after the end of paclitaxel infusion. Antiemetics, including 5HT3 antagonists, are recommended prior to and for 48 hours following AP1 administration. Administration of 500 to 1000 mL of oral or IV fluid is required following each AP1 infusion, unless clinically contraindicated.

Duration of treatment: Patients receive treatment with AP1 plus paclitaxel until disease progression, unacceptable toxicity, or until any of the other criteria for treatment discontinuation are met. However, in case of clinical benefit, treatment beyond first radiologic disease progression is allowed.

Dose levels during escalation: The dose levels of paclitaxel and AP1 to be evaluated during dose escalation are shown in TABLE 10. Paclitaxel doses are calculated based on body mass index at the start of each cycle. The AP1 dose for individual patients is calculated based on body weight at the start of each cycle. If toxicity is observed at the starting dose level (L1), two dose de-escalation levels are possible.

TABLE 10

| | Paclitaxel (mg/m²) IV | AP1 (mg/kg) IV |
|---|---|---|
| Dose Level | D1, D8, D15 | D1, D8, D15 |
| L-2 | 60 | 0.64 |
| L-1 | 60 | 1.25 |
| L1 | 80 | 1.25 |
| L2 | 80 | 2.1 |
| L3 | 80 | 3.1 |

Dose level during expansion: Patients enrolled in the expansion stage are treated at the MTD determined during the dose escalation phase.

i. Dose Modifications in Response to Toxicities

Dose reduction doses: During the dose escalation stage, if a patient experiences a DLT, treatment continuation at a lower dose level is permitted as long as the toxicity has returned to ≤Grade 1 or baseline within 2 weeks. Upon recovery, patients may restart at one dose level lower per TABLE 10. Patients who do not recover within 2 weeks are not eligible for resumption of treatment.

During the dose escalation stage, intra-patient dose escalation may be allowed after Cycle 1 if the next higher dose level has been shown to be safe and the Investigator determines that the patient is tolerating treatment and could benefit from a higher dose. During dose expansion, if a patient experiences toxicities requiring dose reduction, the dose level reductions for re-treatment are as follows: Paclitaxel dose level reductions 80 mg/m²→60 mg/m²; AP1 dose level reductions 3.1 mg/kg→2.1 mg/kg→1.25 mg/kg→0.64 mg/kg.

The dose of either paclitaxel or AP1 is reduced separately if the toxicity is determined to be specifically related to that treatment. Up to two dose reductions are permitted; a third dose reduction will require evidence of clinical benefit and approval by the Principal Investigator. If a patient has a dose reduction due to toxicity, escalation back to the original dose level may be permitted if thought to be of clinical benefit, pending approval by the Principal Investigator. In the event that administration of one of the two study drugs are discontinued due to toxicity, the patient may continue to receive the other study drug at the discretion of the Principal Investigator.

Hematologic toxicities: For hematologic toxicities attributed to AP1 or paclitaxel, patients discontinue treatment if: Neutrophil counts<0.5×10⁹/L for >5 days, in absence of response to GCSF; Platelet counts<10×10⁹/L (despite platelet transfusions); or Hemoglobin<6 g/dL (despite red blood cell [RBC] transfusions). Patients interrupt treatment if: Neutrophil counts<0.5×10⁹/L for ≤5 days; Platelet counts<25×10⁹/L and >10×10⁹/L; or Hemoglobin<8 g/dL and >6 g/dL. After resolution of hematologic toxicity (i.e., return to Grade≤1 or pre-toxicity level), patients may continue at a reduced dose. Relevant labs are repeated as medically indicated.

Management of paclitaxel-related hematologic toxicities: Initial treatment modifications consist of cycle delay and/or dose reduction as indicated in TABLE 11. Patients do not receive prophylactic growth factors [filgrastim (G-CSF), sargramostim (GM-CSF), pegfilgrastim (Neulasta)] unless the patients experience recurrent neutropenic complications after treatment modifications. Patients do not receive prophylactic thrombopoietic agents. Patients may receive iron supplements, erythropoietin and/or transfusions as clinically indicated for management of anemia. Treatment decisions are based on the absolute neutrophil count (ANC), rather than the total white cell count (WBC).

For Cycle 1 Day 1, the ANC is ≥1500/mm³ and the platelet count is ≥100,000/mm³. Subsequent cycles of therapy do not begin (Day 1 of each cycle) until the ANC is ≥1000/mm³ and the platelet count is ≥75,000/mm³. Therapy is be delayed for a maximum of 2 weeks until the ANC and platelet values are achieved. Patients who fail to recover adequate counts within a 2-week delay are removed from study therapy. Day 8 and Day 15 paclitaxel treatments are not be given unless the ANC is ≥1000/mm³ and the platelet count is ≥75,000/mm³. If Day 8 or Day 15 paclitaxel is held, treatment is not be made up.

TABLE 11 shows paclitaxel dose hold and resumption criteria in response to hematologic toxicities. Patients requiring greater than two dose reductions of paclitaxel for any cause are removed from the study treatment. A third dose reduction could be discussed case by case, in presence of clinical benefit and after approval by the Principal Investigator.

TABLE 11

| Cycle | ANC | Platelet count | |
|---|---|---|---|
| Day | (cells/mm³) | (cells/mm³) | ACTION |
| Day 1 | <1000 | <75,000 | Delay. Monitor counts weekly |
| | | | until adequate for treatment. |
| | | | Restart when counts are adequate |
| | | | for treatment; reduce one dose level. |
| | | | If counts do not recover after 2 |
| | | | weeks delay, remove from study. |
| Day 8 | <1000 | <75,000 | Hold dose |
| Day 15 | <1000 | <75,000 | Hold dose |

Non-hematologic toxicities: In the event a non-hematologic Grade 4 AE considered related to AP1 and/or paclitaxel is observed, the patient is discontinued from the study. Exceptions include nausea/emesis, diarrhea or electrolyte abnormalities that resolve within 3 days on optimum treatment. For these exceptions, treatment may be delayed for up to 2 weeks during Cycle 1 (up to 4 weeks for later cycles) to allow resolution of the toxicity (i.e., return to Grade≤1 or pre-toxicity level), followed by re-treatment at a reduced dose. Relevant labs are repeated as medically indicated.

In the event a non-hematologic Grade 3 AE considered related to AP1 and/or paclitaxel is observed (exceptions are Grade 3 fatigue, nausea, emesis, diarrhea or clinically insignificant electrolyte abnormalities that resolve within 3 days on optimum treatment), treatment may be delayed for up to 2 weeks during Cycle 1 (up to 4 weeks for later cycles) to allow resolution of the toxicity, followed by re-treatment at a reduced dose. Relevant labs are repeated as medically indicated.

Grade 2 (or greater) peripheral neuropathy requires reduction of one dose level of paclitaxel and delay in subsequent therapy for a maximum of 2 weeks until recovered to Grade 1. If no recovery is observed after 2 weeks, the patient is removed from the study. No dose modifications are made for patients with alopecia or fatigue.

j. Concomitant Therapy

All concomitant medications taken within 28 days of beginning study treatment through the End-of-Treatment Visit (or start of alternative therapy) re-recorded in the electronic case report form (eCRF).

Required and recommended medications: Prior to paclitaxel administration, all patients receive premedication per institutional guideline with corticosteroids, H2 receptor antagonists, and diphenhydramine to prevent hypersensitivity reactions. No prophylactic GCSF are allowed; however, if the patient experiences a Grade 4 neutropenia or Grade 3 febrile neutropenia, GCSF for secondary prevention is allowed at subsequent cycles.

Prohibited medications and medications requiring special consideration: Concurrent anti-tumor therapy of any kind or any other investigational agent is prohibited. Any concomitant medications that are predominantly cleared by hepatobiliary transporters, OATP members OATP1B1 and OATP1B3, on the day of the AP1 infusion and within 48 hours after an AP1 infusion are prohibited, including the sartan class of angiotensin receptor blockers (ARBs).

The use of alternative antihypertensive agents is recommended in place of angiotensin converting enzyme (ACE) inhibitors and ARBs during treatment with AP1. Concomitant treatment with ACE inhibitors and ARBs with AP1 may increase the risk for developing angioedema. The use of alternative antihypertensive agents does not change the requirement to hold ARBs for 48 hours following the administration of AP1, due to a known pharmacokinetic interaction that decreases clearance of the ARB.

No antiretroviral medications that are CYP3A4 substrates are allowed. Caution is exercised when paclitaxel is concomitantly administered with known substrates, inhibitors, and inducers of CYP3A4. Caution is exercised when paclitaxel is concomitantly administered with known substrates, inhibitors, and inducers of CYP2C8.

Use of any immunosuppressive agents during the study is confirmed by the Principal Investigator. Palliative radiation to the bone is allowed. Study treatment is held 1 week prior and 1 week after radiation treatment. Other investigational agents are not be used during the study. If patients develop CNS metastasis with systemic disease control, patients are allowed to have CNS radiation and continue therapy if clinical benefits exist for the patient. Concomitant treatment for bone metastatses (such as bisphosphonates or anti-RANK-L antibodies) is allowed. Transfusions are permitted at the discretion of the Principal Investigator.

k. Study Intervention Discontinuation and Participant Discontinuation or Withdrawal

Participants are free to withdraw from participation in the study at any time upon request. Consent may be withdrawn for study treatment, survival follow-up, or both. A patient may be removed from the study treatment for a variety of reasons, including: disease progression that is either symptomatic, rapidly progressive, required urgent intervention, or associated with a decline in performance status; unacceptable toxicity; intercurrent illness that prevents further participation; patient refusal to continue treatment through the study and/or consent withdrawal for study participation; patient unable or unwilling to comply with study requirements; pregnancy or failure to use adequate birth control; general or specific changes in the patient's condition that render the patient unacceptable for further treatment in this study in the judgment of the Investigator.

The reason for discontinuation of study treatment is recorded in the eCRF. When a patient discontinues study treatment or is withdrawn, the Investigator performs the procedures indicated for end of study treatment within 28 days after discontinuation of study treatment and prior to initiation of alternative anti-cancer therapy. After treatment discontinuation, patients are followed for survival.

l. Study Assessments and Procedures

Biopsies: Tumor biopsies are performed pre-treatment and after start of treatment (Day 8-10 of Cycle 1, after 2^{nd} dose of paclitaxel and AP1) for identification of potential biomarkers of response. Tumor biopsies are optional for patients in dose escalation; however, tumor biopsies are mandatory in the dose expansion cohort if they can be safely performed. All patients require archived tissue sample (if no archived tissue is available, pre-treatment tumor biopsy is required).

Whole exome sequencing is performed on the tissue sample from the pre-treatment biopsy to test for TP53 status, and to evaluate the association of response with any particular genomic alterations (e.g., MDM2/MDMX amplification). In addition, RNAseq is performed to assess association with baseline gene expression (e.g., expression of MDM2/X and relative expression of MDMX splicing isoforms) and modulation of gene expression with therapy, including p53 target gene PHLDA3. Cell proliferation and apoptosis assays (Ki67, cleaved caspase3) are performed to test our hypothesis that AP1 in addition to paclitaxel induces apoptosis in cancer cells WT TP53. Immunohistochemistry (IHC) and RPPA will also be used to assess expression of p53, p21, and MDM2.

Efficacy assessments: Tumor assessments are performed by CT (and MRI as needed) approximately every 8 weeks during treatment. Breast cancer patients also undergo bone scans at reimaging if bone metastases were present at baseline and baseline bone scan was positive. Response determinations are based on RECIST 1.1. Following the discontinuation of study treatment, patients are followed for survival. Patients are contacted approximately every 2 months for 1 year following the end of treatment visit, and every 3 months thereafter, to assess survival status and collect information on subsequent therapies.

Pharmacokinetic and pharmacodynamic assessments: Blood samples for pharmacokinetic assessments are collected on Cycle 1 Days 1 and 2, Cycle 1 Day 15, and Cycle 2 Day 1 at the time points shown in TABLE 12. Where sampling time points for paclitaxel and AP1 overlap, blood collection may be coordinated to maximize patient comfort. TABLE 12 shows blood sample collection time points for pharmacokinetic analyses (cycles 1 and 2)

TABLE 12

| Cycle, | Clock time | | PK Sampling | |
|---|---|---|---|---|
| Day | (hr)^{a} | Time relative to infusion(s) | Paclitaxel | AP1 |
| Cycle 1, | 0 | Prior to start of paclitaxel infusion | X | X |
| Day 1 | | (predose) | | |
| | 1 | End of paclitaxel infusion (EOI) | X | — |
| | | (+5 min) | | |
| | 2 | 1 hr after paclitaxel EOI (±5 min) | X | — |
| | 3 | 2 hr after paclitaxel EOI (±10 min) | X | — |
| | 4 | 3 hr after paclitaxel EOI (±10 min)/ | | |
| | | End of AP1 infusion (+5 min) | X | X |
| | 5 | 4 hr after paclitaxel EOI (±10 min)/ | | |
| | | 1 hr after AP1 EOI (±5 min) | X | X |
| | 7 | 6 hr after paclitaxel EOI (±10 min)/ | | |
| | | 3 hr after AP1 EOI (±10 min) | X | X |
| Cycle 1, | 25 | 24 hrs (±2 hr) after paclitaxel EOI/ | | |
| Day 2 | | 21 hrs (±2 hr) after AP1 EOI | X | X |
| Cycle 1, | 0 | Prior to start of paclitaxel infusion | | |
| Day 15 | | (predose) | X | X |
| | 1 | End of paclitaxel infusion (EOI) | | |
| | | (+5 min) | X | — |
| | 2 | 1 hr after paclitaxel EOI (±5 min) | X | — |
| | 4 | 3 hr after paclitaxel EOI (±10 min)/ | | |
| | | End of AP1 infusion (+5 min) | X | X |
| | 5 | 4 hr after paclitaxel EOI (±10 min)/ | | |
| | | 1 hr after AP1 EOI (±5 min) | X | X |
| Cycle 2, | 0 | Prior to start of paclitaxel infusion | | |
| Day 1 | | (predose) | X | X |
| | 1 | End of paclitaxel infusion (EOI) | | |
| | | (+5 min) | X | — |
| | 2 | 1 hr after paclitaxel EOI (±5 min) | X | — |
| | 4 | 3 hr after paclitaxel EOI (±10 min)/ | | |
| | | End of AP1 infusion (+5 min) | X | X |
| | 5 | 4 hr after paclitaxel EOI (±10 min)/ | | |
| | | 1 hr after AP1 EOI ±(5 min) | X | X |

^{a}The clock times assume that the pre-dose sampling occurs at time 0 hr and the paclitaxel 1-hr infusion starts immediately. EOI of paclitaxel is at 1 hr after predose sampling. At 3 hr post predose (2-hr after the end of paclitaxel infusion), AP1 1-hr infusion starts. AP1 EOI is 4 hrs after predose sampling.

Cell-free circulating DNA (cfDNA) is performed. Samples for cfDNA are collected prior to the start of each cycle and at the end of treatment. The cfDNA monitoring is important in observing early tumor response dynamics and in the discovery of resistance mechanisms and new acquired mutations. Serum concentrations of MIC-1 are assessed as an additional pharmacodynamic marker.

### Example 4

### Efficacy of AP1 Alone and in Combination with Abraxane in the MCF-7.1 Human Breast Carcinoma Xenograft Model Using Female Athymic Nude Mice

Efficacy studies of AP1 alone and in combination with Abraxane® (albumin-bound paclitaxel) were conducted in the MCF-7.1 human breast carcinoma xenograft model using female athymic nude mice. The mice were divided into 8 test groups, as summarized in TABLE 13.

TABLE 13

| Group # | Dosing Regimen |
|---|---|
| 1* | Vehicle (i.v., days 2, 5, 9, 12, 16, 19, 23, 26) |
| 2 | AP1 5 mg/kg (i.v., days 2, 5, 9, 12, 16, 19, 23, 26) |
| 3 | Abraxane ® 15 mg/kg (i.v., qwk × 4 starting on day 2) |
| 4 | AP1 5 mg/kg (i.v., days 2, 5, 9, 12, 16, 19, 23, 26) + |
| | Abraxane ®15 mg/kg (i.v., qwk × 4 starting on day 4) |
| 5 | AP1 5 mg/kg (i.v., days 2, 5, 9, 12, 16, 19, 23, 26; |
| | dose 6 hours prior to Abraxane ® |
| | Abraxane ® 15 mg/kg (i.v., qwk × 4 starting on day 2) |
| 6 | Abraxane ® 15 mg/kg (i.v., qwk × 4 starting on day 2) + |
| | AP1 5 mg/kg (i.v., days 2, 5, 9, 12, 16, 19, 23, 26; |
| | dose 6 hours post-Abraxane ®) |
| 7 | Abraxane ® 15 mg/kg (i.v., qwk × 4) + |
| | AP1 5 mg/kg (i.v., days 2, 5, 9, 12, 16, 19, 23, 26; |
| | dose 24 hours post-Abraxane ®) |
| 8 | AP1 5 mg/kg (i.v., days 2, 5, 9, 12, 16, 19, 23, 26; |
| | dose 24 hours prior to Abraxane ® |
| | Abraxane ® 15 mg/kg (i.v., qwk × 4 starting on day 3) |

*Control group; Vehicle = PBS

General procedure: The mice were provided with drinking water with 10 μg/mL of 17 beta estradiol supplementation 3 days prior to cell implantation and for the duration of the study. 160 CR female NCr nu/nu mice were subcutaneously implanted with 1×10⁷ MCF-7.1 tumor cells using Matrigel. Tumor cell injection volume was 0.1 mL/mouse. At start date of the study, the mice were about 8 to 12 weeks old. When tumors reached an average size of about 100-150 mm³, a pair match was performed prior to start of treatment.

Any individual animal with a single observation of >30% body weight loss or three consecutive measurements of >25% body weight loss were euthanized. Dosing was terminated for any group with a mean body weight loss of >20% or >10% mortality. The group was not euthanized, and recovery was allowed. Within a group with >20% weight loss, individuals hitting the individual body weight loss endpoint were euthanized. If the group treatment-related body weight loss was recovered to within 10% of the original weights, dosing was resumed at a lower dose or less frequent dosing schedule. Exceptions to non-treatment body weight % recovery were allowed on a case-by-case basis.

Animals were monitored individually for endpoint tumor growth delay (TGD). The endpoint of the experiment was a tumor volume of 1000 mm³ or 60 days, whichever occurred first. Responders were followed for a longer period of time. Animals were euthanized when the endpoint was reached.

Dosing instructions: Paclitaxel was prepared by reconstituting in a vial per manufacturer instructions (Celgene, Lot No. 6115306). The stock was aliquoted for each day of dosing and stored at −80° C. On each day of dosing, one vial of stock was thawed prior to dilution to prepare the dosing solution. AP1 was formulated in a phosphate-buffered aqueous solution. Dosing volume was 10 mL/kg (0.200 mL/20 g mouse). Volume was adjusted accordingly based on body weight.

Mice: Female athymic nude mice (Crl:NU(Ncr)-Foxn1^{nu}, Charles River) were nine to ten weeks old with a body weight (BW) range of 17.3-28.6 g on Day 1 of the study. The animals were fed ad libitum water (reverse osmosis, 1 ppm Cl), and NIH 31 Modified and Irradiated Lab Diet® consisting of 18.0% crude protein, 5.0% crude fat, and 5.0% crude fiber. The mice were housed five per cage on irradiated Enrich-o'cobs™ Laboratory Animal Bedding in static microisolators on a 12-hour light cycle at 20-22° C. (68-72° F.) and 40-60% humidity.

Tumor Cell Culture: In vivo selected MCF-7.1 human breast carcinoma cells cultured in RPMI-1640 medium containing 10% fetal bovine serum, 2 mM glutamine, 10 mM HEPES, 0.075% sodium bicarbonate, 100 units/mL penicillin G, 100 μg/mL streptomycin sulfate, and 25 μg/mL gentamicin. Cells were cultured in tissue culture flasks in a humidified incubator at 37° C., in an atmosphere of 5% CO₂ and 95% air.

In Vivo Implantation and Tumor Growth: Three days prior to tumor cell implantation and for the duration of the study, the drinking water of all cages was supplemented with 10 μg/mL with 17 beta estradiol.

The MCF-7.1 cells used for implantation were harvested during exponential growth and were resuspended in phosphate buffered saline (PBS) at a concentration of 1×10⁸ cells/mL. On the day of tumor implant, each test mouse was injected into the right flank with 1×10⁷ cells (0.1 mL cell suspension), and tumor growth was monitored as the average size approached the target range of 100 to 150 mm³. Tumors were measured in two dimensions using calipers, and volume was calculated using the formula:

$$Tumor Volume ( {mm}^{3} ) = \frac{{w}^{2} \times l}{2}$$

where w=width and l=length, in mm, of the tumor.

Seventeen days after tumor implantation, designated as Day 1 of the study, the animals were sorted into eight groups (n=10/group). Individual tumor volumes ranged from about 75-196 mm³ and group mean tumor volumes were about 107-111 mm³.

Agents: AP1 was formulated at 0.5 mg/mL and stored at 4° C. The 0.5 mg/mL solution provided the 5 mg/kg dosage in a dosing volume of 10 mL/kg. Vehicle and AP1 solutions were allowed to warm to room temperature and mixed by gentle inversion prior to each administration. Paclitaxel was reconstituted to 5 mg/mL per manufacturer's instructions. The paclitaxel solution was aliquoted for each day of dosing and stored at −20° C. On each day of dosing, an aliquot of stock was thawed and diluted to 1.5 mg/mL in saline. The 1.5 mg/mL dosing solution provided the 15 mg/kg dose in a dosing volume of 10 mL/kg based on individual body weight. AP1 was dosed at the same time of day (˜12:30 PM) and paclitaxel dosing was adjusted as necessary (6:30 AM, 12:30 PM, and 6:30 PM).

Treatment: On Day 1 of the study, female nude mice bearing established subcutaneous MCF-7.1 xenografts were sorted into eight groups (n=10), and dosing was initiated according to the treatment plan summarized in TABLE 13. The dosing volume was 0.2 mL per 20 grams of body weight (10 mL/kg), and adjusted according to individual body weight of each animal. All vehicle and AP1 treatments were administered intravenously (i.v.) twice weekly for four weeks, starting on Day 2. Paclitaxel was administered i.v. once weekly for four weeks (qwk×4), starting on Day 1 or 2.

Group 1 mice received vehicle i.v. on Days 2, 5, 9, 12, 16, 19, 23, and 26, and served as the control group for TGD analysis. Group 2 mice received AP1 at 5 mg/kg i.v. on Days 2, 5, 9, 12, 16, 19, 23, and 26. Group 3 received paclitaxel at 15 mg/kg i.v., qwk×4, starting on Day 2. Group 4 received AP1 at 5 mg/kg i.v. on Days 2, 5, 9, 12, 16, 19, 23, and 26 in combination with paclitaxel at 15 mg/kg i.v. qwk×4, starting on Day 2. Group 5 received AP1 at 5 mg/kg on Days 2, 5, 9, 12, 16, 19, 23, and 26 in combination with paclitaxel at 15 mg/kg i.v. qwk×4, starting on Day 2. On days when both agents were dosed in Group 5, the paclitaxel dose was administered six hours following the AP1 dose. Group 6 received the same treatments as Group 5, but the order of administration was reversed so that on days when both agents were administered, paclitaxel was dosed first followed by AP1 six hours later. Group 7 received paclitaxel at 15 mg/kg i.v. qwk×4, starting on Day 1 (Days 1, 8, 15, and 22) in combination with AP1 at 5 mg/kg i.v. starting 24 hours after the first dose of paclitaxel on Days 2, 5, 9, 12, 16, 19, 23, and 26. Group 8 received AP1 at 5 mg/kg i.v. on Days 2, 5, 9, 12, 16, 19, 23 and 26 in combination with paclitaxel at 15 mg/kg i.v. qwk×4, starting twenty-four hours later on Day 3 (Days 3, 10, 17, and 24).

Endpoint and Tumor Growth Delay (TGD) Analysis: Tumors were measured using calipers twice per week, and each animal was euthanized when its tumor reached the endpoint volume of 1000 mm³ or at the end of the study (Day 64), whichever came first. Animals that exited the study for tumor volume endpoint were documented as euthanized for tumor progression (TP), with the date of euthanasia. The time to endpoint (TTE) for analysis was calculated for each mouse by the following equation:

$$T T E = \frac{{log}_{10} ( endpoint volume ) - b}{m}$$

where TTE is expressed in days, endpoint volume is expressed in mm³, b is the intercept, and m is the slope of the line obtained by linear regression of a log-transformed tumor growth data set. The data set consisted of the first observation that exceeded the endpoint volume used in analysis and the three consecutive observations that immediately preceded the attainment of this endpoint volume. The calculated TTE is usually less than the TP date, the day on which the animal was euthanized for tumor size. Animals with tumors that did not reach the endpoint volume were assigned a TTE value equal to the last day of the study (Day 64). In instances in which the log-transformed calculated TTE preceded the day prior to reaching endpoint or exceeded the day of reaching tumor volume endpoint, a linear interpolation was performed to approximate the TTE. Any animal classified as having died from NTR (non-treatment-related) causes due to accident (NTRa) or due to unknown etiology (NTRu) were excluded from TTE calculations (and all further analyses). Animals classified as TR (treatment-related) deaths or NTRm (non-treatment-related death due to metastasis) were assigned a TTE value equal to the day of death.

Treatment outcome was evaluated from tumor growth delay (TGD), which is defined as the increase in the median time to endpoint (TTE) in a treatment group compared to the control group:

TGD=T−C

expressed in days, or as a percentage of the median TTE of the control group:

$$\% T G D = \frac{T - C}{C} \times 100$$

where:
T=median TTE for a treatment group, and
C=median TTE for the designated control group.

MTV and Criteria for Regression Responses: Treatment efficacy was determined from the tumor volumes of animals remaining in the study on the last day. The MTV (n) was defined as the median tumor volume on the last day of the study in the number of animals remaining (n) whose tumors had not attained the endpoint volume.

Treatment efficacy was also determined from the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study. Animals were scored only once during the study for a PR or CR event and only as CR if both PR and CR criteria were satisfied. An animal with a CR response at the termination of a study was additionally classified as a tumor-free survivor (TFS). Animals were monitored for regression responses.

Toxicity: Animals were weighed daily on Days 1-5, then twice per week until the completion of the study. The mice were observed frequently for overt signs of any adverse, treatment-related (TR) side effects, and clinical signs were recorded when observed. Individual body weight was monitored as per protocol, and any animal with weight loss exceeding 30% for one measurement or exceeding 25% for three consecutive measurements was euthanized as a TR death. Group mean body weight loss was also monitored according to CR Discovery Services protocol. Acceptable toxicity was defined as a group mean body weight (BW) loss of less than 20% during the study and no more than 10% TR deaths. Dosing was suspended in any group where mean weight loss exceeded acceptable limits. If group mean body weight recovered to acceptable levels, then dosing was modified to lower levels and/or reduced frequency then resumed. Deaths were classified as TR if attributable to treatment side effects as evidenced by clinical signs and/or necropsy. A TR classification was also assigned to deaths by unknown causes during the dosing period or within 14 days of the last dose. A death was classified as non-treatment-related (NTR) if no evidence that death was related to treatment side effects was observed. NTR deaths are further categorized as follows: NTRa describes deaths due to accidents or human error; NTRm is assigned to deaths thought to result from tumor dissemination by invasion and/or metastasis based on necropsy results; NTRu describes deaths of unknown causes that lack available evidence of death related to metastasis, tumor progression, accident or human error. Treatment side effects cannot be excluded from deaths classified as NTRu.

Statistical and Graphical Analyses: Study groups experiencing toxicity beyond acceptable limits (>20% group mean body weight loss or greater than 10% treatment-related deaths) or having fewer than five evaluable observations, were not included in the statistical analysis.

The logrank test, which evaluates overall survival experience, was used to analyze the significance of the differences between the TTE values of two groups. Logrank analysis includes the data for all animals in a group except those assessed as NTR deaths. Statistical tests were not adjusted for multiple comparisons. Two-tailed statistical analyses were conducted at significance level P=0.05. Prism summarizes test results as not significant (ns) at P>0.05, significant (symbolized by “*”) at 0.01<P≤0.05, very significant (“*”) at 0.001<P≤0.01, and extremely significant (“***”) at P≤0.001. All levels of significance were described as either significant or not significant.

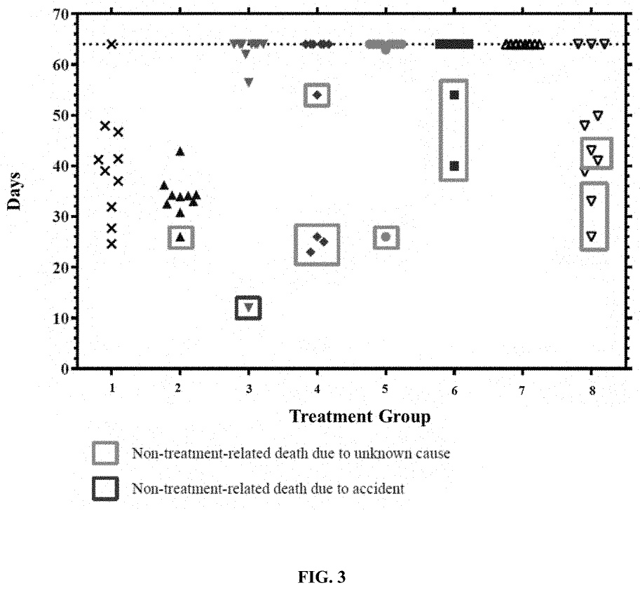

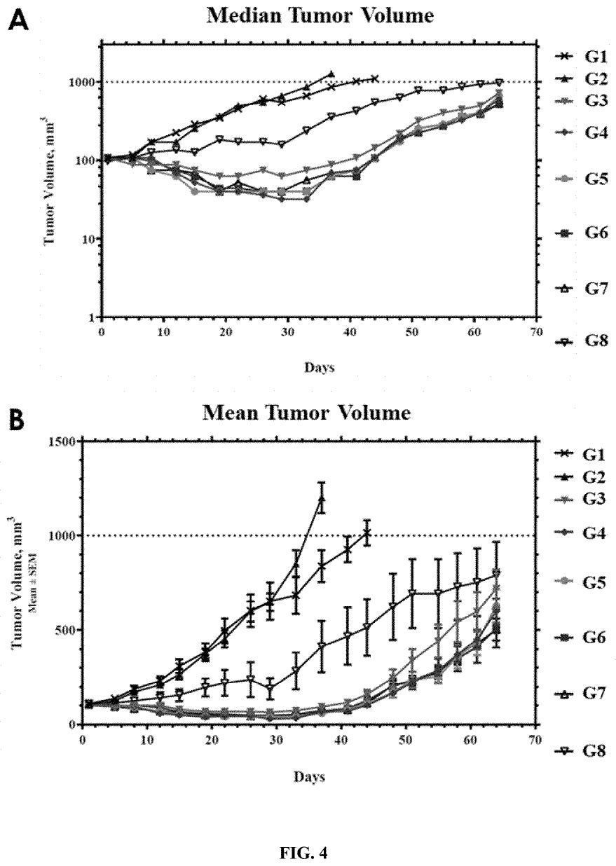

FIG. 3 shows a scatter plot of TTE values for individual mice, by treatment group as summarized in TABLE 13. Group median and mean tumor volumes were plotted as a function of time (FIGS. 4A and 4B). The data show that Groups 3, 4, 5, and 6 exhibited a reduction in median tumor volume followed by a growth delay in the first 30 days of treatment. Group 7 had the highest reduction in tumor volume 5 days after treatment. The data show that Groups 3, 4, 5, and 6 also resulted in a reduction in mean tumor volume followed by a growth delay in the first 30 days of treatment.

The response summary of the study is shown in TABLE 14. Groups 3, 4, 5, 6, and 7 each exhibited the greatest delay in tumor growth with a 60% TGD. Group 8 exhibited a 42% TGD.

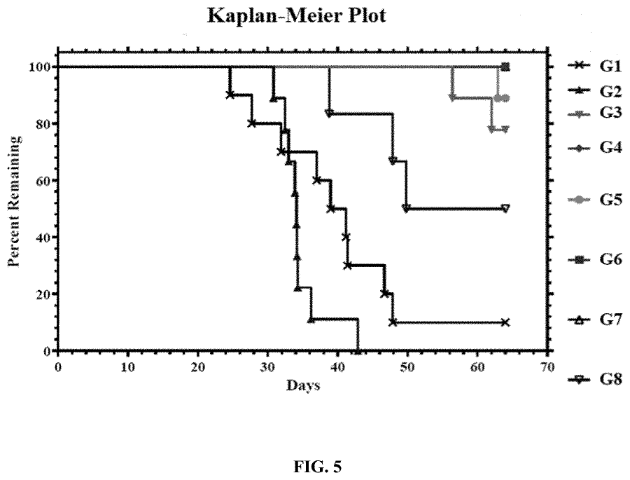

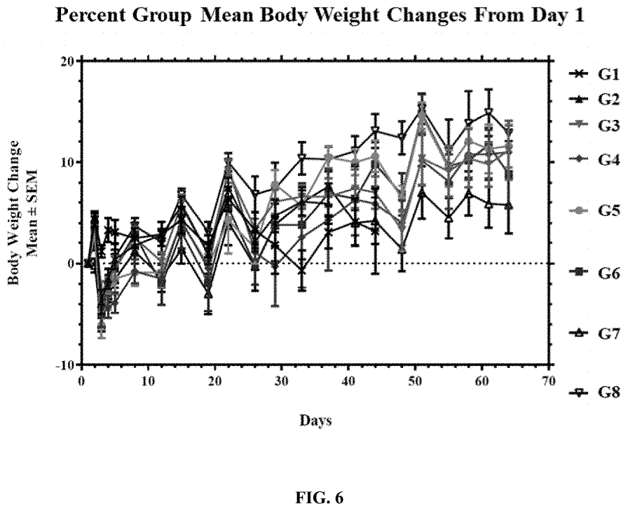

When an animal was removed from the study due to tumor size, the final tumor volume recorded for the animal was included with the data used to calculate the mean volume at subsequent time points. The Kaplan-Meier plot shows the percentage of animals in each group remaining in the study versus time (FIG. 5). The Kaplan-Meier plot and logrank test share the same TTE data sets. Group body weight changes over the course of the study were plotted as percent mean change from Day 1 (FIG. 6). Tumor growth and body weight plots excluded the data for animals assessed as NTR deaths, and were truncated when fewer than 50% of the animals in a group remained in the study.

TABLE 14
Treatment Response Summary

| | | Treatment Regimen | | | Median | | | Statistical Significance | | | | | | MTV (m) | Regression | | | Mean BW | Deaths | | |
|---|---|---|---|---|---|---|---|---|---|---|---|---|---|---|---|---|---|---|---|---|---|
| Group | n | Agent | mg/kg | Schedule | TTE | T—C | % TGD | vs G1 | vs G2 | vs G3 | vs G4 | vs G5 | vs G7 | Day 64 | PR | CR | TES | Nadir | TR | NT | NTRa |
| 1 | 10 | Vehicle | *** | Days 2,5,9,12,16,19,22,26 | 40.1 | — | — | — | ns | *** | ** | *** | *** | 486 (1) | 0 | 0 | 0 | −0.7% | 0 | 0 | 0 |
| 2 | 9 | AP1 | 5 | Days 2,5,9,12,16,19,23,26 | 34.1 | −6.0 | −15 | ns | — | — | — | *** | *** | *** | 0 | 0 | 0 | −3.8% | 0 | 0 | 1 |
| 3 | 9 | Abraxame | 15 | qwk × 4 (start on Day 2) | 64.0 | 23.9 | 60 | *** | — | — | — | ns | ns | 600 (7) | 1 | 0 | 0 | −2.8% | 0 | 1 | 0 |
| 4 | 6 | AP1 | 5 | Days 2,5,9,12,16,19,25,26 | 64.0 | 23.9 | 60 | ** | *** | ns | — | ns | ns | 608 (6) | 4 | 0 | 0 | −5.2% | 0 | 0 | 4 |
| | | Abraxame | 15 | qwk × 4 (start on Day 2) | | | | | | | | | | | | | | Day 3 | | | |
| 5 | 9 | AP1 | 5 | Days 2,5,9,12,16,19,23,26 (dose 6 hours | 64.0 | 23.9 | 60 | *** | *** | ns | ns | — | — | 583 (8) | 7 | 0 | 0 | −6.1% | 0 | 0 | 1 |
| | | Abraxame | 15 | prior to abraxame) | | | | | | | | | | | | | | Day 3 | | | |
| | | | | qwk × 4 (start on Day 2) | | | | | | | | | | | | | | | | | |
| 6 | 8 | Abraxame | 15 | qwk × 4 (start on Day 2) | 64.0 | 23.9 | 60 | *** | *** | ns | ns | ns | — | 525 (8) | 6 | 0 | 0 | −5.0% | 0 | 0 | 2 |
| | | AP1 | 5 | Days 2,5,9,12,16,19,22,26 (dose 6 hours | | | | | | | | | | | | | | | | | |
| | | | | post abraxame) | | | | | | | | | | | | | | | | | |
| 7 | 10 | Abraxane | 15 | qwk × 4 | 64.0 | 23.9 | 60 | *** | *** | ns | ns | — | * | 525 (10) | 6 | 0 | 0 | −6.0% | 0 | 0 | 0 |
| | | AP1 | 5 | Days 2,5,9,12,16,19,23,26 (dose 24 hours | | | | | | | | | | | | | | Day 3 | | | |
| | | | | post abraxame) | | | | | | | | | | | | | | | | | |
| 8 | 6 | AP1 | 5 | Days2,5,9,12,16,19,23,26 | 56.9 | 16.8 | 42 | * | *** | ns | ns | — | — | 600 (3) | 0 | 1 | 1 | −4.6% | 0 | 0 | 4 |
| | | Abraxane | 15 | prior to abraxame) | | | | | | | | | | | | | | Day 3 | | | |
| | | | | qwk × 4 (start on Day 3) | | | | | | | | | | | | | | | | | |

Table 2 displays the scheduled treatment regimen at completion of the study.
vehicle = PBS
Study Endpoint = 1000 mm³: Study Duration = 64 Days
n = number of animals in a group not dead from accidental or unknown causes, or euthanized for sampling
TTE = time to endpoint,
T—C = difference between media
TTE (Days) of treated versus control group,
% TGD = [(T—C)/C] × 100 The maximum T—C in this study is 23.9 Days (60%). compared to Group 1
Statistical Significance (Logrank test):
ne = not evaluable,
ns = not significant,
*= P < 0.05.
**= P < 0.01,
***= P < 0.001. compared to group indicated
MTV (n) = median tumor volume (mm³) for the number of animals on the Day of TGD analysis (excludes animals with tumor volume at endpoint)
PR = partial regressions;
CR = total number complete regressions;
TFS = tumor free survivors, i.e., CRs at end of study
Mean BW Nadir = lowest group mean body weight, as % change from Day 1: — indicates no decrease in mean body weight was observed
TR = treatment-related death:
NTR = non-treatment-related death

What is claimed is:

1. A method of treating a condition in a subject in need thereof, comprising administering to the subject a combination therapy comprising a therapeutically-effective amount of a peptidomimetic macrocycle and a therapeutically-effective amount of paclitaxel, wherein the combination therapy has a combination index of less than 1.

2. The method of claim 1, wherein the combination therapy has a combination index of less than 0.9.

3. The method of claim 1, wherein the combination therapy has a combination index of about 0.8 to about 0.9.

4. The method of claim 1, wherein the combination index is calculated from a half maximal inhibitory concentration (IC5o).

5. The method of claim 1, wherein the combination index is calculated from an IC₇₅ value.

6. The method of claim 1, wherein the combination index is calculated from an in vitro cell proliferation assay.

7. The method of claim 1, wherein the combination index is calculated from an in vivo animal study.

8. The method of claim 1, wherein the condition is cancer.

9. The method of claim 8, wherein the cancer expresses wild type p53.

10. The method of claim 8, wherein the cancer is an advanced or metastatic solid tumor.

11. The method of claim 8, wherein the cancer is breast cancer.

12. The method of claim 8, wherein the cancer is estrogen receptor-positive breast cancer.

13. The method of claim 8, wherein the combination therapy delays tumor growth in the subject by at least 30 days.

14. The method of claim 8, wherein the combination therapy delays tumor growth in the subject by at least 20 days.

15. The method of claim 8, wherein the combination therapy delays tumor growth in the subject by at least 23.9 days.

16. The method of claim 8, wherein the combination therapy results in a percentage tumor growth delay that is at least about 50%.

17. The method of claim 8, wherein the combination therapy results in a percentage tumor growth delay that is at least about 60%.

18. The method of claim 8, wherein the percentage tumor growth delay (% TGD) is determined by the equation:
$$\% T G D = \frac{T - C}{C} \times 100 ,$$
wherein:
T is a median time to endpoint (TTE) for a combination therapy group, and
C is a median TTE for a no combination therapy group.

19. The method of claim 1, wherein the peptidomimetic macrocycle inhibits HDMX.

20. The method of claim 1, wherein the peptidomimetic macrocycle inhibits HDM2.

21. The method of claim 1, wherein the peptidomimetic macrocycle stabilizes or increases a concentration of active p53 in the subject.

22. The method of claim 1, wherein the peptidomimetic macrocycle is a compound of Formula (I): 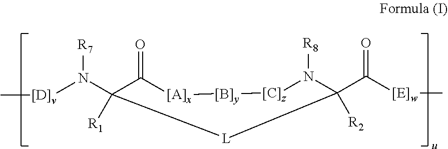
wherein:
each A, C, D, and E is independently a natural or non-natural amino acid or an amino acid analog, and each terminal D and E independently optionally includes a capping group;
each B is independently a natural or non-natural amino acid, an amino acid analog, 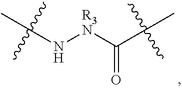
[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently —H, alkyl, alkenyl, alkynyl, arylalkyl, cycloalkylalkyl, heterocycloalkyl, a fluorescent moiety, a radioisotope, or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with Rs, or part of a cyclic structure with an E residue;
each v and w is independently an integer from 1-1000;
u is an integer from 1-10;
each x, y, and z is independently an integer from 0-10; and
n is an integer from 1-5.

23. The method of claim 22, wherein v is 3-10.

24. The method of claim 22, wherein v is 3.

25. The method of claim 22, wherein w is 3-10.

26. The method of claim 22, wherein w is 6.

27. The method of claim 22, wherein x+y+z=6.

28. The method of claim 22, wherein each L₁ and L₂ is independently alkylene, alkenylene, alkynylene, heteroalkylene, cycloalkylene, heterocycloalkylene, arylene, or heteroarylene.

29. The method of claim 22, wherein each L₁ and L₂ is independently alkylene or alkenylene.

30. The method of claim 22, wherein each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-.

31. The method of claim 22, wherein each R₁ and R₂ is independently hydrogen.

32. The method of claim 22, wherein each R₁ and R₂ is independently alkyl.

33. The method of claim 22, wherein each R₁ and R₂ is independently methyl.

34. The method of claim 22, wherein u is 1.

35. The method of claim 22, wherein each E is Ser or Ala, or an analogue thereof.

36. The method of claim 1, wherein the peptidomimetic macrocycle comprises an amino acid sequence that is at least 60% identical to an amino acid sequence listed in Table 1, Table 1a, Table 1b, Table 1c, Table 2a, or Table 2b.

37. The method of claim 1, wherein the peptidomimetic macrocycle comprises an amino acid sequence that is at least 70% identical to an amino acid sequence listed in Table 1, Table 1a, Table 1b, Table 1c, Table 2a, or Table 2b.

38. The method of claim 1, wherein the peptidomimetic macrocycle comprises an amino acid sequence that is at least 80% identical to an amino acid sequence listed in Table 1, Table 1a, Table 1b, Table 1c, Table 2a, or Table 2b.

39. The method of claim 1, wherein the peptidomimetic macrocycle is at least 60% identical to SP-153, SP-303, SP-331, or SP-671.

40. The method of claim 1, wherein the paclitaxel is nanoparticle albumin-bound paclitaxel.

41. The method of claim 1, wherein the therapeutically-effective amount of the peptidomimetic macrocycle is about 0.01 mg/kg to about 1000 mg/kg per day.

42. The method of claim 1, wherein the therapeutically-effective amount of the peptidomimetic macrocycle is about 5 mg/kg per day.

43. The method of claim 1, wherein the therapeutically-effective amount of the paclitaxel is about 0.01 mg/kg to about 1000 mg/kg per day.

44. The method of claim 1, wherein the therapeutically-effective amount of the paclitaxel is about 15 mg/kg per day.

45. The method of claim 1, wherein the peptidomimetic macrocycle is administered by intravenous injection.

46. The method of claim 1, wherein the paclitaxel is administered by intravenous injection.

47. The method of claim 1, wherein the peptidomimetic macrocycle is administered weekly.

48. The method of claim 1, wherein the paclitaxel is administered weekly.

49. The method of claim 1, wherein the peptidomimetic macrocycle and the paclitaxel are administered simultaneously.

50. The method of claim 1, wherein the peptidomimetic macrocycle and the paclitaxel are administered sequentially.

51. The method of claim 1, wherein the peptidomimetic macrocycle and the paclitaxel are administered in the same composition.

52. The method of claim 1, wherein the peptidomimetic macrocycle and the paclitaxel are administered in separate compositions.

53. The method of claim 1, wherein the subject is murine.

54. The method of claim 1, wherein the subject is human.
